(19) United States
(12) Patent Application Publication
(10) Pub. No.: US 2022/0241466 A1
(43) Pub. Date: Aug. 4, 2022

(54) DEGRADABLE POLYMERIC TISSUE SCAFFOLD

(71) Applicant: Mosaic Biosciences, Inc., Boulder, CO (US)

(72) Inventors: Peter D. MARINER, Superior, CO (US); Alexei KAZANTSEV, Erie, CO (US); Martin STANTON, Boulder, CO (US); Zheng ZHANG, Broomfield, CO (US); Chrysa LATRICK, Louisville, CO (US)

(21) Appl. No.: 17/611,111

(22) PCT Filed: May 14, 2020
(86) PCT No.: PCT/US20/32932

§ 371 Date: Nov. 12, 2021

Related U.S. Application Data
(60) Provisional application No. 62/847,864, filed on May 14, 2019.
(60) Provisional application No. 62/847,866, filed on May 14, 2019.

(51) Int. Cl. A61L 27/52; A61L 27/58; A61L 27/26; A61K 8/64; A61K 8/04; A61Q 19/08

(52) U.S. Cl. CPC A61L 27/52; A61L 27/58; A61L 27/26; A61L 2430/34; A61K 8/042; A61Q 19/08; A61L 2400/06; A61K 8/64

(57) ABSTRACT

The present disclosure relates generally to degradable polymeric scaffolds, methods of making them, and their use in the biomedical field.

### CROSS REFERENCE TO RELATED APPLICATIONS

This application claims the priority benefit of U.S. Provisional Patent Application Nos. 62/847,864, filed May 14, 2019, and 62/847,866, filed May 14, 2019, the disclosures of which are hereby incorporated herein by reference in their entireties.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 700032000940SEQLIST.TXT, date recorded: May 11, 2020, size: 15 KB).

### FIELD

The present disclosure relates generally to degradable polymeric scaffolds, methods of making them, and their use in the biomedical field, such as in the tissue engineering field and in wound healing.

### BACKGROUND

The field of tissue engineering aims to create biomaterials that can support or enhance tissue regeneration. These biomaterials are generally designed to provide provisional scaffolds for cellular migration and infiltration, allowing cells from the body to invade into the biomaterial and deposit new extracellular matrix, or tissue. In this scenario, the provisional biomaterial is degraded and cleared by the body as new tissue is formed. Rarely is the biomaterial designed with the intention that it remain in the body permanently.

In many biomaterial systems, the ability of cells to move into the provisional tissue scaffold depends on the ability of cells to degrade the material. That is, a matrix is originally formed with a mesh size that is smaller than the cells, requiring that cells degrade the matrix locally to create space for cellular invasion. One theoretical benefit of these systems is that control over cellular migration rates can be tuned by optimizing the polymer formulation. For example, network densities can be increased to slow cellular infiltration while decreasing network densities would have the opposite effect. Alternatively, the network can be tuned to be susceptible to specific enzymes that are present in wound environments to degrade the matrix and allow cellular infiltration.

In theory, this approach should work; however, several concepts that are prevalent in the field of tissue engineering run counter to this approach. It is commonly thought in the field that biomaterial systems that are to be used for tissue engineering or tissue regeneration should have physical properties that are similar to those of the target tissue. In the case of skin wound healing, for example, it would be commonly believed that biomaterial systems designed for these applications should have physical properties that are similar to native skin. By its nature, however, skin is highly cross-linked tissue with very strong physical properties. Polymer systems that replicate or mimic the physical properties of skin will be inherently dense and present more of an obstacle to cellular infiltration and tissue regeneration than a scaffold to enhance wound healing. It could theoretically be argued that matrices could be designed to have an initial stiffness that is similar to skin and, at the same time, incorporate highly degradable units to allow efficient cellular invasion. Unfortunately, matrix degradation is accompanied by a loss in physical strength unless new matrix is deposited in its place. If matrix degradation is too slow, the material will present an obstacle to cells and inhibit tissue regeneration. If the material degrades too quickly, the integrity of the scaffold will be lost before new tissue is formed, resulting in a failure of the material to scaffold the process. Though theoretically possible, designing a biomaterial system that achieves the perfect balance between degradation and structural integrity is difficult in practice.

Even if such balance between degradation and structural integrity was to be achieved, an additional problem would remain. Namely, variability between wounds, both within the context of the same subject and between subjects, makes it difficult to find a specific formulation that would have the appropriate degradation profile for every situation. Differences in skin thickness, vascularity, wound depth, etc., impact the rate of cellular activity and, therefore, the rate of material degradation within the wound. Wounds come in different sizes, shapes, depths, and locations, and present different levels of chronicity in patients with different ages and disease states. As an example, diabetic foot ulcers (DFUs) are a clinical indication in which provisional tissue scaffolds are used to bolster tissue regeneration and wound closing. Patients with DFUs range in age and the severity of their diabetic condition, both of which impact wound healing rates, and can have significantly different wounds with different levels of protease activity (a common attribute of chronic wounds). A one-formulation-fits-all product is not likely to work across the board if the material's efficacy is completely dependent on displaying the appropriate degradation rate.

Given the challenges of balancing physical properties with cellular infiltration, and the wide variability of wound environments, there is a need to identify a way to circumvent solving the material degradation rate balancing problem.

Another problem to address is the tendency of degradable polymers to swell significantly when placed into aqueous environments. In the context of the wound healing environment, swelling is an important issue. Polymers that swell upon placement into a wound environment will create physical strains on the tissue. Additionally, polymers that are susceptible to swelling will continue to swell as the network is degraded as the network loses strength and more water is able to move into the material. While this degradation-dependent swelling effect will not necessarily put a similar physical/osmotic strain on the wound environment that occurs with initial swelling, it does have the effect of causing material displacement in and around the newly forming tissue. Accordingly, there is a need for materials with controlled swelling properties upon degradation.

There is a need for alternative materials for biomedical applications, including materials for use in tissue engineering and wound healing. Materials that address one or more of the existing challenges in tissue engineering and wound healing would be particularly beneficial.

### BRIEF SUMMARY

The present disclosure relates generally to degradable polymeric scaffolds, methods of making them, and their use in the tissue engineering field.

### BRIEF DESCRIPTION OF THE DRAWINGS

FIG. 1A and FIG. 1B show a comparison of the clotting properties of native fibrinogen (Fgn) and denatured, inactivated fibrinogen derivatized with PEG-norbornene.

FIG. 2A and FIG. 2B show pictures of exemplary hydrogel microspheres having a diameter of about 0.5 mm to about 0.7 mm. FIG. 2C and FIG. 2D show pictures of exemplary hydrogel microspheres having a diameter of about 0.2 mm to about 0.3 mm. FIG. 2E and FIG. 2F show pictures of exemplary hydrogel microspheres having a diameter of about 0.1 mm to about 0.2 mm.

FIG. 3 shows a picture of exemplary hydrogel microspheres having a diameter of about 0.005 to about 0.1 mm (scale bar in FIG. 3=0.05 mm).

FIG. 4 shows an SDS-PAGE analysis of the Fgn-gamma chain purification from E. coli.

FIG. 5 shows an SDS-PAGE analysis of the Fgn-alpha chain purification from E. coli.

FIG. 6 shows an SDS-PAGE analysis of input reduced denatured fibrinogen (lane F) and purified subunits alfa (lane c, 67.7 kDa), beta (lane P, 52.3 kDa) and gamma (lane y, 48.5 kDa). Lane M contains molecular weight marker with approximate molecular weights of the constituents labeled on the right.

FIG. 7 shows an SDS-PAGE analysis of the PEGylation time course of recombinant Fgn-beta-PEG-NB

FIG. 8 shows an SDS-PAGE analysis of human derived Fgn-beta-PEG-NB, human derived Fgn-gamma-PEG-NB, recombinant Fgn-gamma-PEG-NB, and DI-Fgn-PEG-NB where all chains are present and human-derived.

FIG. 9 shows equilibrium swollen masses for the hydrogels prepared under denaturing and non-denaturing conditions. Average mass from three replicas for each series is shown. Error bars represent +/−1 standard deviation of measurement per series.

FIG. 10 shows equilibrium swollen masses for the hydrogels prepared from donor plasma-derived human fibrinogen (Fgn+20k-4, Fgn+20KL, Fgn+10KL, Fgn+6KL) compared to recombinantly expressed and purified Fibrinogen Gamma chain (Gamma+20k-4, Gamma+20KL, Gamma+10KL, Gamma+6KL) and to the hydrogels prepared from DI-Fgn-PEG-NB under non-denaturing conditions (DI-Fgn-PEG-NB+DTT). Average mass from three replicas for each series is shown. Error bars represent +/−1 standard deviation of measurement per series.

FIG. 11 shows swollen masses of scaffolding hydrogels prepared without lyoprotectant (“no lyoprotectant”), with the lyoprotectant but not freeze-dried (“freshly made”), or with the lyoprotectant, freeze-dried and rehydrated prior to photopolymerization. Error bars represent one standard deviation of measurement.

FIG. 12A and FIG. 12B show pictures of exemplary cellular infiltration assay experiments. FIGS. 12C and 12D present pictures of cellular infiltration assay experiments for materials with 20K4A PEG-NB (FIG. 12C) and with 30k6A PEG-NB (FIG. 12D).

FIG. 13A and FIG. 13B show an example of a Trichrome-stained tissue section identifying key features of a partially healed wound in an acute, full-thickness wound healing model.

FIGS. 14A-14G present pictures of cellular infiltration assay experiments for materials with varying concentration of DIFgn-PEG-NB in the polymer formulation.

FIGS. 15A-15E present pictures of histological studies in acute, full-thickness wound healing models for materials with varying concentration of DIFgn-PEG-NB in the polymer formulation: 3.0% wt./vol. (FIG. 15A), 2.25% wt./vol. (FIG. 15B), 1.5% wt./vol. (FIG. 15C), 0.75% wt./vol. (FIG. 15D), 0% wt./vol. (FIG. 15E).

FIG. 16 illustrates the influence of the various formulation components on the mechanical properties and swelling properties of the materials.

FIG. 17A and FIG. 17B present pictures of histological studies in acute, full-thickness wound healing models for materials prepared with an inert fibrinogen derivative encapsulated in the hydrogel material.

FIG. 17C and FIG. 17D present pictures of histological studies in acute, full-thickness wound healing models for materials prepared with a fibrinogen component covalently incorporated in the hydrogel material.

FIG. 18 shows an image taken from a standard light microscope of a cell-covered hydrogel microsphere.

FIG. 19A presents a histological study in an acute, full-thickness wound healing model for materials for bulk in situ polymerized formulations. FIG. 19B presents a histological study in an acute, full-thickness wound healing model for materials for hydrogel microspheres of about 0.5 mm diameter. FIG. 19C presents a histological study in an acute, full-thickness wound healing model for materials for hydrogel microspheres of about 0.1 mm diameter. FIG. 19D presents a histological study in an acute, full-thickness wound healing model for materials for hydrogel microspheres of about 0.1 mm diameter.

FIG. 20A presents a histological study in an acute, full-thickness wound healing model for materials for bulk in situ polymerized formulations 3 days after application. FIG. 20B presents a histological study in an acute, full-thickness wound healing model for materials for bulk in situ polymerized formulations 8 days after application. FIG. 20C presents a histological study in an acute, full-thickness wound healing model for materials for bulk in situ polymerized formulations 14 days after application.

FIG. 21A presents a histological study in an acute, full-thickness wound healing model for hydrogel microspheres having a diameter of about 0.1 mm to about 0.2 mm, 3 days after application. FIG. 21B presents a histological study in an acute, full-thickness wound healing model for hydrogel microspheres having a diameter of about 0.1 mm to about 0.2 mm, 8 days after application. FIG. 21C presents a histological study in an acute, full-thickness wound healing model for hydrogel microspheres having a diameter of about 0.1 mm to about 0.2 mm, 14 days after application.

FIG. 22A presents a histological study in an acute, full-thickness wound healing model for hydrogel microspheres having a diameter of about 0.2 mm to about 0.3 mm, 3 days after application. FIG. 22B presents a histological study in an acute, full-thickness wound healing model for hydrogel microspheres having a diameter of about 0.2 mm to about 0.3 mm, 8 days after application. FIG. 22C presents a histological study in an acute, full-thickness wound healing model for hydrogel microspheres having a diameter of about 0.2 mm to about 0.3 mm, 14 days after application.

FIG. 23A presents a histological study in an acute, full-thickness wound healing model for hydrogel microspheres having a diameter of about 0.5 mm to about 0.7 mm, 3 days after application. FIG. 23B presents a histological study in an acute, full-thickness wound healing model for hydrogel microspheres having a diameter of about 0.5 mm to about 0.7 mm, 8 days after application. FIG. 23C presents a histological study in an acute, full-thickness wound healing model for hydrogel microspheres having a diameter of about 0.5 mm to about 0.7 mm, 14 days after application.

FIG. 24A, FIG. 24B and FIG. 24C show cellular invasion assays. In FIG. 24A, no Fgn is incorporated into the polymer matrix. In FIG. 24B, native derived Fgn-gamma-PEG-NB is incorporated into the polymer, and in FIG. 24C, recombinant Fgn-gamma-PEG-NG is incorporated into the polymer matrix.

FIG. 25 shows a Trichrome stained wound with an in-situ polymerized Fgn-gamma-PEG-NB polymer on day 5.

FIG. 26 shows a Trichrome stained partially healed wound with microparticles containing Fgn-gamma-PEG-NB on day 8.

FIG. 27A and FIG. 27B shows cellular invasion assays. In FIG. 27A no Fgn is incorporated into the polymer matrix. In FIG. 27B, native-derived Fgn-beta-PEG-NB is incorporated into the polymer matrix.

FIG. 28 shows a Trichrome stained partially healed wound with an in-situ polymerized Fgn-beta-PEG-NB containing polymer on day 8.

FIG. 29 shows a Trichrome stained partially healed wound with direct-to-polymer DI-Fgn-PEG-NB containing microparticles on day 5.

FIG. 30 shows a Trichrome stained partially healed wound with direct-to-polymer Fgn-gamma-PEG-NB microparticles on day 5.

FIG. 31 shows a Trichrome stained wound with an in-situ polymerized hydrogel containing DI-Fgn-PEG-NB and linear and 4-arm PEG norbornenes crosslinked with the Matrix Metalloprotease-degradable hCysMMPA polypeptide on day 7 post-application.

FIG. 32 shows a Trichrome stained wound with an in-situ polymerized hydrogel containing DI-Fgn-PEG-NB and linear and 4-arm PEG norbornenes crosslinked with the plasmin-degradable hCys2xPC polypeptide on day 7 post-application.

FIG. 33. Two Fgn subunits, native derived Fgn-Beta-PEG-NB and recombinant Fgn-gamma-PEG-NB can be combined together to support cellular invasion.

### DETAILED DESCRIPTION

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

As used herein, “a fibrinogen component” intends and refers to a fibrinogen molecule, the product or products of denaturing a fibrinogen molecule, or a fragment of the foregoing. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

As used herein, “a microparticle” refers to a particle having a longest dimension from about 0.001 mm to about 1 mm. A microparticle can present in various three-dimensional forms. A microparticle can be of spherical shape (spheres and beads) and it will be understood in this context that the longest dimension of the microparticle is the diameter of the sphere. A microparticle can be of ellipsoidal shape and it will be understood in this context that the longest dimension of the microparticle is the length of the ellipsoid's longest axis. A microparticle can be of parallelepiped shape and it will be understood that in this context the longest dimension of the microparticle is the diameter of the smallest sphere able to contain the parallelepiped microparticle in its entirety. A microparticle can be of a rod-like shape and it will be understood that in this context the longest dimension of the microparticle is the diameter of the smallest sphere able to contain the microparticle in its entirety. A microparticle can be of irregular shape and it will be understood that in this context the longest dimension of the microparticle is the diameter of the smallest sphere able to contain the microparticle in its entirety. Further, it is understood that the microparticles of the present disclosure may be of a specific shape prior to application to a patient but may be deformed in situ due to pressures resulting of the wound environment. Unless otherwise indicated, it is understood that the microparticle's shape refers to the shape of the microparticle prior to application. Unless otherwise indicated, it is understood that the microparticle's longest dimension refers to the longest dimension of the microparticle prior to application.

As used herein, “particle-size distribution” refers to the distribution of individual particle size in a sample or composition containing a plurality of particles. As used herein, a composition comprising a plurality of microparticles can be defined as containing about X % of microparticles with a longest dimension of between about Y mm and about Z mm. Identification of particle size distribution can be achieved through various methods known in the art such as, for non-limiting example, sieve analysis, photoanalysis, analysis with a Coulter counter, and optical counting methods or the like.

Unless clearly indicated otherwise, “an individual”, “a patient”, and “a subject” as used herein intends a mammal, including but not limited to a primate, human, bovine, horse, suid, feline, canine, or rodent. In one variation, the individual is a human.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease or injury, diminishing the extent of the disease or injury, stabilizing the disease or injury (e.g., preventing or delaying the worsening of the disease or injury), decreasing the dose of one or more other medications required to treat the disease, enhancing effect of another medication, delaying the progression of the disease or injury, increasing the quality of life, and/or prolonging survival. The methods of the present disclosure contemplate any one or more of these aspects of treatment.

As used herein, “treatment of an injury” or “treating an injury” includes, but is not limited to, one or more of the following: permitting the healing of an injury, facilitating the healing of an injury, accelerating the healing of an injury, enhancing the rate of tissue regeneration in an injury, enhancing the amount of tissue regeneration in an injury, and/or reducing the damage to tissue in an injury.

As used herein, the term “a wound” intends damage to any tissue in a subject. The tissue may be an internal tissue such as a cartilage or a bone, or an external tissue such as skin. A wound may be in soft tissue or in hard tissue. Further, a wound may have been caused by any agent, such as for non-limiting example traumatic injury, infection, a disease or condition, or surgical intervention. Lastly, a wound can be a chronic wound or an acute wound.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the terms “a drug” or “drugs’ intends articles intended for use in the diagnosis, cure, mitigation, treatment, or prevention of a disease and articles intended to affect the structure or any function of the body of a subject.

As used herein, the term “cosmetics” intends articles intended to be rubbed, poured, sprinkled, sprayed on, introduced into, or otherwise applied to the human body for cleansing, beautifying, promoting attractiveness, or altering the appearance. For non-limiting exemplary purposes, cosmetics can be skin lotions, perfumes, lipsticks, fingernail polishes, eye and face makeup, cleansing shampoos, hair dyes, and deodorants.

As used herein, the term “device” intends an instrument, apparatus, implement, machine, contrivance, implant, in vitro reagent, or other similar or related article, including a component part, or accessory. In some embodiments, a device is: (1) recognized in the official National Formulary, or the United States Pharmacopoeia, or any supplement to them, or recognized by another national registry as a medical device; (2) intended for use in the diagnosis of a disease or condition, or in the cure, mitigation, treatment, or prevention of a disease or condition in a subject; or (3) intended to affect the structure or any function of the body of a subject, and which does not achieve its primary intended purposes through chemical action within or on the body of a subject, and which is not dependent upon being metabolized for the achievement of its primary intended purposes.

As used herein, the term “biological product” intends a virus, therapeutic serum, toxin, antitoxin, vaccine, blood, blood component or derivative, allergenic product, protein (except any chemically synthesized polypeptide), or analogous product, or arsphenamine or derivative of arsphenamine (or any other trivalent organic arsenic compound), applicable to the diagnosis of a disease or condition, or in the cure, mitigation, treatment, or prevention of a disease or condition in a subject.

As used herein, the term “a reactive ene group” intends a suitable ethylenically unsaturated group such as, but not limited to, vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

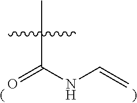 [SMILES: *C(=O)NC=C]

and norborn-2-en-5-yl

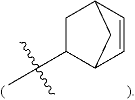 [SMILES: *C1CC2C=CC1C2]

As used herein, the term “substantially swell”, or any grammatical variation thereof, intends that the physical dimensions of the object of the swelling are increased by more than 10%, 25%, 50%, 75%, 100%, 150%, or 200%. Conversely, as used herein, the term “does not substantially swell” intends that the physical dimensions of the object of the swelling are increased by no more than 10%, no more than 25%, no more than 50%, no more than 75%, no more than 100%, no more than 150%, or no more than 200%.

It is understood that aspects and embodiments described herein as “comprising” include “consisting of” and “consisting essentially of” embodiments.

When a composition is described as “consisting essentially of” the listed components, the composition contains the components expressly listed, and may contain other components which do not substantially affect the disease or condition being treated such as trace impurities. However, the composition either does not contain any other components which do substantially affect the disease or condition being treated other than those components expressly listed; or, if the composition does contain extra components other than those listed which substantially affect the disease or condition being treated, the composition does not contain a sufficient concentration or amount of those extra components to substantially affect the disease or condition being treated. When a method is described as “consisting essentially of” the listed steps, the method contains the steps listed, and may contain other steps that do not substantially affect the disease or condition being treated, but the method does not contain any other steps which substantially affect the disease or condition being treated other than those steps expressly listed.

### Materials and Compositions

The disclosure provides hydrogel biomaterials which are useful for treatment of an injury. The hydrogel biomaterials can be administered to subjects in need thereof. The hydrogel biomaterials provided herein remain substantially intact and provide a bulk-filling role during the early phases of injury healing and are subsequently degraded as injury healing progresses. As they degrade, the hydrogel biomaterials are replaced with granulation tissue.

The materials and methods detailed herein are an advance over existing materials and methods for use e.g., in treating a tissue defect in an individual, such as an injury (e.g. a wound). Specifically, the hydrogel biomaterials provided herein (i) have reproducible and defined mechanical properties; (ii) have degradation properties conducive to cellular infiltration; and (iii) have advantageous swelling properties.

The disclosure further provides biomaterials comprising hydrogel microparticles which are useful for treatment of an injury. The biomaterials can be administered to subjects in need thereof. The microparticles provided herein allow rapid migration of cells into the injury volume via degradation-independent pathways (over and between microparticles). The microparticles remain substantially intact and provide a bulk-filling role during the early phases of injury healing and are subsequently degraded as injury healing progresses. As they degrade, the microparticles are replaced with granulation tissue.

The materials and methods detailed herein are an advance over existing materials and methods for use e.g., in treating a tissue defect in an individual, such as an injury (e.g. a wound). Specifically, the microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In a first facet, provided herein is a hydrogel biomaterial comprising

at least one linker component;

at least one protease-cleavable component; and

a fibrinogen component derivatized with a plurality of polymeric linkers;

wherein at least a portion of the at least one linker component, at least a portion of the at least one protease-cleavable component, and at least a portion of the fibrinogen component are crosslinked via a plurality of crosslink units each independently comprising a carbon-sulfur covalent bond.

In a first variation of the first facet, the at least one linker component comprises at least one polymer component. In such embodiments, provided herein is a hydrogel biomaterial comprising

at least one polymer component;

at least one protease-cleavable component; and

a fibrinogen component derivatized with a plurality of polymeric linkers;

wherein at least a portion of the at least one polymer component, at least a portion of the at least one protease-cleavable component, and at least a portion of the fibrinogen component are crosslinked via a plurality of crosslink units each independently comprising a carbon-sulfur covalent bond.

In some embodiments, the crosslink units of the plurality of crosslink units are selected from the group consisting of

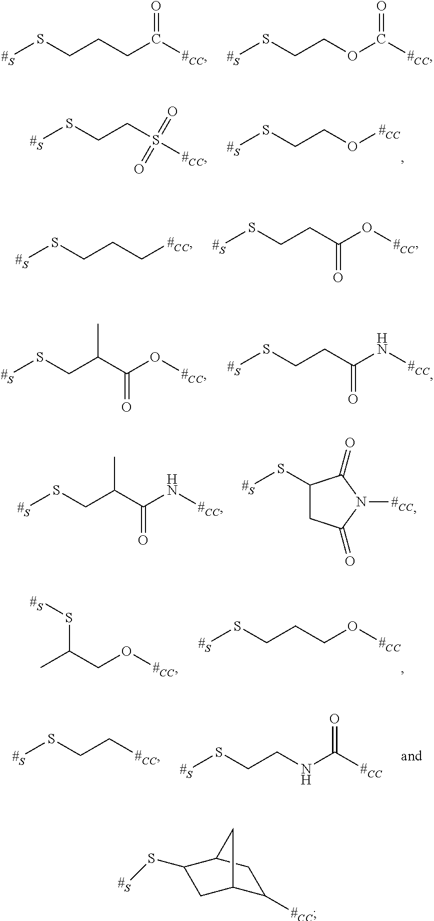

wherein #s and #cc represent attachment points to the at least one polymer component, the at least one protease-cleavable component, or a polymeric linker of the fibrinogen component. In some embodiments, all crosslink units of the plurality of crosslink units are identical. In some embodiments, all crosslink units of the plurality of crosslink units are

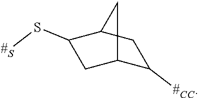

In a first aspect, at least a portion of the at least one polymer component is connected to at least a portion of the at least one protease-cleavable component via a first crosslink unit and at least a portion of the fibrinogen component is connected to at least a portion of the at least one protease-cleavable component via a second crosslink unit. In some embodiments of the first aspect, the at least one polymer component is connected to the at least one protease-cleavable component via a first crosslink unit and the fibrinogen component is connected to the at least one protease-cleavable component via a second crosslink unit.

In a first variation of the first aspect, the first crosslink unit is selected from the group consisting of

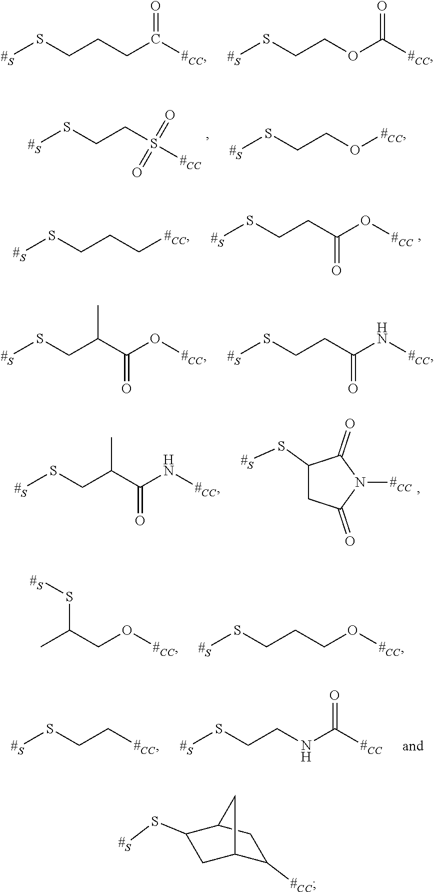

wherein #s represents the attachment point to the at least one protease-cleavable component and #cc represents the attachment point to the at least one polymer component. In some embodiments of the first variation of the first aspect, the first crosslink unit is

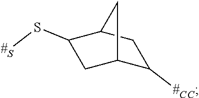

wherein #s represents the attachment point to the at least one protease-cleavable component and #cc represents the attachment point to the at least one polymer component.

In the first variation of the first aspect, the second crosslink unit is selected from the group consisting of

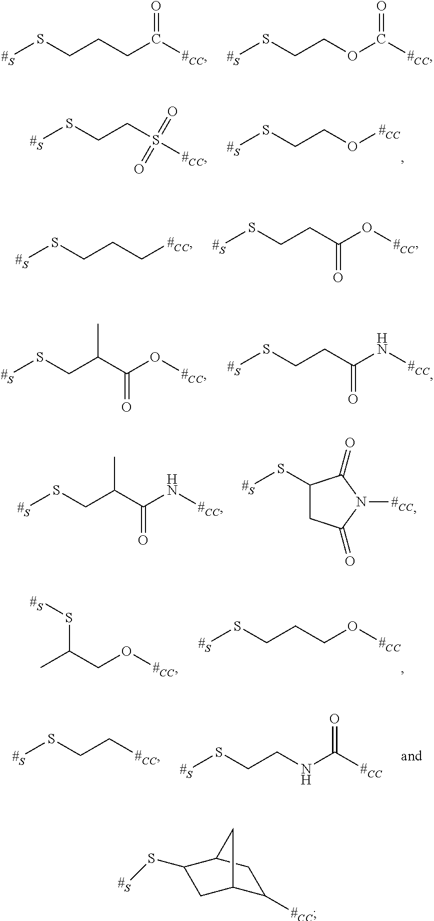

wherein #s represents the attachment point to the at least one protease-cleavable component and #cc represents the attachment point to a polymeric linker of the fibrinogen component. In some embodiments of the first variation of the first aspect, the second crosslink unit is

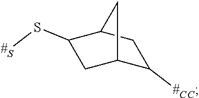

wherein #s represents the attachment point to the at least one protease-cleavable component and #cc represents the attachment point to a polymeric linker of the fibrinogen component.

In a second aspect, at least a portion of the at least one polymer component is connected to at least a portion of the at least one protease-cleavable component via a first crosslink unit and at least a portion of the fibrinogen component is connected to at least a portion of the at least one polymer component via a second crosslink unit. In some embodiments of the second aspect, the at least one polymer component is connected to the at least one protease-cleavable component via a first crosslink unit and the fibrinogen component is connected to at least one polymer component via a second crosslink unit.

In some embodiments of the second aspect, the first crosslink unit is selected from the group consisting of

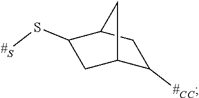

wherein #s represents the attachment point to the at least one polymer component and #cc represents the attachment point to the at least one protease-cleavable component. In some embodiments of the second aspect, the first crosslink unit is

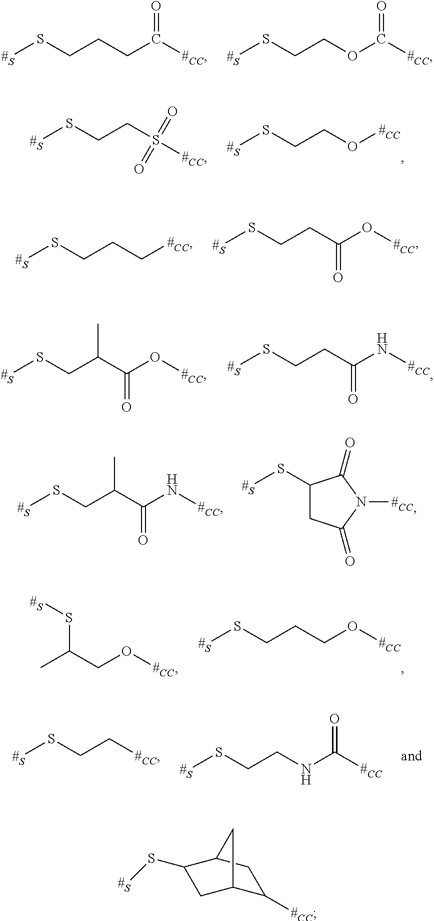

wherein #s represents the attachment point to the at least one polymer component and #cc represents the attachment point to the at least one protease-cleavable component.

In some embodiments of the second aspect, the second crosslink unit is selected from the group consisting of

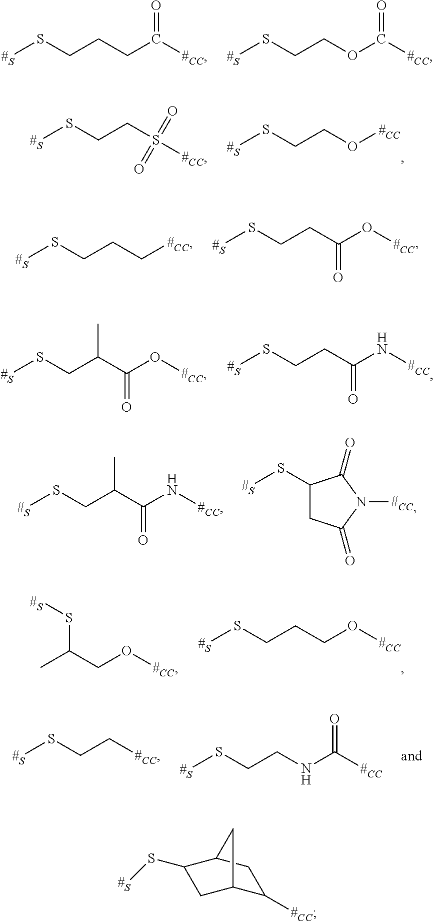

wherein #s represents the attachment point to the at least one polymer component and #cc represents the attachment point to a polymeric linker of the fibrinogen component. In some embodiments of the second aspect, the second crosslink unit

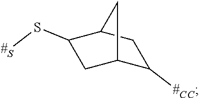

wherein #s represents the attachment point to the at least one polymer component and #cc represents the attachment point to a polymeric linker of the fibrinogen component.

In a third aspect, at least a portion of the at least one polymer component is connected to at least a portion of the fibrinogen component via a first crosslink unit and at least a portion of the fibrinogen component is connected to at least a portion of the at least one protease-cleavable component via a second crosslink unit. In some embodiments of the third aspect, the at least one polymer component is connected to the fibrinogen component via a first crosslink unit and the fibrinogen component is connected to the at least one protease-cleavable component via a second crosslink unit.

In some embodiments of the third aspect, the first crosslink unit is selected from the group consisting of

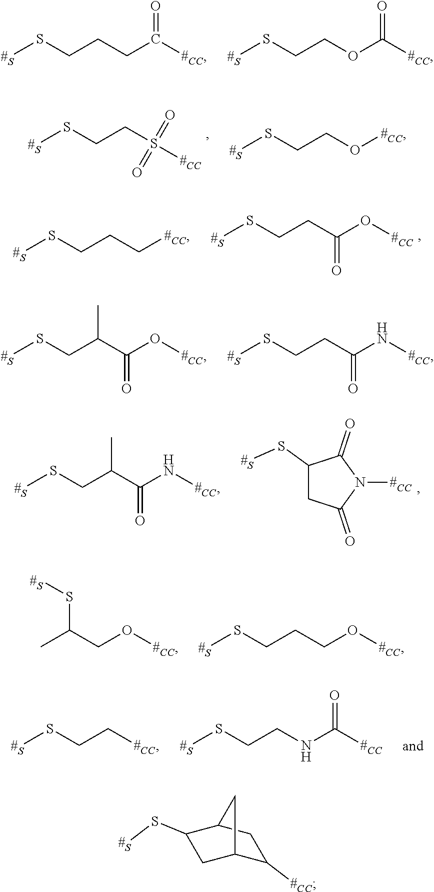

wherein #s represents the attachment point to the at least one polymer component and #cc represents the attachment point to a polymeric linker of the fibrinogen component. In some embodiments of the third aspect, the first crosslink unit

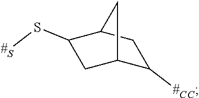

wherein #s represents the attachment point to the at least one polymer component and #cc represents a polymeric linker of the fibrinogen component.

In some embodiments of the third aspect, the second crosslink unit is selected from the group consisting of

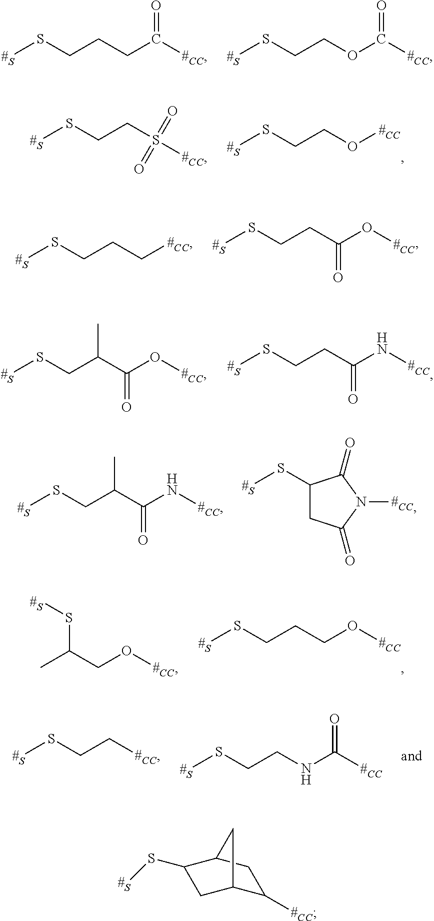

wherein #s represents the attachment point to the at least one protease-cleavable component and #cc represents the attachment point to a polymeric linker of the fibrinogen component. In some embodiments of the third aspect, the second crosslink unit is

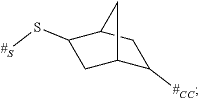

wherein #s represents the attachment point to the at least one protease-cleavable component and #cc represents the attachment point to a polymeric linker of the fibrinogen component.

In some embodiments of any of the preceding aspects of the first variation of the first facet, the at least one polymer component comprises a linear polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of about 3 kDa, about 6 kDa, and about 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of any of the preceding aspects of the first variation of the first facet, the at least one polymer component comprises a branched polymer component. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of any of the preceding aspects of the first variation of the first facet, the at least one polymer component comprises a linear polymer component and a branched polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of 3 kDa, 6 kDa, and 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a poly(ethylene glycol) moiety, wherein the poly(ethylene glycol) moiety has a molecular weight selected from the group consisting of 3 kDa, 6 kDa, and 10 kDa. In some embodiments, the linear polymer component comprises a poly(ethylene glycol) moiety, wherein the poly(ethylene glycol) moiety has a molecular weight of about 10 kDa. In some embodiments, the linear polymer component comprises a poly(ethylene glycol) moiety, wherein the poly(ethylene glycol) moiety has a molecular weight of about 6 kDa. In some embodiments, the linear polymer component comprises a poly(ethylene glycol) moiety, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of about 30 kDa.

In a second variation of the first facet, the at least one linker component comprises at least one multivalent linker component. In such embodiments, provided herein is a hydrogel biomaterial comprising

at least one multivalent linker component;

at least one protease-cleavable component; and

a fibrinogen component derivatized with a plurality of polymeric linkers;

wherein at least a portion of the at least one multivalent linker component, at least a portion of the at least one protease-cleavable component, and at least a portion of the fibrinogen component are crosslinked via a plurality of crosslink units each independently comprising a carbon-sulfur covalent bond.

In some embodiments, the crosslink units of the plurality of crosslink units are selected from the group consisting of

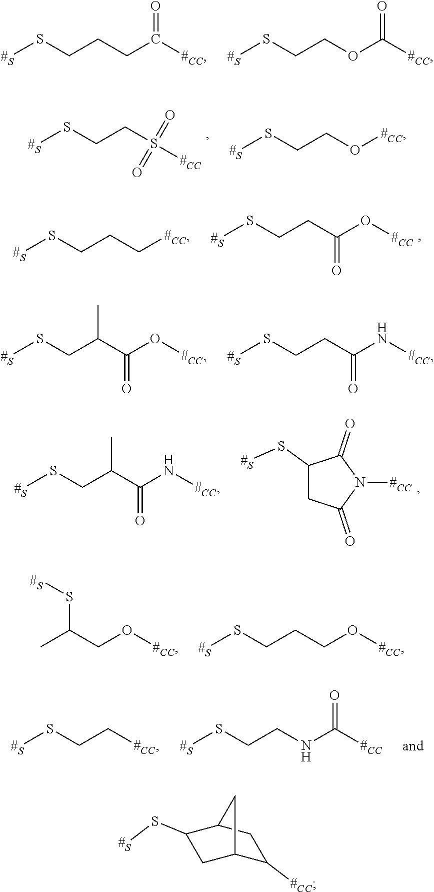

wherein #s and #cc represent attachment points to the at least one multivalent linker component, the at least one protease-cleavable component, or a polymeric linker of the fibrinogen component. In some embodiments, all crosslink units of the plurality of crosslink units are identical. In some embodiments, all crosslink units of the plurality of crosslink units are

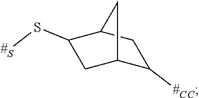

In a first aspect, at least a portion of the at least one multivalent linker component is connected to at least a portion of the at least one protease-cleavable component via a first crosslink unit and at least a portion of the fibrinogen component is connected to at least a portion of the at least one multivalent linker component via a second crosslink unit. In some embodiments of the second aspect, the at least one multivalent linker component is connected to the at least one protease-cleavable component via a first crosslink unit and the fibrinogen component is connected to at least one multivalent linker component via a second crosslink unit.

In some embodiments of the first aspect, the first crosslink unit is selected from the group consisting of

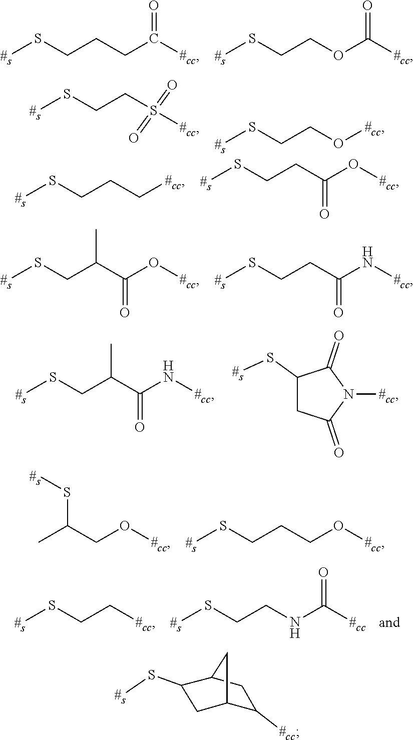

wherein #s represents the attachment point to the at least one multivalent linker component and #cc represents the attachment point to the at least one protease-cleavable component. In some embodiments of the first aspect, the first crosslink unit is

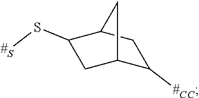

wherein #s represents the attachment point to the at least one multivalent linker component and #cc represents the attachment point to the at least one protease-cleavable component.

In some embodiments of the second aspect, the second crosslink unit is selected from the group consisting of

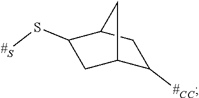

wherein #s represents the attachment point to the at least one multivalent linker component and #cc represents the attachment point to a polymeric linker of the fibrinogen component. In some embodiments of the first aspect, the second crosslink unit is

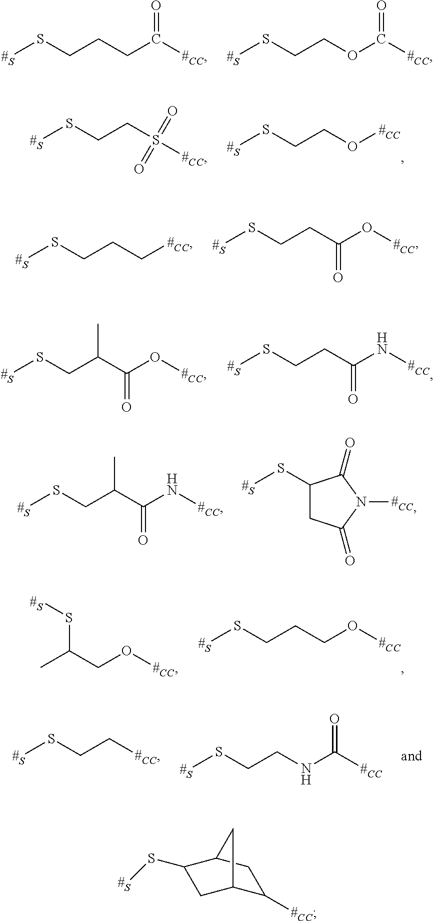

wherein #s represents the attachment point to the at least one multivalent linker component and #cc represents the attachment point to a polymeric linker of the fibrinogen component.

In a second aspect, at least a portion of the at least one multivalent linker component is connected to at least a portion of the fibrinogen component via a first crosslink unit and at least a portion of the fibrinogen component is connected to at least a portion of the at least one protease-cleavable component via a second crosslink unit. In some embodiments of the third aspect, the at least one multivalent linker component is connected to the fibrinogen component via a first crosslink unit and the fibrinogen component is connected to the at least one protease-cleavable component via a second crosslink unit.

In some embodiments of the second aspect, the first crosslink unit is selected from the group consisting of

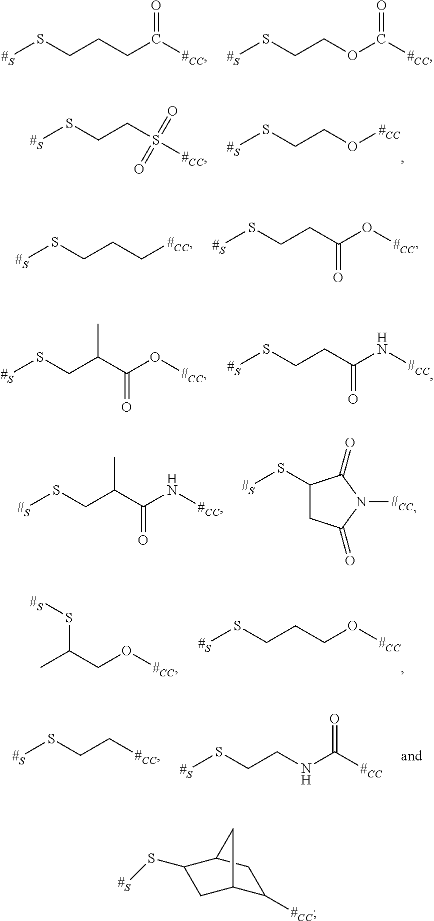

wherein #s represents the attachment point to the at least one multivalent linker component and #cc represents the attachment point to a polymeric linker of the fibrinogen component. In some embodiments of the second aspect, the first crosslink unit is

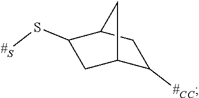

wherein #s represents the attachment point to the at least one multivalent linker component and #cc represents a polymeric linker of the fibrinogen component.

In some embodiments of the second aspect, the second crosslink unit is selected from the group consisting of

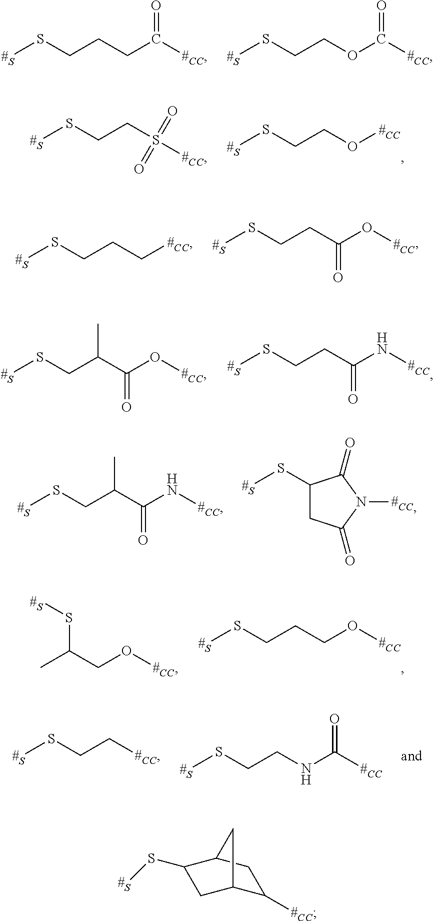

wherein #s represents the attachment point to the at least one protease-cleavable component and #cc represents the attachment point to a polymeric linker of the fibrinogen component. In some embodiments of the second aspect, the second crosslink unit is

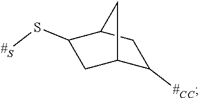

wherein #s represents the attachment point to the at least one protease-cleavable component and #cc represents the attachment point to a polymeric linker of the fibrinogen component.

In some embodiments of any of the preceding aspects of the second variation of the first facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of any of the preceding aspects of the second variation of the first facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of any of the preceding aspects and variations of the first facet, the at least one protease-cleavable component is a synthetic peptide. In some embodiments, each of the at least one protease-cleavable component is a peptide having a sequence of between 3 and 25 amino acids. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2.

TABLE 1
PLASMIN CLEAVABLE SEQUENCES

| | |
|---|---|
| SEQ ID NO: 1 | CWKC |
| SEQ ID NO: 2 | CALKC |
| SEQ ID NO: 3 | CALKVLKGC |
| SEQ ID NO: 4 | CALKVLKGCG-amide |
| SEQ ID NO: 5 | (hC)ALKVLKG(hC) |
| SEQ ID NO: 6 | (hC)ALKVLKG(hC)G |
| SEQ ID NO: 7 | (hC)ALKVLKG(hC)G-amide |
| SEQ ID NO: 8 | K(hC)ALKVLKG(hC)G-amide |
| SEQ ID NO: 9 | K(hC)GALKVLKG(hC)G-amide |
| SEQ ID NO: 10 | G(hC)GALKVLKG(hC)G-amide |
| SEQ ID NO: 11 | K(hC)ALKVLKG(hC)G-amide |
| SEQ ID NO: 12 | K(hC)PALKVLKG(hC)G-amide |
| SEQ ID NO: 13 | (hC)PALKVLKG(hC)G-amide |
| SEQ ID NO: 14 | K(hC)PGALKVLKG(hC)G-amide |

TABLE 2
MATRIX METALLOPROTEINASE CLEAVABLE SEQUENCES

| | |
|---|---|
| SEQ ID NO: 15 | KKCGGPQGIAGQGCKK |
| SEQ ID NO: 16 | KKCGGPQGIWGQGCKK |
| SEQ ID NO: 17 | KCGPLGLWARGCK |
| SEQ ID NO: 18 | KCGPQGIAGQCK |
| SEQ ID NO: 19 | K(hC)GPQGIAGQ(hC)K |

In Tables 1 and 2 above, (hC) denotes a homocysteine residue. In Tables 1 and 2 above, it is understood that a sequence terminating in “X-amide” denotes that the COOH terminus of the residue X is modified to be a CONH₂ group. For example, SEQ ID Nos: 4 and 7-14 are terminated by “G-amide”, in those sequences the COOH terminus of the terminal glycine is modified to be a CONH₂ group.

It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers. For further non-limiting exemplary purposes, it is understood that additional suitable protease-cleavable components may be prepared by modifying the amino acids sequences described above (SEQ ID NOs: 1-19) via the addition of flanking lysine residues which have been modified on the lysine's side chain. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877. In some embodiments, each of the at least one protease-cleavable component is independently of each other a matrix metalloproteinase (MMP) cleavable component or a plasmin cleavable component. In some embodiments, the hydrogel biomaterial comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the hydrogel biomaterial comprises two protease-cleavable components. In some embodiments, the hydrogel biomaterial comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of any of the preceding aspects and variations of the first facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, each individual polymeric linker of the plurality of polymeric linkers comprises a poly(ethylene glycol) moiety, wherein the poly(ethylene glycol) moiety has a molecular weight of between about 1 kDa and 40 kDa. In some embodiments, each individual polymeric linker of the plurality of polymeric linkers comprises a poly(ethylene glycol) moiety, wherein the poly(ethylene glycol) moiety has a molecular weight selected from the group consisting of 3.5 kDa, 6 kDa, and 10 kDa. In some embodiments, each individual polymeric linker of the plurality of polymeric linkers comprises a poly(ethylene glycol) moiety, wherein the poly(ethylene glycol) moiety has a molecular weight of about 3.5 kDa. In some embodiments, each individual polymeric linker of the plurality of polymeric linkers comprises a poly(ethylene glycol) moiety, wherein the poly(ethylene glycol) moiety has a molecular weight of about 6 kDa. In some embodiments, each individual polymeric linker of the plurality of polymeric linkers comprises a poly(ethylene glycol) moiety, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of any of the preceding aspects and variations of the first facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of any of the preceding aspects of the first facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a multiple armed PEG moiety comprising n arms, wherein the PEG moiety has an average molecular weight of 20 kDa, and n is 3, 4, 6 or 8;

0-6% wt. of a first protease-cleavable component comprising a MMP-cleavable peptide K(hC)GPQGIAGQ(hC)K (SEQ ID NO: 19);

0-6% wt. of a second protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the first facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a 4-armed PEG moiety, wherein the 4-armed PEG moiety has an average molecular weight of 20 kDa;

0-6% wt. of a first protease-cleavable component comprising a MMP-cleavable peptide K(hC)GPQGIAGQ(hC)K (SEQ ID NO: 19);

0-6% wt. of a second protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the first facet, the hydrogel biomaterial comprises between about 0.25% wt. and about 5% wt. of the fibrinogen component. In some embodiments, the hydrogel biomaterial comprises about 0.75% wt. of the fibrinogen component. In some embodiments, the hydrogel biomaterial comprises about 1.50% wt. of the fibrinogen component. In some embodiments, the hydrogel biomaterial comprises about 3.00% wt. of the fibrinogen component.

In some embodiments of any of the preceding aspects of the first facet, the hydrogel biomaterial comprises between about 0.1% wt. and 5% wt. of the linear polymer component. In some embodiments, the hydrogel biomaterial comprises between about 0.5% wt. and 2% wt. of the linear polymer component. In some embodiments, the hydrogel biomaterial comprises about 1% wt. of the linear polymer component.

In some embodiments of any of the preceding aspects of the first facet, the hydrogel biomaterial comprises between about 0.1% wt. and 5% wt. of the branched polymer component. In some embodiments, the hydrogel biomaterial comprises between about 0.5% wt. and 2% wt. of the branched polymer component. In some embodiments, the hydrogel biomaterial comprises about 1% wt. of the branched polymer component.

In some embodiments of any of the preceding aspects of the first facet, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some embodiments of the preceding embodiments and aspects of the first facet, the hydrogel microparticle has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some embodiments of the preceding embodiments and aspects of the first facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some embodiments of the preceding embodiments and aspects of the first facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments and aspects of the first facet, the hydrogel biomaterial does not substantially swell when exposed to a protease capable of cleaving the protease-cleavable component.

In some variations of the preceding embodiments and aspects of the first facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the first facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the first facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the first facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the first facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the first facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the first facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the first facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the first facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the first facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a second facet, provided herein is a hydrogel biomaterial, wherein the hydrogel biomaterial is prepared by a polymerization reaction of a mixture comprising:
at least one linker component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers, wherein each polymeric linker comprises a reactive ene group, and
   provided that at least one of the first reactive group and the second reactive group is a reactive thiol group.

In a first variation of the second facet, the at least one linker component comprises at least one polymer component. In such embodiments, provided herein is a hydrogel biomaterial, wherein the hydrogel biomaterial is prepared by a polymerization reaction of a mixture comprising:

at least one polymer component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers, wherein each polymeric linker comprises a reactive ene group, and
provided that at least one of the first reactive group and the second reactive group is a reactive thiol group.

In a first aspect of the first variation of the second facet, the first reactive group is a reactive ene group and the second reactive group is a reactive thiol group. In this first aspect of the first variation of the second facet, provided is a hydrogel biomaterial wherein the hydrogel biomaterial is prepared by a polymerization reaction of an aqueous mixture comprising:

at least one polymer component comprising reactive ene groups;
at least one protease-cleavable component comprising reactive thiol groups; and
a fibrinogen component derivatized with a plurality of polymeric linkers with reactive ene groups.

In some embodiments of the first aspect of the first variation of the second facet, the first reactive group is a reactive ene group and the second reactive group is a reactive thiol group. In this first aspect of the first variation of the second facet, provided is a hydrogel biomaterial wherein the hydrogel biomaterial is prepared by a polymerization reaction of an aqueous mixture comprising:

at least one polymer component comprising reactive ene groups;
at least one protease-cleavable component comprising reactive thiol groups; and
a fibrinogen component derivatized with a plurality of polymeric linkers with reactive ene groups; and
wherein the fibrinogen component does not possess the clotting activity of a native fibrinogen molecule.

In some embodiments of the first aspect of the first variation of the second facet, the at least one polymer component comprises a linear polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of about 3 kDa, about 6 kDa, and about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

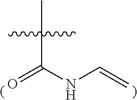 [SMILES: *C(=O)NC=C]

and norborn-2-en-5-yl

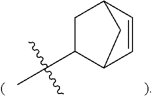 [SMILES: *C1CC2C=CC1C2]

In some embodiments, wherein the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of about 3 kDa, about 6 kDa, and about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the first aspect of the first variation of the second facet, the at least one polymer component comprises a branched polymer with reactive ene groups. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

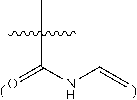 [SMILES: *C(=O)NC=C]

and norborn-2-en-5-yl

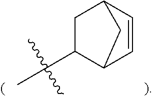 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 3-arm poly(ethylene glycol) moiety and three terminal norborn-2-en-5-yl groups (one at each terminus). In some embodiments, the 3-arm poly(ethylene glycol) moiety has a molecular weight of between about 1 kDa and about 40 kDa. In some embodiments, the 3-arm poly(ethylene glycol) moiety has a molecular weight of about 15 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the first aspect of the first variation of the second facet, the at least one polymer component comprises a linear polymer with reactive ene groups and a branched polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of 3 kDa, 6 kDa, and 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

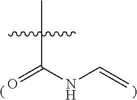 [SMILES: *C(=O)NC=C]

and norborn-2-en-5-yl

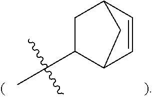 [SMILES: *C1CC2C=CC1C2]

In some embodiments, the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of 3 kDa, 6 kDa, and 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

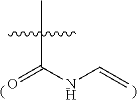 [SMILES: *C(=O)NC=C]

and norborn-2-en-5-yl

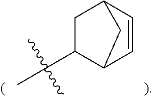 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of about 30 kDa.

In some embodiments of the first aspect of the first variation of the second facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2.

It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the matrix metalloproteinase (NIP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (NIP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (IMP) cleavable peptide is a peptide of SEQ ID NO: 18, and the plasmin cleavable peptide is a peptide of SEQ ID NO: 4. In some embodiments, the matrix metalloproteinase (IMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the first aspect of the first variation of the second facet, the hydrogel biomaterial is prepared by the free radical mediated polymerization reaction of a mixture comprising:

0-50% wt./vol. of a linear PEG with terminal norbornene groups, wherein the linear PEG has an average molecular weight of 6 kDa;

0.1-50% wt. of a branched polymer component comprising a multiple armed PEG moiety comprising n arms with terminal norbornene groups, wherein the PEG moiety has an average molecular weight of 20 kDa, and n is 3, 4, 6 or 8;

0-6% wt. of a first protease-cleavable component comprising a MMP-cleavable peptide K(hC)GPQGIAGQ(hC)K (SEQ ID NO: 19);

0-6% wt. of a second protease-cleavable component comprising a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the first aspect of the first variation of the second facet, the hydrogel biomaterial is prepared by the free radical mediated polymerization reaction of a mixture comprising:

0-50% wt./vol. of a linear PEG with terminal norbornene groups, wherein the linear PEG has an average molecular weight of 6 kDa;

0.1-50% wt./vol. of a 4-armed PEG with terminal norbornene groups having an average molecular weight of 20 kDa;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the first aspect of the first variation of the second facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the first aspect of the first variation of the second facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the first aspect of the first variation of the second facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the first aspect of the first variation of the second facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the first aspect of the first variation of the second facet, the molar ratio of the linear PEG to the IMP-cleavable peptide is between 0 and infinity. In some embodiments, the molar ratio of the linear PEG to the IMP-cleavable peptide is between about 20:1 and about 1:1. In some embodiments, the molar ratio of the linear PEG to the IMP-cleavable peptide is between about 2:1 and about 1:1. In some embodiments, the molar ratio of the linear PEG to the IMP-cleavable peptide is about 5:4.

In some embodiments of the first aspect of the first variation of the second facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the first aspect of the first variation of the second facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second aspect of the first variation of the second facet, the first reactive group is a reactive thiol group and the second reactive group is a reactive ene group. In this second aspect of the second facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive ene groups;
a fibrinogen component derivatized with a plurality of polymeric linkers with reactive ene groups.

In some embodiments of the second aspect of the first variation of the second facet, the first reactive group is a reactive thiol group and the second reactive group is a reactive ene group. In this second aspect of the first variation of the second facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive ene groups;
a fibrinogen component derivatized with a plurality of polymeric linkers with reactive ene groups, wherein the fibrinogen component does not possess the clotting activity of a native fibrinogen molecule.

In some embodiments of the second aspect of the first variation of the second facet, the at least one polymer component comprises a linear polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of about 3 kDa, about 6 kDa, and about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the second aspect of the first variation of the second facet, the at least one polymer component comprises a branched polymer with reactive thiol groups. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 3-arm poly(ethylene glycol) moiety and three terminal thiol groups. In some embodiments, the 3-arm poly(ethylene glycol) moiety has a molecular weight of between about 1 kDa and about 40 kDa. In some embodiments, the 4-arm poly(ethylene glycol) moiety has a molecular weight of about 15 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the second aspect of the first variation of the second facet, the at least one polymer component comprises a linear polymer with reactive thiol groups and a branched polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with reactive thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of 3 kDa, 6 kDa, and 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive thiol groups of the branched polymer are terminal reactive thiol groups. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of about 30 kDa.

In some embodiments of the first variation of the second aspect of the second facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (NIMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

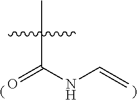 [SMILES: *C(=O)NC=C]

and norborn-2-en-5-yl

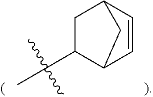 [SMILES: *C1CC2C=CC1C2]

In some embodiments of the second aspect of the first variation of the second facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-6% wt./vol. of a MP-cleavable peptide comprising a reactive ene group;

0-6% wt./vol. of a plasmin-cleavable peptide comprising a reactive ene group;

0-10% wt./vol. of a fibrinogen component derivatized at cysteine residues with 3.5 kDa linear PEG with terminal norbornene groups; and

water.

In some embodiments of the second aspect of the first variation of the second facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt./vol. of a 4-armed PEG with terminal thiol groups having an average molecular weight of 20 kDa;

0-6% wt./vol. of a IMP-cleavable peptide comprising a reactive ene group;

0-6% wt./vol. of a plasmin-cleavable peptide comprising a reactive ene group;

0-10% wt./vol. of a fibrinogen component derivatized at cysteine residues with 3.5 kDa linear PEG with terminal norbornene groups; and

water.

In some embodiments of the second aspect of the first variation of the second facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the second aspect of the first variation of the second facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the second aspect of the first variation of the second facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the second aspect of the first variation of the second facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the second aspect of the first variation of the second facet, the molar ratio of the linear PEG to the MMP-cleavable peptide is between 0 and infinity. In some embodiments, the molar ratio of the linear PEG to the MMP-cleavable peptide is between about 20:1 and about 1:1. In some embodiments, the molar ratio of the linear PEG to the MMP-cleavable peptide is between about 2:1 and about 1:1. In some embodiments, the molar ratio of the linear PEG to the IMP-cleavable peptide is about 5:4.

In some embodiments of the second aspect of the first variation of the second facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the second aspect of the first variation of the second facet, the overall stoichiometric ratio of the norbornene moieties to the free thiol moieties prior to polymerization is selected such that the average branch order of either the norbornene bearing entities or free thiol bearing entities prior to polymerization is greater than 2.

In a third aspect of the first variation of the second facet, the first reactive group is a reactive thiol group and the second reactive group is a reactive thiol group. In this third aspect of the first variation of the second facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive thiol groups;
a fibrinogen component derivatized with a plurality of polymeric linkers with reactive ene groups.

In some embodiments of the third aspect of the first variation of the second facet, the first reactive group is a reactive thiol group and the second reactive group is a reactive thiol group. In this third aspect of the first variation of the second facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive thiol groups;
a fibrinogen component derivatized with a plurality of polymeric linkers with reactive ene groups, wherein the fibrinogen component does not possess the clotting activity of a native fibrinogen molecule.

In some embodiments of the third aspect of the first variation of the second facet, the at least one polymer component comprises a linear polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of about 3 kDa, about 6 kDa, and about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the third aspect of the first variation of the second facet, the at least one polymer component comprises a branched polymer with reactive thiol groups. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 3-arm poly(ethylene glycol) moiety and three terminal thiol groups. In some embodiments, the 3-arm poly(ethylene glycol) moiety has a molecular weight of between about 1 kDa and about 40 kDa. In some embodiments, the 4-arm poly(ethylene glycol) moiety has a molecular weight of about 15 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the third aspect of the first variation of the second facet, the at least one polymer component comprises a linear polymer with reactive thiol groups and a branched polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with reactive thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of 3 kDa, 6 kDa, and 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive thiol groups of the branched polymer are terminal reactive thiol groups. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of about 30 kDa.

In some embodiments of the third aspect of the first variation of the second facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the third aspect of the first variation of the second facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the third aspect of the first variation of the second facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt./vol. of a 4-armed PEG with terminal thiol groups having an average molecular weight of 20 kDa;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the third aspect of the first variation of the second facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the third aspect of the first variation of the second facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the third aspect of the first variation of the second facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the third aspect of the first variation of the second facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the third aspect of the first variation of the second facet, the molar ratio of the linear PEG to the IMP-cleavable peptide is between 0 and infinity. In some embodiments, the molar ratio of the linear PEG to the IMP-cleavable peptide is between about 20:1 and about 1:1. In some embodiments, the molar ratio of the linear PEG to the IMP-cleavable peptide is between about 2:1 and about 1:1. In some embodiments, the molar ratio of the linear PEG to the IMP-cleavable peptide is about 5:4.

In some embodiments of the third aspect of the first variation of the second facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the third aspect of the first variation of the second facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second variation of the second facet, the at least one linker component comprises at least one multivalent linker component. In such embodiments, provided herein is a hydrogel biomaterial, wherein the hydrogel biomaterial is prepared by a polymerization reaction of a mixture comprising:

at least multivalent linker component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers, wherein each polymeric linker comprises a reactive ene group, and
provided that at least one of the first reactive group and the second reactive group is a reactive thiol group.

In a first aspect of the second variation of the second facet, the first reactive group is a reactive thiol group and the second reactive group is a reactive ene group. In this first aspect of the second variation the second facet, provided is a hydrogel biomaterial wherein the hydrogel biomaterial is prepared by a polymerization reaction of an aqueous mixture comprising:

at least one multivalent linker component comprising reactive thiol groups;
at least one protease-cleavable component comprising reactive ene groups;
a fibrinogen component derivatized with a plurality of polymeric linkers with reactive ene groups.

In some embodiments of the first aspect of the second variation of the second facet, the first reactive group is a reactive thiol group and the second reactive group is a reactive ene group. In this first aspect of the second variation of the second facet, provided is a hydrogel biomaterial wherein the hydrogel biomaterial is prepared by a polymerization reaction of an aqueous mixture comprising:

at least one multivalent linker component comprising reactive thiol groups;
at least one protease-cleavable component comprising reactive ene groups;
a fibrinogen component derivatized with a plurality of polymeric linkers with reactive ene groups, wherein the fibrinogen component does not possess the clotting activity of a native fibrinogen molecule.

In some embodiments of the first aspect of the second variation of the second facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

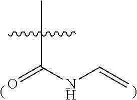 [SMILES: *C(=O)NC=C]

and norborn-2-en-5-yl

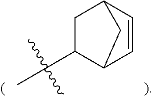 [SMILES: *C1CC2C=CC1C2]

In some embodiments of the second aspect of the first variation of the second facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the second aspect of the first variation of the second facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second aspect of the second variation of the second facet, the first reactive group is a reactive thiol group and the second reactive group is a reactive thiol group. In this second aspect of the second variation of the second facet, provided is a hydrogel biomaterial wherein the hydrogel biomaterial is prepared by a polymerization reaction of an aqueous mixture comprising:

at least one multivalent linker component comprising reactive thiol groups;
at least one protease-cleavable component comprising reactive thiol groups;
a fibrinogen component derivatized with a plurality of polymeric linkers with reactive ene groups.

In some embodiments of the second aspect of the second variation of the second facet, the first reactive group is a reactive thiol group and the second reactive group is a reactive thiol group. In this second aspect of the second variation of the second facet, provided is a hydrogel biomaterial wherein the hydrogel biomaterial is prepared by a polymerization reaction of an aqueous mixture comprising:

at least one multivalent linker component comprising reactive thiol groups;
at least one protease-cleavable component comprising reactive thiol groups;
a fibrinogen component derivatized with a plurality of polymeric linkers with reactive ene groups, wherein the fibrinogen component does not possess the clotting activity of a native fibrinogen molecule.

In some embodiments of the second aspect of the second variation of the second facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the second aspect of the second variation of the second facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the second aspect of the second variation of the second facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of any of the preceding aspects of the second variation of the second facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of any of the preceding aspects of the second variation of the second facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of the preceding aspects and variations of the second facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

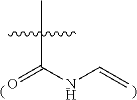 [SMILES: *C(=O)NC=C]

and norborn-2-en-5-yl

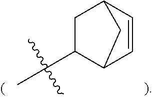 [SMILES: *C1CC2C=CC1C2]

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of the preceding aspects of the second facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the preceding aspects of the second facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the second facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the second facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the second facet, the hydrogel microparticle has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments and aspects of the second facet, the hydrogel biomaterial does not substantially swell when exposed to a protease capable of cleaving the protease-cleavable component.

In some variations of the preceding embodiments and aspects of the second facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the second facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the second facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the second facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the second facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the second facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the second facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the second facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the second facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the second facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a third facet, provided herein is a hydrogel biomaterial wherein the hydrogel biomaterial is prepared by a polymerization reaction of an aqueous mixture comprising:

at least one linker component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial has a storage modulus of between about 100 Pa and about 10,000 Pa; and
provided that at least one of the first reactive group and the second reactive group is a reactive thiol group.

In a first variation of the third facet, the at least one linker component comprises at least one polymer component. In such embodiments, provided herein is a hydrogel biomaterial wherein the hydrogel biomaterial is prepared by a polymerization reaction of an aqueous mixture comprising:

at least one polymer component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial has a storage modulus of between about 100 Pa and about 10,000 Pa; and
provided that at least one of the first reactive group and the second reactive group is a reactive thiol group.

In a first aspect of the first variation of the third facet, the first reactive group is a reactive ene group and the second reactive group is a reactive thiol group. In this first aspect of the first variation of the third facet, provided is a hydrogel biomaterial wherein the hydrogel biomaterial is prepared by a polymerization reaction of an aqueous mixture comprising:

at least one polymer component comprising reactive ene groups;
at least one protease-cleavable component comprising reactive thiol groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the first aspect of the first variation of the third facet, the at least one polymer component comprises a linear polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of about 3 kDa, about 6 kDa, and about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

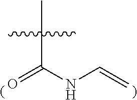 [SMILES: *C(=O)NC=C]

and norborn-2-en-5-yl

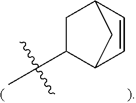 [SMILES: *C1CC2C=CC1C2]

In some embodiments, wherein the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of about 3 kDa, about 6 kDa, and about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the first aspect of the first variation of the third facet, the at least one polymer component comprises a branched polymer with reactive ene groups. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

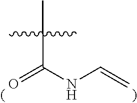 [SMILES: *C(=O)NC=C]

and norborn-2-en-5-yl

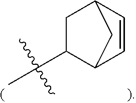 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 3-arm poly(ethylene glycol) moiety and three terminal norborn-2-en-5-yl groups (one at each terminus). In some embodiments, the 3-arm poly(ethylene glycol) moiety has a molecular weight of between about 1 kDa and about 40 kDa. In some embodiments, the 3-arm poly(ethylene glycol) moiety has a molecular weight of about 15 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the first aspect of the first variation of the third facet, the at least one polymer component comprises a linear polymer with reactive ene groups and a branched polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of 3 kDa, 6 kDa, and 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

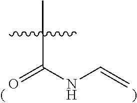 [SMILES: *C(=O)NC=C]

and norborn-2-en-5-yl

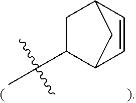 [SMILES: *C1CC2C=CC1C2]

In some embodiments, the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of 3 kDa, 6 kDa, and 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

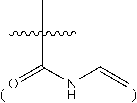 [SMILES: *C(=O)NC=C]

and norborn-2-en-5-yl

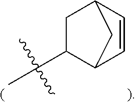 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of about 30 kDa.

In some embodiments of the first aspect of the first variation of the third facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the first aspect of the first variation of the third facet, the hydrogel biomaterial is prepared by the free radical mediated polymerization reaction of a mixture comprising:

0-50% wt./vol. of a linear PEG with terminal norbornene groups, wherein the linear PEG has an average molecular weight of 6 kDa;

0.1-50% wt. of a branched polymer component comprising a multiple armed PEG moiety comprising n arms with terminal norbornene groups, wherein the PEG moiety has an average molecular weight of 20 kDa, and n is 3, 4, 6 or 8;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the first aspect of the first variation of the third facet, the hydrogel biomaterial is prepared by the free radical mediated polymerization reaction of a mixture comprising:

0-50% wt./vol. of a linear PEG with terminal norbornene groups, wherein the linear PEG has an average molecular weight of 6 kDa;

0.1-50% wt./vol. of a 4-armed PEG with terminal norbornene groups having an average molecular weight of 20 kDa;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the first aspect of the first variation of the third facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the first aspect of the first variation of the third facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the first aspect of the first variation of the third facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the first aspect of the first variation of the third facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the first aspect of the first variation of the third facet, the molar ratio of the linear PEG to the IMP-cleavable peptide is between 0 and infinity. In some embodiments, the molar ratio of the linear PEG to the MP-cleavable peptide is between about 20:1 and about 1:1. In some embodiments, the molar ratio of the linear PEG to the IMP-cleavable peptide is between about 2:1 and about 1:1. In some embodiments, the molar ratio of the linear PEG to the IMP-cleavable peptide is about 5:4.

In some embodiments of the first aspect of the first variation of the third facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the first aspect of the first variation of the third facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second aspect of the first variation of the third facet, the first reactive group is a reactive thiol group and the second reactive group is a reactive ene group. In this second aspect of the third facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive ene groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the second aspect of the first variation of the third facet, the at least one polymer component comprises a linear polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of about 3 kDa, about 6 kDa, and about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the second aspect of the first variation of the third facet, the at least one polymer component comprises a branched polymer with reactive thiol groups. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 3-arm poly(ethylene glycol) moiety and three terminal thiol groups. In some embodiments, the 3-arm poly(ethylene glycol) moiety has a molecular weight of between about 1 kDa and about 40 kDa. In some embodiments, the 4-arm poly(ethylene glycol) moiety has a molecular weight of about 15 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the second aspect of the first variation of the third facet, the at least one polymer component comprises a linear polymer with reactive thiol groups and a branched polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with reactive thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of 3 kDa, 6 kDa, and 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive thiol groups of the branched polymer are terminal reactive thiol groups. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of about 30 kDa.

In some embodiments of the second aspect of the first variation of the third facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

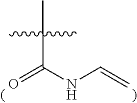 [SMILES: *C(=O)NC=C]

and norborn-2-en-5-yl

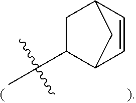 [SMILES: *C1CC2C=CC1C2]

In some embodiments of the second aspect of the first variation of the third facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-6% wt./vol. of a IMP-cleavable peptide comprising a reactive ene group;

0-6% wt./vol. of a plasmin-cleavable peptide comprising a reactive ene group;

0-10% wt./vol. of a fibrinogen component derivatized at cysteine residues with 3.5 kDa linear PEG with terminal norbornene groups; and

water.

In some embodiments of the second aspect of the first variation of the third facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt./vol. of a 4-armed PEG with terminal thiol groups having an average molecular weight of 20 kDa;

0-6% wt./vol. of a IMP-cleavable peptide comprising a reactive ene group;

0-6% wt./vol. of a plasmin-cleavable peptide comprising a reactive ene group;

0-10% wt./vol. of a fibrinogen component derivatized at cysteine residues with 3.5 kDa linear PEG with terminal norbornene groups; and

water.

In some embodiments of the second aspect of the first variation of the third facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the second aspect of the first variation of the third facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the second aspect of the first variation of the third facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the second aspect of the first variation of the third facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the second aspect of the first variation of the third facet, the molar ratio of the linear PEG to the IMP-cleavable peptide is between 0 and infinity. In some embodiments, the molar ratio of the linear PEG to the IMP-cleavable peptide is between about 20:1 and about 1:1. In some embodiments, the molar ratio of the linear PEG to the IMP-cleavable peptide is between about 2:1 and about 1:1. In some embodiments, the molar ratio of the linear PEG to the IMP-cleavable peptide is about 5:4.

In some embodiments of the second aspect of the first variation of the third facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the second aspect of the first variation of the third facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of the second aspect of the first variation of the third facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In a third aspect of the first variation of the third facet, the first reactive group is a reactive thiol group and the second reactive group is a reactive thiol group. In this third aspect of the first variation of the third facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive thiol groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the third aspect of the first variation of the third facet, the at least one polymer component comprises a linear polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of about 3 kDa, about 6 kDa, and about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the third aspect of the first variation of the third facet, the at least one polymer component comprises a branched polymer with reactive thiol groups. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 3-arm poly(ethylene glycol) moiety and three terminal thiol groups. In some embodiments, the 3-arm poly(ethylene glycol) moiety has a molecular weight of between about 1 kDa and about 40 kDa. In some embodiments, the 4-arm poly(ethylene glycol) moiety has a molecular weight of about 15 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the third aspect of the first variation of the third facet, the at least one polymer component comprises a linear polymer with reactive thiol groups and a branched polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with reactive thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of 3 kDa, 6 kDa, and 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive thiol groups of the branched polymer are terminal reactive thiol groups. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of about 30 kDa.

In some embodiments of the third aspect of the first variation of the third facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the third aspect of the first variation of the third facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the third aspect of the first variation of the third facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt./vol. of a 4-armed PEG with terminal thiol groups having an average molecular weight of 20 kDa;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the third aspect of the first variation of the third facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the third aspect of the first variation of the third facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the third aspect of the first variation of the third facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the third aspect of the first variation of the third facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the third aspect of the first variation of the third facet, the molar ratio of the linear PEG to the IMP-cleavable peptide is between 0 and infinity. In some embodiments, the molar ratio of the linear PEG to the MP-cleavable peptide is between about 20:1 and about 1:1. In some embodiments, the molar ratio of the linear PEG to the IMP-cleavable peptide is between about 2:1 and about 1:1. In some embodiments, the molar ratio of the linear PEG to the IMP-cleavable peptide is about 5:4.

In some embodiments of the third aspect of the first variation of the third facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the third aspect of the first variation of the third facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second variation of the third facet, the at least one linker component comprises at least one multivalent linker component. In such embodiments, provided herein is a hydrogel biomaterial wherein the hydrogel biomaterial is prepared by a polymerization reaction of an aqueous mixture comprising:

at least one multivalent linker component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial has a storage modulus of between about 100 Pa and about 10,000 Pa; and
provided that at least one of the first reactive group and the second reactive group is a reactive thiol group.

In a first aspect of the second variation of the third facet, the first reactive group is a reactive thiol group and the second reactive group is a reactive ene group. In this first aspect of the second variation of the third facet, provided is a hydrogel biomaterial wherein the hydrogel biomaterial is prepared by a polymerization reaction of an aqueous mixture comprising:

at least one multivalent linker comprising reactive thiol groups;
at least one protease-cleavable component comprising reactive ene groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the first aspect of the second variation of the third facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

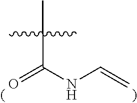 [SMILES: *C(=O)NC=C]

and norborn-2-en-5-yl

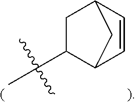 [SMILES: *C1CC2C=CC1C2]

In some embodiments of the first aspect of the second variation of the third facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the first aspect of the second variation of the third facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second aspect of the second variation of the third facet, the first reactive group is a reactive thiol group and the second reactive group is a reactive thiol group. In this second aspect of the second variation of the third facet, provided is a hydrogel biomaterial wherein the hydrogel biomaterial is prepared by a polymerization reaction of an aqueous mixture comprising:

at least one multivalent linker component comprising reactive thiol groups;
at least one protease-cleavable component comprising reactive thiol groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the second aspect of the second variation of the third facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the second aspect of the second variation of the third facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the second aspect of the second variation of the third facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of any of the preceding aspects of the second variation of the third facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of any of the preceding aspects of the second variation of the first facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of the preceding aspects and variations of the third facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

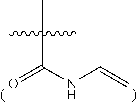 [SMILES: *C(=O)NC=C]

and norborn-2-en-5-yl

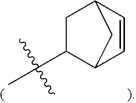 [SMILES: *C1CC2C=CC1C2]

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of the preceding aspects of the third facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the preceding aspects of the third facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the third facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the third facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the third facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments and aspects of the third facet, the hydrogel biomaterial does not substantially swell when exposed to a protease capable of cleaving the protease-cleavable component.

In some variations of the preceding embodiments and aspects of the third facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the third facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the third facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the third facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the third facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the third facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the third facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the third facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the third facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the third facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a fourth facet, provided herein is a hydrogel biomaterial wherein the hydrogel biomaterial is prepared by a polymerization reaction of an aqueous mixture comprising:

at least one linker component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial does not substantially swell when exposed to a protease capable of cleaving the protease-cleavable component;
and
provided that at least one of the first reactive group and the second reactive group is a reactive thiol group.

In a first variation of the fourth facet, the at least one linker component comprises at least one polymer component. In such embodiments, provided herein is a hydrogel biomaterial wherein the hydrogel biomaterial is prepared by a polymerization reaction of an aqueous mixture comprising:

at least one polymer component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial does not substantially swell when exposed to a protease capable of cleaving the protease-cleavable component;
and
provided that at least one of the first reactive group and the second reactive group is a reactive thiol group.

In a first aspect of the first variation of the fourth facet, the first reactive group is a reactive ene group and the second reactive group is a reactive thiol group. In this first aspect of the first variation of the fourth facet, provided is a hydrogel biomaterial wherein the hydrogel biomaterial is prepared by a polymerization reaction of an aqueous mixture comprising:

at least one polymer component comprising reactive ene groups;
at least one protease-cleavable component comprising reactive thiol groups;
a fibrinogen component derivatized with a plurality of polymeric linkers with reactive ene groups;
and
wherein the hydrogel biomaterial does not substantially swell when exposed to a protease capable of cleaving the protease-cleavable component.

In some embodiments of the first aspect of the first variation of the fourth facet, the at least one polymer component comprises a linear polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of about 3 kDa, about 6 kDa, and about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

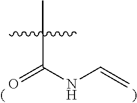 [SMILES: *C(=O)NC=C]

and norborn-2-en-5-yl

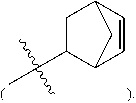 [SMILES: *C1CC2C=CC1C2]

In some embodiments, wherein the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of about 3 kDa, about 6 kDa, and about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the first aspect of the first variation of the fourth facet, the at least one polymer component comprises a branched polymer with reactive ene groups. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

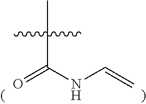 [SMILES: *C(=O)NC=C]

and norborn-2-en-5-yl

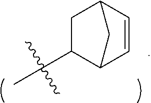

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 3-arm poly(ethylene glycol) moiety and three terminal norborn-2-en-5-yl groups (one at each terminus). In some embodiments, the 3-arm poly(ethylene glycol) moiety has a molecular weight of between about 1 kDa and about 40 kDa. In some embodiments, the 3-arm poly(ethylene glycol) moiety has a molecular weight of about 15 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the first aspect of the first variation of the fourth facet, the at least one polymer component comprises a linear polymer with reactive ene groups and a branched polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of 3 kDa, 6 kDa, and 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

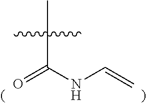 [SMILES: *C(=O)NC=C]

and norborn-2-en-5-yl

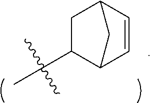

In some embodiments, the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of 3 kDa, 6 kDa, and 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

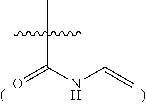 [SMILES: *C(=O)NC=C]

and norborn-2-en-5-yl

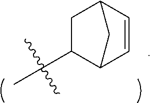

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of about 30 kDa.

In some embodiments of the first aspect of the first variation of the fourth facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the first aspect of the first variation of the fourth facet, the hydrogel biomaterial is prepared by the free radical mediated polymerization reaction of a mixture comprising:

0-50% wt./vol. of a linear PEG with terminal norbornene groups, wherein the linear PEG has an average molecular weight of 6 kDa;

0.1-50% wt. of a branched polymer component comprising a multiple armed PEG moiety comprising n arms with terminal norbornene groups, wherein the PEG moiety has an average molecular weight of 20 kDa, and n is 3, 4, 6 or 8;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the first aspect of the first variation of the fourth facet, the hydrogel biomaterial is prepared by the free radical mediated polymerization reaction of a mixture comprising:

0-50% wt./vol. of a linear PEG with terminal norbornene groups, wherein the linear PEG has an average molecular weight of 6 kDa;

0.1-50% wt./vol. of a 4-armed PEG with terminal norbornene groups having an average molecular weight of 20 kDa;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the first aspect of the first variation of the fourth facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the first aspect of the first variation of the fourth facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the first aspect of the first variation of the fourth facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the first aspect of the first variation of the fourth facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the first aspect of the first variation of the fourth facet, the molar ratio of the linear PEG to the IMP-cleavable peptide is between 0 and infinity. In some embodiments, the molar ratio of the linear PEG to the IMP-cleavable peptide is between about 20:1 and about 1:1. In some embodiments, the molar ratio of the linear PEG to the IMP-cleavable peptide is between about 2:1 and about 1:1. In some embodiments, the molar ratio of the linear PEG to the IMP-cleavable peptide is about 5:4.

In some embodiments of the first aspect of the first variation of the fourth facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the first aspect of the first variation of the fourth facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second aspect of the first variation of the fourth facet, the first reactive group is a reactive thiol group and the second reactive group is a reactive ene group. In this second aspect of the first variation of the fourth facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive ene groups;
a fibrinogen component derivatized with a plurality of polymeric linkers with reactive ene groups;
and
wherein the hydrogel biomaterial does not substantially swell when exposed to a protease capable of cleaving the protease-cleavable component.

In some embodiments of the second aspect of the first variation of the fourth facet, the at least one polymer component comprises a linear polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of about 3 kDa, about 6 kDa, and about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the second aspect of the first variation of the fourth facet, the at least one polymer component comprises a branched polymer with reactive thiol groups. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 3-arm poly(ethylene glycol) moiety and three terminal thiol groups. In some embodiments, the 3-arm poly(ethylene glycol) moiety has a molecular weight of between about 1 kDa and about 40 kDa. In some embodiments, the 4-arm poly(ethylene glycol) moiety has a molecular weight of about 15 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the second aspect of the first variation of the fourth facet, the at least one polymer component comprises a linear polymer with reactive thiol groups and a branched polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with reactive thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of 3 kDa, 6 kDa, and 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive thiol groups of the branched polymer are terminal reactive thiol groups. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of about 30 kDa.

In some embodiments of the second aspect of the first variation of the fourth facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

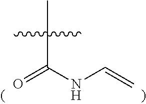 [SMILES: *C(=O)NC=C]

and norborn-2-en-5-yl

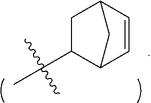

In some embodiments of the second aspect of the first variation of the fourth facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-6% wt./vol. of a IMP-cleavable peptide comprising a reactive ene group;

0-6% wt./vol. of a plasmin-cleavable peptide comprising a reactive ene group;

0-10% wt./vol. of a fibrinogen component derivatized at cysteine residues with 3.5 kDa linear PEG with terminal norbornene groups; and

water.

In some embodiments of the second aspect of the first variation of the fourth facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt./vol. of a 4-armed PEG with terminal thiol groups having an average molecular weight of 20 kDa;

0-6% wt./vol. of a IMP-cleavable peptide comprising a reactive ene group;

0-6% wt./vol. of a plasmin-cleavable peptide comprising a reactive ene group;

0-10% wt./vol. of a fibrinogen component derivatized at cysteine residues with 3.5 kDa linear PEG with terminal norbornene groups; and

water.

In some embodiments of the second aspect of the first variation of the fourth facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the second aspect of the first variation of the fourth facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the second aspect of the first variation of the fourth facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the second aspect of the first variation of the fourth facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the second aspect of the first variation of the fourth facet, the molar ratio of the linear PEG to the MMP-cleavable peptide is between 0 and infinity. In some embodiments, the molar ratio of the linear PEG to the MMP-cleavable peptide is between about 20:1 and about 1:1. In some embodiments, the molar ratio of the linear PEG to the MMP-cleavable peptide is between about 2:1 and about 1:1. In some embodiments, the molar ratio of the linear PEG to the MMP-cleavable peptide is about 5:4.

In some embodiments of the second aspect of the first variation of the fourth facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the second aspect of the first variation of the fourth facet, the overall stoichiometric ratio of the norbornene moieties to the free thiol moieties prior to polymerization is selected such that the average branch order of either the norbornene bearing entities or free thiol bearing entities prior to polymerization is greater than 2.

In a third aspect of the first variation of the fourth facet, the first reactive group is a reactive thiol group and the second reactive group is a reactive thiol group. In this third aspect of the first variation of the fourth facet, provided is a hydrogel biomaterial wherein the hydrogel biomaterial is prepared by a polymerization reaction of an aqueous mixture comprising:

at least one polymer component comprising reactive thiol groups;
at least one protease-cleavable component comprising reactive thiol groups;
a fibrinogen component derivatized with a plurality of polymeric linkers with reactive ene groups;
and
wherein the hydrogel biomaterial does not substantially swell when exposed to a protease capable of cleaving the protease-cleavable component.

In some embodiments of the third aspect of the first variation of the fourth facet, the at least one polymer component comprises a linear polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of about 3 kDa, about 6 kDa, and about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the third aspect of the first variation of the fourth facet, the at least one polymer component comprises a branched polymer with reactive thiol groups. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 3-arm poly(ethylene glycol) moiety and three terminal thiol groups. In some embodiments, the 3-arm poly(ethylene glycol) moiety has a molecular weight of between about 1 kDa and about 40 kDa. In some embodiments, the 4-arm poly(ethylene glycol) moiety has a molecular weight of about 15 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the third aspect of the first variation of the fourth facet, the at least one polymer component comprises a linear polymer with reactive thiol groups and a branched polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with reactive thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of 3 kDa, 6 kDa, and 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive thiol groups of the branched polymer are terminal reactive thiol groups. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of about 30 kDa.

In some embodiments of the third aspect of the first variation of the fourth facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MM P) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MM P) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MM P) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the third aspect of the first variation of the fourth facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the third aspect of the first variation of the fourth facet, the hydrogel biomaterial is prepared by the free radical mediated polymerization reaction of a mixture comprising:

0-50% wt./vol. of a linear PEG with terminal thiol groups, wherein the linear PEG has an average molecular weight of 6 kDa;

0.1-50% wt./vol. of a 4-armed PEG with terminal thiol groups having an average molecular weight of 20 kDa;

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the third aspect of the first variation of the fourth facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the third aspect of the first variation of the fourth facet, the hydrogel biomaterial comprises between about 0.1% wt./vol. and 5% wt./vol. of the linear PEG. In some embodiments, the hydrogel biomaterial comprises between about 0.5% wt./vol. and 2% wt./vol. of the linear PEG. In some embodiments, the hydrogel biomaterial comprises about 1% wt./vol. of the linear PEG.

In some embodiments of the third aspect of the first variation of the fourth facet, the hydrogel biomaterial comprises between about 0.1% wt./vol. and 5% wt./vol. of the branched PEG. In some embodiments, the hydrogel biomaterial comprises between about 0.5% wt./vol. and 2% wt./vol. of the branched PEG. In some embodiments, the hydrogel biomaterial comprises about 1% wt./vol. of the branched PEG.

In some embodiments of the third aspect of the first variation of the fourth facet, the molar ratio of the linear PEG to the 4-armed PEG is between 0 and infinity. In some embodiments, the molar ratio of the linear PEG to the 4-armed PEG is between about 20:1 and about 1:1. In some embodiments, the molar ratio of the linear PEG to the 4-armed PEG is between about 4:1 and about 2:1. In some embodiments, the molar ratio of the linear PEG to the 4-armed PEG is about 20:6.

In some embodiments of the third aspect of the first variation of the fourth facet, the molar ratio of the linear PEG to the IMP-cleavable peptide is between 0 and infinity. In some embodiments, the molar ratio of the linear PEG to the IMP-cleavable peptide is between about 20:1 and about 1:1. In some embodiments, the molar ratio of the linear PEG to the IMP-cleavable peptide is between about 2:1 and about 1:1. In some embodiments, the molar ratio of the linear PEG to the IMP-cleavable peptide is about 5:4.

In some embodiments of the third aspect of the first variation of the fourth facet, the molar ratio of the linear PEG to the plasmin-cleavable peptide is between 0 and infinity. In some embodiments, the molar ratio of the linear PEG to the plasmin-cleavable peptide is between about 20:1 and about 1:1. In some embodiments, the molar ratio of the linear PEG to the plasmin-cleavable peptide is between about 2:1 and about 1:1. In some embodiments, the molar ratio of the linear PEG to the plasmin-cleavable peptide is about 5:4.

In some embodiments of the third aspect of the first variation of the fourth facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second variation of the fourth facet, the at least one linker component comprises at least one multivalent linker component. In such embodiments, provided herein is a hydrogel biomaterial wherein the hydrogel biomaterial is prepared by a polymerization reaction of an aqueous mixture comprising:

at least one multivalent linker component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial does not substantially swell when exposed to a protease capable of cleaving the protease-cleavable component;
and
provided that at least one of the first reactive group and the second reactive group is a reactive thiol group.

In a first aspect of the second variation of the fourth facet, the first reactive group is a reactive thiol group and the second reactive group is a reactive ene group. In this first aspect of the second variation of the fourth facet, provided is a hydrogel biomaterial wherein the hydrogel biomaterial is prepared by a polymerization reaction of an aqueous mixture comprising:

at least one multivalent linker component comprising reactive thiol groups;
at least one protease-cleavable component comprising reactive ene groups;
a fibrinogen component derivatized with a plurality of polymeric linkers with reactive ene groups;
and
wherein the hydrogel biomaterial does not substantially swell when exposed to a protease capable of cleaving the protease-cleavable component.

In some embodiments of the first aspect of the second variation of the fourth facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

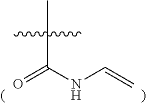 [SMILES: *C(=O)NC=C]

and norborn-2-en-5-yl

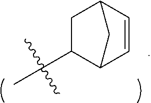

In some embodiments of the first aspect of the second variation of the fourth facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the first aspect of the second variation of the fourth facet, the overall stoichiometric ratio of the norbornene moieties to the free thiol moieties prior to polymerization is selected such that the average branch order of either the norbornene bearing entities or free thiol bearing entities prior to polymerization is greater than 2.

In a second aspect of the second variation of the fourth facet, the first reactive group is a reactive thiol group and the second reactive group is a reactive thiol group. In this second aspect of the second variation of the fourth facet, provided is a hydrogel biomaterial wherein the hydrogel biomaterial is prepared by a polymerization reaction of an aqueous mixture comprising:

at least one multivalent linker component comprising reactive thiol groups;
at least one protease-cleavable component comprising reactive thiol groups;
a fibrinogen component derivatized with a plurality of polymeric linkers with reactive ene groups;
and
wherein the hydrogel biomaterial does not substantially swell when exposed to a protease capable of cleaving the protease-cleavable component.

In some embodiments of the second aspect of the second variation of the fourth facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the second aspect of the second variation of the fourth facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the second aspect of the second variation of the fourth facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of any of the preceding aspects of the second variation of the fourth facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of any of the preceding aspects of the second variation of the first facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of the preceding aspects and variations of the fourth facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

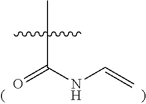 [SMILES: *C(=O)NC=C]

and norborn-2-en-5-yl

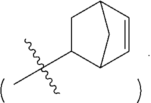

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of the preceding aspects and variations of the fourth facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the preceding aspects of the fourth facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the fourth facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the fourth facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the fourth facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments and aspects of the fourth facet, the hydrogel biomaterial does not substantially swell when exposed to a protease capable of cleaving the protease-cleavable component.

In some variations of the preceding embodiments and aspects of the fourth facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the fourth facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the fourth facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the fourth facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the fourth facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the fourth facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the fourth facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the fourth facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the fourth facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the fourth facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a fifth facet, provided herein is a hydrogel biomaterial comprising

at least one protease-cleavable component; and

a fibrinogen component derivatized with a plurality of polymeric linkers;

wherein at least a portion of the at least one protease-cleavable component and at least a portion of the fibrinogen component are crosslinked via a plurality of crosslink units each independently comprising a carbon-sulfur covalent bond.

In some embodiments, the crosslink units of the plurality of crosslink units are selected from the group consisting of

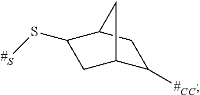

wherein #s and #cc represent attachment points to the at least one protease-cleavable component or a polymeric linker of the fibrinogen component. In some embodiments, all crosslink units of the plurality of crosslink units are identical. In some embodiments, all crosslink units of the plurality of crosslink units are

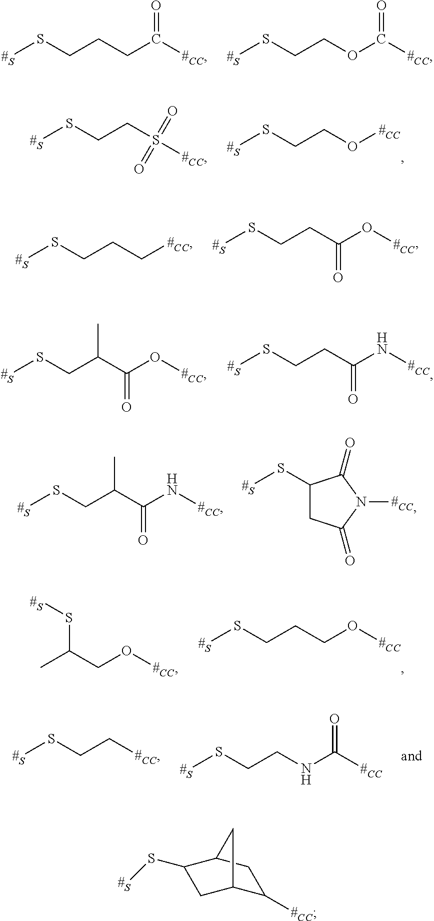

wherein #s and #cc represent attachment points to the at least one protease-cleavable component or a polymeric linker of the fibrinogen component.

In a first aspect, at least a portion of the fibrinogen component is connected to at least a portion of the at least one protease-cleavable component via a first crosslink unit. In some embodiments of the first aspect, the fibrinogen component is connected to the at least one protease-cleavable component via a first crosslink unit.

In a first variation of the first aspect, the first crosslink unit is selected from the group consisting of

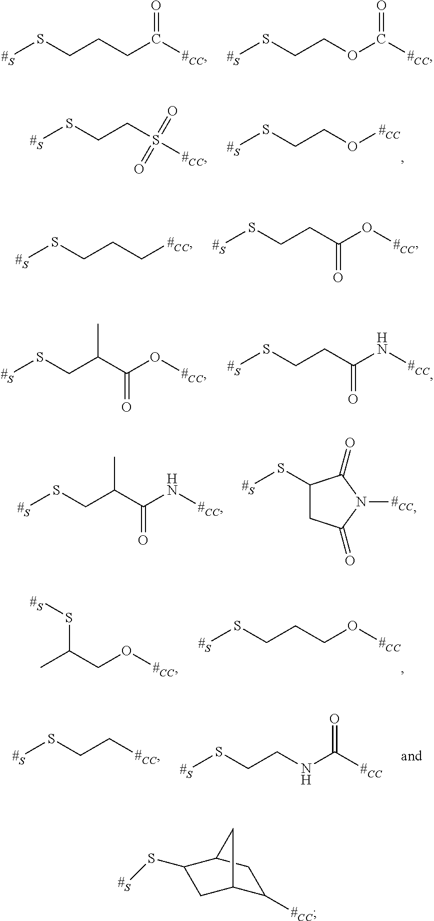

wherein #s represents the attachment point to the at least one protease-cleavable component and #cc represents the attachment point to the fibrinogen component. In some embodiments of the first variation of the first aspect, the first crosslink unit is

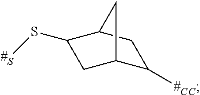

wherein #s represents the attachment point to the at least one protease-cleavable component and #cc represents the attachment point to the fibrinogen component.

In some embodiments of any of the preceding aspects of the fifth facet, the at least one protease-cleavable component is a synthetic peptide. In some embodiments, each of the at least one protease-cleavable component is a peptide having a sequence of between 3 and 25 amino acids. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2.

It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers. For further non-limiting exemplary purposes, it is understood that additional suitable protease-cleavable components may be prepared by modifying the amino acids sequences described above (SEQ ID NOs: 1-19) via the addition of flanking lysine residues which have been modified on the lysine's side chain. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877. In some embodiments, each of the at least one protease-cleavable component is independently of each other a matrix metalloproteinase (MMP) cleavable component or a plasmin cleavable component. In some embodiments, the hydrogel biomaterial comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the hydrogel biomaterial comprises two protease-cleavable components. In some embodiments, the hydrogel biomaterial comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of any of the preceding aspects of the fifth facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, each individual polymeric linker of the plurality of polymeric linkers comprises a poly(ethylene glycol) moiety, wherein the poly(ethylene glycol) moiety has a molecular weight of between about 1 kDa and 40 kDa. In some embodiments, each individual polymeric linker of the plurality of polymeric linkers comprises a poly(ethylene glycol) moiety, wherein the poly(ethylene glycol) moiety has a molecular weight selected from the group consisting of 3.5 kDa, 6 kDa, and 10 kDa. In some embodiments, each individual polymeric linker of the plurality of polymeric linkers comprises a poly(ethylene glycol) moiety, wherein the poly(ethylene glycol) moiety has a molecular weight of about 3.5 kDa. In some embodiments, each individual polymeric linker of the plurality of polymeric linkers comprises a poly(ethylene glycol) moiety, wherein the poly(ethylene glycol) moiety has a molecular weight of about 6 kDa. In some embodiments, each individual polymeric linker of the plurality of polymeric linkers comprises a poly(ethylene glycol) moiety, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of any of the preceding aspects of the fifth facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of any of the preceding aspects of the fifth facet, the hydrogel biomaterial comprises:

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the fifth facet, the hydrogel biomaterial comprises:

0-12% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the fifth facet, the hydrogel biomaterial comprises:

0-6% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the fifth facet, the hydrogel biomaterial comprises:

0-12% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the fifth facet, the hydrogel biomaterial comprises:

0-6% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments, the hydrogel microparticle comprises between about 0.25% wt. and about 5% wt. of the fibrinogen component. In some embodiments, the hydrogel biomaterial comprises about 0.75% wt. of the fibrinogen component. In some embodiments, the hydrogel biomaterial comprises about 1.50% wt. of the fibrinogen component. In some embodiments, the hydrogel biomaterial comprises about 3.00% wt. of the fibrinogen component.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the fifth facet, the hydrogel microparticle has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the fifth facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the fifth facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments and aspects of the fifth facet, the hydrogel biomaterial does not substantially swell when exposed to a protease capable of cleaving the protease-cleavable component.

In some variations of the preceding embodiments and aspects of the fifth facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the fifth facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the fifth facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the fifth facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the fifth facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the fifth facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the fifth facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the fifth facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the fifth facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the fifth facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a sixth facet, provided herein is a hydrogel biomaterial wherein the hydrogel biomaterial is prepared by a polymerization reaction of a mixture comprising:

at least one protease-cleavable component comprising reactive thiol groups;
a fibrinogen component derivatized with a plurality of polymeric linkers with reactive ene groups, wherein the fibrinogen component does not possess the clotting activity of a native fibrinogen molecule.

In some embodiments of the sixth facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the sixth facet, the hydrogel biomaterial is prepared by the free radical mediated polymerization reaction of a mixture comprising:

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the sixth facet, the hydrogel biomaterial comprises:

0-12% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water.

In some embodiments of the sixth facet, the hydrogel biomaterial comprises:

0-6% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water.

In some embodiments of the sixth facet, the hydrogel biomaterial comprises:

0-12% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of the sixth facet, the hydrogel biomaterial comprises:

0-6% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of the sixth facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the sixth facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of the sixth facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the sixth facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments of the sixth facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the sixth facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments of the sixth facet, the hydrogel microparticle has a storage modulus of between about 100 Pa and about 10,000 Pa. In some embodiments, the hydrogel microparticle has a storage modulus of between about 100 Pa and about 5,000 Pa. In some embodiments, the hydrogel microparticle has a storage modulus of between about 100 Pa and about 2,500 Pa. In some embodiments, the hydrogel microparticle has a storage modulus of between about 100 Pa and about 2,000 Pa. In some embodiments, the hydrogel microparticle has a storage modulus of between about 100 Pa and about 1,500 Pa. In some embodiments, the hydrogel microparticle has a storage modulus of between about 100 Pa and about 1,000 Pa. In some embodiments, the hydrogel microparticle has a storage modulus of between about 100 Pa and about 750 Pa. In some embodiments, the hydrogel microparticle has a storage modulus of between about 100 Pa and about 500 Pa. In some embodiments, the hydrogel microparticle has a storage modulus of between about 100 Pa and about 250 Pa. In some embodiments, the hydrogel microparticle has a storage modulus of between about 250 Pa and about 10,000 Pa. In some embodiments, the hydrogel microparticle has a storage modulus of between about 250 Pa and about 5,000 Pa. In some embodiments, the hydrogel microparticle has a storage modulus of between about 250 Pa and about 2,500 Pa. In some embodiments, the hydrogel microparticle has a storage modulus of between about 250 Pa and about 2,000 Pa. In some embodiments, the hydrogel microparticle has a storage modulus of between about 250 Pa and about 1,500 Pa. In some embodiments, the hydrogel microparticle has a storage modulus of between about 250 Pa and about 1,000 Pa. In some embodiments, the hydrogel microparticle has a storage modulus of between about 250 Pa and about 750 Pa. In some embodiments, the hydrogel microparticle has a storage modulus of between about 250 Pa and about 500 Pa. In some embodiments, the hydrogel microparticle has a storage modulus of between about 500 Pa and about 10,000 Pa. In some embodiments, the hydrogel microparticle has a storage modulus of between about 500 Pa and about 5,000 Pa. In some embodiments, the hydrogel microparticle has a storage modulus of between about 500 Pa and about 2,500 Pa. In some embodiments, the hydrogel microparticle has a storage modulus of between about 500 Pa and about 2,000 Pa. In some embodiments, the hydrogel microparticle has a storage modulus of between about 500 Pa and about 1,500 Pa. In some embodiments, the hydrogel microparticle has a storage modulus of between about 500 Pa and about 1,000 Pa. In some embodiments, the hydrogel microparticle has a storage modulus of between about 500 Pa and about 750 Pa. In some embodiments, the hydrogel microparticle has a storage modulus of between about 750 Pa and about 10,000 Pa. In some embodiments, the hydrogel microparticle has a storage modulus of between about 750 Pa and about 5,000 Pa. In some embodiments, the hydrogel microparticle has a storage modulus of between about 750 Pa and about 2,500 Pa. In some embodiments, the hydrogel microparticle has a storage modulus of between about 750 Pa and about 2,000 Pa. In some embodiments, the hydrogel microparticle has a storage modulus of between about 750 Pa and about 1,500 Pa. In some embodiments, the hydrogel microparticle has a storage modulus of between about 750 Pa and about 1,000 Pa. In some embodiments, the hydrogel microparticle has a storage modulus of between about 1,000 Pa and about 10,000 Pa. In some embodiments, the hydrogel microparticle has a storage modulus of between about 1,000 Pa and about 5,000 Pa. In some embodiments, the hydrogel microparticle has a storage modulus of between about 1,000 Pa and about 2,500 Pa. In some embodiments, the hydrogel microparticle has a storage modulus of between about 1,000 Pa and about 2,000 Pa. In some embodiments, the hydrogel microparticle has a storage modulus of between about 1,000 Pa and about 1,500 Pa. In some embodiments, the hydrogel microparticle has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel microparticle has a storage modulus of about 100 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some embodiments of the sixth facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

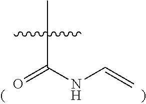 [SMILES: *C(=O)NC=C]

and norborn-2-en-5-yl

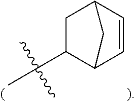 [SMILES: *C1CC2C=CC1C2]

). In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some variations of the preceding embodiments of the sixth facet, the hydrogel biomaterial does not substantially swell when exposed to a protease capable of cleaving the protease-cleavable component.

In some variations of the preceding embodiments of the sixth facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the sixth facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the sixth facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the sixth facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the sixth facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the sixth facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the sixth facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the sixth facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the sixth facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the sixth facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a seventh facet, provided herein is a hydrogel biomaterial wherein the hydrogel biomaterial is prepared by a polymerization reaction of a mixture comprising:

at least one protease-cleavable component comprising reactive thiol groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the seventh facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MMP) cleavable peptide is a peptide of SEQ ID NO: 18, and the plasmin cleavable peptide is a peptide of SEQ ID NO: 4. In some embodiments, the matrix metalloproteinase (MMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the seventh facet, the hydrogel biomaterial is prepared by the free radical mediated polymerization reaction of a mixture comprising:

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the seventh facet, the hydrogel biomaterial comprises:

0-12% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water. In some embodiments of the seventh facet, the hydrogel biomaterial comprises:

0-6% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water.

In some embodiments of the seventh facet, the hydrogel biomaterial comprises:

0-12% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of the seventh facet, the hydrogel biomaterial comprises:

0-6% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of the seventh facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the seventh facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of the seventh facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

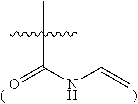 [SMILES: *C(=O)NC=C]

and norborn-2-en-5-yl

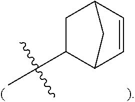 [SMILES: *C1CC2C=CC1C2]

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of the seventh facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the seventh facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the seventh facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the seventh facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the seventh facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments of the seventh facet, the hydrogel biomaterial does not substantially swell when exposed to a protease capable of cleaving the protease-cleavable component.

In some variations of the preceding embodiments of the seventh facet, the hydrogel biomaterial does not substantially swell when exposed to a protease capable of cleaving the protease-cleavable component. In some variations of the preceding embodiments of the seventh facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the seventh facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the seventh facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the seventh facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the seventh facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the seventh facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the seventh facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the seventh facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the seventh facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the seventh facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a eighth facet, provided herein is a hydrogel biomaterial wherein the hydrogel biomaterial is prepared by a polymerization reaction of an aqueous mixture comprising:

at least one protease-cleavable component comprising reactive thiol groups;
a fibrinogen component derivatized with a plurality of polymeric linkers with reactive ene groups;
wherein the hydrogel biomaterial does not substantially swell when exposed to a protease capable of cleaving the protease-cleavable component.

In some embodiments of the eighth facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877.

In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MM P) cleavable peptide is a peptide comprising a sequence selected from the group consisting of SEQ ID NOs: 15-19. In some embodiments, the matrix metalloproteinase (IMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MM P) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (MM P) cleavable peptide is a peptide of SEQ ID NO: 18, and the plasmin cleavable peptide is a peptide of SEQ ID NO: 4. In some embodiments, the matrix metalloproteinase (MM P) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In some embodiments of the eighth facet, the hydrogel biomaterial is prepared by the free radical mediated polymerization reaction of a mixture comprising:

0-6% wt./vol. of a MMP-cleavable peptide K(hC)GPQGIAGQ(hC)K (SEQ ID NO: 19);

0-6% wt./vol. of a plasmin-cleavable peptide (hC)ALKVLKG(hC)G-amide (SEQ ID NO: 7);

0-10% wt./vol. of a fibrinogen component derivatized at cysteine residues with 3.5 kDa linear PEG with terminal norbornene groups; and

water.

In some embodiments of the seventh facet, the hydrogel biomaterial comprises:

0-12% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water.

In some embodiments of the seventh facet, the hydrogel biomaterial comprises:

0-6% wt. of a protease-cleavable component comprising a MMP-cleavable peptide K(hC)GPQGIAGQ(hC)K (SEQ ID NO: 19);

0-10% wt. of a fibrinogen component consisting of a fibrinogen molecule derivatized with a plurality of polymeric linkers each comprising a 3.5 kDa linear PEG; and

water.

In some embodiments of the seventh facet, the hydrogel biomaterial comprises:

0-12% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of the seventh facet, the hydrogel biomaterial comprises:

0-6% wt. of a protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

In some embodiments of the eighth facet, the hydrogel biomaterial comprises between about 0.25% wt./vol. and about 5% wt./vol. of the fibrinogen component. In some embodiments, the hydrogel biomaterial comprises about 0.75% wt./vol. of the fibrinogen component. In some embodiments, the hydrogel biomaterial comprises about 1.50% wt./vol. of the fibrinogen component. In some embodiments, the hydrogel biomaterial comprises about 3.00% wt./vol. of the fibrinogen component.

In some embodiments of the eighth facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of the eighth facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

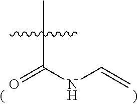 [SMILES: *C(=O)NC=C]

and norborn-2-en-5-yl

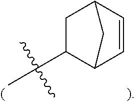 [SMILES: *C1CC2C=CC1C2]

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of the eighth facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the eighth facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the eighth facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the eighth facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the eighth facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments of the eighth facet, the hydrogel biomaterial does not substantially swell when exposed to a protease capable of cleaving the protease-cleavable component.

In some variations of the preceding embodiments of the eighth facet, the hydrogel microparticle does not substantially swell when exposed to a protease capable of cleaving the protease-cleavable component. In some variations of the preceding embodiments and aspects of the eighth facet, the longest dimension of the hydrogel biomaterial increases by less than about twofold when 50% of the protease-cleavable component has been cleaved by a protease capable of cleaving the protease-cleavable component. In some variations of the preceding embodiments and aspects of the eighth facet, the longest dimension of the hydrogel biomaterial increases by less than about 200% when 50% of the protease-cleavable component has been cleaved by a protease capable of cleaving the protease-cleavable component. In some variations of the preceding embodiments and aspects of the eighth facet, the longest dimension of the hydrogel biomaterial increases by less than about 150% when 50% of the protease-cleavable component has been cleaved by a protease capable of cleaving the protease-cleavable component. In some variations of the preceding embodiments and aspects of the eighth facet, the longest dimension of the hydrogel biomaterial increases by less than about 100% when 50% of the protease-cleavable component has been cleaved by a protease capable of cleaving the protease-cleavable component. In some variations of the preceding embodiments and aspects of the eighth facet, the longest dimension of the hydrogel biomaterial increases by less than about 75% when 50% of the protease-cleavable component has been cleaved by a protease capable of cleaving the protease-cleavable component. In some variations of the preceding embodiments and aspects of the eighth facet, the longest dimension of the hydrogel biomaterial increases by less than about 50% when 50% of the protease-cleavable component has been cleaved by a protease capable of cleaving the protease-cleavable component. In some variations of the preceding embodiments and aspects of the eighth facet, the longest dimension of the hydrogel biomaterial increases by less than about 25% when 50% of the protease-cleavable component has been cleaved by a protease capable of cleaving the protease-cleavable component. In some variations of the preceding embodiments and aspects of the eighth facet, the longest dimension of the hydrogel biomaterial increases by less than about 10% when 50% of the protease-cleavable component has been cleaved by a protease capable of cleaving the protease-cleavable component.

In some variations of the preceding embodiments and aspects of the eighth facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the eighth facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a ninth facet, provided herein is a hydrogel biomaterial comprising

at least one linker component; and

a fibrinogen component derivatized with a plurality of polymeric linkers;

wherein at least a portion of the at least one polymer component and at least a portion of the fibrinogen component are crosslinked via a plurality of crosslink units each independently comprising a carbon-sulfur covalent bond.

In a first variation of the ninth facet, the at least one linker component is at least one polymer component. In such embodiments, provided herein is a hydrogel biomaterial comprising

at least one polymer component; and

a fibrinogen component derivatized with a plurality of polymeric linkers;

wherein at least a portion of the at least one polymer component and at least a portion of the fibrinogen component are crosslinked via a plurality of crosslink units each independently comprising a carbon-sulfur covalent bond.

In some embodiments, the crosslink units of the plurality of crosslink units are selected from the group consisting of

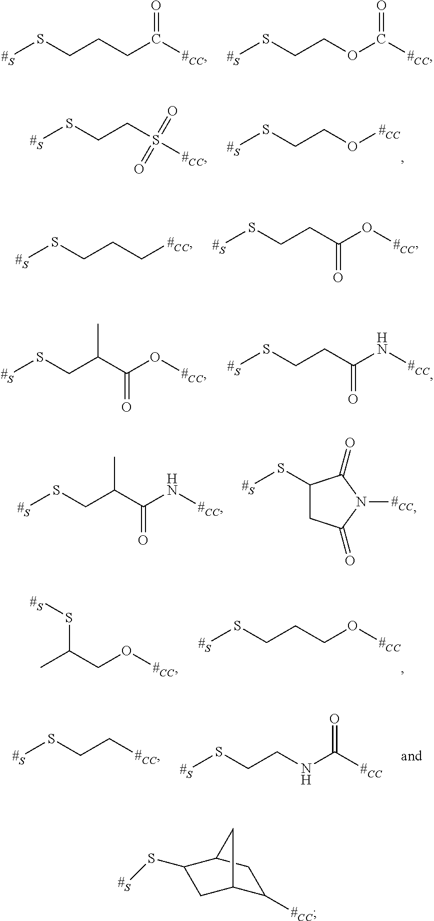

wherein #s and #cc represent attachment points to the at least one polymer component or a polymeric linker of the fibrinogen component. In some embodiments, all crosslink units of the plurality of crosslink units are identical. In some embodiments, all crosslink units of the plurality of crosslink units are

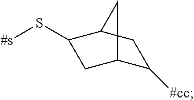

wherein #s and #cc represent attachment points to the at least one polymer component or a polymeric linker of the fibrinogen component.

In a first aspect of the first variation of the ninth facet, at least a portion of the fibrinogen component is connected to at least a portion of the at least one polymer component via a first crosslink unit. In some embodiments of the first aspect of the first variation of the ninth facet, the fibrinogen component is connected to the at least one polymer component via a first crosslink unit.

In a first variation of the first aspect of the first variation of the ninth facet, the first crosslink unit is selected from the group consisting of

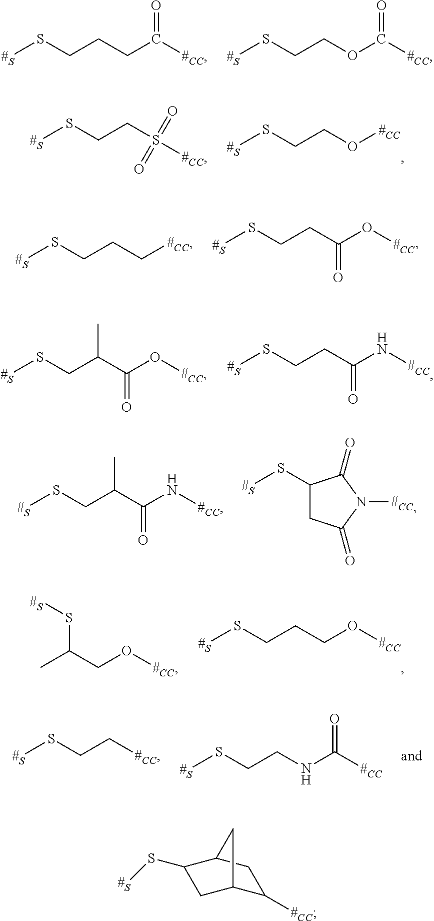

wherein #s represents the attachment point to the at least one polymer component and #cc represents the attachment point to the fibrinogen component. In some embodiments of the first variation of the first aspect of the first variation of the ninth facet, the first crosslink unit is

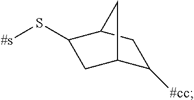

wherein #s represents the attachment point to the at least one polymer component and #cc represents the attachment point to the fibrinogen component.

In some embodiments of any of the preceding aspects of the first variation of the ninth facet, the at least one polymer component comprises a linear polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of about 3 kDa, about 6 kDa, and about 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of any of the preceding aspects of the first variation of the ninth facet, the at least one polymer component comprises a branched polymer component. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of any of the preceding aspects of the first variation of the ninth facet, the at least one polymer component comprises a linear polymer component and a branched polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of 3 kDa, 6 kDa, and 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a poly(ethylene glycol) moiety, wherein the poly(ethylene glycol) moiety has a molecular weight selected from the group consisting of 3 kDa, 6 kDa, and 10 kDa. In some embodiments, the linear polymer component comprises a poly(ethylene glycol) moiety, wherein the poly(ethylene glycol) moiety has a molecular weight of about 10 kDa. In some embodiments, the linear polymer component comprises a poly(ethylene glycol) moiety, wherein the poly(ethylene glycol) moiety has a molecular weight of about 6 kDa. In some embodiments, the linear polymer component comprises a poly(ethylene glycol) moiety, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of about 30 kDa.

In a second variation of the ninth facet, the at least one linker component is at least one multivalent linker component. In such embodiments, provided herein is a hydrogel biomaterial comprising

at least one multivalent linker component; and

a fibrinogen component derivatized with a plurality of polymeric linkers;

wherein at least a portion of the at least one multivalent linker component and at least a portion of the fibrinogen component are crosslinked via a plurality of crosslink units each independently comprising a carbon-sulfur covalent bond.

In some embodiments, the crosslink units of the plurality of crosslink units are selected from the group consisting of

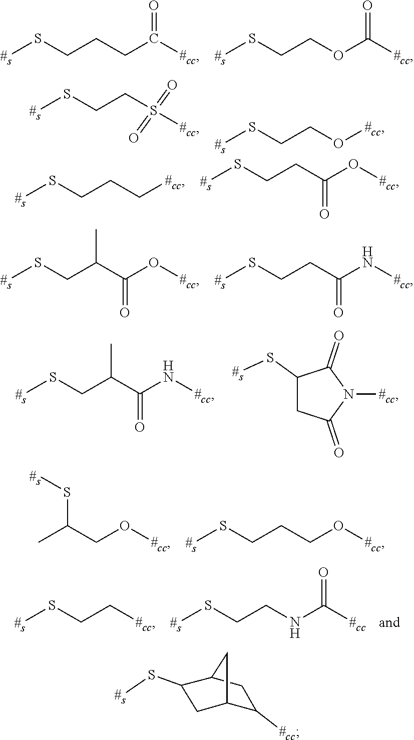

wherein #s and #cc represent attachment points to the at least one multivalent linker component or a polymeric linker of the fibrinogen component. In some embodiments, all crosslink units of the plurality of crosslink units are identical. In some embodiments, all crosslink units of the plurality of crosslink units are

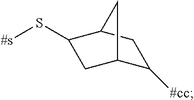

wherein #s and #cc represent attachment points to the at least one multivalent linker component or a polymeric linker of the fibrinogen component.

In a first aspect of the second variation of the ninth facet, at least a portion of the fibrinogen component is connected to at least a portion of the at least one multivalent linker component via a first crosslink unit. In some embodiments of the first aspect of the first variation of the ninth facet, the fibrinogen component is connected to the at least one multivalent linker component via a first crosslink unit.

In a first variation of the first aspect of the second variation of the ninth facet, the first crosslink unit is selected from the group consisting of

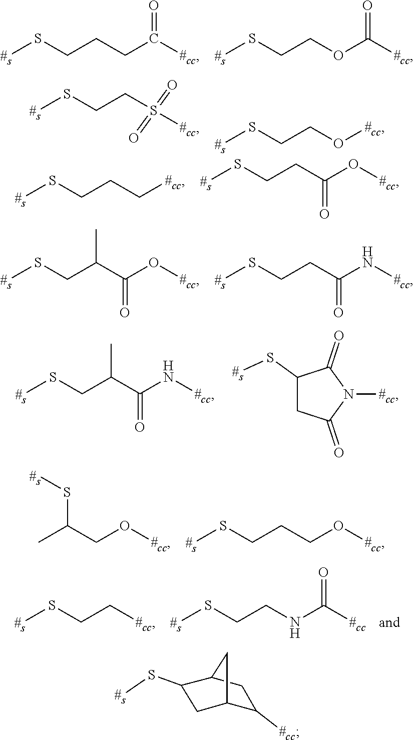

wherein #s represents the attachment point to the at least one multivalent linker component and #cc represents the attachment point to the fibrinogen component. In some embodiments of the first variation of the first aspect of the second variation of the ninth facet, the first crosslink unit is

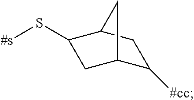

wherein #s represents the attachment point to the at least one multivalent linker component and #cc represents the attachment point to the fibrinogen component.

In some embodiments of the second variation of the ninth facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of the second variation of the first facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of any of the preceding aspects of the ninth facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, each individual polymeric linker of the plurality of polymeric linkers comprises a poly(ethylene glycol) moiety, wherein the poly(ethylene glycol) moiety has a molecular weight of between about 1 kDa and 40 kDa. In some embodiments, each individual polymeric linker of the plurality of polymeric linkers comprises a poly(ethylene glycol) moiety, wherein the poly(ethylene glycol) moiety has a molecular weight selected from the group consisting of 3.5 kDa, 6 kDa, and 10 kDa. In some embodiments, each individual polymeric linker of the plurality of polymeric linkers comprises a poly(ethylene glycol) moiety, wherein the poly(ethylene glycol) moiety has a molecular weight of about 3.5 kDa. In some embodiments, each individual polymeric linker of the plurality of polymeric linkers comprises a poly(ethylene glycol) moiety, wherein the poly(ethylene glycol) moiety has a molecular weight of about 6 kDa. In some embodiments, each individual polymeric linker of the plurality of polymeric linkers comprises a poly(ethylene glycol) moiety, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of any of the preceding aspects of the ninth facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of any of the preceding aspects of the ninth facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the ninth facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a 4-armed PEG moiety, wherein the 4-armed PEG moiety has an average molecular weight of 20 kDa;

0-10% wt. of a fibrinogen component consisting of a fibrinogen molecule derivatized with a plurality of polymeric linkers each comprising a 3.5 kDa linear PEG; and

water.

In some embodiments of any of the preceding aspects of the ninth facet, the hydrogel microparticle comprises between about 0.25% wt. and about 5% wt. of the fibrinogen component. In some embodiments, the hydrogel biomaterial comprises about 0.75% wt. of the fibrinogen component. In some embodiments, the hydrogel biomaterial comprises about 1.50% wt. of the fibrinogen component. In some embodiments, the hydrogel biomaterial comprises about 3.00% wt. of the fibrinogen component.

In some embodiments of any of the preceding aspects of the ninth facet, the hydrogel biomaterial comprises between about 0.1% wt. and 5% wt. of the linear polymer component. In some embodiments, the hydrogel biomaterial comprises between about 0.5% wt. and 2% wt. of the linear polymer component. In some embodiments, the hydrogel biomaterial comprises about 1% wt. of the linear polymer component.

In some embodiments of any of the preceding aspects of the ninth facet, the hydrogel biomaterial comprises between about 0.1% wt. and 5% wt. of the branched polymer component. In some embodiments, the hydrogel biomaterial comprises between about 0.5% wt. and 2% wt. of the branched polymer component. In some embodiments, the hydrogel biomaterial comprises about 1% wt. of the branched polymer component.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the ninth facet, the hydrogel microparticle has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the ninth facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the ninth facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments of the ninth facet, the hydrogel biomaterial does not substantially swell when exposed to a protease capable of cleaving the fibrinogen component.

In some variations of the preceding embodiments and aspects of the ninth facet, the longest dimension of the hydrogel biomaterial increases by less than about twofold when 50% of the fibrinogen component has been cleaved by a protease capable of cleaving the fibrinogen component. In some variations of the preceding embodiments and aspects of the ninth facet, the longest dimension of the hydrogel biomaterial increases by less than about 200% when 50% of the fibrinogen component has been cleaved by a protease capable of cleaving the fibrinogen component. In some variations of the preceding embodiments and aspects of the ninth facet, the longest dimension of the hydrogel biomaterial increases by less than about 150% when 50% of the fibrinogen component has been cleaved by a protease capable of cleaving the fibrinogen component. In some variations of the preceding embodiments and aspects of the ninth facet, the longest dimension of the hydrogel biomaterial increases by less than about 100% when 50% of the fibrinogen component has been cleaved by a protease capable of cleaving the fibrinogen component. In some variations of the preceding embodiments and aspects of the ninth facet, the longest dimension of the hydrogel biomaterial increases by less than about 75% when 50% of the fibrinogen component has been cleaved by a protease capable of cleaving the fibrinogen component. In some variations of the preceding embodiments and aspects of the ninth facet, the longest dimension of the hydrogel biomaterial increases by less than about 50% when 50% of the fibrinogen component has been cleaved by a protease capable of cleaving the fibrinogen component. In some variations of the preceding embodiments and aspects of the ninth facet, the longest dimension of the hydrogel biomaterial increases by less than about 25% when 50% of the fibrinogen component has been cleaved by a protease capable of cleaving the fibrinogen component. In some variations of the preceding embodiments and aspects of the ninth facet, the longest dimension of the hydrogel biomaterial increases by less than about 10% when 50% of the fibrinogen component has been cleaved by a protease capable of cleaving the fibrinogen component.

In some variations of the preceding embodiments and aspects of the ninth facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the ninth facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a tenth facet, provided herein is a hydrogel biomaterial wherein the hydrogel biomaterial is prepared by a polymerization reaction of a mixture comprising:

at least one linker component comprising reactive thiol groups;
a fibrinogen component derivatized with a plurality of polymeric linkers with reactive ene groups, wherein the fibrinogen component does not possess the clotting activity of a native fibrinogen molecule.

In a first variation of the tenth facet, the at least one linker component is at least one polymer component. In such embodiments, provided herein is a hydrogel biomaterial wherein the hydrogel biomaterial is prepared by a polymerization reaction of a mixture comprising:

at least one polymer component comprising reactive thiol groups;
a fibrinogen component derivatized with a plurality of polymeric linkers with reactive ene groups, wherein the fibrinogen component does not possess the clotting activity of a native fibrinogen molecule.

In some embodiments of the first variation of the tenth facet, the at least one polymer component comprises a linear polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of about 3 kDa, about 6 kDa, and about 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the first variation of the tenth facet, the at least one polymer component comprises a branched polymer component. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the first variation of the tenth facet, the at least one polymer component comprises a linear polymer component and a branched polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of 3 kDa, 6 kDa, and 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a poly(ethylene glycol) moiety, wherein the poly(ethylene glycol) moiety has a molecular weight selected from the group consisting of 3 kDa, 6 kDa, and 10 kDa. In some embodiments, the linear polymer component comprises a poly(ethylene glycol) moiety, wherein the poly(ethylene glycol) moiety has a molecular weight of about 10 kDa. In some embodiments, the linear polymer component comprises a poly(ethylene glycol) moiety, wherein the poly(ethylene glycol) moiety has a molecular weight of about 6 kDa. In some embodiments, the linear polymer component comprises a poly(ethylene glycol) moiety, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of about 30 kDa.

In some embodiments of the first variation of the tenth facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-10% wt. of a fibrinogen component consisting of a fibrinogen molecule derivatized with a plurality of polymeric linkers each comprising a 3.5 kDa linear PEG; and

water.

In some embodiments of the first variation of the tenth facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a 4-armed PEG moiety, wherein the 4-armed PEG moiety has an average molecular weight of 20 kDa;

0-10% wt. of a fibrinogen component consisting of a fibrinogen molecule derivatized with a plurality of polymeric linkers each comprising a 3.5 kDa linear PEG; and

water.

In a second variation of the tenth facet, the at least one linker component is at least one multivalent linker component. In such embodiments, provided herein is a hydrogel biomaterial wherein the hydrogel biomaterial is prepared by a polymerization reaction of a mixture comprising:

at least one multivalent linker component comprising reactive thiol groups;
a fibrinogen component derivatized with a plurality of polymeric linkers with reactive ene groups, wherein the fibrinogen component does not possess the clotting activity of a native fibrinogen molecule.

In some embodiments of the second variation of the tenth facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of the second variation of the first facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of any of the preceding aspects of the tenth facet, the hydrogel microparticle comprises between about 0.25% wt. and about 5% wt. of the fibrinogen component. In some embodiments, the hydrogel biomaterial comprises about 0.75% wt. of the fibrinogen component. In some embodiments, the hydrogel biomaterial comprises about 1.50% wt. of the fibrinogen component. In some embodiments, the hydrogel biomaterial comprises about 3.00% wt. of the fibrinogen component.

In some embodiments of any of the preceding aspects of the tenth facet, the hydrogel biomaterial comprises between about 0.1% wt. and 5% wt. of the linear polymer component. In some embodiments, the hydrogel biomaterial comprises between about 0.5% wt. and 2% wt. of the linear polymer component. In some embodiments, the hydrogel biomaterial comprises about 1% wt. of the linear polymer component.

In some embodiments of any of the preceding aspects of the tenth facet, the hydrogel biomaterial comprises between about 0.1% wt. and 5% wt. of the branched polymer component. In some embodiments, the hydrogel biomaterial comprises between about 0.5% wt. and 2% wt. of the branched polymer component. In some embodiments, the hydrogel biomaterial comprises about 1% wt. of the branched polymer component.

In some embodiments of the tenth facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of the tenth facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the tenth facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments of the tenth facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the tenth facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments of the tenth facet, the hydrogel microparticle has a storage modulus of between about 100 Pa and about 10,000 Pa. In some embodiments, the hydrogel microparticle has a storage modulus of between about 100 Pa and about 5,000 Pa. In some embodiments, the hydrogel microparticle has a storage modulus of between about 100 Pa and about 2,500 Pa. In some embodiments, the hydrogel microparticle has a storage modulus of between about 100 Pa and about 2,000 Pa. In some embodiments, the hydrogel microparticle has a storage modulus of between about 100 Pa and about 1,500 Pa. In some embodiments, the hydrogel microparticle has a storage modulus of between about 100 Pa and about 1,000 Pa. In some embodiments, the hydrogel microparticle has a storage modulus of between about 100 Pa and about 750 Pa. In some embodiments, the hydrogel microparticle has a storage modulus of between about 100 Pa and about 500 Pa. In some embodiments, the hydrogel microparticle has a storage modulus of between about 100 Pa and about 250 Pa. In some embodiments, the hydrogel microparticle has a storage modulus of between about 250 Pa and about 10,000 Pa. In some embodiments, the hydrogel microparticle has a storage modulus of between about 250 Pa and about 5,000 Pa. In some embodiments, the hydrogel microparticle has a storage modulus of between about 250 Pa and about 2,500 Pa. In some embodiments, the hydrogel microparticle has a storage modulus of between about 250 Pa and about 2,000 Pa. In some embodiments, the hydrogel microparticle has a storage modulus of between about 250 Pa and about 1,500 Pa. In some embodiments, the hydrogel microparticle has a storage modulus of between about 250 Pa and about 1,000 Pa. In some embodiments, the hydrogel microparticle has a storage modulus of between about 250 Pa and about 750 Pa. In some embodiments, the hydrogel microparticle has a storage modulus of between about 250 Pa and about 500 Pa. In some embodiments, the hydrogel microparticle has a storage modulus of between about 500 Pa and about 10,000 Pa. In some embodiments, the hydrogel microparticle has a storage modulus of between about 500 Pa and about 5,000 Pa. In some embodiments, the hydrogel microparticle has a storage modulus of between about 500 Pa and about 2,500 Pa. In some embodiments, the hydrogel microparticle has a storage modulus of between about 500 Pa and about 2,000 Pa. In some embodiments, the hydrogel microparticle has a storage modulus of between about 500 Pa and about 1,500 Pa. In some embodiments, the hydrogel microparticle has a storage modulus of between about 500 Pa and about 1,000 Pa. In some embodiments, the hydrogel microparticle has a storage modulus of between about 500 Pa and about 750 Pa. In some embodiments, the hydrogel microparticle has a storage modulus of between about 750 Pa and about 10,000 Pa. In some embodiments, the hydrogel microparticle has a storage modulus of between about 750 Pa and about 5,000 Pa. In some embodiments, the hydrogel microparticle has a storage modulus of between about 750 Pa and about 2,500 Pa. In some embodiments, the hydrogel microparticle has a storage modulus of between about 750 Pa and about 2,000 Pa. In some embodiments, the hydrogel microparticle has a storage modulus of between about 750 Pa and about 1,500 Pa. In some embodiments, the hydrogel microparticle has a storage modulus of between about 750 Pa and about 1,000 Pa. In some embodiments, the hydrogel microparticle has a storage modulus of between about 1,000 Pa and about 10,000 Pa. In some embodiments, the hydrogel microparticle has a storage modulus of between about 1,000 Pa and about 5,000 Pa. In some embodiments, the hydrogel microparticle has a storage modulus of between about 1,000 Pa and about 2,500 Pa. In some embodiments, the hydrogel microparticle has a storage modulus of between about 1,000 Pa and about 2,000 Pa. In some embodiments, the hydrogel microparticle has a storage modulus of between about 1,000 Pa and about 1,500 Pa. In some embodiments, the hydrogel microparticle has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel microparticle has a storage modulus of about 100 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some embodiments of the tenth facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

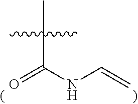 [SMILES: *C(=O)NC=C]

and norborn-2-en-5-yl

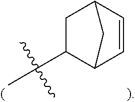 [SMILES: *C1CC2C=CC1C2]

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some variations of the preceding embodiments of the tenth facet, the hydrogel biomaterial does not substantially swell when exposed to a protease capable of cleaving the fibrinogen component.

In some variations of the preceding embodiments of the tenth facet, the longest dimension of the hydrogel biomaterial increases by less than about twofold when 50% of the fibrinogen component has been cleaved by a protease capable of cleaving the fibrinogen component. In some variations of the preceding embodiments and aspects of the tenth facet, the longest dimension of the hydrogel biomaterial increases by less than about 200% when 50% of the fibrinogen component has been cleaved by a protease capable of cleaving the fibrinogen component. In some variations of the preceding embodiments and aspects of the tenth facet, the longest dimension of the hydrogel biomaterial increases by less than about 150% when 50% of the fibrinogen component has been cleaved by a protease capable of cleaving the fibrinogen component. In some variations of the preceding embodiments and aspects of the tenth facet, the longest dimension of the hydrogel biomaterial increases by less than about 100% when 50% of the fibrinogen component has been cleaved by a protease capable of cleaving the fibrinogen component. In some variations of the preceding embodiments and aspects of the tenth facet, the longest dimension of the hydrogel biomaterial increases by less than about 75% when 50% of the fibrinogen component has been cleaved by a protease capable of cleaving the fibrinogen component. In some variations of the preceding embodiments and aspects of the tenth facet, the longest dimension of the hydrogel biomaterial increases by less than about 50% when 50% of the fibrinogen component has been cleaved by a protease capable of cleaving the fibrinogen component. In some variations of the preceding embodiments and aspects of the tenth facet, the longest dimension of the hydrogel biomaterial increases by less than about 25% when 50% of the fibrinogen component has been cleaved by a protease capable of cleaving the fibrinogen component. In some variations of the preceding embodiments and aspects of the tenth facet, the longest dimension of the hydrogel biomaterial increases by less than about 10% when 50% of the fibrinogen component has been cleaved by a protease capable of cleaving the fibrinogen component.

In some variations of the preceding embodiments and aspects of the tenth facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the tenth facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a eleventh facet, provided herein is a hydrogel biomaterial wherein the hydrogel biomaterial is prepared by a polymerization reaction of a mixture comprising:

at least one linker component comprising reactive thiol groups;
a fibrinogen component derivatized with a plurality of polymeric linkers with reactive ene groups;
and
wherein the hydrogel biomaterial has a storage modulus of between about 100 Pa and about 10,000 Pa.

In a first variation of the eleventh facet, the at least one linker component is at least one polymer component. In such embodiments, provided herein is a hydrogel biomaterial wherein the hydrogel biomaterial is prepared by a polymerization reaction of a mixture comprising:

at least one polymer component comprising reactive thiol groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the first variation of the eleventh facet, the at least one polymer component comprises a linear polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of about 3 kDa, about 6 kDa, and about 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the first variation of the eleventh facet, the at least one polymer component comprises a branched polymer component. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the first variation of the eleventh facet, the at least one polymer component comprises a linear polymer component and a branched polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of 3 kDa, 6 kDa, and 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a poly(ethylene glycol) moiety, wherein the poly(ethylene glycol) moiety has a molecular weight selected from the group consisting of 3 kDa, 6 kDa, and 10 kDa. In some embodiments, the linear polymer component comprises a poly(ethylene glycol) moiety, wherein the poly(ethylene glycol) moiety has a molecular weight of about 10 kDa. In some embodiments, the linear polymer component comprises a poly(ethylene glycol) moiety, wherein the poly(ethylene glycol) moiety has a molecular weight of about 6 kDa. In some embodiments, the linear polymer component comprises a poly(ethylene glycol) moiety, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of about 30 kDa.

In some embodiments of the first variation of the eleventh facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-10% wt. of a fibrinogen component consisting of a fibrinogen molecule derivatized with a plurality of polymeric linkers each comprising a 3.5 kDa linear PEG; and

water.

In some embodiments of the first variation of the eleventh facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a 4-armed PEG moiety, wherein the 4-armed PEG moiety has an average molecular weight of 20 kDa;

0-10% wt. of a fibrinogen component consisting of a fibrinogen molecule derivatized with a plurality of polymeric linkers each comprising a 3.5 kDa linear PEG; and

water.

In a second variation of the eleventh facet, the at least one linker component is at least one multivalent linker component. In such embodiments, provided herein is a hydrogel biomaterial wherein the hydrogel biomaterial is prepared by a polymerization reaction of a mixture comprising:

at least one multivalent linker component comprising reactive thiol groups;
a fibrinogen component derivatized with a plurality of polymeric linkers with reactive ene groups;
and
wherein the hydrogel biomaterial has a storage modulus of between about 100 Pa and about 10,000 Pa.

In some embodiments of the second variation of the eleventh facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments any of the preceding aspects of the second variation of the first facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of any of the preceding aspects of the eleventh facet, the hydrogel microparticle comprises between about 0.25% wt. and about 5% wt. of the fibrinogen component. In some embodiments, the hydrogel biomaterial comprises about 0.75% wt. of the fibrinogen component. In some embodiments, the hydrogel biomaterial comprises about 1.50% wt. of the fibrinogen component. In some embodiments, the hydrogel biomaterial comprises about 3.00% wt. of the fibrinogen component.

In some embodiments of any of the preceding aspects of the eleventh facet, the hydrogel biomaterial comprises between about 0.1% wt. and 5% wt. of the linear polymer component. In some embodiments, the hydrogel biomaterial comprises between about 0.5% wt. and 2% wt. of the linear polymer component. In some embodiments, the hydrogel biomaterial comprises about 1% wt. of the linear polymer component.

In some embodiments of any of the preceding aspects of the eleventh facet, the hydrogel biomaterial comprises between about 0.1% wt. and 5% wt. of the branched polymer component. In some embodiments, the hydrogel biomaterial comprises between about 0.5% wt. and 2% wt. of the branched polymer component. In some embodiments, the hydrogel biomaterial comprises about 1% wt. of the branched polymer component.

In some embodiments of the eleventh facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of the eleventh facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

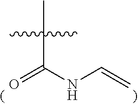 [SMILES: *C(=O)NC=C]

and norborn-2-en-5-yl

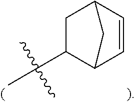 [SMILES: *C1CC2C=CC1C2]

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of the eleventh facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the eleventh facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the eleventh facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the eleventh facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the eleventh facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments of the eleventh facet, the hydrogel biomaterial does not substantially swell when exposed to a protease capable of cleaving the fibrinogen component.

In some variations of the preceding embodiments of the eleventh facet, the hydrogel biomaterial does not substantially swell when exposed to a protease capable of cleaving the fibrinogen component. In some variations of the preceding embodiments of the eleventh facet, the longest dimension of the hydrogel biomaterial increases by less than about twofold when 50% of the fibrinogen component has been cleaved by a protease capable of cleaving the fibrinogen component. In some variations of the preceding embodiments and aspects of the eleventh facet, the longest dimension of the hydrogel biomaterial increases by less than about 200% when 50% of the fibrinogen component has been cleaved by a protease capable of cleaving the fibrinogen component. In some variations of the preceding embodiments and aspects of the eleventh facet, the longest dimension of the hydrogel biomaterial increases by less than about 150% when 50% of the fibrinogen component has been cleaved by a protease capable of cleaving the fibrinogen component. In some variations of the preceding embodiments and aspects of the eleventh facet, the longest dimension of the hydrogel biomaterial increases by less than about 100% when 50% of the fibrinogen component has been cleaved by a protease capable of cleaving the fibrinogen component. In some variations of the preceding embodiments and aspects of the eleventh facet, the longest dimension of the hydrogel biomaterial increases by less than about 75% when 50% of the fibrinogen component has been cleaved by a protease capable of cleaving the fibrinogen component. In some variations of the preceding embodiments and aspects of the eleventh facet, the longest dimension of the hydrogel biomaterial increases by less than about 50% when 50% of the fibrinogen component has been cleaved by a protease capable of cleaving the fibrinogen component. In some variations of the preceding embodiments and aspects of the eleventh facet, the longest dimension of the hydrogel biomaterial increases by less than about 25% when 50% of the fibrinogen component has been cleaved by a protease capable of cleaving the fibrinogen component. In some variations of the preceding embodiments and aspects of the eleventh facet, the longest dimension of the hydrogel biomaterial increases by less than about 10% when 50% of the fibrinogen component has been cleaved by a protease capable of cleaving the fibrinogen component.

In some variations of the preceding embodiments and aspects of the eleventh facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the eleventh facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In a twelfth facet, provided herein is a hydrogel biomaterial wherein the hydrogel biomaterial is prepared by a polymerization reaction of an aqueous mixture comprising:

at least one linker component comprising reactive thiol groups;
a fibrinogen component derivatized with a plurality of polymeric linkers with reactive ene groups;
wherein the hydrogel biomaterial does not substantially swell when exposed to a protease capable of cleaving the fibrinogen component.

In a first variation of the twelfth facet, the at least one linker component is at least one polymer component. In such embodiments, provided herein is a hydrogel biomaterial wherein the hydrogel biomaterial is prepared by a polymerization reaction of an aqueous mixture comprising:

at least one polymer component comprising reactive thiol groups;
a fibrinogen component derivatized with a plurality of polymeric linkers with reactive ene groups;
wherein the hydrogel biomaterial does not substantially swell when exposed to a protease capable of cleaving the fibrinogen component.

In some embodiments of the first variation of the twelfth facet, the at least one polymer component comprises a linear polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of about 3 kDa, about 6 kDa, and about 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the first variation of the twelfth facet, the at least one polymer component comprises a branched polymer component. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the first variation of the twelfth facet, the at least one polymer component comprises a linear polymer component and a branched polymer component. In some embodiments, the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer component comprises a linear polymeric moiety, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer component comprises a linear polymeric moiety, wherein the linear polymeric moiety is a poly(ethylene glycol) moiety having a molecular weight of between about 1 kDa and about 40 kDa. In some embodiments, the linear polymer component comprises a linear polymeric moiety, wherein the linear polymeric moiety is a poly(ethylene glycol) moiety having a molecular weight selected from the group consisting of 3 kDa, 6 kDa, and 10 kDa. In some embodiments, the linear polymer component comprises a linear polymeric moiety, wherein the linear polymeric moiety is a poly(ethylene glycol) moiety having a molecular weight of between about 10 kDa. In some embodiments, the linear polymer component comprises a linear polymeric moiety, wherein the linear polymeric moiety is a poly(ethylene glycol) moiety having a molecular weight of between about 6 kDa. In some embodiments, the linear polymer component comprises a linear polymeric moiety, wherein the linear polymeric moiety is a poly(ethylene glycol) moiety having a molecular weight of between about 3 kDa. In some embodiments, the linear polymer component comprises a poly(ethylene glycol) moiety, wherein the poly(ethylene glycol) moiety has a molecular weight selected from the group consisting of 3 kDa, 6 kDa, and 10 kDa. In some embodiments, the linear polymer component comprises a poly(ethylene glycol) moiety, wherein the poly(ethylene glycol) moiety has a molecular weight of about 10 kDa. In some embodiments, the linear polymer component comprises a poly(ethylene glycol) moiety, wherein the poly(ethylene glycol) moiety has a molecular weight of about 6 kDa. In some embodiments, the linear polymer component comprises a poly(ethylene glycol) moiety, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer component comprises a branched polymeric moiety with n polymeric arms, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer component comprises a 3-arm poly(ethylene glycol) moiety. In some embodiments, the 3-arm poly(ethylene glycol) moiety has a molecular weight of about 15 kDa. In some embodiments, the branched polymer component comprises a 4-arm poly(ethylene glycol) moiety. In some embodiments, the 4-arm poly(ethylene glycol) moiety has a molecular weight of about 20 kDa. In some embodiments, the branched polymer component comprises a 6-arm poly(ethylene glycol) moiety. In some embodiments, the 6-arm poly(ethylene glycol) moiety has a molecular weight of about 30 kDa.

In some embodiments of the first variation of the twelfth facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a multiple armed PEG moiety comprising n arms with terminal thiol groups, wherein the PEG moiety has an average molecular weight of 20 kDa, and n is 3, 4, 6 or 8;

0-10% wt. of a fibrinogen component consisting of a fibrinogen molecule derivatized with a plurality of polymeric linkers each comprising a 3.5 kDa linear PEG; and

water.

In some embodiments of the first variation of the twelfth facet, the hydrogel biomaterial comprises:

0-50% wt. of a linear polymer component comprising a linear PEG moiety, wherein the linear PEG moiety has an average molecular weight of 6 kDa;

0.1-50% wt. of a branched polymer component comprising a 4-armed PEG moiety, wherein the 4-armed PEG moiety has an average molecular weight of 20 kDa;

0-10% wt. of a fibrinogen component consisting of a fibrinogen molecule derivatized with a plurality of polymeric linkers each comprising a 3.5 kDa linear PEG; and

water.

In a first variation of the twelfth facet, the at least one linker component is at least one multivalent linker component. In such embodiments, provided herein is a hydrogel biomaterial wherein the hydrogel biomaterial is prepared by a polymerization reaction of an aqueous mixture comprising:

at least one multivalent linker component comprising reactive thiol groups;
a fibrinogen component derivatized with a plurality of polymeric linkers with reactive ene groups;
wherein the hydrogel biomaterial does not substantially swell when exposed to a protease capable of cleaving the fibrinogen component.

In some embodiments of the second variation of the twelfth facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments any of the preceding aspects of the second variation of the first facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of any of the preceding aspects of the twelfth facet, the hydrogel microparticle comprises between about 0.25% wt. and about 5% wt. of the fibrinogen component. In some embodiments, the hydrogel biomaterial comprises about 0.75% wt. of the fibrinogen component. In some embodiments, the hydrogel biomaterial comprises about 1.50% wt. of the fibrinogen component. In some embodiments, the hydrogel biomaterial comprises about 3.00% wt. of the fibrinogen component.

In some embodiments of any of the preceding aspects of the twelfth facet, the hydrogel biomaterial comprises between about 0.1% wt. and 5% wt. of the linear polymer component. In some embodiments, the hydrogel biomaterial comprises between about 0.5% wt. and 2% wt. of the linear polymer component. In some embodiments, the hydrogel biomaterial comprises about 1% wt. of the linear polymer component.

In some embodiments of any of the preceding aspects of the twelfth facet, the hydrogel biomaterial comprises between about 0.1% wt. and 5% wt. of the branched polymer component. In some embodiments, the hydrogel biomaterial comprises between about 0.5% wt. and 2% wt. of the branched polymer component. In some embodiments, the hydrogel biomaterial comprises about 1% wt. of the branched polymer component.

In some embodiments of the twelfth facet, the overall stoichiometric ratio of the norbornene moieties to the free thiol moieties prior to polymerization is selected such that the average branch order of either the norbornene bearing entities or free thiol bearing entities prior to polymerization is greater than 2.

In some embodiments of the twelfth facet, the fibrinogen component derivatized with a plurality of polymeric linkers has been derivatized at cysteine residues. In some embodiments, the fibrinogen component derivatized with a plurality of polymeric linkers has been derivatized at cysteine residues and comprises between 30 moles and 58 moles of polymeric linkers per mole of derivatized fibrinogen. In some embodiments, the fibrinogen component derivatized with a plurality of polymeric linkers has been derivatized at cysteine residues and comprises 58 moles of polymeric linkers per mole of derivatized fibrinogen. In some embodiments, the fibrinogen component derivatized with a plurality of polymeric linkers has been derivatized at cysteine residues and comprises 57±1 moles of polymeric linkers per mole of derivatized fibrinogen. In some embodiments, the fibrinogen component derivatized with a plurality of polymeric linkers has been derivatized at cysteine residues and comprises 56±2 moles of polymeric linkers per mole of derivatized fibrinogen. In some embodiments, the fibrinogen component derivatized with a plurality of polymeric linkers has been derivatized at cysteine residues and comprises 55±3 moles of polymeric linkers per mole of derivatized fibrinogen. In some embodiments, the fibrinogen component derivatized with a plurality of polymeric linkers has been derivatized at cysteine residues and comprises 54±4 moles of polymeric linkers per mole of derivatized fibrinogen. In some embodiments, the fibrinogen component derivatized with a plurality of polymeric linkers has been derivatized at cysteine residues and comprises 53±5 moles of polymeric linkers per mole of derivatized fibrinogen. In some embodiments, the fibrinogen component derivatized with a plurality of polymeric linkers has been derivatized at cysteine residues and comprises 48±10 moles of polymeric linkers per mole of derivatized fibrinogen. In some embodiments, each individual polymeric linker of the plurality of polymeric linkers with reactive ene groups comprises a linear polymeric moiety and a reactive ene group. In some embodiments, each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each individual polymeric linker of the plurality of polymeric linkers is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the reactive ene groups of the plurality of polymeric linkers with reactive ene groups are terminal reactive ene groups. In some embodiments, the reactive ene group of each individual polymeric linker of the plurality of polymeric linkers with reactive ene groups is independently selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

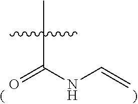 [SMILES: *C(=O)NC=C]

and norborn-2-en-5-yl

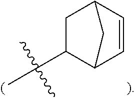 [SMILES: *C1CC2C=CC1C2]

In some embodiments, each individual polymeric linker of the plurality of polymeric linkers with reactive ene groups comprises a poly(ethylene glycol) moiety and a terminal norborn-2-en-5-yl group. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of between about 1 kDa and 40 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight selected from the group consisting of 3.5 kDa, 6 kDa, and 10 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 3.5 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 6 kDa. In some embodiments, each individual polymeric linker of the plurality of polymeric linkers with terminal reactive ene groups comprises a poly(ethylene glycol) moiety and a terminal norborn-2-en-5-yl group, wherein the poly(ethylene glycol) moiety has a molecular weight of about 10 kDa. In some embodiments, each individual polymeric linker of the plurality of polymeric linkers has a molecular weight of between about 500 Da and about 40 kDa. In some embodiments, each individual polymeric linker of the plurality of polymeric linkers has a molecular weight of between about 1 kDa and about 40 kDa. In some embodiments, each individual polymeric linker of the plurality of polymeric linkers has a molecular weight selected from the group consisting of about 3 kDa, about 6 kDa, and about 10 kDa. In some embodiments, each individual polymeric linker of the plurality of polymeric linkers has a molecular weight of about 3 kDa. In some embodiments, each individual polymeric linker of the plurality of polymeric linkers has a molecular weight of about 6 kDa. In some embodiments, each individual polymeric linker of the plurality of polymeric linkers has a molecular weight of about 10 kDa.

In some embodiments of the twelfth facet, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the twelfth facet, the polymerization reaction is a free radical mediated thiol-ene polymerization reaction.

In some embodiments, the hydrogel biomaterial is a hydrogel microparticle. The hydrogel microparticles provided herein (i) have reproducible and defined mechanical properties; (ii) provide a degradation-independent pathway for rapid cell migration through the injury volume; (iii) have degradation properties conducive to cellular infiltration; and (iv) have advantageous swelling properties.

In some variations of the preceding embodiments and aspects of the twelfth facet, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments and aspects of the twelfth facet, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some variations of the preceding embodiments and aspects of the twelfth facet, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some variations of the preceding embodiments of the twelfth facet, the hydrogel biomaterial does not substantially swell when exposed to a protease capable of cleaving the fibrinogen component.

In some variations of the preceding embodiments of the twelfth facet, the hydrogel microparticle does not substantially swell when exposed to a protease capable of cleaving the fibrinogen component. In some variations of the preceding embodiments and aspects of the twelfth facet, the longest dimension of the hydrogel biomaterial increases by less than about twofold when 50% of the fibrinogen component has been cleaved by a protease capable of cleaving the fibrinogen component. In some variations of the preceding embodiments and aspects of the twelfth facet, the longest dimension of the hydrogel biomaterial increases by less than about 200% when 50% of the fibrinogen component has been cleaved by a protease capable of cleaving the fibrinogen component. In some variations of the preceding embodiments and aspects of the twelfth facet, the longest dimension of the hydrogel biomaterial increases by less than about 150% when 50% of the fibrinogen component has been cleaved by a protease capable of cleaving the fibrinogen component. In some variations of the preceding embodiments and aspects of the twelfth facet, the longest dimension of the hydrogel biomaterial increases by less than about 100% when 50% of the fibrinogen component has been cleaved by a protease capable of cleaving the fibrinogen component. In some variations of the preceding embodiments and aspects of the twelfth facet, the longest dimension of the hydrogel biomaterial increases by less than about 75% when 50% of the fibrinogen component has been cleaved by a protease capable of cleaving the fibrinogen component. In some variations of the preceding embodiments and aspects of the twelfth facet, the longest dimension of the hydrogel biomaterial increases by less than about 50% when 50% of the fibrinogen component has been cleaved by a protease capable of cleaving the fibrinogen component. In some variations of the preceding embodiments and aspects of the twelfth facet, the longest dimension of the hydrogel biomaterial increases by less than about 25% when 50% of the fibrinogen component has been cleaved by a protease capable of cleaving the fibrinogen component. In some variations of the preceding embodiments and aspects of the twelfth facet, the longest dimension of the hydrogel biomaterial increases by less than about 10% when 50% of the fibrinogen component has been cleaved by a protease capable of cleaving the fibrinogen component.

In some variations of the preceding embodiments and aspects of the twelfth facet, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some variations of the preceding embodiments and aspects of the twelfth facet, the hydrogel biomaterial is derivatized with a biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

Also provided herein is a pharmaceutical composition comprising a hydrogel microparticle of any of the preceding embodiments, embodiments, aspects and facets. Also provided herein is a pharmaceutical composition comprising a plurality of hydrogel microparticles of any of the preceding embodiments, embodiments, aspects and facets. Also provided herein is a tissue scaffold comprising a plurality of hydrogel microparticles of any of the preceding embodiments, embodiments, aspects and facets.

In some embodiments, the pharmaceutical composition comprises a plurality of hydrogel microparticles of any one of the preceding embodiments, embodiments, aspects and facets, wherein at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.05 mm and about 1.0 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.25 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.5 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.75 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.05 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.05 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.05 mm and about 0.25 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.25 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.1 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.1 mm and about 0.2 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.2 mm and about 0.3 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.3 mm and about 0.4 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.4 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.5 mm and about 0.6 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.6 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.5 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microparticles have a longest dimension of between about 0.1 mm and about 0.5 mm.

In some embodiments, the pharmaceutical composition comprises a plurality of hydrogel microspheres. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.05 mm and about 1.0 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.25 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.5 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.75 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.05 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.05 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.05 mm and about 0.25 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.25 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.1 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.1 mm and about 0.2 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.2 mm and about 0.3 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.3 mm and about 0.4 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.4 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.5 mm and about 0.6 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.6 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.5 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the hydrogel microspheres have a diameter of between about 0.1 mm and about 0.5 mm.

In some embodiments, the pharmaceutical composition comprises a plurality of hydrogel microparticles of any one of the preceding embodiments, embodiments, aspects and facets, wherein:

each individual hydrogel microparticle of the plurality of hydrogel microparticles has an individual volume;

the plurality of hydrogel microparticles has a bulk volume of hydrogel material;

the sum of the individual volumes of all individual microparticles is the bulk volume of hydrogel material; and

at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension between about 0.05 mm and about 1.0 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.25 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.5 mm and about 1 mm. In some embodiments, at least 50%, at least 60%, at least 70%, at least 80%, at least 90%, at least 95%, at least 96%, at least 97%, at least 98%, at least 99%, at least 99.90, or at least 99.99% of the bulk volume of hydrogel material is made of individual hydrogel microparticles having a longest dimension of between about 0.75 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.05 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.05 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.05 mm and about 0.25 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.25 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.1 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.1 mm and about 0.2 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.2 mm and about 0.3 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.3 mm and about 0.4 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.4 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.5 mm and about 0.6 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.6 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.5 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension of between about 0.1 mm and about 0.5 mm.

In some embodiments, the pharmaceutical composition comprises a plurality of hydrogel microspheres, wherein:

each individual hydrogel microsphere of the plurality of hydrogel microspheres has an individual volume;

the plurality of hydrogel microsphere has a bulk volume of hydrogel material;

the sum of the individual volumes of all individual microsphere is the bulk volume of hydrogel material; and

at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.05 mm and about 1.0 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.25 mm and about 1 mm. In some embodiments, at least 50%, at least 60%, at least 70%, at least 80%, at least 90%, at least 95%, at least 96%, at least 97%, at least 98%, at least 99%, at least 99.90, or at least 99.99% of the bulk volume of hydrogel material is made of individual hydrogel microspheres having a diameter of between about 0.5 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.75 mm and about 1 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.05 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.05 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.05 mm and about 0.25 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.25 mm and about 0.75 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.1 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.1 mm and about 0.2 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.2 mm and about 0.3 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.3 mm and about 0.4 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.4 mm and about 0.5 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.5 mm and about 0.6 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.6 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.5 mm and about 0.7 mm. In some embodiments, at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter of between about 0.1 mm and about 0.5 mm.

In some embodiments, the pharmaceutical composition comprises a plurality of hydrogel microparticles of any one of the preceding embodiments, embodiments, aspects and facets, wherein:

each individual hydrogel microparticle of the plurality of hydrogel microparticles has an individual volume;

the plurality of hydrogel microparticles has a bulk volume of hydrogel material;

the sum of the individual volumes of all individual microparticles is the bulk volume of hydrogel material; and

at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microparticles having a longest dimension as described in any one of the preceding embodiments, embodiments, aspects and facets.

In some embodiments, the pharmaceutical composition comprises a plurality of hydrogel microspheres, wherein:

each individual hydrogel microsphere of the plurality of hydrogel microspheres has an individual volume;

the plurality of hydrogel microsphere has a bulk volume of hydrogel material;

the sum of the individual volumes of all individual microsphere is the bulk volume of hydrogel material; and

at least 50%, at least 60%, at least 70%, at least 80%, at least 90%, at least 95%, at least 96%, at least 97%, at least 98%, at least 99%, at least 99.9%, or at least 99.99% of the bulk volume of hydrogel material is made of individual hydrogel microspheres having a diameter as described in any one of the preceding embodiments, embodiments, aspects and facets.

In the descriptions herein, it is understood that every description, variation, embodiment or aspect of a facet may be combined with every description, variation, embodiment or aspect of other facets the same as if each and every combination of descriptions is specifically and individually listed.

Also provided herein is a pharmaceutical composition comprising a hydrogel biomaterial of any of the preceding embodiments, embodiments, aspects and facets. Also provided herein is a tissue scaffold comprising a plurality of hydrogel biomaterial of any of the preceding embodiments, embodiments, aspects and facets.

Compositions comprising a hydrogel biomaterial described herein for treating an injury are also provided. In some embodiments, the hydrogel biomaterial comprises a plurality of hydrogel microparticles. In some embodiments, the injury is a wound, a nerve injury, a muscle injury, a cartilage defect, a bone defect, or an osteochondral defect. In some embodiments, the wound is an acute wound. In some embodiments, the acute wound is a traumatic wound, a cut, a surgical wound (such as a tissue resection, a biopsy, a MOHS surgery, or a dental extraction socket), or a burn (such as a thermal burn or a chemical burn). In some specific embodiments, the acute wound is a traumatic wound. In some specific embodiments, the acute wound is a cut. In some specific embodiments, the acute wound is a surgical wound. In some specific embodiments, the acute wound is a tissue resection. In some specific embodiments, the acute wound is a biopsy. In some specific embodiments, the acute wound is a dental extraction socket. In some specific embodiments, the acute wound is a burn. In some specific embodiments, the acute wound is a thermal burn. In some specific embodiments, the acute wound is a chemical burn. In some embodiments, the wound is a chronic wound. In some embodiments, the chronic wound is a venous ulcer, a diabetic ulcer, a pressure ulcer, or a wound caused by radiation poisoning. In some specific embodiments, the chronic wound is a venous ulcer. In some specific embodiments, the chronic wound is a diabetic ulcer. In some specific embodiments, the chronic wound is a pressure ulcer. In some specific embodiments, the chronic wound is a wound caused by radiation poisoning. In some embodiments, the cartilage defect is osteoarthritis. In some embodiments, the cartilage defect is a mechanical injury. In some embodiments, the bone defect is a traumatic bone defect, a bone defect caused by surgery, or a bone defect caused by a disease or condition. In some embodiments, the bone defect is a traumatic bone defect. In some specific embodiments, the traumatic bone defect is a cracked bone or a bone fracture. In some specific embodiments, the traumatic bone defect is a cracked bone. In some specific embodiments, the traumatic bone defect is a bone fracture. In some embodiments, the bone defect is a bone defect caused by surgery. In some specific embodiments, the bone defect caused by surgery is a bone defect caused by tumor resection. In some specific embodiments, the bone defect caused by surgery is a bone defect caused by revision surgery such as knee replacement surgery or hip replacement surgery. In some specific embodiments, the bone defect caused by surgery is a bone defect caused by removal of surgical fixation devices. In some embodiments, the bone defect is a bone defect caused by a disease or condition. In some specific embodiments, the bone defect caused by a disease or condition is a bone defect caused by infection. In some specific embodiments, the bone defect caused by a disease or condition is a bone defect caused by osteoarthritis. In some specific embodiments, the bone defect caused by a disease or condition is a bone defect caused by a neoplasm. In some embodiments, the osteochondral defect is a focal area of damage that involves both the cartilage and a piece of underlying bone. In some embodiments, the osteochondral defect is an osteochondral defect occurring from an acute traumatic injury. In some embodiments, the osteochondral defect is an osteochondral defect occurring from an underlying disorder of a bone.

Compositions comprising a hydrogel biomaterial described herein for treating a tissue defect or improving a cosmetic outcome are also provided. In some embodiments, the hydrogel biomaterial comprises a plurality of hydrogel microparticles. In some embodiments, the method is a method of treating a tissue defect. In some embodiments, the method is a method of reconstructive surgery of soft tissue. In some embodiments, the method is a method of reconstructive surgery of bone. In some embodiments, the method is a method of reconstructive surgery of cartilage. In some embodiments, the cartilage is ear cartilage. In some embodiments, the cartilage is nose cartilage. In some embodiments, the method is a method of reconstructive surgery of muscle. In some embodiments, the method is a method of improving a cosmetic outcome. In some embodiments, the method is a method of cosmetic surgery of soft tissue. In some embodiments, the method is a method of cosmetic surgery of bone. In some embodiments, the method is a method of cosmetic surgery of cartilage. In some embodiments, the cartilage is ear cartilage. In some embodiments, the cartilage is nose cartilage. In some embodiments, the method is a method of cosmetic surgery of muscle.

Compositions comprising a hydrogel biomaterial described herein for creating a filler to fill a tissue void are also provided. In some embodiments, the hydrogel biomaterial comprises a plurality of hydrogel microparticles. In some embodiments, the filler is a dermal filler. In some embodiments, the filler is a bone filler.

Compositions comprising a hydrogel biomaterial described herein for treating pain are also provided. In some embodiments, the hydrogel biomaterial comprises a plurality of hydrogel microparticles. Compositions comprising a hydrogel biomaterial described herein for treating an orthopedic condition are also provided. In some embodiments, the hydrogel biomaterial comprises a plurality of hydrogel microparticles.

Compositions comprising a hydrogel biomaterial described herein for use in a method for treating an injury are also provided. In some embodiments, the hydrogel biomaterial comprises a plurality of hydrogel microparticles. In some embodiments, the injury is a wound, a nerve injury, a muscle injury, a cartilage defect, a bone defect, or an osteochondral defect. In some embodiments, the wound is an acute wound. In some embodiments, the acute wound is a traumatic wound, a cut, a surgical wound (such as a tissue resection, a biopsy, a MOHS surgery, or a dental extraction socket), or a burn (such as a thermal burn or a chemical burn). In some specific embodiments, the acute wound is a traumatic wound. In some specific embodiments, the acute wound is a cut. In some specific embodiments, the acute wound is a surgical wound. In some specific embodiments, the acute wound is a tissue resection. In some specific embodiments, the acute wound is a biopsy. In some specific embodiments, the acute wound is a dental extraction socket. In some specific embodiments, the acute wound is a burn. In some specific embodiments, the acute wound is a thermal burn. In some specific embodiments, the acute wound is a chemical burn. In some embodiments, the wound is a chronic wound. In some embodiments, the chronic wound is a venous ulcer, a diabetic ulcer, a pressure ulcer, or a wound caused by radiation poisoning. In some specific embodiments, the chronic wound is a venous ulcer. In some specific embodiments, the chronic wound is a diabetic ulcer. In some specific embodiments, the chronic wound is a pressure ulcer. In some specific embodiments, the chronic wound is a wound caused by radiation poisoning. In some embodiments, the cartilage defect is osteoarthritis. In some embodiments, the cartilage defect is a mechanical injury. In some embodiments, the bone defect is a traumatic bone defect, a bone defect caused by surgery, or a bone defect caused by a disease or condition. In some embodiments, the bone defect is a traumatic bone defect. In some specific embodiments, the traumatic bone defect is a cracked bone or a bone fracture. In some specific embodiments, the traumatic bone defect is a cracked bone. In some specific embodiments, the traumatic bone defect is a bone fracture. In some embodiments, the bone defect is a bone defect caused by surgery. In some specific embodiments, the bone defect caused by surgery is a bone defect caused by tumor resection. In some specific embodiments, the bone defect caused by surgery is a bone defect caused by revision surgery such as knee replacement surgery or hip replacement surgery. In some specific embodiments, the bone defect caused by surgery is a bone defect caused by removal of surgical fixation devices. In some embodiments, the bone defect is a bone defect caused by a disease or condition. In some specific embodiments, the bone defect caused by a disease or condition is a bone defect caused by infection. In some specific embodiments, the bone defect caused by a disease or condition is a bone defect caused by osteoarthritis. In some specific embodiments, the bone defect caused by a disease or condition is a bone defect caused by a neoplasm. In some embodiments, the osteochondral defect is a focal area of damage that involves both the cartilage and a piece of underlying bone. In some embodiments, the osteochondral defect is an osteochondral defect occurring from an acute traumatic injury. In some embodiments, the osteochondral defect is an osteochondral defect occurring from an underlying disorder of a bone.

Compositions comprising a hydrogel biomaterial described herein for use in a method for treating a tissue defect or improving a cosmetic outcome are also provided. In some embodiments, the hydrogel biomaterial comprises a plurality of hydrogel microparticles. In some embodiments, the method is a method of treating a tissue defect. In some embodiments, the method is a method of reconstructive surgery of soft tissue. In some embodiments, the method is a method of reconstructive surgery of bone. In some embodiments, the method is a method of reconstructive surgery of cartilage. In some embodiments, the cartilage is ear cartilage. In some embodiments, the cartilage is nose cartilage. In some embodiments, the method is a method of reconstructive surgery of muscle. In some embodiments, the method is a method of improving a cosmetic outcome. In some embodiments, the method is a method of cosmetic surgery of soft tissue. In some embodiments, the method is a method of cosmetic surgery of bone. In some embodiments, the method is a method of cosmetic surgery of cartilage. In some embodiments, the cartilage is ear cartilage. In some embodiments, the cartilage is nose cartilage. In some embodiments, the method is a method of cosmetic surgery of muscle.

Compositions comprising a hydrogel biomaterial described herein for use in a method for creating a filler to fill a tissue void are also provided. In some embodiments, the hydrogel biomaterial comprises a plurality of hydrogel microparticles. In some embodiments, the filler is a dermal filler. In some embodiments, the filler is a bone filler.

Compositions comprising a hydrogel biomaterial described herein for use in a method for treating pain are also provided. In some embodiments, the hydrogel biomaterial comprises a plurality of hydrogel microparticles. Compositions comprising a hydrogel biomaterial described herein for use in a method for treating an orthopedic condition are also provided. In some embodiments, the hydrogel biomaterial comprises a plurality of hydrogel microparticles.

### Direct-to-Polymer Methods

Provided herein is a method for making a hydrogel biomaterial comprising

at least one linker component; and

a fibrinogen component;

wherein at least a portion of the at least one linker component, and at least a portion of the fibrinogen component are crosslinked via a plurality of crosslink units each independently comprising a carbon-sulfur covalent bond;
the method comprising:
subjecting a mixture comprising the at least one linker component and the fibrinogen component to a chemical reaction to create carbon-sulfur covalent bonds of the plurality of crosslink units.

In a first facet, the at least one linker component comprises at least one polymer component and at least one protease-cleavable component. In such embodiments, provided herein is a method for making a hydrogel biomaterial comprising

at least one polymer component;

at least one protease-cleavable component; and

a fibrinogen component;

wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component, the at least one protease-cleavable component, and the fibrinogen component to a chemical reaction to create the carbon-sulfur covalent bonds of the plurality of crosslink units.

In a first aspect of the first facet, provided herein is a method for making a hydrogel biomaterial comprising

at least one polymer component;

at least one protease-cleavable component; and

a fibrinogen component;

wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive thiol groups, wherein the at least one protease-cleavable component comprises reactive ene groups, and at least a portion of the fibrinogen component wherein the fibrinogen component comprises reactive thiol groups, to a chemical reaction to create the carbon-sulfur covalent bonds of the plurality of crosslink units.

In some embodiments of the first aspect of the first facet, the at least one polymer component comprises a linear polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of about 3 kDa, about 6 kDa, and about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the first aspect of the first facet, the at least one polymer component comprises a branched polymer with reactive thiol groups. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 3-arm poly(ethylene glycol) moiety and three terminal thiol groups. In some embodiments, the 3-arm poly(ethylene glycol) moiety has a molecular weight of between about 1 kDa and about 40 kDa. In some embodiments, the 4-arm poly(ethylene glycol) moiety has a molecular weight of about 15 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the first aspect of the first facet, the at least one polymer component comprises a linear polymer with reactive thiol groups and a branched polymer with reactive thiol groups. In some embodiments, the linear polymer with reactive thiol groups comprises a linear polymeric moiety and two reactive thiol groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive thiol groups comprises a linear polymeric moiety and two reactive thiol groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive thiol groups comprises a linear polymeric moiety and two reactive thiol groups, wherein the linear polymeric moiety is a poly(ethylene glycol) moiety having a molecular weight of between about 1 kDa and about 40 kDa. In some embodiments, the linear polymer with reactive thiol groups comprises a linear polymeric moiety and two reactive thiol groups, wherein the linear polymeric moiety is a poly(ethylene glycol) moiety having a molecular weight selected from the group consisting of 3 kDa, 6 kDa, and 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 10 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 6 kDa. In some embodiments, the linear polymer with reactive thiol groups comprises a linear polymeric moiety and two reactive thiol groups, wherein the linear polymeric moiety is a poly(ethylene glycol) moiety having a molecular weight of between about 3 kDa. In some embodiments, the two reactive thiol groups of the linear polymer with reactive thiol groups are terminal reactive thiol groups. In some embodiments, the linear polymer with reactive thiol groups comprises a poly(ethylene glycol) moiety and two terminal thiol groups. In some embodiments, the linear polymer with terminal reactive thiol groups comprises a poly(ethylene glycol) moiety and two terminal thiol groups, wherein the poly(ethylene glycol) moiety has a molecular weight selected from the group consisting of 3 kDa, 6 kDa, and 10 kDa. In some embodiments, the linear polymer with terminal reactive thiol groups comprises a poly(ethylene glycol) moiety and two terminal thiol groups, wherein the poly(ethylene glycol) moiety has a molecular weight of about 10 kDa. In some embodiments, the linear polymer with terminal reactive thiol groups comprises a poly(ethylene glycol) moiety and two terminal thiol groups, wherein the poly(ethylene glycol) moiety has a molecular weight of about 6 kDa. In some embodiments, the linear polymer with terminal reactive thiol groups comprises a poly(ethylene glycol) moiety and two terminal thiol groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive thiol groups comprises a branched polymeric moiety with n polymeric arms and n reactive thiol groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive thiol groups of the branched polymer are terminal reactive thiol groups. In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive thiol groups comprises a 4-arm poly(ethylene glycol) moiety and four terminal thiol groups. In some embodiments, the 4-arm poly(ethylene glycol) moiety has a molecular weight of about 20 kDa. In some embodiments, the branched polymer with reactive thiol groups comprises a 6-arm poly(ethylene glycol) moiety and six terminal thiol groups. In some embodiments, the 6-arm poly(ethylene glycol) moiety has a molecular weight of about 30 kDa.

In some embodiments of the first aspect of the first facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

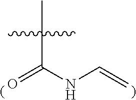 [SMILES: *C(=O)NC=C]

and norborn-2-en-5-yl

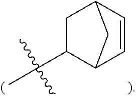 [SMILES: *C1CC2C=CC1C2]

In a second aspect of the first facet, provided herein is a method for making a hydrogel biomaterial comprising

at least one polymer component;

at least one protease-cleavable component; and

a fibrinogen component;

wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive ene groups, wherein the at least one protease-cleavable component comprises reactive ene groups, and at least a portion of the fibrinogen component wherein the fibrinogen component comprises reactive thiol groups, to a chemical reaction to create the carbon-sulfur covalent bonds of the plurality of crosslink units.

In some embodiments of the second aspect of the first facet, the at least one polymer component comprises a linear polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of about 3 kDa, about 6 kDa, and about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

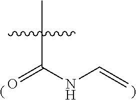 [SMILES: *C(=O)NC=C]

and norborn-2-en-5-yl

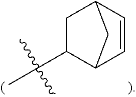 [SMILES: *C1CC2C=CC1C2]

In some embodiments, wherein the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of about 3 kDa, about 6 kDa, and about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the second aspect of the first facet, the at least one polymer component comprises a branched polymer with reactive ene groups. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

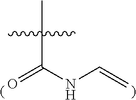 [SMILES: *C(=O)NC=C]

and norborn-2-en-5-yl

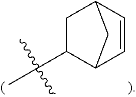 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 3-arm poly(ethylene glycol) moiety and three terminal norborn-2-en-5-yl groups (one at each terminus). In some embodiments, the 3-arm poly(ethylene glycol) moiety has a molecular weight of between about 1 kDa and about 40 kDa. In some embodiments, the 3-arm poly(ethylene glycol) moiety has a molecular weight of about 15 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the second aspect of the first facet, the at least one polymer component comprises a linear polymer with reactive ene groups and a branched polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of 3 kDa, 6 kDa, and 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

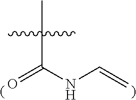 [SMILES: *C(=O)NC=C]

and norborn-2-en-5-yl

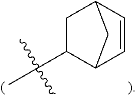 [SMILES: *C1CC2C=CC1C2]

In some embodiments, the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of 3 kDa, 6 kDa, and 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

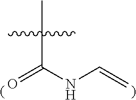 [SMILES: *C(=O)NC=C]

and norborn-2-en-5-yl

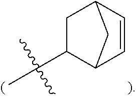 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of about 30 kDa.

In some embodiments of second aspect of the first facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

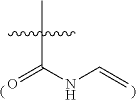 [SMILES: *C(=O)NC=C]

and norborn-2-en-5-yl

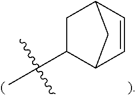 [SMILES: *C1CC2C=CC1C2]

In a third aspect of the first facet, provided herein is a method for making a hydrogel biomaterial comprising

at least one polymer component;

at least one protease-cleavable component; and

a fibrinogen component;

wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive ene groups, wherein the at least one protease-cleavable component comprises reactive thiol groups, and at least a portion of the fibrinogen component wherein the fibrinogen component comprises reactive thiol groups, to a chemical reaction to create the carbon-sulfur covalent bonds of the plurality of crosslink units.

In some embodiments of the third aspect of the first facet, the at least one polymer component comprises a linear polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of about 3 kDa, about 6 kDa, and about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

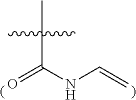 [SMILES: *C(=O)NC=C]

and norborn-2-en-5-yl

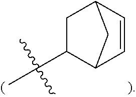 [SMILES: *C1CC2C=CC1C2]

In some embodiments, wherein the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of about 3 kDa, about 6 kDa, and about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the third aspect of the first facet, the at least one polymer component comprises a branched polymer with reactive ene groups. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

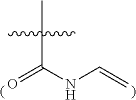 [SMILES: *C(=O)NC=C]

and norborn-2-en-5-yl

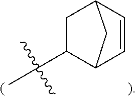 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 3-arm poly(ethylene glycol) moiety and three terminal norborn-2-en-5-yl groups (one at each terminus). In some embodiments, the 3-arm poly(ethylene glycol) moiety has a molecular weight of between about 1 kDa and about 40 kDa. In some embodiments, the 3-arm poly(ethylene glycol) moiety has a molecular weight of about 15 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the third aspect of the first facet, the at least one polymer component comprises a linear polymer with reactive ene groups and a branched polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of 3 kDa, 6 kDa, and 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

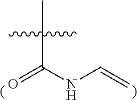 [SMILES: *C(=O)NC=C]

and norborn-2-en-5-yl

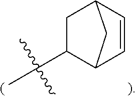 [SMILES: *C1CC2C=CC1C2]

In some embodiments, the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of 3 kDa, 6 kDa, and 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

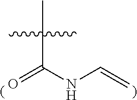 [SMILES: *C(=O)NC=C]

and norborn-2-en-5-yl

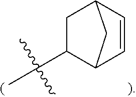 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of about 30 kDa.

In some embodiments of the third aspect of the first facet, the at least one protease-cleavable component is a peptide (such as a synthetic peptide) comprising at least two reactive thiol groups. In some embodiments, each of the at least one protease-cleavable component is a peptide having a sequence of between 3 and 25 amino acids, and wherein the peptide comprises at least two reactive thiol groups. In some embodiments, each of the at least one protease-cleavable component is a peptide having a sequence of 4, 9, 10, 11, 12, 13, or 16 amino acids. In some embodiments, each of the at least one protease-cleavable component comprises a homocysteine residue. In some embodiments, each of the at least one protease-cleavable component comprises two homocysteine residues. In some embodiments, each of the at least one protease-cleavable component comprises a peptide chain comprising a sequence selected from the group consisting of SEQ ID NOs: 1-19 as shown in Tables 1 and 2.

It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising thiol groups suitable for thiol-ene photoinitiated radical chemistry (such as cysteine or homocysteine). The protease-cleavable component may optionally comprise other additional flanking amino acids. Additionally, it is understood that homocysteine-analogs of protease-cleavable sequences containing cysteine (C) residues can be prepared by replacing cysteine residues with homocysteine residues. For non-limiting example, the peptide of sequence SEQ ID NO: 5 is a homocysteine analog of the cysteine-containing peptide of sequence SEQ ID NO: 3. Alternatively, it is understood for non-limiting exemplary purpose additional suitable protease-cleavable components may be prepared by modifying the amino acids sequences described above (SEQ ID NOs: 1-19) via the addition of flanking groups comprising thiol groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive thiol groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive thiol group on the lysine's side chain by reaction of the lysine's side chain's amino group with a thiolating agent such as, for non-limiting example, N-succinimidyl-S-acetylthiopropionate (SATP), N-acetyl homocysteine thiolactone, and NHS-PEG-SH. The protease-cleavable component may optionally comprise other additional flanking amino acids. It is further understood that peptides comprising multiple cleavage sites within one peptide can be prepared. For non-limiting example, the peptide CALKVLKGC (SEQ ID NO: 3) comprises two cleavage sites as the ALK and VLK sub-units are both plasmin substrates and both result in a cleavage site in the peptide of sequence SEQ ID NO: 3. Additionally, it is understood that peptides comprising suitable protease-cleavable sequences (such as for non-limiting example comprising the amino acids sequences described above (SEQ ID NOs: 1-19)), can be modified via terminal modification either one or both of the NH₂ or COOH termini. For non-limiting example, the peptide of sequence SEQ ID NO: 7 corresponds to the peptide of sequence SEQ ID NO: 6 wherein the COOH terminus of the peptide of sequence SEQ ID NO: 6 is modified to be a CONH₂ group in the peptide of sequence SEQ ID NO: 7. Lastly, it is understood that numerous protease-cleavable sequences are known in the literature to a person of skill in the art, such as for non-limiting example, in Hervio et al., Chemistry & Biology 2000, Vol. 7 No. 6, pp. 44-52; Eckhard et al., Matrix Biol. (2016) 49, pp. 37-60; and Jo et al., J Biomed. Mat. Res., (2010) Vol. 93A, Iss. 3, pp. 870-877. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (MMP) cleavable component. In some embodiments, the matrix metalloproteinase (MMP) cleavable component is a matrix metalloproteinase (IMP) cleavable peptide. In some embodiments, the matrix metalloproteinase (PNI) cleavable peptide is a peptide comprising a sequence selected from the group consisting of SEQ ID NOs: 15-19. In some embodiments, the matrix metalloproteinase (MMP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (NIP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MIP) cleavable peptide is a peptide comprising a sequence selected from the group consisting of SEQ ID NOs: 15-19. In some embodiments, the matrix metalloproteinase (NIP) cleavable peptide is a peptide with a sequence selected from the group consisting of KCGPQGIAGQCK (SEQ ID NO: 18) and K(hC)GPQGIAGQ(hC)K (SEQ ID NO: 19). In some embodiments, the matrix metalloproteinase (MMP) cleavable peptide is a peptide KCGPQGIAGQCK (SEQ ID NO: 18). In some embodiments, the matrix metalloproteinase (MMP) cleavable peptide is a peptide K(hC)GPQGIAGQ(hC)K (SEQ ID NO: 19). In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the plasmin cleavable peptide is a peptide of SEQ ID NO: 4. In some embodiments, the plasmin cleavable peptide is a peptide of SEQ ID NO: 7. In some embodiments, the matrix metalloproteinase (NIP) cleavable peptide is a peptide with a sequence selected from the group consisting of KCGPQGIAGQCK (SEQ ID NO: 18) and K(hC)GPQGIAGQ(hC)K (SEQ ID NO: 19), and the plasmin cleavable peptide is a peptide with a sequence selected from the group consisting of CALKVLKGCG-amide (SEQ ID NO: 4) and (hC)ALKVLKG(hC)G-amide (SEQ ID NO: 7). In some embodiments, the matrix metalloproteinase (IMP) cleavable peptide is a peptide of SEQ ID NO: 18, and the plasmin cleavable peptide is a peptide of SEQ ID NO: 4. In some embodiments, the matrix metalloproteinase (IMP) cleavable peptide is a peptide of SEQ ID NO: 19, and the plasmin cleavable peptide is a peptide of SEQ ID NO: 7. It is understood that the protease-cleavable peptides recited above are non-limiting examples and that a person of ordinary skill in the art would recognize that numerous protease-cleavable peptides can be suitable for the intended purpose.

In a second facet, provided herein is a method for making a hydrogel biomaterial comprising

at least one polymer component;

at least one multivalent linker component; and

a fibrinogen component;

wherein at least a portion of the at least one polymer component, at least a portion of the at least one multivalent linker component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive ene groups, wherein the at least one multivalent linker component comprises reactive thiol groups, and at least a portion of the fibrinogen component wherein the fibrinogen component comprises reactive thiol groups, to a chemical reaction to create the carbon-sulfur covalent bonds of the plurality of crosslink units.

In some embodiments of the second facet, the at least one polymer component comprises a linear polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of about 3 kDa, about 6 kDa, and about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

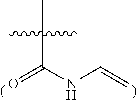 [SMILES: *C(=O)NC=C]

and norborn-2-en-5-yl

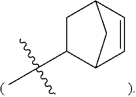 [SMILES: *C1CC2C=CC1C2]

In some embodiments, wherein the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of about 3 kDa, about 6 kDa, and about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa.

In some embodiments of the second facet, the at least one polymer component comprises a branched polymer with reactive ene groups. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

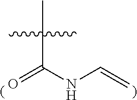 [SMILES: *C(=O)NC=C]

and norborn-2-en-5-yl

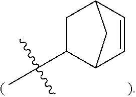 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 3-arm poly(ethylene glycol) moiety and three terminal norborn-2-en-5-yl groups (one at each terminus). In some embodiments, the 3-arm poly(ethylene glycol) moiety has a molecular weight of between about 1 kDa and about 40 kDa. In some embodiments, the 3-arm poly(ethylene glycol) moiety has a molecular weight of about 15 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of between about 1 kDa and about 40 kDa. In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of between about 1 kDa and about 40 kDa. In some embodiments, the 6-arm poly(ethylene glycol) moiety has a molecular weight of about 30 kDa.

In some embodiments of the second facet, the at least one polymer component comprises a linear polymer with reactive ene groups and a branched polymer with reactive ene groups. In some embodiments, the linear polymer with reactive ene groups comprises a linear polymeric moiety and two reactive ene groups, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, the linear polymer with reactive ene groups comprises a linear polymeric moiety and two reactive ene groups, wherein the linear polymeric moiety is selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the linear polymer with reactive ene groups comprises a linear polymeric moiety and two reactive ene groups, wherein the linear polymeric moiety is a poly(ethylene glycol) moiety having a molecular weight of between about 1 kDa and about 40 kDa. In some embodiments, the linear polymer with reactive ene groups comprises a linear polymeric moiety and two reactive ene groups, wherein the linear polymeric moiety is a poly(ethylene glycol) moiety having a molecular weight selected from the group consisting of 3 kDa, 6 kDa, and 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 10 kDa. In some embodiments, the linear polymer with reactive ene groups comprises a linear polymeric moiety and two reactive ene groups, wherein the linear polymeric moiety is a poly(ethylene glycol) moiety having a molecular weight of between about 6 kDa. In some embodiments, the linear polymer with reactive ene groups comprises a linear polymeric moiety and two reactive ene groups, wherein the linear polymeric moiety is a poly(ethylene glycol) moiety having a molecular weight of between about 3 kDa. In some embodiments, the two reactive ene groups of the linear polymer with reactive ene groups are terminal reactive ene groups. In some embodiments, the two terminal reactive ene groups of the linear polymer with reactive ene groups are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

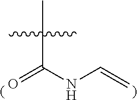 [SMILES: *C(=O)NC=C]

and norborn-2-en-5-yl

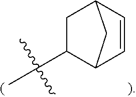 [SMILES: *C1CC2C=CC1C2]

In some embodiments, the linear polymer with reactive ene groups comprises a poly(ethylene glycol) moiety and two terminal norborn-2-en-5-yl groups (one at each terminus). In some embodiments, the linear polymer with terminal reactive ene groups comprises a poly(ethylene glycol) moiety and two terminal norborn-2-en-5-yl groups, wherein the poly(ethylene glycol) moiety has a molecular weight selected from the group consisting of 3 kDa, 6 kDa, and 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 10 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 6 kDa. In some embodiments, the linear polymer with terminal reactive ene groups comprises a poly(ethylene glycol) moiety and two terminal norborn-2-en-5-yl groups, wherein the poly(ethylene glycol) moiety has a molecular weight of about 3 kDa. In some embodiments, the linear polymer component comprises a linear polymeric moiety having a molecular weight of between about 500 Da and about 40 kDa. In some embodiments, the linear polymer component comprises a linear polymeric moiety having a molecular weight of between about 1 kDa and about 40 kDa. In some embodiments, the linear polymer component comprises a linear polymeric moiety having a molecular weight selected from the group consisting of about 3 kDa, about 6 kDa, and about 10 kDa. In some embodiments, the branched polymer with reactive ene groups comprises a branched polymeric moiety with n polymeric arms and n reactive ene groups, wherein n is an integer selected from the group consisting of 3, 4, 5, 6, 7, 8, 9, and 10. In some embodiments, n is 3. In some embodiments, n is 4. In some embodiments, n is 6. In some embodiments, n is 8. In some embodiments, each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing. In some embodiments, each of the n polymeric arms is independently selected from the group consisting of polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), and copolymers thereof, and blends of the foregoing. In some embodiments, the n reactive ene groups of the branched polymer are terminal reactive ene groups. In some embodiments, each of the n reactive ene groups of the branched polymer is, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

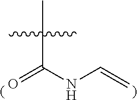 [SMILES: *C(=O)NC=C]

and norborn-2-en-5-yl

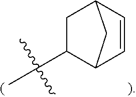 [SMILES: *C1CC2C=CC1C2]

In some embodiments, the branched polymeric moiety has a molecular weight of between about 500 Da and about 40 kDa. In some embodiments, the branched polymeric moiety has a molecular weight of between about 1 kDa and about 40 kDa. In some embodiments, the branched polymer with reactive ene groups comprises a 4-arm poly(ethylene glycol) moiety and four terminal norborn-2-en-5-yl groups (one at each terminus). In some embodiments, the 4-arm poly(ethylene glycol) moiety has a molecular weight of about 20 kDa. In some embodiments, the branched polymer with reactive ene groups comprises a 6-arm poly(ethylene glycol) moiety and six terminal norborn-2-en-5-yl groups (one at each terminus). In some embodiments, the 6-arm poly(ethylene glycol) moiety has a molecular weight of about 30 kDa.

In some embodiments of second facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of the second facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In a third facet, provided herein is a method for making a hydrogel biomaterial comprising

at least one multivalent linker component;

at least one protease-cleavable component; and

a fibrinogen component;

wherein at least a portion of the at least one multivalent linker component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one multivalent linker component wherein the at least one multivalent linker component comprises reactive thiol groups, wherein the at least one protease-cleavable component comprises reactive ene groups, and at least a portion of the fibrinogen component wherein the fibrinogen component comprises reactive thiol groups, to a chemical reaction to create the carbon-sulfur covalent bonds of the plurality of crosslink units.

In some embodiments of the third facet, the at least one multivalent linker component comprises a polythiol component. In some embodiments, the polythiol component has a molecular weight of less than about 2000 g/mol. In some embodiments, the polythiol component has a molecular weight of less than about 1000 g/mol. In non-limiting examples, the at least one multivalent linker component can be DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, or a mixture of any of the foregoing. In some embodiments of the third facet, the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

In some embodiments of the third facet, the at least one protease-cleavable component is a peptide (such as a synthetic peptide) comprising at least two reactive ene groups. In some embodiments, each of the at least one protease-cleavable component is a peptide having a sequence of between 3 and 25 amino acids, and wherein the peptide comprises at least two reactive ene groups. In some embodiments, each of the at least one protease-cleavable component is a peptide having a sequence of 4, 9, 10, 11, 12, 13, or 16 amino acids. In some embodiments, each of the at least one protease-cleavable component comprises a peptide chain comprising a sequence selected from the group consisting of SEQ ID NOs: 1-19 as shown in Tables 1 and 2. It is understood that the protease-cleavable sequences described in Tables 1 and 2 are presented as non-limiting examples. A person of ordinary skill in the art would recognize that other protease-cleavable sequences are readily available either commercially or through synthesis. In addition, it is understood that sequences comprising the amino acids sequences described above (SEQ ID NOs: 1-19) and additional flanking amino acids (on either or both side of the peptide) are equally suitable. Further, it is understood for non-limiting exemplary purpose that additional suitable protease-cleavable components may be prepared by modifying the amino acids sequences described above (SEQ ID NOs: 1-19) via the addition of flanking amino acids comprising reactive ene groups suitable for thiol-ene photoinitiated radical chemistry. The protease-cleavable component may optionally comprise other additional flanking amino acids. Alternatively, it is understood for non-limiting exemplary purpose additional suitable protease-cleavable components may be prepared by modifying the amino acids sequences described above (SEQ ID NOs: 1-19) via the addition of flanking groups comprising reactive ene groups suitable for thiol-ene photoinitiated radical chemistry, such as for non-limiting example by modifying the carboxylic acid terminus and amino terminus of a peptide having an amino acid sequence selected from the group consisting of SEQ ID NOs: 1-19 with synthetic spacers comprising reactive ene groups. For further non-limiting exemplary purposes, it is understood that additional suitable protease-cleavable components may be prepared by modifying the amino acids sequences described above (SEQ ID NOs: 1-19) via the addition of flanking lysine residues which have been modified via introduction of a reactive ene group on the lysine's side chain by reaction of the lysine's side chain's amino group with a suitable reagent such as, for non-limiting example, 5-norbornene-2-carboxylic acid, the ester of N-hydroxysuccinimide and 5-norbornene-2-carboxylic acid, an allyl halide such as allyl chloride, a vinylacetyl halide such as vinylacetyl chloride, a vinylsulfonyl halide such as vinylsulfonyl chloride, a acryloyl halide such as acryloyl chloride, a methacryloyl halide such as methacryloyl chloride, a maleic anhydride, or the likes. Alternatively, a skilled artisan can prepare suitable protease-cleavable peptides by introducing non-natural amino acid analogs comprising a reactive ene group (such as for non-limiting example norbornene) in the course of solid-phase synthesis. The protease-cleavable component may optionally comprise other additional flanking amino acids. In some embodiments, each of the at least one protease-cleavable component is independently of each other a matrix metalloproteinase (MMP) cleavable component or a plasmin cleavable component. In some embodiments, the mixture comprises one protease-cleavable component. In some embodiments, the protease-cleavable component is a matrix metalloproteinase (IMP) cleavable component. In some embodiments, the matrix metalloproteinase (MMP) cleavable component is a matrix metalloproteinase (MMP) cleavable peptide. In some embodiments, the matrix metalloproteinase (PNI) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. In some embodiments, the mixture comprises two protease-cleavable components. In some embodiments, the mixture comprises two protease-cleavable components, wherein one protease-cleavable component is a matrix metalloproteinase (MMP) cleavable component, and the other is a plasmin cleavable component. In some embodiments, the protease-cleavable component is a matrix metalloproteinase (MMP) cleavable component. In some embodiments, the matrix metalloproteinase (MMP) cleavable component is a matrix metalloproteinase (MMP) cleavable peptide. In some embodiments, the matrix metalloproteinase (MMP) cleavable peptide is a peptide comprising a sequence selected from the group consisting of SEQ ID NOs: 15-19. In some embodiments, the protease-cleavable component is a plasmin cleavable component. In some embodiments, the plasmin cleavable component is a plasmin cleavable peptide. In some embodiments, the plasmin cleavable peptide is a peptide with a sequence selected from the group consisting of SEQ ID NOs: 1-14. It is understood that the protease-cleavable peptides recited above are non-limiting examples and that a person of ordinary skill in the art would recognize that numerous protease-cleavable peptides can be suitable for the intended purpose. In some embodiments, the reactive ene groups of the protease-cleavable component are, independently of each other, selected from the group consisting of vinylacetyl, vinyl ester, vinylsulfonyl, vinyl ether, allyl, acrylate ester, methacrylate ester, acrylamido, maleimido, propenyl ether, allyl ether, alkenyl, unsaturated ester, dienyl, N-vinylcarbamoyl

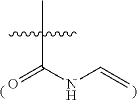 [SMILES: *C(=O)NC=C]

and norborn-2-en-5-yl

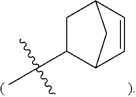 [SMILES: *C1CC2C=CC1C2]

In some embodiments of the preceding facets, the fibrinogen component is not capable of refolding into the conformation of a native fibrinogen molecule. In some embodiments, the fibrinogen component does not possess the clotting activity of a native fibrinogen molecule. In some embodiments, the fibrinogen component is a native fibrinogen molecule. In some embodiments, the fibrinogen component is the product or products of denaturing a native fibrinogen molecule. In some embodiments, the fibrinogen component is an alpha chain of fibrinogen. In some embodiments, the fibrinogen component is a recombinant alpha chain of fibrinogen. In some embodiments, the fibrinogen component is a beta chain of fibrinogen. In some embodiments, the fibrinogen component is a recombinant beta chain of fibrinogen. In some embodiments, the fibrinogen component is a gamma chain of fibrinogen. In some embodiments, the fibrinogen component is a recombinant gamma chain of fibrinogen. In some embodiments, the fibrinogen component is a mixture of an alpha chain of fibrinogen and a beta chain of fibrinogen. In some such embodiments, the mixture is an approximately equimolar mixture of an alpha chain of fibrinogen and a beta chain of fibrinogen. In some embodiments, the fibrinogen component is a mixture of a recombinant alpha chain of fibrinogen and a recombinant beta chain of fibrinogen. In some such embodiments, the mixture is an approximately equimolar mixture of a recombinant alpha chain of fibrinogen and a recombinant beta chain of fibrinogen. In some embodiments, the fibrinogen component is a mixture of an alpha chain of fibrinogen and a gamma chain of fibrinogen. In some such embodiments, the mixture is an approximately equimolar mixture of an alpha chain of fibrinogen and a gamma chain of fibrinogen. In some embodiments, the fibrinogen component is a mixture of a recombinant alpha chain of fibrinogen and a recombinant gamma chain of fibrinogen. In some such embodiments, the mixture is an approximately equimolar mixture of a recombinant alpha chain of fibrinogen and a recombinant gamma chain of fibrinogen. In some embodiments, the fibrinogen component is a mixture of a beta chain of fibrinogen and a gamma chain of fibrinogen. In some such embodiments, the mixture is an approximately equimolar mixture of a beta chain of fibrinogen and a gamma chain of fibrinogen. In some embodiments, the fibrinogen component is a mixture of a recombinant beta chain of fibrinogen and a recombinant gamma chain of fibrinogen. In some such embodiments, the mixture is an approximately equimolar mixture of a recombinant beta chain of fibrinogen and a recombinant gamma chain of fibrinogen. In some embodiments, the fibrinogen compound is a mixture of an alpha chain of fibrinogen, a beta chain of fibrinogen, and a gamma chain of fibrinogen. In some such embodiments, the mixture is an equimolar mixture of an alpha chain of fibrinogen, a beta chain of fibrinogen, and a gamma chain of fibrinogen. In some embodiments, the fibrinogen compound is a mixture of a recombinant alpha chain of fibrinogen, a recombinant beta chain of fibrinogen, and a recombinant gamma chain of fibrinogen. In some such embodiments, the mixture is an equimolar mixture of a recombinant alpha chain of fibrinogen, a recombinant beta chain of fibrinogen, and a recombinant gamma chain of fibrinogen. In some variations of all the preceding embodiments, the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

In some embodiments of the preceding facets, the chemical reaction is a free radical mediated thiol-ene reaction. In some embodiments, the thiol-ene reaction is chemically or photochemically initiated. In some embodiments, the thiol-ene reaction is initiated by light (e.g., UV light or sun light) without an initiator compound. In some embodiments, the thiol-ene reaction is started by a radical initiator. In some embodiments, the radical initiator is a photoinitiator compound. In some embodiments, the photoinitiator is lithium phenyl-2,4,6-trimethylbenzoylphosphinate (LAP) or sodium phenyl-2,4,6-trimethylbenzoylphosphinate (NAP). In some embodiments, the photoinitiator is 2-hydroxy-1-[4-(2-hydroxyethoxy)phenyl]-2-methyl-1-propanone (e.g., Irgacure® 2959), 1-hydroxy-cyclohexyl-phenyl-ketone (e.g., Irgacure® 184), 2,2-dimethoxy-1,2-diphenylethan-1-one (e.g., Irgacure® 651), or 2′,4′,5′,7′-Tetrabromofluorescein (Eosyn Y). In some embodiments, the thiol-ene reaction is initiated by exposing the photoinitiator to a light having a wavelength matching the excitation wavelength of the photoinitiator. In the case of LAP or NAP, the wavelength of the light is approximately 372 nm, with 360 nm, 380 nm, or 385 nm being within acceptable range. In the case of 2-hydroxy-1-[4-(2-hydroxyethoxy)phenyl]-2-methyl-1-propanone (e.g., Irgacure® 2959), the wavelength of the light is approximately 276 nm or 331 nm. In the case of 1-hydroxy-cyclohexyl-phenyl-ketone (e.g., Irgacure® 184), the wavelength of the light is approximately 246 nm, 280 nm or 333 nm. In the case of 2,2-dimethoxy-1,2-diphenylethan-1-one (e.g., Irgacure® 651), the wavelength of the light is approximately 254 nm or 337 nm. In the case of Eosyn Y, the light is preferably near 520 nm, with 480 and 540 nm being within acceptable range. A skilled artisan would recognize that the wavelength used to initiate the reaction is selected to provide sufficient free radical formation to drive the reaction. In some embodiments, the minimum photoinitiator amount and exposure time is chosen such that the thiol-ene reaction reaches (or nearly reaches) a desired completion. In particular embodiments the exposure time is chosen so that the thiol-ene reaction reaches less than 100% completion, for example about 98%, about 95%, about 90%, about 80%, or about 70% completion. In one embodiment, network formation is monitored by rheology as a function of light intensity, exposure time, or initiator concentration. In another embodiment, the reaction is monitored via the consumption of free thiol using Elman's assay.

In some embodiments of the preceding facets, the hydrogel biomaterial is a hydrogel microparticle. In some embodiments, the hydrogel biomaterial is a hydrogel microparticle that has a longest dimension of between about 0.05 mm and about 1.0 mm. In some embodiments, the hydrogel microparticle has a longest dimension of between about 0.25 mm and about 1 mm. In some embodiments, the hydrogel microparticle has a longest dimension of between about 0.5 mm and about 1 mm. In some embodiments, the hydrogel microparticle has a longest dimension of between about 0.75 mm and about 1 mm. In some embodiments, the hydrogel microparticle has a longest dimension of between about 0.05 mm and about 0.75 mm. In some embodiments, the hydrogel microparticle has a longest dimension of between about 0.05 mm and about 0.5 mm. In some embodiments, the hydrogel microparticle has a longest dimension of between about 0.05 mm and about 0.25 mm. In some embodiments, the hydrogel microparticle has a longest dimension of between about 0.25 mm and about 0.75 mm. In some embodiments, the hydrogel microparticle has a longest dimension of between about 0.1 mm and about 0.7 mm. In some embodiments, the hydrogel microparticle has a longest dimension of between about 0.1 mm and about 0.2 mm. In some embodiments, the hydrogel microparticle has a longest dimension of between about 0.2 mm and about 0.3 mm. In some embodiments, the hydrogel microparticle has a longest dimension of between about 0.3 mm and about 0.4 mm. In some embodiments, the hydrogel microparticle has a longest dimension of between about 0.4 mm and about 0.5 mm. In some embodiments, the hydrogel microparticle has a longest dimension of between about 0.5 mm and about 0.6 mm. In some embodiments, the hydrogel microparticle has a longest dimension of between about 0.6 mm and about 0.7 mm. In some embodiments, the hydrogel microparticle has a longest dimension of between about 0.5 mm and about 0.7 mm. In some embodiments, the hydrogel microparticle has a longest dimension of between about 0.1 mm and about 0.5 mm. In some embodiments, the hydrogel microparticle has a longest dimension of about 0.05 mm. In some embodiments, the hydrogel microparticle has a longest dimension of about 0.1 mm. In some embodiments, the hydrogel microparticle has a longest dimension of about 0.2 mm. In some embodiments, the hydrogel microparticle has a longest dimension of about 0.25 mm. In some embodiments, the hydrogel microparticle has a longest dimension of about 0.3 mm. In some embodiments, the hydrogel microparticle has a longest dimension of about 0.4 mm. In some embodiments, the hydrogel microparticle has a longest dimension of about 0.5 mm. In some embodiments, the hydrogel microparticle has a longest dimension of about 0.6 mm. In some embodiments, the hydrogel microparticle has a longest dimension of about 0.7 mm. In some embodiments, the hydrogel microparticle has a longest dimension of about 1 mm.

In some variations of the preceding embodiments, the hydrogel microparticle is a hydrogel microsphere. In some embodiments, the hydrogel microsphere has a diameter of between about 0.05 mm and about 1.0 mm. In some embodiments, the hydrogel microsphere has a diameter of between about 0.25 mm and about 1 mm. In some embodiments, the hydrogel microsphere has a diameter of between about 0.5 mm and about 1 mm. In some embodiments, the hydrogel microsphere has a diameter of between about 0.75 mm and about 1 mm. In some embodiments, the hydrogel microsphere has a diameter of between about 0.05 mm and about 0.75 mm. In some embodiments, the hydrogel microsphere has a diameter of between about 0.05 mm and about 0.5 mm. In some embodiments, the hydrogel microsphere has a diameter of between about 0.05 mm and about 0.25 mm. In some embodiments, the hydrogel microsphere has a diameter of between about 0.25 mm and about 0.75 mm. In some embodiments, the hydrogel microsphere has a diameter of between about 0.1 mm and about 0.7 mm. In some embodiments, the hydrogel microsphere has a diameter of between about 0.1 mm and about 0.2 mm. In some embodiments, the hydrogel microsphere has a diameter of between about 0.2 mm and about 0.3 mm. In some embodiments, the hydrogel microsphere has a diameter of between about 0.3 mm and about 0.4 mm. In some embodiments, the hydrogel microsphere has a diameter of between about 0.4 mm and about 0.5 mm. In some embodiments, the hydrogel microsphere has a diameter of between about 0.5 mm and about 0.6 mm. In some embodiments, the hydrogel microsphere has a diameter of between about 0.6 mm and about 0.7 mm. In some embodiments, the hydrogel microsphere has a diameter of between about 0.5 mm and about 0.7 mm. In some embodiments, the hydrogel microsphere has a diameter of between about 0.1 mm and about 0.5 mm. In some embodiments, the hydrogel microsphere has a diameter of about 0.05 mm. In some embodiments, the hydrogel microsphere has a diameter of about 0.1 mm. In some embodiments, the hydrogel microsphere has a diameter of about 0.2 mm. In some embodiments, the hydrogel microsphere has a diameter of about 0.25 mm. In some embodiments, the hydrogel microsphere has a diameter of about 0.3 mm. In some embodiments, the hydrogel microsphere has a diameter of about 0.4 mm. In some embodiments, the hydrogel microsphere has a diameter of about 0.5 mm. In some embodiments, the hydrogel microsphere has a diameter of about 0.6 mm. In some embodiments, the hydrogel microsphere has a diameter of about 0.7 mm. In some embodiments, the hydrogel microsphere has a diameter of about 1 mm.

In some embodiments of the preceding facets, the hydrogel biomaterial has a storage modulus of between about 100 Pa and about 10,000 Pa. In some embodiments, the hydrogel biomaterial has a storage modulus of between about 100 Pa and about 5,000 Pa. In some embodiments, the hydrogel biomaterial has a storage modulus of between about 100 Pa and about 2,500 Pa. In some embodiments, the hydrogel biomaterial has a storage modulus of between about 100 Pa and about 2,000 Pa. In some embodiments, the hydrogel biomaterial has a storage modulus of between about 100 Pa and about 1,500 Pa. In some embodiments, the hydrogel biomaterial has a storage modulus of between about 100 Pa and about 1,000 Pa. In some embodiments, the hydrogel biomaterial has a storage modulus of between about 100 Pa and about 750 Pa. In some embodiments, the hydrogel biomaterial has a storage modulus of between about 100 Pa and about 500 Pa. In some embodiments, the hydrogel biomaterial has a storage modulus of between about 100 Pa and about 250 Pa. In some embodiments, the hydrogel biomaterial has a storage modulus of between about 250 Pa and about 10,000 Pa. In some embodiments, the hydrogel biomaterial has a storage modulus of between about 250 Pa and about 5,000 Pa. In some embodiments, the hydrogel biomaterial has a storage modulus of between about 250 Pa and about 2,500 Pa. In some embodiments, the hydrogel biomaterial has a storage modulus of between about 250 Pa and about 2,000 Pa. In some embodiments, the hydrogel biomaterial has a storage modulus of between about 250 Pa and about 1,500 Pa. In some embodiments, the hydrogel biomaterial has a storage modulus of between about 250 Pa and about 1,000 Pa. In some embodiments, the hydrogel biomaterial has a storage modulus of between about 250 Pa and about 750 Pa. In some embodiments, the hydrogel biomaterial has a storage modulus of between about 250 Pa and about 500 Pa. In some embodiments, the hydrogel biomaterial has a storage modulus of between about 500 Pa and about 10,000 Pa. In some embodiments, the hydrogel biomaterial has a storage modulus of between about 500 Pa and about 5,000 Pa. In some embodiments, the hydrogel biomaterial has a storage modulus of between about 500 Pa and about 2,500 Pa. In some embodiments, the hydrogel biomaterial has a storage modulus of between about 500 Pa and about 2,000 Pa. In some embodiments, the hydrogel biomaterial has a storage modulus of between about 500 Pa and about 1,500 Pa. In some embodiments, the hydrogel biomaterial has a storage modulus of between about 500 Pa and about 1,000 Pa. In some embodiments, the hydrogel biomaterial has a storage modulus of between about 500 Pa and about 750 Pa. In some embodiments, the hydrogel biomaterial has a storage modulus of between about 750 Pa and about 10,000 Pa. In some embodiments, the hydrogel biomaterial has a storage modulus of between about 750 Pa and about 5,000 Pa. In some embodiments, the hydrogel biomaterial has a storage modulus of between about 750 Pa and about 2,500 Pa. In some embodiments, the hydrogel biomaterial has a storage modulus of between about 750 Pa and about 2,000 Pa. In some embodiments, the hydrogel biomaterial has a storage modulus of between about 750 Pa and about 1,500 Pa. In some embodiments, the hydrogel biomaterial has a storage modulus of between about 750 Pa and about 1,000 Pa. In some embodiments, the hydrogel biomaterial has a storage modulus of between about 1,000 Pa and about 10,000 Pa. In some embodiments, the hydrogel biomaterial has a storage modulus of between about 1,000 Pa and about 5,000 Pa. In some embodiments, the hydrogel biomaterial has a storage modulus of between about 1,000 Pa and about 2,500 Pa. In some embodiments, the hydrogel biomaterial has a storage modulus of between about 1,000 Pa and about 2,000 Pa. In some embodiments, the hydrogel biomaterial has a storage modulus of between about 1,000 Pa and about 1,500 Pa. In some embodiments, the hydrogel biomaterial has a storage modulus selected from the group consisting of about 100 Pa, about 250 Pa, 500 Pa, about 750 Pa, about 1,000 Pa, about 1,500 Pa, about 2,000 Pa, about 2,500 Pa, about 5,000 Pa, and about 10,000 Pa. In some embodiments, the hydrogel biomaterial has a storage modulus of about 100 Pa. In some embodiments, the hydrogel biomaterial has a storage modulus of about 250 Pa. In some embodiments, the hydrogel biomaterial has a storage modulus of about 500 Pa. In some embodiments, the hydrogel biomaterial has a storage modulus of about 750 Pa. In some embodiments, the hydrogel biomaterial has a storage modulus of about 1,000 Pa. In some embodiments, the hydrogel biomaterial has a storage modulus of about 1,500 Pa. In some embodiments, the hydrogel biomaterial has a storage modulus of about 2,500 Pa. In some embodiments, the hydrogel biomaterial has a storage modulus of about 5,000 Pa. In some embodiments, the hydrogel biomaterial has a storage modulus of about 10,000 Pa.

In some embodiments of the preceding facets, the hydrogel biomaterial does not substantially swell when exposed to a protease capable of cleaving the protease-cleavable component.

In some embodiments of the preceding facets, the longest dimension of the hydrogel biomaterial increases by less than about twofold when 50% of the protease-cleavable component has been cleaved by a protease capable of cleaving the protease-cleavable component. In some embodiments of the preceding facets, the longest dimension of the hydrogel biomaterial increases by less than about 200% when 50% of the protease-cleavable component has been cleaved by a protease capable of cleaving the protease-cleavable component. In some embodiments of the preceding facets, the longest dimension of the hydrogel biomaterial increases by less than about 150% when 50% of the protease-cleavable component has been cleaved by a protease capable of cleaving the protease-cleavable component. In some embodiments of the preceding facets, the longest dimension of the hydrogel biomaterial increases by less than about 100% when 50% of the protease-cleavable component has been cleaved by a protease capable of cleaving the protease-cleavable component. In some embodiments of the preceding facets, the longest dimension of the hydrogel biomaterial increases by less than about 75% when 50% of the protease-cleavable component has been cleaved by a protease capable of cleaving the protease-cleavable component. In some embodiments of the preceding facets, the longest dimension of the hydrogel biomaterial increases by less than about 50% when 50% of the protease-cleavable component has been cleaved by a protease capable of cleaving the protease-cleavable component. In some embodiments of the preceding facets, the longest dimension of the hydrogel biomaterial increases by less than about 25% when 50% of the protease-cleavable component has been cleaved by a protease capable of cleaving the protease-cleavable component. In some embodiments of the preceding facets, the longest dimension of the hydrogel biomaterial increases by less than about 10% when 50% of the protease-cleavable component has been cleaved by a protease capable of cleaving the protease-cleavable component.

In some embodiments of the preceding facets, the hydrogel biomaterial further comprises a biologically active material encapsulated within the hydrogel biomaterial. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules. In such embodiments, the reaction mixture further comprises the biologically active material. In some embodiments, the biologically active material is a cell selected from the group consisting of chondrocytes, immortalized cell lines, stem cells, hormone producing cells, fibroblasts, and the likes. In some embodiments, the biologically active material is a tissue or a tissue aggregate. In some embodiments, the biologically active material is a signalling molecule, a hormone, or a growth factor. In some embodiments, the biologically active material is a protein, a pharmacologically active agent, or an agricultural chemical. In some embodiments, the biologically active material is a protein selected from the group consisting of adhesion peptides (such as RGD adhesion sequence), growth factors, hormones, antihormones, signaling compounds, enzymes, serum proteins, albumins, macroglobulins, globulins, agglutinins, lectins, extracellular matrix proteins, antibodies, and antigens. In some embodiments, the biologically active material is a pharmacologically active agent selected from the group consisting of analgesics, antipyretics, nonsteroidal antiinflammatory drugs, antiallergics, antibacterial drugs, antianemia drugs, cytotoxic drugs, antihypertensive drugs, dermatological drugs, psychotherapeutic drugs, vitamins, minerals, anorexiants, dietetics, antiadiposity drugs, carbohydrate metabolism drugs, protein metabolism drugs, thyroid drugs, antithyroid drugs, and coenzymes. In some embodiments, the biologically active material is an agricultural chemical selected from the group consisting of fungicides, herbicides, fertilizers, pesticides, carbohydrates, nucleic acids, organic molecules, and inorganic biologically active molecules.

In some embodiments of the preceding facets, the method is performed under denaturing conditions. In such embodiments, the reaction mixture further comprises a denaturant. In some embodiments, the denaturant can be selected from the group consisting of urea, thiourea, tetramethyl urea, guanidinium salts such as guanidinium chloride and guanidinium thiocyanate, formamide, acetamide, and the likes. In some embodiments, the denaturant is selected from the group consisting of urea, thiourea, tetramethyl urea, guanidinium salts, guanidinium chloride, guanidinium thiocyanate, formamide, and acetamide. In some embodiments, the denaturant is urea. In some embodiments, the denaturant is guanidinium chloride. In some such embodiments, the method further comprises a step of removing the denaturant from the hydrogel biomaterial after completion of the chemical reaction. In some embodiments, removing the denaturant is achieved by soaking the hydrogel biomaterial in an aqueous media. In some embodiments the aqueous media may include one or more salts, one or more buffers, or mixtures thereof. In some embodiments, the aqueous media comprises a buffer. In some embodiments, the buffer may be selected from the group consisting of phosphate buffers, acetate buffers, tris buffers, or other buffers well known to those skilled in the art. In some embodiments, the buffer is selected from the group consisting of phosphate buffers, acetate buffers, and tris buffers. In some embodiments, the buffer is phosphate buffered saline (PBS). In some embodiments, the aqueous media comprises a salt selected from sodium, potassium, magnesium, calcium, and ammonium salts

### Methods of Use and Uses

Methods of treating an injury comprising administering to a subject in need thereof a hydrogel biomaterial described herein are also provided. In some embodiments, the hydrogel biomaterial comprises a plurality of hydrogel microparticles. In some embodiments, the injury is a wound, a nerve injury, a muscle injury, a cartilage defect, or a bone defect. In some embodiments, the injury is a wound, a nerve injury, a muscle injury, a cartilage defect, a bone defect, or an osteochondral defect. In some embodiments, the wound is an acute wound. In some embodiments, the acute wound is a traumatic wound, a cut, a surgical wound (such as a tissue resection, a biopsy, a MOHS surgery, or a dental extraction socket), or a burn (such as a thermal burn or a chemical burn). In some specific embodiments, the acute wound is a traumatic wound. In some specific embodiments, the acute wound is a cut. In some specific embodiments, the acute wound is a surgical wound. In some specific embodiments, the acute wound is a tissue resection. In some specific embodiments, the acute wound is a biopsy. In some specific embodiments, the acute wound is a dental extraction socket. In some specific embodiments, the acute wound is a burn. In some specific embodiments, the acute wound is a thermal burn. In some specific embodiments, the acute wound is a chemical burn. In some embodiments, the wound is a chronic wound. In some embodiments, the chronic wound is a venous ulcer, a diabetic ulcer, a pressure ulcer, or a wound caused by radiation poisoning. In some specific embodiments, the chronic wound is a venous ulcer. In some specific embodiments, the chronic wound is a diabetic ulcer. In some specific embodiments, the chronic wound is a pressure ulcer. In some specific embodiments, the chronic wound is a wound caused by radiation poisoning. In some embodiments, the cartilage defect is osteoarthritis. In some embodiments, the cartilage defect is a mechanical injury. In some embodiments, the bone defect is a traumatic bone defect, a bone defect caused by surgery, or a bone defect caused by a disease or condition. In some embodiments, the bone defect is a traumatic bone defect. In some specific embodiments, the traumatic bone defect is a cracked bone or a bone fracture. In some specific embodiments, the traumatic bone defect is a cracked bone. In some specific embodiments, the traumatic bone defect is a bone fracture. In some embodiments, the bone defect is a bone defect caused by surgery. In some specific embodiments, the bone defect caused by surgery is a bone defect caused by tumor resection. In some specific embodiments, the bone defect caused by surgery is a bone defect caused by revision surgery such as knee replacement surgery or hip replacement surgery. In some specific embodiments, the bone defect caused by surgery is a bone defect caused by removal of surgical fixation devices. In some embodiments, the bone defect is a bone defect caused by a disease or condition. In some specific embodiments, the bone defect caused by a disease or condition is a bone defect caused by infection. In some specific embodiments, the bone defect caused by a disease or condition is a bone defect caused by osteoarthritis. In some specific embodiments, the bone defect caused by a disease or condition is a bone defect caused by a neoplasm. In some embodiments, the osteochondral defect is a focal area of damage that involves both the cartilage and a piece of underlying bone. In some embodiments, the osteochondral defect is an osteochondral defect occurring from an acute traumatic injury. In some embodiments, the osteochondral defect is an osteochondral defect occurring from an underlying disorder of a bone.

Methods of treating a tissue defect or improving a cosmetic outcome, comprising administering to a subject in need thereof a hydrogel biomaterial described herein are also provided. In some embodiments, the hydrogel biomaterial comprises a plurality of hydrogel microparticles. In some embodiments, the method is a method of treating a tissue defect. In some embodiments, the method is a method of reconstructive surgery of soft tissue. In some embodiments, the method is a method of reconstructive surgery of bone. In some embodiments, the method is a method of reconstructive surgery of cartilage. In some embodiments, the cartilage is ear cartilage. In some embodiments, the cartilage is nose cartilage. In some embodiments, the method is a method of reconstructive surgery of muscle. In some embodiments, the method is a method of improving a cosmetic outcome. In some embodiments, the method is a method of cosmetic surgery of soft tissue. In some embodiments, the method is a method of cosmetic surgery of bone. In some embodiments, the method is a method of cosmetic surgery of cartilage. In some embodiments, the cartilage is ear cartilage. In some embodiments, the cartilage is nose cartilage. In some embodiments, the method is a method of cosmetic surgery of muscle.

Methods of creating a filler to fill a tissue void comprising administering the hydrogel biomaterial to the tissue void are also provided. In some embodiments, the hydrogel biomaterial comprises a plurality of hydrogel microparticles. In some embodiments, the filler is a dermal filler. In some embodiments, the filler is a bone filler.

Methods of treating pain comprising administering to a subject in need thereof a hydrogel biomaterial described herein are also provided. In some embodiments, the hydrogel biomaterial comprises a plurality of hydrogel microparticles. Methods of treating an orthopedic condition comprising administering to a subject in need thereof a hydrogel biomaterial described herein are also provided. In some embodiments, the hydrogel biomaterial comprises a plurality of hydrogel microparticles.

Uses of a hydrogel biomaterial described herein for treating an injury are also provided. In some embodiments, the hydrogel biomaterial comprises a plurality of hydrogel microparticles. In some embodiments, the injury is a wound, a nerve injury, a muscle injury, a cartilage defect, or a bone defect. In some embodiments, the injury is a wound, a nerve injury, a muscle injury, a cartilage defect, a bone defect, or an osteochondral defect. In some embodiments, the wound is an acute wound. In some embodiments, the acute wound is a traumatic wound, a cut, a surgical wound (such as a tissue resection, a biopsy, a MOHS surgery, or a dental extraction socket), or a burn (such as a thermal burn or a chemical burn). In some specific embodiments, the acute wound is a traumatic wound. In some specific embodiments, the acute wound is a cut. In some specific embodiments, the acute wound is a surgical wound. In some specific embodiments, the acute wound is a tissue resection. In some specific embodiments, the acute wound is a biopsy. In some specific embodiments, the acute wound is a dental extraction socket. In some specific embodiments, the acute wound is a burn. In some specific embodiments, the acute wound is a thermal burn. In some specific embodiments, the acute wound is a chemical burn. In some embodiments, the wound is a chronic wound. In some embodiments, the chronic wound is a venous ulcer, a diabetic ulcer, a pressure ulcer, or a wound caused by radiation poisoning. In some specific embodiments, the chronic wound is a venous ulcer. In some specific embodiments, the chronic wound is a diabetic ulcer. In some specific embodiments, the chronic wound is a pressure ulcer. In some specific embodiments, the chronic wound is a wound caused by radiation poisoning. In some embodiments, the cartilage defect is osteoarthritis. In some embodiments, the cartilage defect is a mechanical injury. In some embodiments, the bone defect is a traumatic bone defect, a bone defect caused by surgery, or a bone defect caused by a disease or condition. In some embodiments, the bone defect is a traumatic bone defect. In some specific embodiments, the traumatic bone defect is a cracked bone or a bone fracture. In some specific embodiments, the traumatic bone defect is a cracked bone. In some specific embodiments, the traumatic bone defect is a bone fracture. In some embodiments, the bone defect is a bone defect caused by surgery. In some specific embodiments, the bone defect caused by surgery is a bone defect caused by tumor resection. In some specific embodiments, the bone defect caused by surgery is a bone defect caused by revision surgery such as knee replacement surgery or hip replacement surgery. In some specific embodiments, the bone defect caused by surgery is a bone defect caused by removal of surgical fixation devices. In some embodiments, the bone defect is a bone defect caused by a disease or condition. In some specific embodiments, the bone defect caused by a disease or condition is a bone defect caused by infection. In some specific embodiments, the bone defect caused by a disease or condition is a bone defect caused by osteoarthritis. In some specific embodiments, the bone defect caused by a disease or condition is a bone defect caused by a neoplasm. In some embodiments, the osteochondral defect is a focal area of damage that involves both the cartilage and a piece of underlying bone. In some embodiments, the osteochondral defect is an osteochondral defect occurring from an acute traumatic injury. In some embodiments, the osteochondral defect is an osteochondral defect occurring from an underlying disorder of a bone.

Uses of a hydrogel biomaterial described herein for treating a tissue defect or improving a cosmetic outcome are also provided. In some embodiments, the hydrogel biomaterial comprises a plurality of hydrogel microparticles. In some embodiments, the use is a use for treating a tissue defect. In some embodiments, the use is a use for reconstructive surgery of soft tissue. In some embodiments, the use is a use for reconstructive surgery of bone. In some embodiments, the use is a use for reconstructive surgery of cartilage. In some embodiments, the cartilage is ear cartilage. In some embodiments, the cartilage is nose cartilage. In some embodiments, the use is a use for reconstructive surgery of muscle. In some embodiments, the use is a use for improving a cosmetic outcome. In some embodiments, the use is a use for cosmetic surgery of soft tissue. In some embodiments, the use is a use for cosmetic surgery of bone. In some embodiments, the use is a use for cosmetic surgery of cartilage. In some embodiments, the cartilage is ear cartilage. In some embodiments, the cartilage is nose cartilage. In some embodiments, the use is a use for cosmetic surgery of muscle. Uses of a hydrogel biomaterial described herein for creating a filler to fill a tissue void comprising administering the hydrogel biomaterial to the tissue void. In some embodiments, the hydrogel biomaterial comprises a plurality of hydrogel microparticles. In some embodiments, the filler is a dermal filler. In some embodiments, the filler is a bone filler.

Uses of a hydrogel biomaterial described herein for treating pain are also provided. In some embodiments, the hydrogel biomaterial comprises a plurality of hydrogel microparticles. Uses of a hydrogel biomaterial described herein for treating an orthopedic condition are also provided. In some embodiments, the hydrogel biomaterial comprises a plurality of hydrogel microparticles.

Uses of a hydrogel biomaterial described herein in the manufacture of a medicament for treating an injury are also provided. In some embodiments, the hydrogel biomaterial comprises a plurality of hydrogel microparticles. In some embodiments, the injury is a wound, a nerve injury, a muscle injury, a cartilage defect, or a bone defect. In some embodiments, the injury is a wound, a nerve injury, a muscle injury, a cartilage defect, a bone defect, or an osteochondral defect. In some embodiments, the wound is an acute wound. In some embodiments, the acute wound is a traumatic wound, a cut, a surgical wound (such as a tissue resection, a biopsy, a MOHS surgery, or a dental extraction socket), or a burn (such as a thermal burn or a chemical burn). In some specific embodiments, the acute wound is a traumatic wound. In some specific embodiments, the acute wound is a cut. In some specific embodiments, the acute wound is a surgical wound. In some specific embodiments, the acute wound is a tissue resection. In some specific embodiments, the acute wound is a biopsy. In some specific embodiments, the acute wound is a dental extraction socket. In some specific embodiments, the acute wound is a burn. In some specific embodiments, the acute wound is a thermal burn. In some specific embodiments, the acute wound is a chemical burn. In some embodiments, the wound is a chronic wound. In some embodiments, the chronic wound is a venous ulcer, a diabetic ulcer, a pressure ulcer, or a wound caused by radiation poisoning. In some specific embodiments, the chronic wound is a venous ulcer. In some specific embodiments, the chronic wound is a diabetic ulcer. In some specific embodiments, the chronic wound is a pressure ulcer. In some specific embodiments, the chronic wound is a wound caused by radiation poisoning. In some embodiments, the cartilage defect is osteoarthritis. In some embodiments, the cartilage defect is a mechanical injury. In some embodiments, the bone defect is a traumatic bone defect, a bone defect caused by surgery, or a bone defect caused by a disease or condition. In some embodiments, the bone defect is a traumatic bone defect. In some specific embodiments, the traumatic bone defect is a cracked bone or a bone fracture. In some specific embodiments, the traumatic bone defect is a cracked bone. In some specific embodiments, the traumatic bone defect is a bone fracture. In some embodiments, the bone defect is a bone defect caused by surgery. In some specific embodiments, the bone defect caused by surgery is a bone defect caused by tumor resection. In some specific embodiments, the bone defect caused by surgery is a bone defect caused by revision surgery such as knee replacement surgery or hip replacement surgery. In some specific embodiments, the bone defect caused by surgery is a bone defect caused by removal of surgical fixation devices. In some embodiments, the bone defect is a bone defect caused by a disease or condition. In some specific embodiments, the bone defect caused by a disease or condition is a bone defect caused by infection. In some specific embodiments, the bone defect caused by a disease or condition is a bone defect caused by osteoarthritis. In some specific embodiments, the bone defect caused by a disease or condition is a bone defect caused by a neoplasm. In some embodiments, the osteochondral defect is a focal area of damage that involves both the cartilage and a piece of underlying bone. In some embodiments, the osteochondral defect is an osteochondral defect occurring from an acute traumatic injury. In some embodiments, the osteochondral defect is an osteochondral defect occurring from an underlying disorder of a bone.

Uses of a hydrogel biomaterial described herein in the manufacture of a medicament for treating a tissue defect or improving a cosmetic outcome are also provided. In some embodiments, the hydrogel biomaterial comprises a plurality of hydrogel microparticles. In some embodiments, the use is a use in the manufacture of a medicament for treating a tissue defect. In some embodiments, the use is a use in the manufacture of a medicament for reconstructive surgery of soft tissue. In some embodiments, the use is a use in the manufacture of a medicament for reconstructive surgery of bone. In some embodiments, the use is a use in the manufacture of a medicament for reconstructive surgery of cartilage. In some embodiments, the cartilage is ear cartilage. In some embodiments, the cartilage is nose cartilage. In some embodiments, the use is a use in the manufacture of a medicament for reconstructive surgery of muscle. In some embodiments, the use is a use in the manufacture of a medicament for improving a cosmetic outcome. In some embodiments, the use is a use in the manufacture of a medicament for cosmetic surgery of soft tissue. In some embodiments, the use is a use in the manufacture of a medicament for cosmetic surgery of bone. In some embodiments, the use is a use in the manufacture of a medicament for cosmetic surgery of cartilage. In some embodiments, the cartilage is ear cartilage. In some embodiments, the cartilage is nose cartilage. In some embodiments, the use is a use in the manufacture of a medicament for cosmetic surgery of muscle.

Uses of a hydrogel biomaterial described herein in the manufacture of a medicament for treating pain are also provided. In some embodiments, the hydrogel biomaterial comprises a plurality of hydrogel microparticles. Uses of a hydrogel biomaterial described herein in the manufacture of a medicament for treating an orthopedic condition are also provided. In some embodiments, the hydrogel biomaterial comprises a plurality of hydrogel microparticles.

### Additional Pharmaceutical Agents, Additional Biologic Medical Products, and Combination Therapies

In certain aspects of the present disclosure, a hydrogel biomaterial described herein can be combined with additional treatments including, but not limited to cosmetics, drugs, biological products, devices, or combinations of the foregoing. In some embodiments, the hydrogel biomaterial comprises a plurality of hydrogel microparticles. In some embodiments, a cosmetics, a drug, a biological product, or a combination of the foregoing is added to the reaction mixture and encapsulated into the hydrogel biomaterial upon polymerization. In some embodiments, the cosmetics, the drug, and/or the biological product is derivatized to incorporate a reactive thiol group or a reactive ene group suitable for thiol-ene photoradical reaction. In such embodiments, the cosmetics, the drug, and/or the biological product is covalently linked and incorporated into the hydrogel biomaterial. In some embodiments, an additional treatment is administered concurrently to treatment with the hydrogel biomaterial described herein. In some embodiments, an additional treatment is administered separately from treatment with the hydrogel biomaterial described herein. In some embodiments, an additional treatment is administered prior to treatment with the hydrogel biomaterial described herein. In some embodiments, an additional treatment is administered subsequently to treatment with the hydrogel biomaterial described herein.

### Articles of Manufacture and Kits

The present disclosure further provides articles of manufacture comprising a container in which a composition provided herein (e.g., a hydrogel biomaterial described herein, a microparticle described herein, a plurality of hydrogel microparticles described herein, or a composition comprising a precursor to a hydrogel biomaterial or a hydrogel microparticle described herein) is contained are provided. The article of manufacture may be a bottle, vial (including a sealed or sealable tube), ampoule, single-use disposable applicator, syringe, or the like. The container may be formed from a variety of materials, such as glass or plastic and in one aspect also contains a label on, or associated with, the container which indicates directions for use in the treatment of an indication provided herein, such as healing a wound. The composition provided herein (e.g., a hydrogel biomaterial described herein, a microparticle described herein, a plurality of hydrogel microparticles described herein, or a composition comprising a precursor to a hydrogel biomaterial or a hydrogel microparticle described herein) can be present in the article of manufacture under various forms such as, for non-limiting example, a liquid composition, a frozen liquid composition, a powder composition, a freeze-dried (lyophilized) composition, a crystalized composition, a precipitated composition, a polymerized composition, and the likes. In some embodiments, the composition is a freeze-dried (lyophilized) composition, further comprising a lyoprotectant. In some embodiments, the lyoprotectant is selected from the group consisting of a non-reducing sugar, a non-ionic detergent, and a mixture of the foregoing. In some embodiments, the non-reducing sugar is sucrose, trehalose, or a mixture of the foregoing. In some embodiments, the non-ionic detergent is Tween 20 (Polyoxyethylene (20) sorbitan monolaurate) or Tween 80 (Polyoxyethylene (80) sorbitan monooleate). In some embodiments, the lyoprotectant is a mixture of sucrose and Tween 80.

In one aspect, the container is a medical device containing a unit dosage form of a composition provided herein. The device may contain an applicator for applying the composition to a damaged site on an individual (e.g., a wound). Alternatively, a container (e.g., a bottle, vial or ampoule) may be packaged together with, or provided with instructions for use in conjunction with, a needle and/or syringe which is used to dispense the composition from the container to the desired site (e.g., to the wound). In one aspect, an article of manufacture comprises precursors to a composition provided herein (e.g., a hydrogel biomaterial described herein, a microparticle described herein, a plurality of hydrogel microparticles described herein) packaged in separate containers. In some embodiments, the fibrinogen component is packaged in a first container, the protease-cleavable component is packaged in a second container, and the polymer component is packaged in a third container. In some embodiments, the fibrinogen component is packaged in a first container; the protease-cleavable component and the polymer component are packaged together in a second container. Each component can be packaged in separate containers or some components can be combined in one container where cross-reactivity and shelf life permit. Each component can be present under various forms such as, for non-limiting example, a liquid composition, a frozen liquid composition, a powder composition, a freeze-dried (lyophilized) composition, a crystalized composition, a precipitated composition, a polymerized composition, and the likes. In some embodiments, a first container contains the at least one polymer component and a fraction of the at least one protease-cleavable component, a second container contains the fibrinogen component and a fraction of the at least one protease-cleavable component, and a third container contains a photoinitiator.

Any composition described herein or precursor to a composition described therein may be used in an article of manufacture, the same as if each and every composition were specifically and individually listed for use in an article of manufacture.

In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits comprising a composition provided herein (e.g., a composition comprising a biomaterial or a precursor to a biomaterial and optional additional agents, such as an additional therapeutic agent) are also provided. In one aspect, the kit comprises instructions for use in the treatment of an injury, such as a wound.

The present disclosure further provides kits comprising a container in which a composition provided herein (e.g., a hydrogel biomaterial described herein, a microparticle described herein, a plurality of hydrogel microparticles described herein, or a composition comprising a precursor to a hydrogel biomaterial or a hydrogel microparticle described herein) is contained are provided. The kit may be a bottle, vial (including a sealed or sealable tube), ampoule, single-use disposable applicator, syringe, or the like. The container may be formed from a variety of materials, such as glass or plastic and in one aspect also contains a label on, or associated with, the container which indicates directions for use in the treatment of an indication provided herein, such as healing a wound. The composition provided herein (e.g., a hydrogel biomaterial described herein, a microparticle described herein, a plurality of hydrogel microparticles described herein, or a composition comprising a precursor to a hydrogel biomaterial or a hydrogel microparticle described herein) can be present in the article of manufacture under various forms such as, for non-limiting example, a liquid composition, a frozen liquid composition, a powder composition, a freeze-dried (lyophilized) composition, a crystalized composition, a precipitated composition, a polymerized composition, and the likes. In some embodiments, the composition is a freeze-dried (lyophilized) composition, further comprising a lyoprotectant. In some embodiments, the lyoprotectant is selected from the group consisting of a non-reducing sugar, a non-ionic detergent, and a mixture of the foregoing. In some embodiments, the non-reducing sugar is sucrose, trehalose, or a mixture of the foregoing. In some embodiments, the non-ionic detergent is Tween 20 (Polyoxyethylene (20) sorbitan monolaurate) or Tween 80 (Polyoxyethylene (80) sorbitan monooleate). In some embodiments, the lyoprotectant is a mixture of sucrose and Tween 80.

In one aspect, the container is a medical device containing a unit dosage form of a composition provided herein. The device may contain an applicator for applying the composition to a damaged site on an individual (e.g., a wound). Alternatively, a container (e.g., a bottle, vial or ampoule) may be packaged together with, or provided with instructions for use in conjunction with, a needle and/or syringe which is used to dispense the composition from the container to the desired site (e.g., to the wound). In one aspect, a kit comprises precursors to a composition provided herein (e.g., a hydrogel biomaterial described herein, a microparticle described herein, a plurality of hydrogel microparticles described herein) packaged in separate containers. In some embodiments, the fibrinogen component is packaged in a first container, the protease-cleavable component is packaged in a second container, and the polymer component is packaged in a third container. In some embodiments, the fibrinogen component is packaged in a first container; the protease-cleavable component and the polymer component are packaged together in a second container. Each component can be packaged in separate containers or some components can be combined in one container where cross-reactivity and shelf life permit. Each component can be packaged in separate containers or some components can be combined in one container where cross-reactivity and shelf life permit. Each component can be present under various forms such as, for non-limiting example, a liquid composition, a frozen liquid composition, a powder composition, a freeze-dried (lyophilized) composition, a crystalized composition, a precipitated composition, a polymerized composition, and the likes. In some embodiments, the composition is a freeze-dried (lyophilized) composition, further comprising a lyoprotectant. In some embodiments, the lyoprotectant is selected from the group consisting of a non-reducing sugar, a non-ionic detergent, and a mixture of the foregoing. In some embodiments, the non-reducing sugar is sucrose, trehalose, or a mixture of the foregoing. In some embodiments, the non-ionic detergent is Tween 20 (Polyoxyethylene (20) sorbitan monolaurate) or Tween 80 (Polyoxyethylene (80) sorbitan monooleate). In some embodiments, the lyoprotectant is a mixture of sucrose and Tween 80.

Any composition described herein or precursor to a composition described therein may be used in a kit, the same as if each and every composition were specifically and individually listed for use in a kit.

In certain embodiments, the kit is for use in any of the methods described herein. Suitable packaging is known in the art and includes, for example, vials, vessels, ampules, bottles, jars, flexible packaging and the like. A kit may further be sterilized and/or sealed.

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### General Synthetic Methods

Thiol-ene polymerizations are photochemically initiated, step growth, free-radical processes that take place between thiols and olefins via a sequential propagation/chain-transfer process. For polymerization to occur, each thiol-containing component should have an average of at least two reactive thiol groups and each ene-containing component should have at least two reactive ene groups, (i.e. the monomer should contain two or more double bonds). Polymerization of a dithiol and a diene results in the formation of a linear polymer, rather than a crosslinked polymer. Crosslinked gels can be readily formed by increasing the functionality, i.e., increasing the degree of branching, of one or both of the monomers to be greater than two. Thiol-ene polymerizations have a number of significant and unique advantages that make them particularly beneficial. These benefits include a step growth polymerization that causes the molecular weight to build up relatively slowly, the ability to photoinitiate the sample without any need for a distinct (and possibly cytotoxic) initiator species or with minimal amounts of an initiator species, the ability to polymerize extremely thick (more than 30 cm) samples because of a self-eliminating light intensity gradient, the very low radical concentration present during polymerizations producing less cellular damage from the free radicals, the reduced oxygen inhibition compared to many other types of free-radical polymerizations and the ease with which monomers of significantly varying chemistry can be copolymerized.

The monomers can vary in size depending upon desired properties for the resulting polymeric material. More particularly, the molecular weight for the monomers can range from about 30 Da to about 150,000 Da. In certain embodiments, prior to formation of the polymeric material, the monomers are derivatized to include thiol or olefin moieties such that they can participate in photo-initiated thiol-ene polymerization. For example, thiolated macromers such as poly(ethylene glycol) dithiol are available commercially or via conventional synthetic routes (e.g. solid-phase peptide synthesis). The olefin moieties can be selected from any suitable compound having a carbon-carbon double bond. For example, the olefin moiety can be selected from any suitable ethylenically unsaturated group such as vinyl, acetyl, vinyl ether, allyl, acrylate, methacrylate, maleimide, and norbornene. If each of the first and second monomers is derivatized with either two thiol or two olefin moieties, the resulting thiol-ene polymer would be a linear copolymer composed of alternating first and second monomer segments. However, in certain embodiments, the thiol-ene polymeric material is formed to contain cross-linking and branching. Thus, the derivatized monomer segments can have more than two thiol or olefin moieties per molecule that can participate in crosslinking and polymerization. The extent of the branching and crosslinking can be controlled by the use of differently derivatized first and second monomer segments and control over the concentration of the starting materials.

In certain embodiments, photoinitiation of the thiol-ene polymerization reaction with these monomeric, oligomeric or polymeric starting materials, leads to a formation of high molecular weight, crosslinked networks in the presence or absence of a chemical initiator within reasonable reaction times. The initiator-less route of photopolymerization can eliminate the adverse effects of chemical initiators and still obtain relatively rapid curing. Because of the step growth nature of the polymerization, it results in the covalently crosslinked networks with physical and mechanical properties like glass transition temperature, degree of swelling in water, stiffness and ductility dependent primarily on the chemical nature and molecular weight of the polymeric chains comprising the network and on the branch order of the physical crosslinks connecting them. Thus, simple changes in molecular weight, number of functional groups, and the chemistry of the monomer between the functional groups allow facile control of the polymer properties over a wide range.

Thiol-ene reactions can be chemically or photochemically initiated. In some embodiments, the reaction is started by a radical initiator. In some instances, the reaction may be started by light (e.g., UV light or sun light) without an initiator compound. In a preferred embodiment, the radical initiator can be a photoinitiator compound. Any suitable photoinitiator may be used, such as those used by Bowman et al., for example the photoinitiators described in U.S. Pat. No. 7,288,608 and WO 2012/103445. In some embodiments, the photoinitiator is lithium phenyl-2,4,6-trimethylbenzoylphosphinate (LAP) or sodium phenyl-2,4,6-trimethylbenzoylphosphinate (NAP). In some embodiments, the photoinitiator is 2-hydroxy-1-[4-(2-hydroxyethoxy)phenyl]-2-methyl-1-propanone (e.g., Irgacure® 2959), 1-hydroxy-cyclohexyl-phenyl-ketone (e.g., Irgacure® 184), 2,2-dimethoxy-1,2-diphenylethan-1-one (e.g., Irgacure® 651), or 2′,4′,5′,7′-Tetrabromofluorescein (Eosyn Y).

The thiol-ene reactions typically are initiated by a photochemical process involving a photoinitiator that results in generating free radicals. Upon absorption of a photon of appropriate energy (wavelength), a photoinitiator can undergo direct photolysis via a homolytic cleavage of a covalent bond, resulting in a formation of a pair of free radicals (Type I photoinitiator, e.g. LAP or NAP). Alternatively, upon absorbing a photon, a photoinitiator can reach an excited electronic state that can react with a co-initiator (alcohol, amine or thiol) to generate a pair of free radicals by a non-homolytic mechanism (Type II photoinitiators, e.g. Eosyn Y). For photo-initiated thiol-ene reactions both types of photoinitiators can be used, and because reactant thiols serve as co-initiators, so no additional co-initiator is necessary in the case of the Type II initiator. Preferably, the wavelength of the light used to initiate reaction is chosen to match the excitation wavelength of the photoinitiator. Thus in some embodiments, the thiol-ene reaction is initiated by exposing the photoinitiator to a light having a wavelength matching the excitation wavelength of the photoinitiator. In the case of LAP or NAP, the wavelength of the light is approximately 372 nm, with 360 nm, 380 nm, or 385 nm being within acceptable range. In the case of 2-hydroxy-1-[4-(2-hydroxyethoxy)phenyl]-2-methyl-1-propanone (e.g., Irgacure® 2959), the wavelength of the light is approximately 276 nm or 331 nm. In the case of 1-hydroxy-cyclohexyl-phenyl-ketone (e.g., Irgacure® 184), the wavelength of the light is approximately 246 nm, 280 nm or 333 nm. In the case of 2,2-dimethoxy-1,2-diphenylethan-1-one (e.g., Irgacure® 651), the wavelength of the light is approximately 254 nm or 337 nm. In the case of Eosyn Y, the light is preferably near 520 nm, with 480 and 540 nm being within acceptable range. A skilled artisan would recognize that the wavelength used to initiate the reaction is selected to provide sufficient free radical formation to drive the reaction. The wavelength may be controlled to avoid undesired side reactions, tissue damage, and/or cell damage.

For optimal results, it is generally advised that the minimum photoinitiator amount and exposure time be chosen such that the thiol-ene reaction reaches (or nearly reaches) a desired completion. In particular embodiments the exposure time is chosen so the thiol-ene or thiol-yne reaction reaches less than 100% completion, for example about 98%, about 95%, about 90%, about 80%, or about 70% completion. This prevents potential radical damage to other components of the reaction mixture. In one embodiment of the current invention, network formation is monitored by rheology as a function of light intensity, exposure time, and initiator concentration. In another embodiment, the reaction is monitored via the consumption of free thiol using Elman's assay. In addition to rheology and thiol consumption, other methods of monitoring the reaction will be clear to those skilled in the art.

The thiol-ene reaction may be carried out in any suitable media or solvents. However, in reactions involving proteins and other water soluble biomaterials, an aqueous media may be advantageous.

Thiol-ene reactions exhibit reduced sensitivity to oxygen inhibition compared to most other radical-mediated polymerizations, such as chain-growth acrylate or methacrylate polymerizations. In some embodiments, however, it is preferred to further reduce or completely remove the oxygen-related inhibition of the reaction. In these cases, the thiol-ene or thiol yne reactions may be carried out in a reduced oxygen environment, such as in the absence of oxygen or in the presence of a reduced amount of oxygen compared to a thiol-ene reaction in which no efforts are made to reduce exposure of the reaction to oxygen. For example, the reaction solution can be degassed prior to initiating the reaction by methods commonly applied in the art. In some embodiments, the method for linking a polypeptide using radical-mediated thiol-ene chemistry comprises removing oxygen from the reaction medium.

The free radical mediated thiol-ene polymerization reaction may be carried out under various conditions to allow the formation of the hydrogel biomaterial described herein. For non-limiting exemplary purpose, the hydrogel biomaterial may be prepared via free radical mediated thiol-ene polymerization of the relevant mixture described above in a microfluidic apparatus, in a mold, as an emulsion in a non-miscible solvent, as an emulsion made of a highly salted aqueous phase and another phase which can be miscible with the aqueous phase once the salt concentration in that aqueous phase is diluted (salting out method), in a template made of water soluble materials, in a template made of materials that dissolve in the organic solvent, via 3D printing, inkjet printing, using membrane with defined size of the channels, spinning disc, via electrospinning and electrospraying, via emulsion polymerization, or other suitable methods known in the art for the production of microparticles. In other non-limiting examples, the relevant mixture is polymerized in bulk and subsequently fragmented into microparticles which are then sorted by different dimensions, mass, or density. In other non-limiting examples, the relevant mixture is polymerized in bulk and subsequently fragmented into microparticles which are then sorted by different longest dimensions. In yet other non-limiting examples, the relevant mixture is polymerized in bulk at the site of application (e.g. at the site of an injury). In other non-limiting examples, the relevant mixture is added to a mold and polymerized in bulk in the mold. In other non-limiting example, the relevant mixture is polymerized using a 3D-printing device.

### ENUMERATED EMBODIMENTS

Embodiment 1A. A hydrogel biomaterial comprising

at least one polymer component;

at least one protease-cleavable component; and

a fibrinogen component derivatized with a plurality of polymeric linkers;

wherein at least a portion of the at least one polymer component, at least a portion of the at least one protease-cleavable component, and at least a portion of the fibrinogen component are crosslinked via a plurality of crosslink units each independently comprising a carbon-sulfur covalent bond.

Embodiment 2A. The hydrogel biomaterial of embodiment 1A, wherein the crosslink units of the plurality of crosslink units are selected from the group consisting of

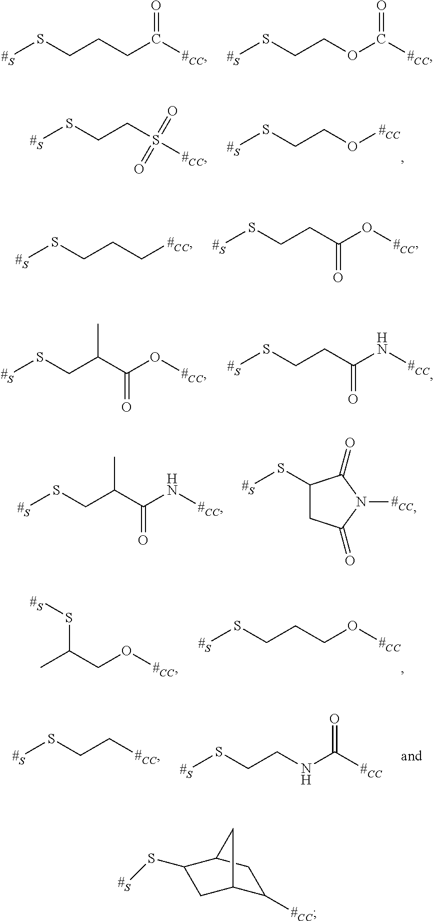

wherein #s and #cc represent attachment points to the at least one polymer component, the at least one protease-cleavable component, or a polymeric linker of the fibrinogen component.

Embodiment 3A. The hydrogel biomaterial of embodiment 1A or 2A, wherein the crosslink units of the plurality of crosslink units are

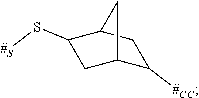

wherein #s and #cc represent attachment points to the at least one polymer component, the at least one protease-cleavable component, or a polymeric linker of the fibrinogen component.

Embodiment 4A. The hydrogel biomaterial of embodiment 1A or 2A, wherein at least a portion of the at least one polymer component is connected to at least a portion of the at least one protease-cleavable component via a first crosslink unit and at least a portion of the fibrinogen component is connected to at least a portion of the at least one protease-cleavable component via a second crosslink unit.

Embodiment 5A. The hydrogel biomaterial of embodiment 4A, wherein the first crosslink unit is selected from the group consisting of

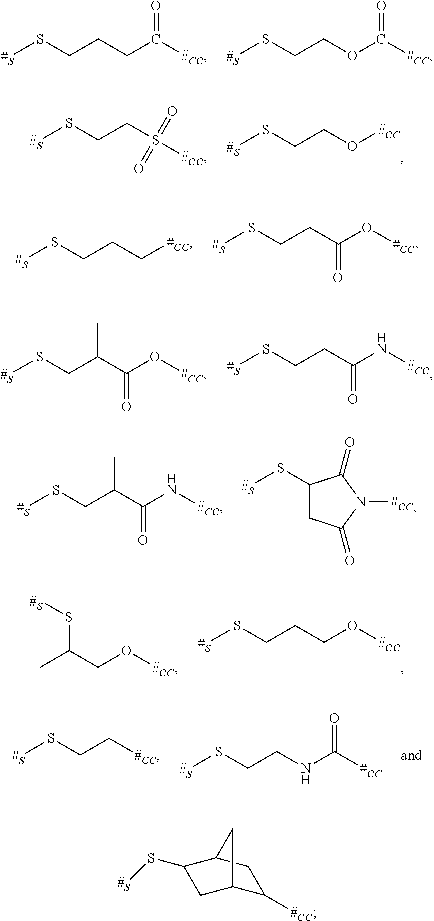

wherein #s represents the attachment point to the at least one protease-cleavable component and #cc represents the attachment point to the at least one polymer component.

Embodiment 6A. The hydrogel biomaterial of embodiment 4A or 5A, wherein the first crosslink unit is

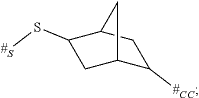

wherein #s represents the attachment point to the at least one protease-cleavable component and #cc represents the attachment point to the at least one polymer component.

Embodiment 7A. The hydrogel biomaterial of any one of embodiments 4A to 6A, wherein the second crosslink unit is selected from the group consisting of

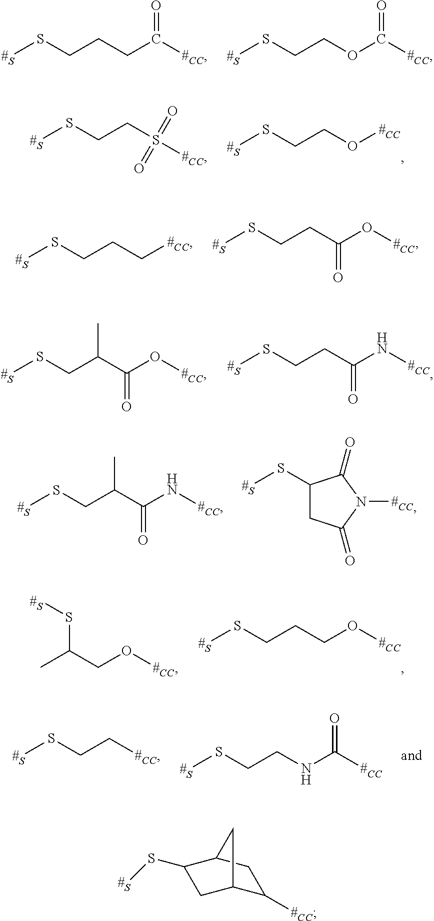

wherein #s represents the attachment point to the at least one protease-cleavable component and #cc represents the attachment point to a polymeric linker of the fibrinogen component.

Embodiment 8A. The hydrogel biomaterial of any one of embodiments 4A to 7A, wherein the second crosslink unit is

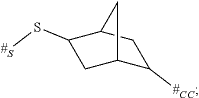

wherein #s represents the attachment point to the at least one protease-cleavable component and #cc represents the attachment point to a polymeric linker of the fibrinogen component.

Embodiment 9A. The hydrogel biomaterial of embodiment 1A or 2A, wherein at least a portion of the at least one polymer component is connected to at least a portion of the at least one protease-cleavable component via a first crosslink unit and at least a portion of the fibrinogen component is connected to at least a portion of the at least one polymer component via a second crosslink unit.

Embodiment 10A. The hydrogel biomaterial of embodiment 9A, wherein the first crosslink unit is selected from the group consisting of

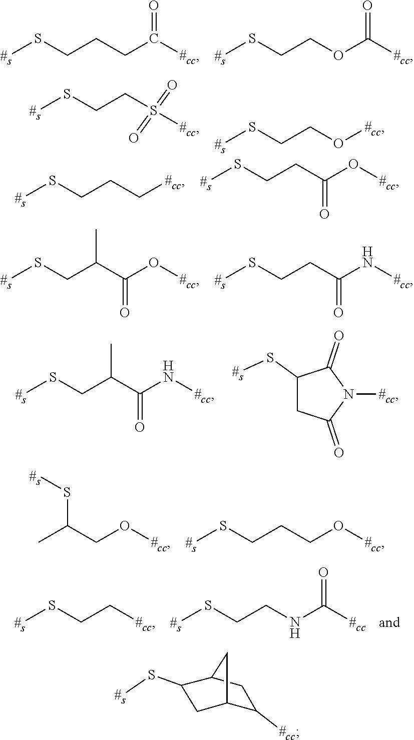

wherein #s represents the attachment point to the at least one polymer component and #cc represents the attachment point to the at least one protease-cleavable component.

Embodiment 11A. The hydrogel biomaterial of embodiment 9A or 10A, wherein the first crosslink unit is

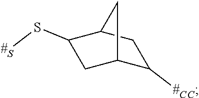

wherein #s represents the attachment point to the at least one polymer component and #cc represents the attachment point to the at least one protease-cleavable component.

Embodiment 12A. The hydrogel biomaterial of any one of embodiments 9A to 11A, wherein the second crosslink unit is selected from the group consisting of

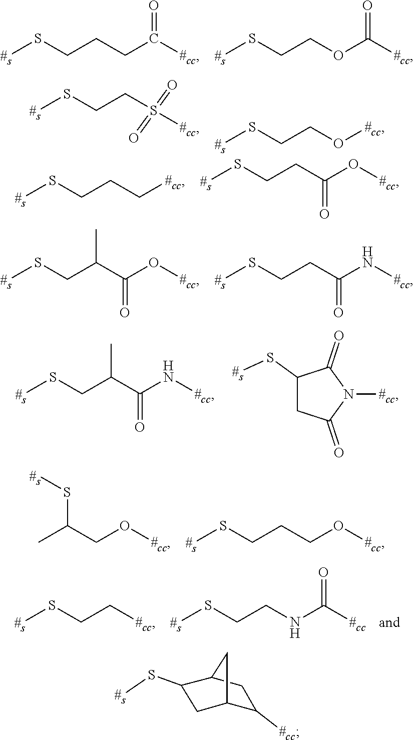

wherein #s represents the attachment point to the at least one polymer component and #cc represents the attachment point to a polymeric linker of the fibrinogen component.

Embodiment 13A. The hydrogel biomaterial of any one of embodiments 9A to 12A, wherein the second crosslink unit is

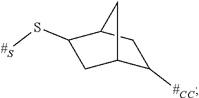

wherein #s represents the attachment point to the at least one polymer component and #cc represents the attachment point to a polymeric linker of the fibrinogen component.

Embodiment 14A. The hydrogel biomaterial of embodiment 1A or 2A, wherein at least a portion of the at least one polymer component is connected to at least a portion of the fibrinogen component via a first crosslink unit and at least a portion of the fibrinogen component is connected to at least a portion of the at least one protease-cleavable component via a second crosslink unit.

Embodiment 15A. The hydrogel biomaterial of embodiment 14A, wherein the first crosslink unit is selected from the group consisting of

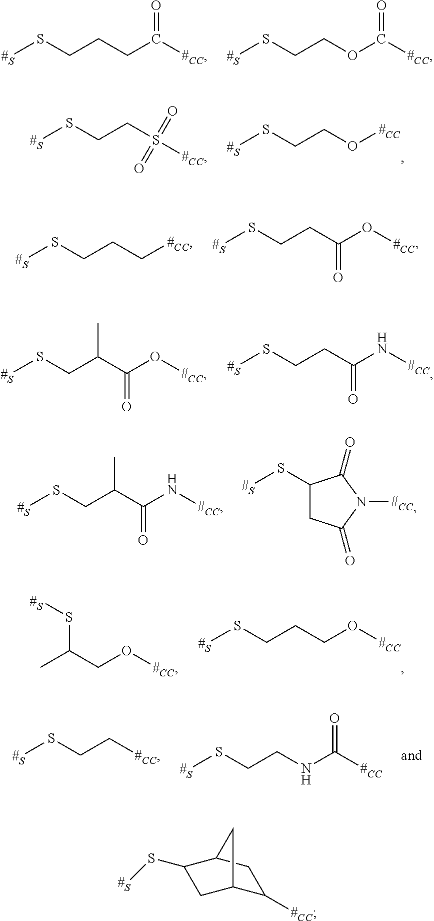

wherein #s represents the attachment point to the at least one polymer component and #cc represents the attachment point to a polymeric linker of the fibrinogen component.

Embodiment 16A. The hydrogel biomaterial of embodiment 14A or 15A, wherein the first crosslink unit is

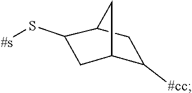

wherein #s represents the attachment point to the at least one polymer component and #cc represents a polymeric linker of the fibrinogen component.

Embodiment 17A. The hydrogel biomaterial of any one of embodiments 14A to 16A, wherein the second crosslink unit is selected from the group consisting of

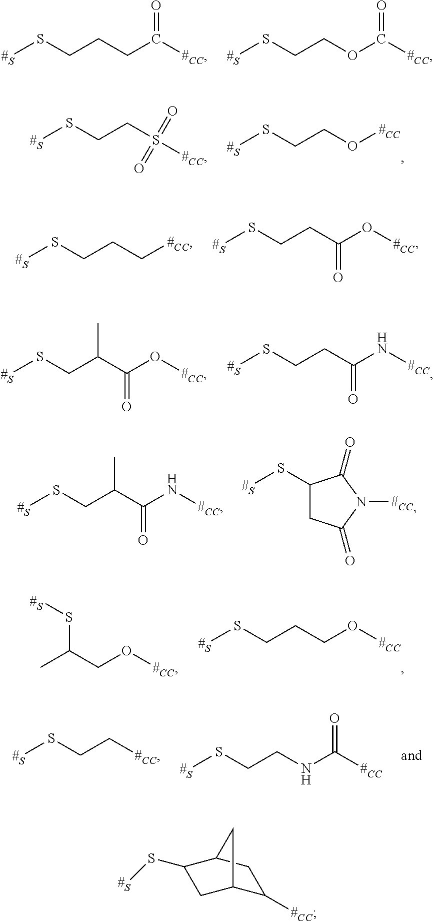

wherein #s represents the attachment point to the at least one protease-cleavable component and #cc represents the attachment point to a polymeric linker of the fibrinogen component.

Embodiment 18A. The hydrogel biomaterial of any one of embodiments 14A to 17A, wherein the second crosslink unit is

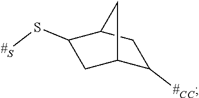

wherein #s represents the attachment point to the at least one protease-cleavable component and #cc represents the attachment point to a polymeric linker of the fibrinogen component.

Embodiment 19A. The hydrogel biomaterial of any one of embodiments 1A to 18A, wherein the at least one polymer component comprises a linear polymer component.

Embodiment 20A. The hydrogel biomaterial of embodiment 19A, wherein the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

Embodiment 21A. The hydrogel biomaterial of embodiment 19A or 20A, wherein the linear polymer component comprises a linear polymeric moiety, wherein the linear polymeric moiety is a poly(ethylene glycol) moiety having a molecular weight of between about 1 kDa and about 40 kDa.

Embodiment 22A. The hydrogel biomaterial of any one of embodiments 19A to 21A, wherein the linear polymer component comprises a linear polymeric moiety, wherein the linear polymeric moiety is a poly(ethylene glycol) moiety having a molecular weight of between about 6 kDa.

Embodiment 23A. The hydrogel biomaterial of any one of embodiments 1A to 18A, wherein the at least one polymer component comprises a branched polymer component.

Embodiment 24A. The hydrogel biomaterial of embodiment 23A, wherein the branched polymer component comprises a branched polymeric moiety with n polymeric arms, wherein n is an integer selected from the group consisting of 3, 4, 5, 6, 7, 8, 9, and 10.

Embodiment 25A. The hydrogel biomaterial of embodiment 24A, wherein the branched polymer component comprises a branched polymeric moiety with n polymeric arms, wherein n is 4.

Embodiment 26A. The hydrogel biomaterial of embodiment 24A or 25A, wherein each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

Embodiment 27A. The hydrogel biomaterial of any one of embodiments 24A to 26A, wherein the branched polymeric moiety has a molecular weight of between about 1 kDa and about 40 kDa.

Embodiment 28A. The hydrogel biomaterial of any one of embodiments 24A to 27A, wherein the branched polymer component comprises a 4-arm poly(ethylene glycol) moiety.

Embodiment 29A. The hydrogel biomaterial of embodiment 28A, wherein the 4-arm poly(ethylene glycol) moiety has a molecular weight of about 20 kDa.

Embodiment 30A. The hydrogel biomaterial of any one of embodiments 1A to 29A, wherein the at least one protease-cleavable component is a synthetic peptide.

Embodiment 31A. The hydrogel biomaterial of any one of embodiments 1A to 30A, wherein each of the at least one protease-cleavable component comprises a peptide chain comprising a sequence selected from the group consisting of SEQ ID NOs: 1-19.

Embodiment 32A. The hydrogel biomaterial of any one of embodiments 1A to 31A, wherein the hydrogel biomaterial comprises two protease-cleavable components, wherein one protease-cleavable component is a matrix metalloproteinase (MMP) cleavable component, and the other is a plasmin cleavable component.

Embodiment 33A. The hydrogel biomaterial of embodiment 32A, wherein the hydrogel biomaterial comprises two protease-cleavable components, wherein the matrix metalloproteinase (MMP) cleavable peptide is a peptide of SEQ ID NO: 19, and the plasmin cleavable peptide is a peptide of SEQ ID NO: 7.

Embodiment 34A. The hydrogel biomaterial of any one of embodiments 1A to 33A, wherein the fibrinogen component derivatized with a plurality of polymeric linkers has been derivatized at cysteine residues.

Embodiment 35A. The hydrogel biomaterial of any one of embodiments 1A to 34A, wherein the fibrinogen component derivatized with a plurality of polymeric linkers has been derivatized at cysteine residues and comprises between 30 moles and 58 moles of polymeric linkers per mole of derivatized fibrinogen.

Embodiment 36A. The hydrogel biomaterial of any one of embodiments 1A to 35A, wherein each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

Embodiment 37A. The hydrogel biomaterial of any one of embodiments 1A to 36A, wherein each individual polymeric linker of the plurality of polymeric linkers comprises a poly(ethylene glycol) moiety, wherein the poly(ethylene glycol) moiety has a molecular weight of about 3.5 kDa.

Embodiment 38A. The hydrogel biomaterial of any one of embodiments 1A to 37A, wherein the hydrogel biomaterial comprises comprising:

0-50% wt. of a linear polymer component comprising a linear PEG moiety, wherein the linear PEG moiety has an average molecular weight of 6 kDa;

0.1-50% wt. of a branched polymer component comprising a 4-armed PEG moiety, wherein the 4-armed PEG moiety has an average molecular weight of 20 kDa;

0-6% wt. of a first protease-cleavable component comprising a IMP-cleavable peptide K(hC)GPQGIAGQ(hC)K (SEQ ID NO: 19);

0-6% wt. of a second protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

Embodiment 39A. The hydrogel biomaterial of embodiment 38A, the hydrogel biomaterial comprises about 0.75% wt. of the fibrinogen component.

Embodiment 40A. The hydrogel biomaterial of embodiment 38A, the hydrogel biomaterial comprises about 1.50% wt. of the fibrinogen component.

Embodiment 41A. The hydrogel biomaterial of embodiment 38A, the hydrogel biomaterial comprises about 3.00% wt. of the fibrinogen component.

Embodiment 42A. The hydrogel biomaterial of any one of embodiments 1A to 41A, wherein the hydrogel biomaterial has a longest dimension of between about 0.05 mm and about 1.0 mm.

Embodiment 43A. The hydrogel biomaterial of any one of embodiments 1A to 42A, wherein the hydrogel biomaterial is a hydrogel microsphere.

Embodiment 44A. The hydrogel biomaterial of embodiment 43A, wherein the hydrogel microsphere has a diameter of between about 0.05 mm and about 1.0 mm.

Embodiment 45A. The hydrogel biomaterial of any one of embodiments 1A to 44A, wherein the hydrogel biomaterial has a storage modulus of between about 100 Pa and about 10,000 Pa.

Embodiment 46A. The hydrogel biomaterial of any one of embodiments 1A to 45A, wherein the hydrogel biomaterial does not substantially swell when exposed to a protease capable of cleaving the protease-cleavable component.

Embodiment 47A. The hydrogel biomaterial of any one of embodiments 1A to 46A, wherein the fibrinogen component does not possess the clotting activity of a native fibrinogen molecule.

Embodiment 48A. A hydrogel biomaterial comprising

at least one polymer component;

at least one protease-cleavable component; and

a fibrinogen component derivatized with a plurality of polymeric linkers;

wherein the at least one polymer component, the at least one protease-cleavable component, and the fibrinogen component are crosslinked via carbon-sulfur covalent bonds.

Embodiment 49A. A hydrogel biomaterial prepared by a polymerization reaction of a mixture comprising:

at least one polymer component comprising at least two of a first reactive group;

wherein the first reactive group is selected from the group consisting of a reactive thiol group and a reactive ene group;

at least one protease-cleavable component comprising at least two of a second reactive group;

wherein the second reactive group is selected from the group consisting of a reactive thiol group and a reactive ene group;

a fibrinogen component derivatized with a plurality of polymeric linkers, wherein each polymeric linker comprises a reactive ene group; and

provided that at least one of the first reactive group and the second reactive group is a reactive thiol group.

Embodiment 50A. A hydrogel biomaterial prepared by a polymerization reaction of a mixture comprising:

at least one polymer component comprising at least two of a first reactive group;

wherein the first reactive group is selected from the group consisting of a reactive thiol group and a reactive ene group;

at least one protease-cleavable component comprising at least two of a second reactive group;

wherein the second reactive group is selected from the group consisting of a reactive thiol group and a reactive ene group;

a fibrinogen component derivatized with a plurality of polymeric linkers, wherein each polymeric linker comprises a reactive ene group;

wherein the hydrogel biomaterial has a storage modulus of between about 100 Pa and about 10,000 Pa; and

provided that at least one of the first reactive group and the second reactive group is a reactive thiol group.

Embodiment 51A. A hydrogel biomaterial prepared by a polymerization reaction of a mixture comprising:

at least one polymer component comprising at least two of a first reactive group;

wherein the first reactive group is selected from the group consisting of a reactive thiol group and a reactive ene group;

at least one protease-cleavable component comprising at least two of a second reactive group;

wherein the second reactive group is selected from the group consisting of a reactive thiol group and a reactive ene group;

a fibrinogen component derivatized with a plurality of polymeric linkers with reactive ene groups;

wherein the hydrogel biomaterial does not substantially swell when exposed to a protease capable of cleaving the protease-cleavable component; and

provided that at least one of the first reactive group and the second reactive group is a reactive thiol group.

Embodiment 1B. A hydrogel microparticle comprising

at least one polymer component;

at least one protease-cleavable component; and

a fibrinogen component derivatized with a plurality of polymeric linkers;

wherein the at least one polymer component, the at least one protease-cleavable component, and the fibrinogen component are crosslinked via a plurality of crosslink units each comprising a carbon-sulfur covalent bond.

Embodiment 2B. The hydrogel microparticle of embodiment 1B, wherein the crosslink units of the plurality of crosslink units are selected from the group consisting of

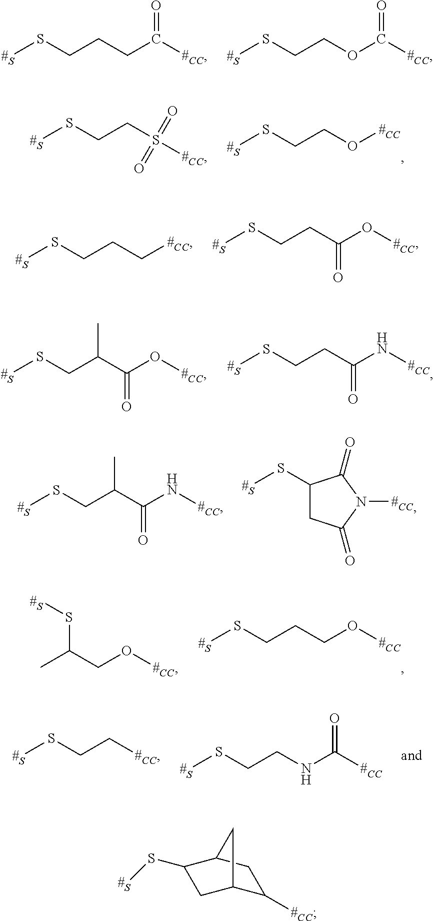

wherein #s and #cc represent attachment points to the at least one polymer component, the at least one protease-cleavable component, or a polymeric linker of the fibrinogen component.

Embodiment 3B. The hydrogel microparticle of embodiment 1B or 2B, wherein the crosslink units of the plurality of crosslink units are

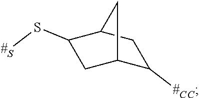

wherein #s and #cc represent attachment points to the at least one polymer component, the at least one protease-cleavable component, or a polymeric linker of the fibrinogen component.

Embodiment 4B. The hydrogel microparticle of embodiment 1B or 2B, wherein the at least one polymer component is connected to the at least one protease-cleavable component via a first crosslink unit and the fibrinogen component is connected to the at least one protease-cleavable component via a second crosslink unit.

Embodiment 5B. The hydrogel microparticle of embodiment 4B, wherein the first crosslink unit is selected from the group consisting of

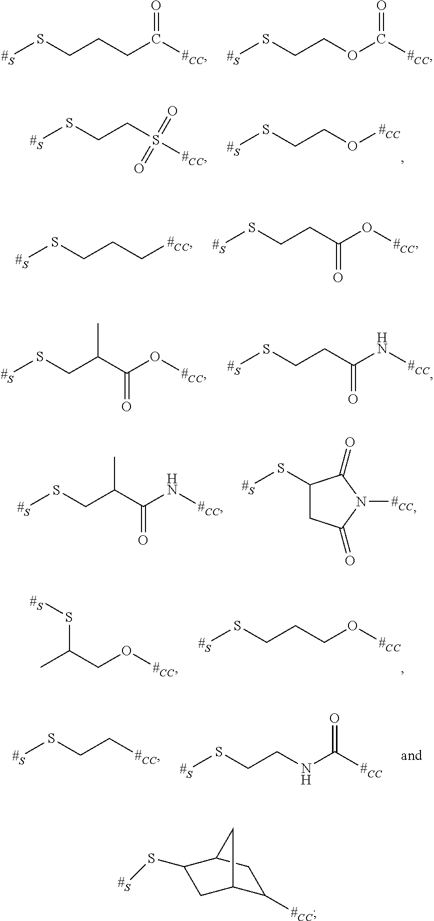

wherein #s represents the attachment point to the at least one protease-cleavable component and #cc represents the attachment point to the at least one polymer component.

Embodiment 6B. The hydrogel microparticle of embodiment 4B or 5B, wherein the first crosslink unit is

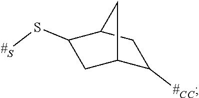

wherein #s represents the attachment point to the at least one protease-cleavable component and #cc represents the attachment point to the at least one polymer component.

Embodiment 7B. The hydrogel microparticle of any one of embodiments 4B to 6B, wherein the second crosslink unit is selected from the group consisting of

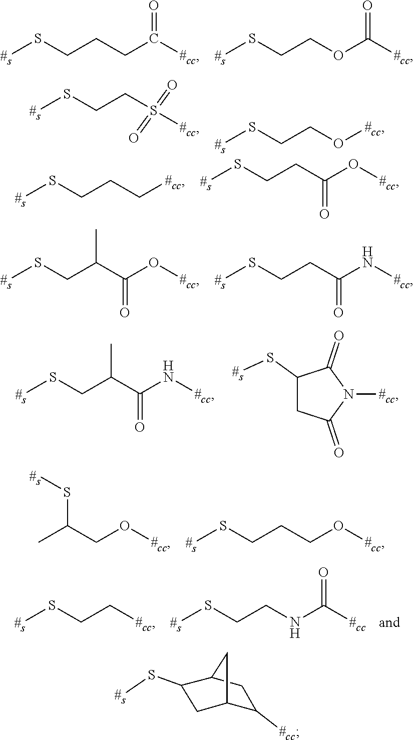

wherein #s represents the attachment point to the at least one protease-cleavable component and #cc represents the attachment point to a polymeric linker of the fibrinogen component.

Embodiment 8B. The hydrogel microparticle of any one of embodiments 4B to 7B, wherein the second crosslink unit is

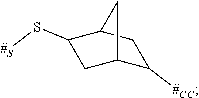

wherein #s represents the attachment point to the at least one protease-cleavable component and #cc represents the attachment point to a polymeric linker of the fibrinogen component.

Embodiment 9B. The hydrogel microparticle of embodiment 1B or 2B, wherein the at least one polymer component is connected to the at least one protease-cleavable component via a first crosslink unit and the fibrinogen component is connected to at least one polymer component via a second crosslink unit.

Embodiment 10B. The hydrogel microparticle of embodiment 9B, wherein the first crosslink unit is selected from the group consisting of

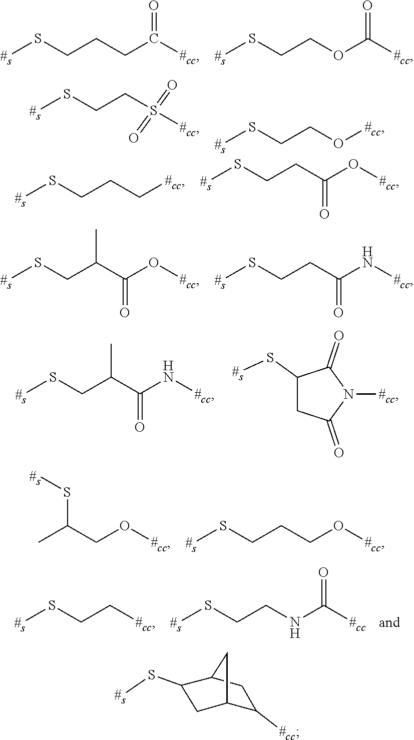

wherein #s represents the attachment point to the at least one polymer component and #cc represents the attachment point to the at least one protease-cleavable component.

Embodiment 11B. The hydrogel microparticle of embodiment 9B or 10B, wherein the first crosslink unit is

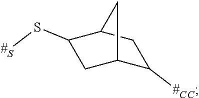

wherein #s represents the attachment point to the at least one polymer component and #cc represents the attachment point to the at least one protease-cleavable component.

Embodiment 12B. The hydrogel microparticle of any one of embodiments 9B to 11B, wherein the second crosslink unit is selected from the group consisting of

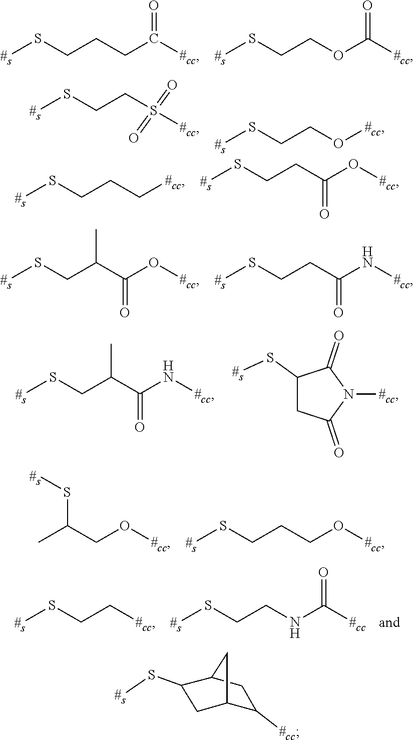

wherein #s represents the attachment point to the at least one polymer component and #cc represents the attachment point to a polymeric linker of the fibrinogen component.

Embodiment 13B. The hydrogel microparticle of any one of embodiments 9B to 12B, wherein the second crosslink unit is

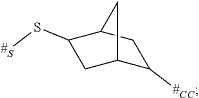

wherein #s represents the attachment point to the at least one polymer component and #cc represents the attachment point to a polymeric linker of the fibrinogen component.

Embodiment 14B. The hydrogel microparticle of embodiment 1B or 2B, wherein the at least one polymer component is connected to the fibrinogen component via a first crosslink unit and the fibrinogen component is connected to the at least one protease-cleavable component via a second crosslink unit.

Embodiment 15B. The hydrogel microparticle of embodiment 14B, wherein the first crosslink unit is selected from the group consisting of

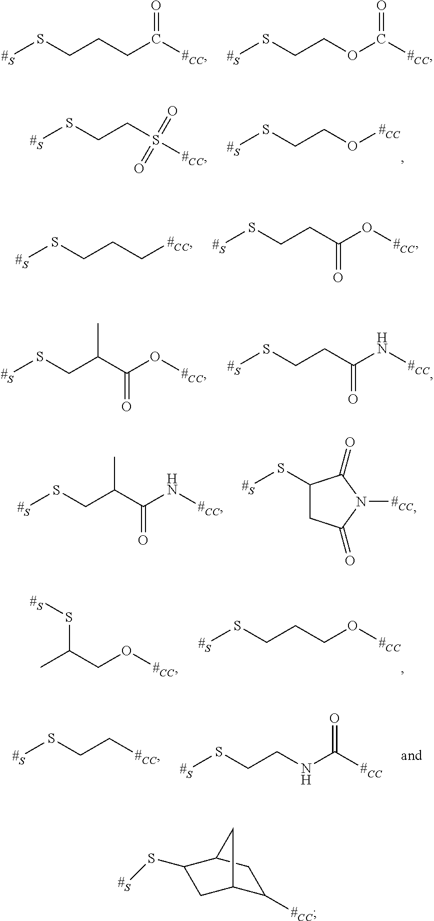

wherein #s represents the attachment point to the at least one polymer component and #cc represents the attachment point to a polymeric linker of the fibrinogen component.

Embodiment 16B. The hydrogel microparticle of embodiment 14B or 15B, wherein the first crosslink unit is

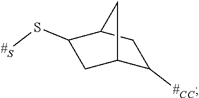

wherein #s represents the attachment point to the at least one polymer component and #cc represents a polymeric linker of the fibrinogen component.

Embodiment 17B. The hydrogel microparticle of any one of embodiments 14B to 16B, wherein the second crosslink unit is selected from the group consisting of

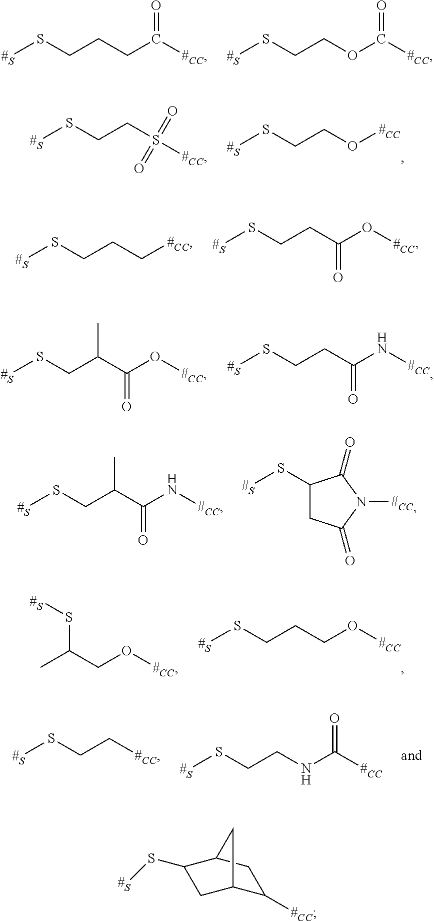

wherein #s represents the attachment point to the at least one protease-cleavable component and #cc represents the attachment point to a polymeric linker of the fibrinogen component.

Embodiment 18B. The hydrogel microparticle of any one of embodiments 14B to 17B, wherein the second crosslink unit is

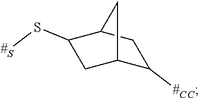

wherein #s represents the attachment point to the at least one protease-cleavable component and #cc represents the attachment point to a polymeric linker of the fibrinogen component.

Embodiment 19B. The hydrogel microparticle of any one of embodiments 1B to 18B, wherein the at least one polymer component comprises a linear polymer component.

Embodiment 20B. The hydrogel microparticle of embodiment 19B, wherein the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

Embodiment 21B. The hydrogel microparticle of embodiment 19B or 20B, wherein the linear polymer component comprises a linear polymeric moiety, wherein the linear polymeric moiety is a poly(ethylene glycol) moiety having a molecular weight of between about 1 kDa and about 40 kDa.

Embodiment 22B. The hydrogel microparticle of any one of embodiments 19B to 21B, wherein the linear polymer component comprises a linear polymeric moiety, wherein the linear polymeric moiety is a poly(ethylene glycol) moiety having a molecular weight of between about 6 kDa.

Embodiment 23B. The hydrogel microparticle of any one of embodiments 1B to 18B, wherein the at least one polymer component comprises a branched polymer component.

Embodiment 24B. The hydrogel microparticle of embodiment 23B, wherein the branched polymer component comprises a branched polymeric moiety with n polymeric arms, wherein n is an integer selected from the group consisting of 3, 4, 5, 6, 7, 8, 9, and 10.

Embodiment 25B. The hydrogel microparticle of embodiment 24B, wherein the branched polymer component comprises a branched polymeric moiety with n polymeric arms, wherein n is 4.

Embodiment 26B. The hydrogel microparticle of embodiment 24B or 25B, wherein each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

Embodiment 27B. The hydrogel microparticle of any one of embodiments 24B to 26B, wherein the branched polymeric moiety has a molecular weight of between about 1 kDa and about 40 kDa.

Embodiment 28B. The hydrogel microparticle of any one of embodiments 24B to 27B, wherein the branched polymer component comprises a 4-arm poly(ethylene glycol) moiety.

Embodiment 29B. The hydrogel microparticle of embodiment 28B, wherein the 4-arm poly(ethylene glycol) moiety has a molecular weight of about 20 kDa.

Embodiment 30B. The hydrogel microparticle of any one of embodiments 1B to 29B, wherein the at least one protease-cleavable component is a synthetic peptide.

Embodiment 31B. The hydrogel microparticle of any one of embodiments 1B to 30B, wherein each of the at least one protease-cleavable component comprises a peptide chain comprising a sequence selected from the group consisting of SEQ ID NOs: 1-19.

Embodiment 32B. The hydrogel microparticle of any one of embodiments 1B to 31B, wherein the hydrogel microparticle comprises two protease-cleavable components, wherein one protease-cleavable component is a matrix metalloproteinase (MMP) cleavable component, and the other is a plasmin cleavable component.

Embodiment 33B. The hydrogel microparticle of embodiment 32B, wherein the hydrogel microparticle comprises two protease-cleavable components, wherein the matrix metalloproteinase (MMP) cleavable peptide is a peptide of SEQ ID NO: 19, and the plasmin cleavable peptide is a peptide of SEQ ID NO: 7.

Embodiment 34B. The hydrogel microparticle of any one of embodiments 1B to 33B, wherein the fibrinogen component derivatized with a plurality of polymeric linkers has been derivatized at cysteine residues.

Embodiment 35B. The hydrogel microparticle of any one of embodiments 1B to 34B, wherein the fibrinogen component derivatized with a plurality of polymeric linkers has been derivatized at cysteine residues and comprises between 30 moles and 58 moles of polymeric linkers per mole of derivatized fibrinogen.

Embodiment 36B. The hydrogel microparticle of any one of embodiments 1B to 35B, wherein each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

Embodiment 37B. The hydrogel microparticle of any one of embodiments 1B to 36B, wherein each individual polymeric linker of the plurality of polymeric linkers comprises a poly(ethylene glycol) moiety, wherein the poly(ethylene glycol) moiety has a molecular weight of about 3.5 kDa.

Embodiment 38B. The hydrogel microparticle of any one of embodiments 1B to 37B, wherein the hydrogel microparticle comprises comprising:

0-50% wt. of a linear polymer component comprising a linear PEG moiety, wherein the linear PEG moiety has an average molecular weight of 6 kDa;

0.1-50% wt. of a branched polymer component comprising a 4-armed PEG moiety, wherein the 4-armed PEG moiety has an average molecular weight of 20 kDa;

0-6% wt. of a first protease-cleavable component comprising a IMP-cleavable peptide K(hC)GPQGIAGQ(hC)K (SEQ ID NO: 19);

0-6% wt. of a second protease-cleavable component comprising a plasmin-cleavable peptide (hC)ALKVLKG(hC)G-amide (SEQ ID NO: 7);

0-10% wt. of a fibrinogen component consisting of a fibrinogen molecule derivatized with a plurality of polymeric linkers each comprising a 3.5 kDa linear PEG; and

water.

Embodiment 39B. The hydrogel microparticle of embodiment 38B, the hydrogel microparticle comprises about 0.75% wt. of the fibrinogen component.

Embodiment 40B. The hydrogel microparticle of embodiment 38B, the hydrogel microparticle comprises about 1.50% wt. of the fibrinogen component.

Embodiment 41B. The hydrogel microparticle of embodiment 38B, the hydrogel microparticle comprises about 3.00% wt. of the fibrinogen component.

Embodiment 42B. The hydrogel microparticle of any one of embodiments 1B to 41B, wherein the hydrogel microparticle has a longest dimension of between about 0.05 mm and about 1.0 mm.

Embodiment 43B. The hydrogel microparticle of any one of embodiments 1B to 42B, wherein the hydrogel microparticle is a hydrogel microsphere.

Embodiment 44B. The hydrogel microparticle of embodiment 43B, wherein the hydrogel microsphere has a diameter of between about 0.05 mm and about 1.0 mm.

Embodiment 45B. The hydrogel microparticle of any one of embodiments 1B to 44B, wherein the hydrogel microparticle has a storage modulus of between about 100 Pa and about 10,000 Pa.

Embodiment 46B. The hydrogel microparticle of any one of embodiments 1B to 45B, wherein the hydrogel microparticle does not substantially swell when exposed to a protease capable of cleaving the protease-cleavable component.

Embodiment 47B. The hydrogel microparticle of any one of embodiments 1B to 46B, wherein the fibrinogen component does not possess the clotting activity of a native fibrinogen molecule.

Embodiment 48B. A hydrogel microparticle wherein the hydrogel microparticle is prepared by a polymerization reaction of a mixture comprising:

at least one polymer component comprising at least two of a first reactive group;

wherein the first reactive group is selected from the group consisting of a reactive thiol group and a reactive ene group;

at least one protease-cleavable component comprising at least two of a second reactive group;

wherein the second reactive group is selected from the group consisting of a reactive thiol group and a reactive ene group;

a fibrinogen component derivatized with a plurality of polymeric linkers, wherein each polymeric linker comprises a reactive ene group;

wherein the hydrogel microparticle has a longest dimension of between about 0.1 mm and about 1 mm; and

provided that at least one of the first reactive group and the second reactive group is a reactive thiol group.

Embodiment 49B. A hydrogel microparticle wherein the hydrogel microparticle is prepared by a polymerization reaction of an aqueous mixture comprising:

at least one polymer component comprising reactive ene groups;

at least one protease-cleavable component comprising reactive thiol groups;

a fibrinogen component derivatized with a plurality of polymeric linkers with reactive ene groups; and

wherein the hydrogel microparticle has a longest dimension of between about 0.1 mm and about 1 mm.

Embodiment 50B. A hydrogel microparticle wherein the hydrogel microparticle is prepared by a polymerization reaction of an aqueous mixture comprising:

at least one polymer component comprising reactive thiol groups;

at least one protease-cleavable component comprising reactive ene groups;

a fibrinogen component derivatized with a plurality of polymeric linkers with reactive ene groups; and

wherein the hydrogel microparticle has a longest dimension of between about 0.1 mm and about 1 mm.

Embodiment 51B. A hydrogel microparticle wherein the hydrogel microparticle is prepared by a polymerization reaction of an aqueous mixture comprising:

at least one polymer component comprising reactive thiol groups;

at least one protease-cleavable component comprising reactive ene groups;

a fibrinogen component derivatized with a plurality of polymeric linkers with reactive ene groups; and

wherein the hydrogel microparticle has a longest dimension of between about 0.1 mm and about 1 mm.

Embodiment 52B. A hydrogel microparticle wherein the hydrogel microparticle is prepared by a polymerization reaction of an aqueous mixture comprising:

at least one polymer component comprising at least two of a first reactive group, wherein the first reactive group is selected from the group consisting of a reactive thiol group and a reactive ene group;

at least one protease-cleavable component comprising at least two of a second reactive group, wherein the second reactive group is selected from the group consisting of a reactive thiol group and a reactive ene group;

a fibrinogen component derivatized with a plurality of polymeric linkers with reactive ene groups;

wherein the hydrogel microparticle has a storage modulus of 100-10,000 Pa; and

provided that at least one of the first reactive group and the second reactive group is a reactive thiol group.

Embodiment 53B. A hydrogel microparticle wherein the hydrogel microparticle is prepared by a polymerization reaction of an aqueous mixture comprising:

at least one polymer component comprising at least two of a first reactive group, wherein the first reactive group is selected from the group consisting of a reactive thiol group and a reactive ene group;

at least one protease-cleavable component comprising at least two of a second reactive group, wherein the second reactive group is selected from the group consisting of a reactive thiol group and a reactive ene group;

a fibrinogen component derivatized with a plurality of polymeric linkers with reactive ene groups;

wherein the hydrogel microparticle does not substantially swell when exposed to a protease capable of cleaving the protease-cleavable component; and

provided that at least one of the first reactive group and the second reactive group is a reactive thiol group.

### EXAMPLES

The chemical reactions in the Examples described can be readily adapted to prepare a number of other components disclosed herein, and alternative methods for preparing the components of this disclosure are deemed to be within the scope of this disclosure. For example, the synthesis of non-exemplified components according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other components of the present disclosure.

The following Examples are provided to illustrate but not to limit the invention.

### Abbreviations

| | |
|---|---|
| Da | Dalton |
| DMEM | Dulbecco's Modified Eagle Medium |
| DIFgn-PEG-NB | Denatured, Inactivated Fibrinogen, |
| | Derivatized with PEG-Norbornene |
| DTP | Direct-to-polymer |
| DTT | Dithiothreitol |
| FBS | Fetal Bovine Serum |
| Fgn | Fibrinogen |
| Fgn-alpha-PEG-NB | Alpha chain of fibrinogen derivatized with |
| | poly(ethylene) glycol (PEG) linkers |
| | containing terminal norbornenes |
| Fgn-beta-PEG-NB | Beta chain of fibrinogen derivatized with |
| | poly(ethylene) glycol (PEG) linkers |
| | containing terminal norbornenes |
| Fgn-gamma-PEG-NB | Gamma chain of fibrinogen derivatized with |
| | poly(ethylene) glycol (PEG) linkers |
| | containing terminal norbornenes |
| g (mass) | Gram |
| HDF | Human Dermal Fibroblasts |
| kDa | Kilodalton |
| L | Litre |
| mg | Milligram |
| mL | Millilitre |
| mmol | Millimoles |
| mM | Millimolar |
| MW | Molecular weight |
| NAP | Sodium acylphosphinate (Sodium phenyl- |
| | 2,4,6-trimethylbenzoylphosphinate) |
| NB | Norbornene |
| PEG | Poly(ethylene)glycol |
| PEG-NB | Poly(ethylene) glycol containing terminal |
| | polymerization-enabling functional groups |
| | (Norbornene). Generic term for all PEGS |
| | terminated with norbornenes |
| 3.5KL PEG-NB2 | 3.5 kDa (Average MW), linear, |
| | poly(ethylene) glycol (PEG) containing |
| | terminal norbornenes |
| 6KL PEG-NB | 6 kDa (Average MW), linear, poly(ethylene) |
| | glycol (PEG) containing terminal norbornenes |
| 15K3A PEG-NB | 15 kDa (Ave MW), 3-armed (branched), |
| | PEG containing terminal norbornenes |
| 20K4A PEG-NB | 20 kDa (Ave MW), 4-armed (branched), |
| | PEG containing terminal norbornenes |
| 30K6A PEG-NB | 30 kDa (Ave MW), 6-armed (branched), |
| | PEG containing terminal norbornenes |
| RT | Room temperature |
| TCEP | tris(2-carboxyethyl)phosphine |
| x g (centrifugation | units of gravity |
| force) | |

### Example 1—Synthesis of a Fibrinogen Component: PEG-Norbornene-Derivatized Fibrinogen (DIFgn-PEG-NB)

Five grams of fibrinogen were chemically denatured in 8M urea, and its cysteines were reduced by addition of a 2-fold excess of TCEP. The unfolded and reduced fibrinogen was conjugated with 25 g of 3.5KL PEG-NB2 utilizing a radical-mediated thiol-ene reaction involving the photo initiator NAP at 0.01%, activated by irradiation with light at 385 nm. After the reaction, free 3.5KL PEG-NB2 and NAP were removed by diafiltration or tangential flow filtration (TFF). The PEGylated fibrinogen (DI-Fgn-PEG-NB) was put into 17.5 mM Na Acetate pH 4.0, 137 mM NaCl, 3 mM KCl by diafiltration or TFF. Subsequently, the DIFgn-PEG-NB was concentrated, 0.2 μm-filtered into sterile bottles, and frozen at ≤−60° C.

### Example 2—Loss of Clotting Activity of PEG-Norbornene-Derivatized Fibrinogen

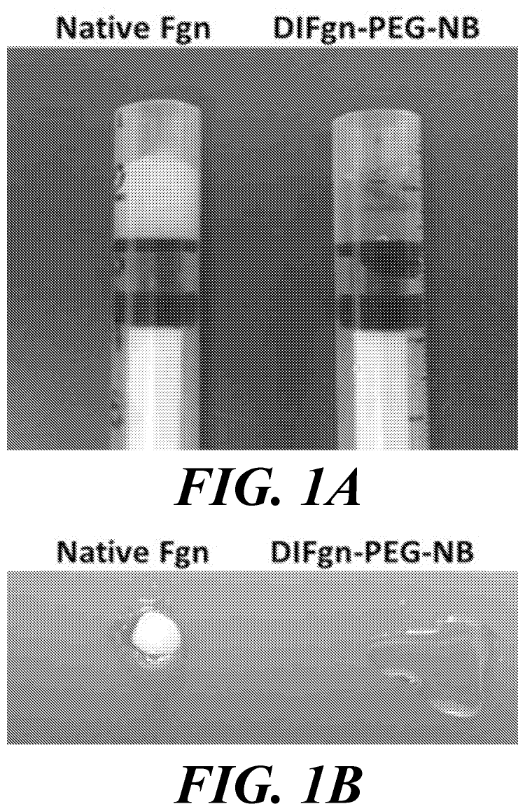

Briefly, 10 mg/mL final concentration native Fibrinogen (input Fgn for DI-Fgn-PEG-NB synthesis) and DI-Fgn-PEG-NB were added to activated Human Thrombin (20 U/mL) and Human Factor XIIIa (10 U/mL) containing 8 mM CaCl₂). Each reaction was mixed by pipetting and transferred to the tip of a cut-off 1 ml syringe. Within 1 minute of mixing, reactions containing native Fgn became opaque while the DIFgn-PEG-NB solution remained visibly clear even after 30 minutes as can be seen in FIG. 1A. When removed from the syringe tips, the native Fgn samples contracted into a white ball, indicating that they had formed a fibrin clot, while the DIFgn-PEG-NB samples remained as a clear liquid as can be seen in FIG. 1B.

This experiment indicates that the modification of native Fgn to create DIFgn-PEG-NB for use in the scaffolding polymer formulation results in the loss of clotting activity.

### Example 3—Synthesis of Polymer Components: PEG-Norbornene

Norbornene acid (2.5 molar equivalent to the —OH content in the PEG), N,N′-diisopropylcarbodiimide (DIC, 2.5 molar equivalent to OH) and 4-dimethylaminopyridine (DMAP, 0.5 g) were added to dehydrated PEG (50 g) in anhydrous toluene. The reaction was stirred at 50° C. for 36 hours under argon. The reaction mixture was filtered through a cinder-glass funnel and the filtrate was concentrated by rotary evaporation to yield about 50 mL of viscous material. Ethanol (150 mL) was added and stirred at RT for 20 minutes to form a clear uniform mixture. Anhydrous ether (250 mL) was then added into the mixture with agitation, and the mixture was cooled to −20° C. for 8 hours, allowing the PEG-NB to precipitate. The precipitated PEG-NB was collected by filtration through a cinder glass funnel. The PEG-NB product was further purified by two consecutive precipitations from methylene chloride with diethyl ether, filtered, and dried under vacuum or by dialysis with water (MWCO 100-500 D) followed by lyophilization.

Using this general procedure, various PEG-norbornene derivatives such as linear, 4-arm, 6-arm, and 8 PEG-norbornene were prepared.

### Example 4—Synthesis of Dithiol Polypeptides

The peptides used in the following examples were custom-synthesized using conventional solid-state peptide synthesis by Bachem corporation. Trifluoroacetic salts of the peptides, commonly obtained at the end of the synthesis, were converted to more biomedically acceptable hydrochloride salts via lyophilization of the purified peptides from diluted solutions of hydrochloric acid. The stock solutions of peptides were prepared by dissolving powdered peptides in ultrapure water at a concentration of about 0.1 M, and the free thiol content in these stock solutions was measured by an Ellman's assay, commonly used in the art. 1 mL aliquots of the peptide stock solutions were stored frozen at −70° C. in tightly capped 2 mL cryogenic vials and slowly thawed on ice immediately prior to assembly of pre-polymer solutions, as described throughout the Examples.

### Example 5—Synthesis of NAP

At room temperature and under argon, 2,4,6-trimethylbenzoyl chloride (3.2 g, 0.018 mol) was added dropwise to an equimolar quantity of dimethyl phenylphosphonite (3.0 g, 0.018 mol). The reaction mixture was stirred for 18 hours, whereupon a four-fold excess of sodium iodide (6.5 g) in 100 mL of 2-butanone was added to the reaction mixture and heated to 50° C. After about 10 min, a solid precipitate formed. The mixture was cooled to ambient temperature, allowed to rest for four hours, and then filtered. The filtrate was washed and filtered three times with 2-butanone to remove unreacted sodium iodide, and excess solvent was removed by vacuum.

### Example 6—Synthesis of Scaffolding Formulation

The scaffolding formulations consists of five components: (1) denatured, inactivated human fibrinogen modified to present polymerization-enabling functional groups (DI-Fgn-PEG-NB), (2) polyethylene glycol (PEG) containing terminal polymerization-enabling functional groups (PEG-NBs), (3) polypeptides containing polymerization-enabling thiol groups (hCysMMPA (SEQ ID NO: 19) and hCysC2XPC (SEQ ID NO: 7)), (4) phosphate buffered saline to provide ionic buffering capacity to the aqueous environment, and (5) a photo-initiator (NAP). To make 1 mL of the pre-polymer solution, stock solutions of the five components were combined as follows: 650 μL DIFgn-PEG-NB (solution at 23 mg/mL), 50 μL 20K4A PEG-NB (solution at 20% wt./vol.), 50 μL 6KL PEG-NB (solution at 20% wt./vol.), 50 μL NAP (solution at 1.0% wt./vol.), 50 μL 10×PBS (pH 7.4), 75 μL hCysMMPA (solution at 50 mM measured thiol concentration), and 75 μL hCysC2XPC (solution at 50 mM measured thiol concentration). After mixing, the formulation was polymerized by exposure to 385 nM light (20 mW/cm²) for 30 seconds.

In this Example, PEG-NB components were 6KL PEG-NB and 20K4A PEG-NB; 6KL PEG-NB is a linear compound and 20K4A PEG-NB is a branched compound with 4 arms. The branched PEG-NB can have n polymeric arms, wherein n is an integer selected from the group consisting of 3, 4, 5, 6, 7, 8, 9, and 10. In some embodiments, n was 3. In some embodiments, n was 4. In some embodiments, n was 6. In some embodiments, n was 8.

The Scaffolding Formulation disclosed in the Example 6 has been designed to allow quick infiltration of various cell types into the scaffold that has been applied into an acute or chronic wound. It is well understood in the art that migratory cell types need adhesion moieties, such as an RGD sequence provided by DI Fibrinogen component for quick infiltration into the synthetic scaffold. To infiltrate a homogeneous scaffold with mesh sizes much smaller than the cell size, such as the scaffold disclosed in Example 6, invading cells need to make space for themselves, which they achieve by proteolytically digesting certain elements of the scaffold. DI Fibrinogen can be digested by cellular types relevant to the wound healing process, such as neutrophils, fibroblasts and keratinocytes, but the rate of digestion of DI Fibrinogen may be not fast enough to allow cellular infiltration at the rate relevant to the wound healing. Therefore, additional proteolysis-sensitive amino acid sequences were introduced into this scaffold to enhance its degradation by invading cells secreting plasmin (typically, neutrophils) or Matrix Metalloproteases (keratinocytes, fibroblasts). These sequences are disclosed in the application as hCysC2XPC and hCysMMPA polypeptides.

It is anticipated that for certain applications it would be beneficial to restrict invading cells to a specific type, or a subset of cell types. For instance, one can envision a scaffold that would favor infiltration of neutrophils, to the (partial) exclusion of keratinocytes and fibroblasts in order to achieve a specific biological or medical outcome. Such selectivity towards a certain cell type can be achieved by controlling the sensitivity of the scaffold to digestion by a specific protease. In this case, substituting a 1:1 stoichiometric mixture of the hCysC2XPC and hCysMMPA with just the hCysC2XPC at appropriate concentration would preclude quick infiltration of keratinocytes and fibroblasts into the construct, while still allowing fast infiltration of plasmin-secreting neutrophils. Conversely, substituting a 1:1 stoichiometric mixture of the hCysC2XPC and hCysMMPA with just the hCysMMPA would promote a faster infiltration of the scaffold by fibroblasts and keratinocytes, to the (partial) exclusion of the neutrophils.

It is also anticipated that for certain applications it would be beneficial to allow a relatively slow cellular infiltration. In such a case, constructing a scaffold entirely of the DI Fibrinogen and crosslinking synthetic polymers like PEG-Norbornenes while omitting the additional protease-sensitive elements (such as the hCysC2XPC and hCysMMPA polypeptides) would help achieve the goal.

Enabling examples demonstrating synthesis and application of such tunable scaffolds are disclosed below.

### Example 7—Synthesis of Scaffolding Formulation Containing 6-Arm PEG-NB

The scaffolding formulations consists of five components: (1) denatured, inactivated human fibrinogen modified to present polymerization-enabling functional groups (DI-Fgn-PEG-NB), (2) polyethylene glycol (PEG) containing terminal polymerization-enabling functional groups (PEG-NBs), (3) polypeptides containing polymerization-enabling thiol groups (hCysMMPA (SEQ ID NO: 19) and hCysC2XPC (SEQ ID NO: 7)), (4) phosphate buffered saline to provide ionic buffering capacity to the aqueous environment, and (5) a photo-initiator (NAP). To make 1 mL of the pre-polymer solution, stock solutions of the five components were combined as follows: 650 μL DIFgn-PEG-NB (solution at 23 mg/mL), 50 μL 30K6A PEG-NB (solution at 20% wt./vol.), 50 μL 6KL PEG-NB (solution at 20% wt./vol.), 50 μL NAP (solution at 1.0% wt./vol.), 50 μL 10×PBS (pH 7.4), 75 μL hCysMMPA (solution at 50 mM measured thiol concentration), and 75 μL hCysC2XPC (solution at 50 mM measured thiol concentration). After mixing, the formulation was polymerized by exposure to 385 nM light (20 mW/cm²) for 30 seconds.

### Example 8—Synthesis of Scaffolding Formulation Containing 3-Arm PEG-NB

The scaffolding formulations consists of five components: (1) denatured, inactivated human fibrinogen modified to present polymerization-enabling functional groups (DI-Fgn-PEG-NB), (2) polyethylene glycol (PEG) containing terminal polymerization-enabling functional groups (PEG-NBs), (3) polypeptides containing polymerization-enabling thiol groups (hCysMMPA (SEQ ID NO: 19) and hCysC2XPC (SEQ ID NO: 7)), (4) phosphate buffered saline to provide ionic buffering capacity to the aqueous environment, and (5) a photo-initiator (NAP). To make 1 mL of the pre-polymer solution, stock solutions of the five components were combined as follows: 650 μL DIFgn-PEG-NB (solution at 23 mg/mL), 50 μL 15K3A PEG-NB (solution at 20% wt./vol.), 50 μL 6KL PEG-NB (solution at 20% wt./vol.), 50 μL NAP (solution at 1.0% wt./vol.), 50 μL 10×PBS (pH 7.4), 75 μL hCysMMPA (solution at 50 mM measured thiol concentration), and 75 μL hCysC2XPC (solution at 50 mM measured thiol concentration). After mixing, the formulation was polymerized by exposure to 385 nM light (20 mW/cm²) for 30 seconds.

Examples 6, 7 and 8, describe the scaffold formulations in which branched PEG-NB with different numbers of arms were used. In Example 6, a four-arm PEG with average molecular weight 20 kg/mol was used; In Example 7, a six-arm PEG with average molecular weight 30 kg/mol was used; In Example 8, a three-arm PEG with average molecular weight 15 kg/mol was used. These branched PEG-NB components have about the same length of PEG segment (about 5 kD) per arm; therefore, their aqueous solutions at a same concentration have a same norbornene content. 20% wt./vol. 15K3A PEG-NB solution, 20% wt./vol. 20K4A PEG-NB solution, and 20% wt./vol. 30K6A PEG-NB solution, all contained about 40 mM of norbornene. Examples 5, 7, and 28 demonstrated that these branched PEG-NB polymer components with different numbers of arms can be used to replace each other.

### Example 9—Synthesis of Microparticles by Microfluidics Method

A microfluidic droplet generation chip was used to create water-in-oil emulsion droplets that were then cured with 385 nm light. Briefly, 10 mL of the pre-polymer solution (described in Example 6, above) and 50 mL of hexanes containing Span 80 (1% wt./vol. final) were prepared as the water (pre-polymer solution) and oil (hexanes+Span 80) phases. An Elvesys Flow Control System was then used to feed the water and oil phases into a Dolomite Droplet T-Junction Chip (260 μm etch depth) flow ratios of approximately 1:3 (water:oil). This creates individual water-in-oil droplets that remain separated as they flow through an outflow tubing (0.5 mm internal diameter), as observed with a high-speed microscope camera (Darwin). It must be noted that flow rates must be continually monitored and adjusted to maintain stable droplet formation and prevent droplet aggregation while traveling through the outflow tubing. A 385 nm light (about 20 mW/cm²) was placed over the outflow tubing to polymerize the droplets before collection. Flow rates were chosen to result in a travel time for the droplets of 30-45 seconds under the polymerizing light before collection of the outflow into 50 mL Falcon tubes containing 5 mL 0.5×PBS. Polymerized microparticles were then cleaned by repeated centrifugation (5 minutes at 1500×g) and washing steps. After the first spin, the oil phase was removed by pipetting. Two washes with 50% isopropanol were then used to remove residual oil phase (hexanes and Span 80). Isopropanol was then removed by repeated washing cycles with 0.5×PBS.

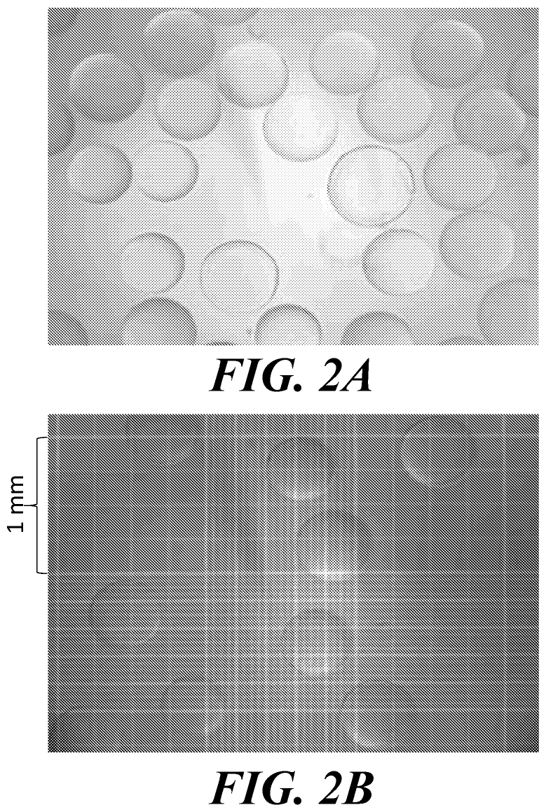

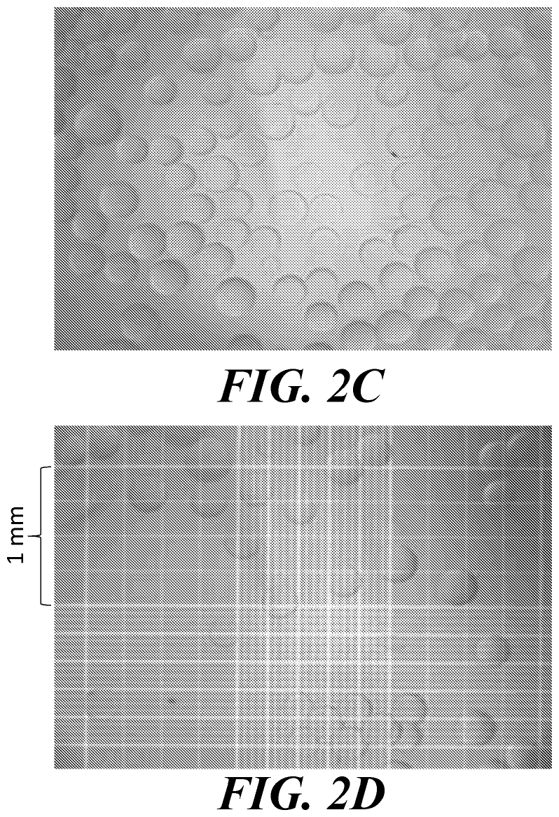

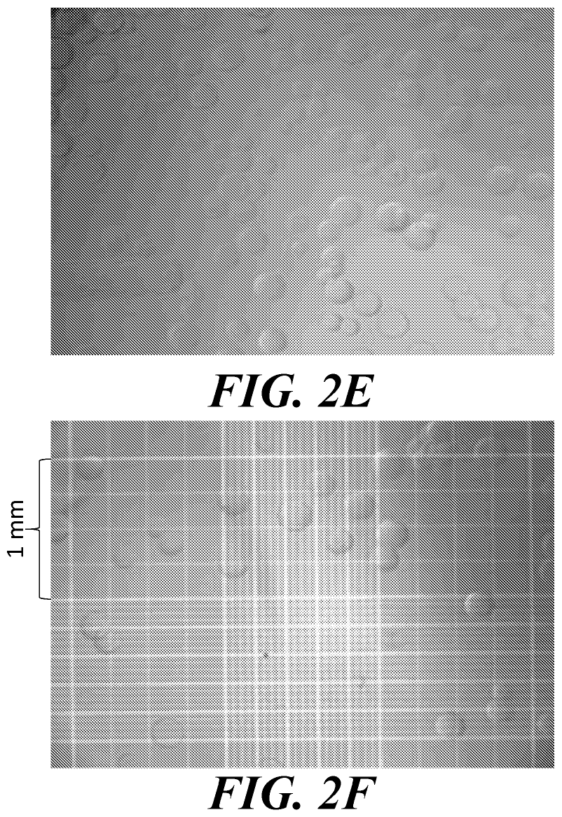

FIG. 2A and FIG. 2B show pictures of exemplary hydrogel microspheres having a diameter of about 0.5 mm to about 0.7 mm. FIG. 2C and FIG. 2D show pictures of exemplary hydrogel microspheres having a diameter of about 0.2 mm to about 0.3 mm. FIG. 2E and FIG. 2F show pictures of exemplary hydrogel microspheres having a diameter of about 0.1 mm to about 0.2 mm.

### Example 10—Synthesis of a Fibrinogen Derivative Incapable of Covalent Incorporation into Hydrogel Materials: PEG-Monomethylether-Derivatized Fibrinogen (DIFgn-PEG-MME)

Following the procedure of Example 1, plasma-derived human Fibrinogen (Fgn) was modified by covalent attachment of 3.5-kDa linear PEG-MonoMethylEther (MME) by replacing the 3.5KL PEG-NB2 reagent of Example 1 with a 3.5-kDa linear PEG having one reactive norbornene end and one inert monomethylether end.

### Example 11—Synthesis of Microparticles by Water-In-Oil Emulsion Method

Microparticles were prepared by water-in-oil emulsion method. Briefly, 10 mL of the water phase was added dropwisely into 100 mL of organic phase that was stirred for emulsification. The water phase was the pre-polymer aqueous solution (described in Example 6, above), and the organic phase was mineral oil containing 2% wt./vol. surfactant. After addition of water phase completed, the water-in-oil emulsion was continuously stirred. Polymerized microparticles were prepared by photocuring with a 385 nm light (about 20 mW/cm²) for 1 min. Polymerized microparticles were then purified by repeated centrifugation and washing steps as described above in Example 9.

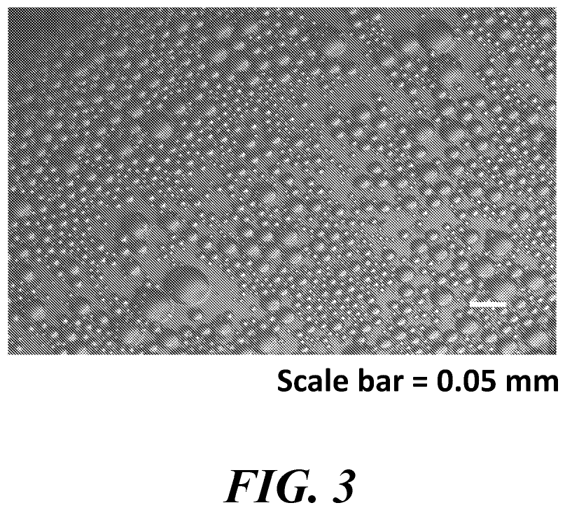

The hydrogel microparticles prepared by water-in-oil emulsion were spherical in shape. FIG. 3 shows picture of exemplary microparticles prepared by single emulsion method: microparticles with diameters ranging between about 0.005 mm and about 0.1 mm were prepared. Clearly, microparticles prepared by water-in-oil emulsion method have broader particle size distribution as compared to the microparticles prepared by microfluidics method (as described in Example 9).

### Example 12—Application of Microparticles Via a 26-Gauge Syringe Needle

A microparticle slurry was prepared by suspending about 1 mL of microparticles with 0.3 mm diameter in 1 mL of phosphate-buffered saline at physiological pH (7.4). The composition of microparticles was identical to the scaffolding formulation described in Example 6. The plunger was removed from a 5 mL syringe with a tip cap (Allegro Medical, Cat. #547161) and the microparticle slurry was loaded into a syringe using a wide-bore pipette. The plunger was re-inserted into the syringe, syringe inverted to point the tip up and the cap was replaced with a 26-gauge stainless steel needle (ID=0.26 mm). The microparticles inside the syringe were allowed to settle to the bottom (top of the plunger), at which point the air and excess buffer were carefully expunged, leaving about 1 mL of packed microparticle slurry in the syringe.

The microparticle slurry was expunged from a syringe through the 26-gauge needle and collected into a 1.5 mL microcentrifuge tube. Expunged microparticles were analyzed via light microscopy, as described in the Example 9. Despite the inner diameter of the needle being somewhat smaller than the diameter of the microparticles, the procedure was not damaging to the microparticles, and while some transient deformation of the shape may have occurred during the passage through the needle, mechanoelastic properties of the formulation allowed the microparticles to revert to their original shape as soon as they emerge from the needle.

This example illustrates the ease of application of the microparticle slurry by means of conventional medical device routinely used in modern practice of medicine.

### Example 13—Synthesis of Scaffold Formulation Using 3-Arm, 4-Arm, or 6-Arm PEG-NB

In Example 6, general approach to synthesize scaffolding formulation was described. The PEG-NBs described in Example 6 was composed of a 1:1 ratio of 20K4A PEG-NB (solution at 20% wt./vol.) and 6KL PEG-NB (solution at 20% wt./vol.). The 20K4A PEG-NB was one example of multiple armed PEG-NB. In some embodiments, the 20K4A PEG-NB can be replaced by other PEG-NBs with different number of arms, wherein the number is an integer selected from the group consisting of 3, 4, 5, 6, 7, 8, 9, and 10. In some embodiments, n is 3. In some embodiments, n is 4. In some embodiments, n is 6.

To make 1 mL of the pre-polymer solution using 15K3A PEG-NB, stock solutions of the five components were combined as follows: 650 μL DIFgn-PEG-NB (solution at 23 mg/mL), 50 μL 15K3A PEG-NB (solution at 20% wt./vol.), 50 μL 6KL PEG-NB (solution at 20% wt./vol.), 50 μL NAP (solution at 1.0% wt./vol.), 50 μL 10×PBS (pH 7.4), 75 μL hCysMMPA (solution at 50 mM measured thiol concentration), and 75 μL hCysC2XPC (solution at 50 mM measured thiol concentration). After mixing, the formulation was polymerized by exposure to 385 nM light (20 mW/cm²) for 30 seconds.

To make 1 mL of the pre-polymer solution using 30K6A PEG-NB, stock solutions of the five components were combined as follows: 650 μL DIFgn-PEG-NB (solution at 23 mg/mL), 50 μL 30K6A PEG-NB (solution at 20% wt./vol.), 50 μL 6KL PEG-NB (solution at 20% wt./vol.), 50 μL NAP (solution at 1.0% wt./vol.), 50 μL 10×PBS (pH 7.4), 75 μL hCysMMPA (solution at 50 mM measured thiol concentration), and 75 μL hCysC2XPC (solution at 50 mM measured thiol concentration). After mixing, the formulation was polymerized by exposure to 385 nM light (20 mW/cm²) for 30 seconds.

Variation in the number of arms of multiple arm PEG-NB did not change photocuring of the scaffold. All the formulations as described above within Example 6 and Example 11 formed hydrogels by 30 seconds of exposure to 385 nM light (20 mW/cm²).

The formulation containing 20K4A PEG-NB and that containing 30K6A PEG-NB were used in cellular invasion assay (see below). No difference was observed in cell invasion between these two formulations in which PEG-NBs with different numbers of arms were used.

### Example 14—Expression and Purification of Gamma Chain of Human Fibrinogen

A person skilled in the arts could devise alternate protocols using other expression, solubility or purification tags, cleavable, or uncleavable such as GST, MBP, SUMO, FH8, etc. or using no tags at all. This purification protocol is not limiting. Alternately, instead of using human fibrinogen, other species such as primate, bovine, horse, suid (porcine), feline, canine, rodent, sheep, chicken, etc. could be used. One could also envisage mutated constructs where some, but not all, of the cysteines have been mutated. These are non-limiting examples.

Human Fibrinogen can be purchased purified and lyophilized, however, one would be required to use clinical grade fibrinogen for any product, which is cost-prohibitive. Expressing the individual subunits in E. coli has the advantage of not only being less expensive, but also that more control over the process can be exercised. The analytics are also simplified because each subunit can be PEGylated separately, refolded separately, and analyzed separately. Finally, one can imagine that because the fibrinogen acts as a scaffold, individual subunits containing an RGD sequence could be used alone, eliminating the need to make all three subunits and simplifying the final product.

A DNA sequence coding the following amino acid sequence of human fibrinogen, gamma chain (SEQ ID NO: 20), was synthesized and cloned into a vector containing a T7 promoter, 6× N-terminal Histidine tag, strong ribosomal binding site, kanamycin resistance gene, and a high copy origin of replication.

Fgn-G:
SEQ ID NO: 20
MAKIK HHHHHH ENLYFQG YVAT RDNCCILDER FGSYCPTTCG
IADFLSTYQT KVDKDLQSLE DILHQVENKT SEVKQLIKAI
QLTYNPDESS KPNMIDAATL KSRKMLEEIM KYEASILTHD
SSIRYLQEIY NSNNQKIVNL KEKVAQLEAQ CQEPCKDTVQ
IHDITGKDCQ DIANKGAKQS GLYFIKPLKA NQQFLVYCEI
DGSGNGWTVF QKRLDGSVDF KKNWIQYKEG FGHLSPTGTT
EFWLGNEKIH LISTQSAIPY ALRVELEDWN GRTSTADYAM
FKVGPEADKY RLTYAYFAGG DAGDAFDGFD FGDDPSDKFF
TSHNGMQFST WDNDNDKFEG NCAEQDGSGW WMNKCHAGHL
NGVYYQGGTY SKASTPNGYD NGIIWATWKT RWYSMKKTTM
KIIPFNRLTI GEGQQHHLGG AKQVRPEHPA ETEYDSLYPE DDL

The plasmid was transformed into E. coli strain BL21(DE3). A single colony was used to inoculate an overnight starter culture, which was diluted 1:100 into 400 mLs of super broth (35 g tryptone, 20 g yeast extract, 5 g NaCl) the next morning. Once the culture reached mid-log phase, protein expression was induced by the introduction of 1 mM IPTG and the culture was harvested by centrifugation 4 hours later. Alternatively, an auto-induction protocol was used to induce protein expression by including 0.2% lactose at the time of dilution of the overnight culture, and growing at 37 C for 18-24 hours before harvesting by centrifugation.

Cell pellets (25 g) were resuspended in 1×PBS, 100 U turbo DNase, 3 mM MgCl2, 0.05 mg/mL lysozyme, 1 mM DTT, 1× pierce protease inhibitor tablets. Resuspended pellets were sonicated to lyse cells and centrifuged at 20,000 g to separate inclusion bodies from soluble protein. The inclusion bodies containing the gamma chain of human fibrinogen were then washed with 1×PBS, 1% triton X-100, 0.5 M NaCl, 1 mM DTT, four times. The washed inclusion bodies were then resuspended in 100 mLs of 8M urea, 50 mM Sodium Phosphate pH 7.4, 0.5 M NaCl, 1 mM DTT, and then centrifuged to remove cellular debris. The solubilized protein was loaded onto a His-trap-HP 5 mL column (formerly GE LifeSciences, now Cytiva). A gradient (0-25% B for 10 CV, then 25-100% B for 5 CV) was run between buffer A (8M urea, 50 mM Sodium Phosphate pH 7.4) and buffer B (8M urea, 50 mM Sodium Phosphate pH 7.4, 0.5 M imidazole) to elute the protein.

After pooling fractions and dialysis against 5 L of 8M urea 20 mM Hepes pH 7.0, gamma Fibrinogen was loaded onto a 26 mL Source 15Q column. The protein was eluted by a gradient between buffer A (8 M urea, 20 mM hepes pH 7.0) and buffer B (8 M urea, 20 mM Hepes pH 7.0, 0.5 M NaCl) as follows: 0-40% B, 10 CV, then 40-100% B, 1 CV. Fractions were run on SDS-PAGE to select the fractions containing gamma Fibrinogen and pooled as shown in FIG. 4.

### Example 15—Expression and Purification of Beta Chain of Human Fibrinogen

A person skilled in the arts could devise alternate protocols using other expression, solubility or purification tags, cleavable, or uncleavable such as GST, MBP, SUMO, FH8, etc. or using no tags at all. This purification protocol is not limiting. Alternately, instead of using human fibrinogen, other species such as primate, bovine, horse, suid (porcine), feline, canine, rodent, sheep, chicken, etc. could be used. One could also envisage mutated constructs where some, but not all, of the cysteines have been mutated. These are non-limiting examples.

A DNA sequence coding the following amino acid sequence of human fibrinogen, beta chain (SEQ ID NO: 21), was synthesized and cloned into a vector containing a T7 promoter, 6× N-terminal Histidine tag, strong ribosomal binding site, kanamycin resistance gene, and a high copy origin of replication.

Fgn-B:
SEQ ID NO: 21
MAKIK HHHHHH ENLYFQG QGVNDNEEGF FSARGHRPLD
KKREEAPSLR PAPPPISGGG YRARPAKAAA TQKKVERKAP
DAGGCLHADP DLGVLCPTGC QLQEALLQQE RPIRNSVDEL
NNNVEAVSQT SSSSFQYMYL LKDLWQKRQK QVKDNENVVN
EYSSELEKHQ LYIDETVNSN IPTNLRVLRS ILENLRSKIQ
KLESDVSAQM EYCRTPCTVS CNIPVVSGKE CEEIIRKGGE
TSEMYLIQPD SSVKPYRVYC DMNTENGGWT VIQNRQDGSV
DFGRKWDPYK QGFGNVATNT DGKNYCGLPG EYWLGNDKIS
QLTRMGPTEL LIEMEDWKGD KVKAHYGGFT VQNEANKYQI
SVNKYRGTAG NALMDGASQL MGENRTMTIH NGMFFSTYDR
DNDGWLTSDP RKQCSKEDGG GWWYNRCHAA NPNGRYYWGG
QYTWDMAKHG TDDGVVWMNW KGSWYSMRKM SMKIRPFFPQ Q

The plasmid was transformed into E. coli strain BL21(DE3). A single colony was used to inoculate an overnight starter culture, which was diluted 1:100 into 400 mLs of super broth (35 g tryptone, 20 g yeast extract, 5 g NaCl) the next morning. Once the culture reached mid-log phase, protein expression was induced by the introduction of 1 mM IPTG and the culture was harvested by centrifugation 4 hours later. Alternatively, an auto-induction protocol was used to induce protein expression by including 0.2% lactose at the time of dilution of the overnight culture, and growing at 37 C for 18-24 hours before harvesting by centrifugation.

Cell pellets (25 g) were resuspended in 1×PBS, 100 U turbo DNase, 3 mM MgCl2, 0.05 mg/mL lysozyme, 1 mM DTT, 1× pierce protease inhibitor tablets. Resuspended pellets were sonicated to lyse cells and centrifuged at 20,000 g to separate inclusion bodies from soluble protein. The inclusion bodies containing the gamma chain of human fibrinogen were then washed with 1×PBS, 1% triton X-100, 0.5 M NaCl, 1 mM DTT, four times. The washed inclusion bodies were then resuspended in 100 mLs of 8M urea, 50 mM Sodium Phosphate pH 7.4, 0.5 M NaCl, 1 mM DTT, and then centrifuged to remove cellular debris. The solubilized protein was loaded onto a His-trap-HP 5 mL column (formerly GE LifeSciences, now Cytiva). Beta Fibrinogen was eluted from the His-trap column using 8 M urea 70 mM Sodium Acetate pH 4.0. Fractions were run on an SDS-PAGE to determine which fractions contained protein to pool.

### Example 16—Expression and Purification of Alfa Chain of Human Fibrinogen

A person skilled in the arts could devise alternate protocols using other expression, solubility or purification tags, cleavable, or uncleavable such as GST, MBP, SUMO, FH8, etc. or using no tags at all. This purification protocol is not limiting. Alternately, instead of using human fibrinogen, other species such as primate, bovine, horse, suid (porcine), feline, canine, rodent, sheep, chicken, etc. could be used. One could also envisage mutated constructs where some, but not all, of the cysteines have been mutated. These are non-limiting examples.

A DNA sequence coding the following truncation amino acid sequence of human fibrinogen, alpha chain (SEQ ID NO: 22), was synthesized and cloned into a vector containing a T7 promoter, 6× N-terminal Histidine tag, strong ribosomal binding site, kanamycin resistance gene, and a high copy origin of replication.

Fgn-A:
SEQ ID NO: 22
MA HHHHHH ENLYFQG GGGVRGPRV VERHQSACKD
SDWPFCSDED WNYKCPSGCR MKGLIDEVNQ DFTNRINKLK
NSLFEYQKNN KDSHSLTTNI MEILRGDFSS ANNRDNTYNR
VSEDLRSRIE VLKRKVIEKV QHIQLLQKNV RAQLVDMKR

The plasmid was transformed into E. coli strain BL21(DE3). A single colony was used to inoculate an overnight starter culture, which was diluted 1:100 into 400 mLs of super broth (35 g tryptone, 20 g yeast extract, 5 g NaCl) the next morning. Once the culture reached mid-log phase, protein expression was induced by the introduction of 1 mM IPTG and the culture was harvested by centrifugation 4 hours later. Alternatively, an auto-induction protocol was used to induce protein expression by including 0.2% lactose at the time of dilution of the overnight culture, and growing at 37 C for 18-24 hours before harvesting by centrifugation.

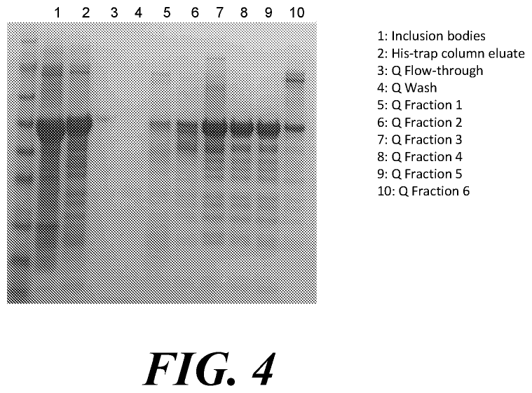

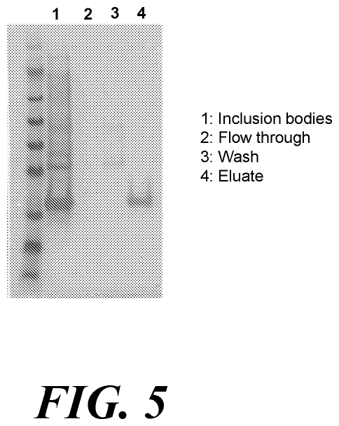

Cell pellets (25 g) were resuspended in 1×PBS, 100 U turbo DNase, 3 mM MgCl2, 0.05 mg/mL lysozyme, 1 mM DTT, 1× pierce protease inhibitor tablets. Resuspended pellets were sonicated to lyse cells and centrifuged at 20,000 g to separate inclusion bodies from soluble protein. The inclusion bodies containing the gamma chain of human fibrinogen were then washed with 1×PBS, 1% triton X-100, 0.5 M NaCl, 1 mM DTT, four times. The washed inclusion bodies were then resuspended in 100 mLs of 8M urea, 50 mM Sodium Phosphate pH 7.4, 0.5 M NaCl, 1 mM DTT, and then centrifuged to remove cellular debris. The solubilized protein was loaded onto a His-trap-HP 5 mL column (formerly GE LifeSciences, now Cytiva). Alpha Fibrinogen was eluted from the His-trap column using 8 M urea, 70 mM Sodium Acetate pH 4.0. Fractions were run on an SDS-PAGE to determine which fractions contained protein to pool as shown in FIG. 5.

### Example 17—Purification of Individual Chains from Blood-Derived Fibrinogen

This example describes separation of individual fibrinogen subunits via denaturing ion-exchange chromatography starting from commercially available fibrinogen derived from donor blood.

2 g of dry human fibrinogen powder (EMD Millipore, catalog number 341576) was dissolved in 80 mL of denaturing buffer (8 M urea, 20 mM Tris-HCl pH 8.9) upon gentle shaking at ambient temperature for 20 minutes. To reduce cysteine bonds in the fibrinogen, tris(2-carboxyethyl)phosphine hydrochloride (TCEP) was added to the final concentration of 10 mM followed by addition of disodium ethylenediaminetetraacetic acid (EDTA) to the final concentration of 1.25 mM and stirring continued for another 20 min at ambient temperature.

To remove unnecessary excipients introduced into the fibrinogen by the vendor, the reduced, denatured fibrinogen solution was transferred into a 250 mL Slide-A-Lyzer dialysis flask (molecular weight cut-off of 20 kDa; Thermo Fisher catalog #87763) and dialyzed against 4 L of 8 M urea, 20 mM Tris-HCl pH 8.9 overnight at +4° C. The dialyzed protein solution was removed from dialysis flask and the volume was adjusted to 120 mL with the dialysis buffer. Protein concentration was calculated to be 14.8 mg/mL at this point by measuring absorbance of the protein solution at 280 nm with a spectrophotometer.

30 mL of the dialyzed protein solution (440 mg reduced denatured fibrinogen) was applied to a 26 mL chromatographic column packed with anion exchange Source Q resin (GE Healthcare) pre-equilibrated with 8 M urea, 20 mM Tris-HCl pH 8.9, and the unbound material collected and set aside for analysis. Column was eluted with a linear gradient of sodium chloride from 0 to 125 mM in 8 M urea, 20 mM Tris-HCl pH 8.9, applied in 20 column volumes (520 mL). 13 mL fractions (0.5 column volume each) were collected throughout the gradient and set aside for analysis.

Protein content of each fraction and unbound material was analyzed by SDS-PAGE followed by staining with Coomassie Brilliant Blue, a method used widely in the art. Unbound material and fractions 1 through 11 contained beta subunit, fractions 14 through 20 contained alfa subunit and fractions 23-35 contained gamma subunit well separated from each other. Fractions 12-13 and 21-22 contained unresolved mixtures of subunits and were discarded.

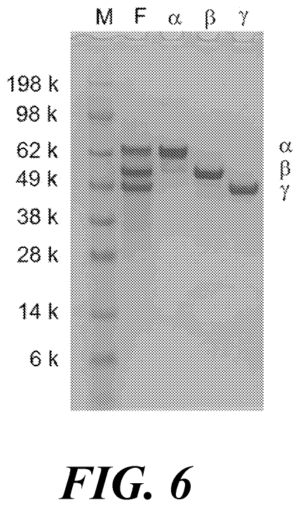

The chromatographic process was repeated three more times, to consume all reduced denatured fibrinogen prepared by the preceding steps, and SDS-PAGE analysis of the chromatographic fractions revealed the results were highly reproducible. In the end, fractions containing each purified subunit were pooled together separately and pH of each solution was adjusted to ˜7 with concentrated hydrochloric acid to yield 380 mg of purified alfa, 412 mg of purified beta and 322 mg of purified gamma subunit, each in the reduced, denatured form suitable for further processing as described below. Purity of the separated chains was analyzed by SDS-PAGE followed by Coomassie Brilliant Blue staining, and is shown in FIG. 6.

### Example 18—PEGylation of Individual Fibrinogen Subunits

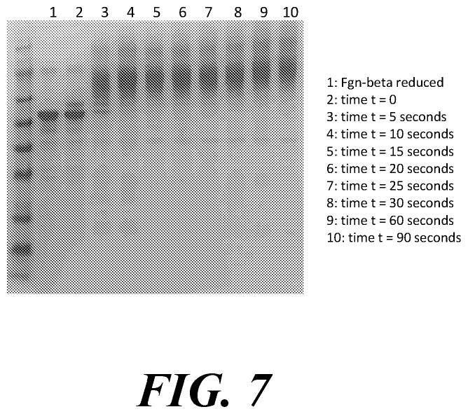

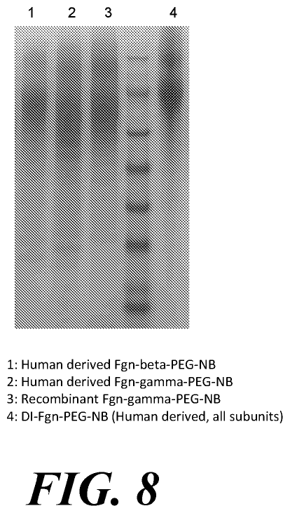

The purified gamma, beta and alpha subunits were individually concentrated to 12 mg/mL. Each subunit was reduced at room temperature for one hour with 3 mols of TCEP for every one mol of cysteines on the Fibrinogen subunit. Ten mols of PEG-diNB were added for every one mol of cysteines on the Fibrinogen subunit. The final concentration of the Fibrinogen subunit was 8 mg/mL in the PEGylation reaction, where NAP was added to a final concentration of 0.01-0.03%. Individual reactions were irradiated for 90 seconds at 20-22 mW/cm2 as shown for recombinant Fgn-beta-PEG-NB in FIG. 7. Free PEG-diNB was removed from the reaction using diafiltration, then the protein was refolded using diafiltration into a buffer composed of 17.5 mM Sodium Acetate pH 4.0, 140 mM NaCl, 2.7 mM KCl. Typical examples of PEGylated protein are shown in FIG. 8.

### Example 19—Synthesis of Scaffolding Formulation Containing a Single Fibrinogen Subunit

This scaffolding formulations consists of five components: (1) denatured, inactivated subunit gamma of human fibrinogen modified to present polymerization-enabling functional groups (Fgn-gamma-PEG-NB), (2) polyethylene glycol (PEG) containing terminal polymerization-enabling functional groups (PEG-NBs), (3) polypeptides containing polymerization-enabling thiol groups (hCysMMPA (SEQ ID NO: 19) and hCysC2XPC (SEQ ID NO: 7)), (4) phosphate buffered saline to provide ionic buffering capacity to the aqueous environment, and (5) a photo-initiator (NAP). To make 1 mL of the pre-polymer solution, stock solutions of the five components were combined as follows: 650 μL DI-Fgn-gamma-PEG-NB (solution at 23 mg/mL), 50 μL 20K4A PEG-NB (solution at 20% wt./vol.), 50 μL 6KL PEG-NB (solution at 20% wt./vol.), 50 μL NAP (solution at 1.0% wt./vol.), 50 μL 10×PBS (pH 7.4), 80 μL hCysIMPA (solution at 50 mM measured thiol concentration), and 80 μL hCysC2XPC (solution at 50 mM measured thiol concentration). After mixing, the formulation was polymerized by exposure to 385 nM light (20 mW/cm²) for 30 seconds.

Note that the major difference in composition between Example 19 and Example 6 is not only the identity of the PEGylated scaffolding protein (Fgn-gamma-PEG-NB versus DI-Fgn-PEG-NB), but the concentration of the dithiol peptides as well. This reflects differences in PEG-norbornene content per a weight unit of the PEGylated protein. In this example, fibrinogen has molecular weight of 336.89 kDa (biological unit prior to PEGylation) and 58 Cysteine residues that are converted to PEG-norbornenes in the synthesis of DI-Fgn-PEG-NB. In turn, the gamma subunit has molecular weight of 48.5 kDa and contains 10 Cysteines that are converted into PEG-norbornenes. Therefore, 15 mg/mL solution of DI-Fgn-PEG-NB contains 2.58 mM norbornene residues, while 15 mg/mL solution of Fgn-gamma-PEG-NB contains 3.09 mM norbornene residues. The increase of 0.49 mM in reactive norbornene was matched by an equivalent increase in reactive thiol, which is equally separated between the two protease-sensitive dithiol peptides.

### Example 20—Synthesis of Microparticles Containing Individual Fibrinogen Subunit

The synthesis of scaffolding microparticles containing Fgn-gamma-PEG-NB was performed following the method disclosed in Example 9, using a modified pre-polymer formulation disclosed in the Example 19.

A microfluidic droplet generation chip was used to create water-in-oil emulsion droplets that were then cured with 385 nm light. Briefly, 10 mL of the pre-polymer solution (described in Example 19, above) and 50 mL of hexanes containing Span 80 (1% wt./vol. final) were prepared as the water (pre-polymer solution) and oil (hexanes+Span 80) phases. An Elvesys Flow Control System was then used to feed the water and oil phases into a Dolomite Droplet T-Junction Chip (260 μm etch depth) flow ratios of approximately 1:3 (water:oil). This creates individual water-in-oil droplets that remain separated as they flow through an outflow tubing (0.5 mm internal diameter), as observed with a high-speed microscope camera (Darwin). It must be noted that flow rates must be continually monitored and adjusted to maintain stable droplet formation and prevent droplet aggregation while traveling through the outflow tubing. A 385 nm light (about 20 mW/cm²) was placed over the outflow tubing to polymerize the droplets before collection. Flow rates were chosen to result in a travel time for the droplets of 30-45 seconds under the polymerizing light before collection of the outflow into 50 mL Falcon tubes containing 5 mL 0.5×PBS. Polymerized beads were then cleaned by repeated centrifugation (5 minutes at 1500×g) and washing steps. After the first spin, the oil phase was removed by pipetting. Two washes with 50% isopropanol were then used to remove residual oil phase (hexanes and Span 80). Isopropanol was then removed by repeated washing cycles with 0.5×PBS.

The quality and size distribution of the prepared microparticles and dependence of their size on the adjustable process parameters, such as internal diameter of tubing used in the preparation, was investigated by light microscopy as described in Example 9 and was found to be indistinguishable from data obtained for microparticles containing full DI-Fgn-PEG-NB.

### Example 21—Direct to Polymer Formation of Slab-Scaffold Using Fibrinogen

Examples 1 through 6 describe typical workflow necessary to prepare a scaffolding formulation comprising synthetic polymers and an ECM protein covalently incorporated into the network. Because ECM proteins tend to have poor solubility in water, especially in their chemically reduced state (e.g. with cysteine bonds converted to free thiols to enable thiol-ene chemistry), the process involves generation of PEG-norbornene-derivatized Fibrinogen or individual PEG-norbornene-derivatized Fibrinogen subunits. This is a multi-step process that is reasonably time-consuming, involves excessive amount of reagents (significant amount of PEG-norbornene is not reacted and a large amount of denaturant is used to remove it via diafiltration prior to refolding), and generates significant amount of chemical waste. The resulting telechelic biopolymers, however, are soluble in water under non-denaturing conditions and physiological pH that makes them ideally suited for encapsulation of sensitive biological cargo, such as proteins and nucleic acids.

It is anticipated that for certain applications it will be more advantageous to synthesize scaffolding formulations via fewer steps, involving fewer intermediate reagents. In a non-limiting example, bypassing synthesis, purification and characterization of PEG-norbornene-derivatized Fibrinogen or individual PEG-norbornene-derivatized Fibrinogen subunits may offer significant advantages to a manufacturer of the scaffold. Such advantages include, but not limited to, reduced process time, reduced costs associated with reagents, analysis, and chemical waste disposal and better control over chemical composition of the resulting material, since prolonged exposure to urea is known to cause chemical modifications in proteins.

In the non-limiting Examples disclosed below, a different approach is taken, where a crosslinked scaffolding formulation is synthesized directly from chemically reduced Fibrinogen, PEG-norbornenes and crosslinking peptides, in a single step via a photoinitiated thiol-ene reaction. Because, as stated above, chemically reduced form of Fibrinogen is poorly soluble in physiological buffers, the reaction is performed under denaturing conditions, and as such the process is not well suited for encapsulation of sensitive biological cargo, such as proteins and nucleic acids. We demonstrate, however, that denaturant can easily be removed post-process, by extraction into water or physiological buffer, to result in a covalently crosslinked scaffolding network with mechano-elastic and biological properties indistinguishable from a network obtained via a thiol-ene photo-crosslinking under non-denaturing conditions, such as disclosed in Examples 1 through 6.

This example describes preparation of a scaffolding formulation directly from human donor-derived fibrinogen and synthetic polymers, bypassing preparation and characterization of DI-Fgn-PEG-NB.

1 g of dry human fibrinogen powder (EMD Millipore, catalog number 341576) was dissolved in 20 mL of denaturing buffer (8 M urea, 50 mM sodium phosphate pH 7.0) upon gentle shaking at ambient temperature for 15 minutes. To reduce cysteine bonds in the fibrinogen, tris(2-carboxyethyl)phosphine hydrochloride (TCEP) was added to the final concentration of 10 mM and the stirring continued for another 20 min at ambient temperature.

To remove unnecessary excipients introduced into the fibrinogen by the vendor, the reduced, denatured fibrinogen solution was transferred into a 30 mL Slide-A-Lyzer dialysis cassette (molecular weight cut-off of 20 kDa; Thermo Fisher catalog #66030) and dialyzed against 4 L of 8 M urea, 50 mM sodium phosphate pH 7.0 overnight at +4° C. The dialyzed protein solution was removed from dialysis cassette, transferred to a 50 mL conical tube and kept on ice during subsequent manipulations. Protein concentration was calculated to be 36.9±0.21 mg/mL at this point by measuring absorbance of the protein solution at 280 nm with a spectrophotometer. Concentration of free thiols was measured using Ellman's assay and was found to be 6.4±0.2 mM, in excellent agreement with theoretical number (6.36 mM) calculated on the bases of measured protein concentration and molecular weight and cysteine content of the human fibrinogen (data from UNIPROT database).

Simplified scaffolding formulations were prepared from reduced, denatured fibrinogen by crosslinking it with synthetic PEG-norbornenes in the course of photoinitiated thiol-ene reaction. Because the reduced denatured fibrinogen provides sufficient source of free thiols for the reaction to occur, the dithiol peptides utilized in other Examples were omitted here for simplicity. Four similar pre-polymer formulations were prepared, each containing 29 mg/mL reduced, denatured fibrinogen, 0.01% photoinitiator NAP, and the following PEG-norbornenes at the concentrations sufficient to achieve 1:1 stoichiometric ratio between the norbornenes and free thiols: 4-arm PEG 20 kDa tetranorbornene (formulation Fgn+20k-4), linear PEG 20 kDa dinorbornene (formulation Fgn+20KL), linear PEG 10 kDa dinorbornene (formulation Fgn+10KL) and linear PEG 6 kDa dinorbornene (formulation Fgn+6KL). The concentration of denaturant (urea) was between 6.5 and 7.8 M, sufficient to completely denature fibrinogen chains. 50 uL hydrogels were photopolymerized from these pre-polymer formulations in triplicate, by exposing the formulations to 20 mW/cm² 385 nm UV light for 90 seconds. All formulations formed robust hydrogels. Upon completion of polymerization, denaturant was removed from the hydrogels via passive diffusion, by incubating each hydrogel with 1 mL phosphate-buffered saline at ambient temperature for a few hours, followed by replacement of the buffer with fresh 1 mL and additional incubation to reach swelling equilibrium.

For comparative reasons, a similar scaffolding network was prepared under non-denaturing conditions from a pre-polymer solution containing DI-Fgn-PEG-NB at 29 mg/mL and a short dithiol crosslinker dithiothreitol at a 1:1 stoichiometric concentration between free thiols and reactive norbornenes (formulation DI-Fgn-PEG-NB+DTT). 50 uL hydrogels (in triplicate) were soaked in phosphate-buffered saline to achieve equilibrium swelling as described above.

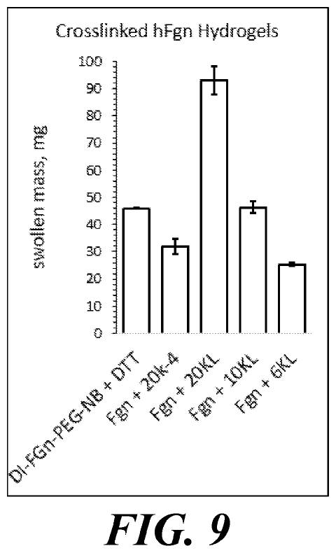

All swollen hydrogels were weighed, replicate observations averaged, and the resulting data are shown in FIG. 9. The following trends were observed and found to be in full agreement with Flory-Rehner theory of swelling of covalently crosslinked polymeric networks (see Metters, A. and Hubbell, J. 2005. Biomacromolecules 6, 1, pp 290-301 for a recent reference):

1. The degree of swelling, as measured by the equilibrium mass of the hydrogels, increased with increase in molecular weight of the linear chains between crosslinks. This was evident from a comparison between series Fgn+6KL, Fgn+10KL and Fgn+20KL, where increase in molecular weight of the linear PEG components from 6 to 10 to 20 kDa correlated with increase in the equilibrium swollen mass.

2. The degree of swelling decreased with increase in the branch order of the network. This was evident from comparing series Fgn+10KL and Fgn+20k-4, which have identical weight concentrations of the protein and polyethylene glycol, but different branch order. 20 kDa 4-arm PEG tetranorbornene contained the same molecular weight per a norbornene residue as the 10 kDa PEG dinorbornene (5 kDa), but had a higher branch order, resulting in smaller degree of hydrogel swelling at equilibrium.

Additional conclusions regarding the protein component of the network:

3. The protein chains inside the hydrogels did bind each other, even if the hydrogel was crosslinked in the presence of denaturant that was subsequently removed. This was evidenced by the fact that Fgn+6KL and Fgn+20k-4 formulations actually shrunk upon reaching equilibrium (equilibrium swollen mass of 30 mg and below, compared to ˜50 mg mass expected if the volume of the hydrogel remained the same as at the time of photopolymerization). This shrinking was most likely due to protein chains binding each other as the denaturant was removed; similar protein-protein interactions were also expected in the series Fgn+10KL and Fgn+20KL, but the swelling there was offset by much higher content of the hydrophilic polymer (PEG) comprising network.

4. The network structure of the hydrogels prepared under non-denaturing conditions was very similar to the network structure of the hydrogels prepared under denaturing conditions once the denaturant was removed and equilibrium swelling was achieved. One can imagine a situation where a PEGylated protein self-assembles into soluble high-order structures under non-denaturing conditions prior to photopolymerization, and photo-crosslinking generates PEG-based mesh around such structures (series DI-Fgn-PEG-NB+DTT). If one crosslinks the same components under denaturing conditions, where protein-protein interactions are disrupted due to the presence of the denaturant, one may expect a different, more homogeneous network structure, where individual protein chains are trapped in the PEG-based mesh away from each other, and perhaps are restricted from re-assembling into the higher-order structures when the denaturant is removed, thus resulting in lesser degree of swelling. The observation #3 presented above argues that in this case the protein chains are not restricted from re-assembling after the crosslinking is completed. Comparison of the equilibrium swelling of the series DI-Fgn-PEG-NB+DTT and Fgn+10KL and Fgn+6KL provided further insight. The network DI-Fgn-PEG-NB+DTT was prepared under non-denaturing conditions, by crosslinking ˜3.7 kDa PEG-norbornene extensions with dithiothreitol, resulting essentially in linear PEG-based chains of ˜7.5 kDa between protein chains. The networks Fgn+10KL and Fgn+6KL were prepared under denaturing conditions, with 10 kDa and 6 kDa PEG chains bridging the same protein chains as in DI-Fgn-PEG-NB+DTT. According to observations #1 and #3 above we expected the equilibrium swollen mass of the DI-Fgn-PEG-NB+DTT network to fall between the masses of Fgn+10KL and Fgn+6KL—if the resulting network structures are the same, and it did. The fact that it is closer to Fgn+10KL than Fgn+6KL was likely due to differences in solvent interaction parameter between linear PEGs (10KL and 6KL) and linear PEG interrupted with the dinorbornene-thioether linkage (DI-Fgn-PEG-NB+DTT).

### Example 22—Direct-to-Polymer Formation of Slab-Scaffold Using Gamma Chain of Human Fibrinogen

This example describes preparation of a scaffolding formulation directly from reduced denatured Gamma chain of human fibrinogen and synthetic polymers, bypassing preparation and characterization of Fgn-Gamma-PEG-NB. The Gamma chain of human Fibrinogen can be obtained recombinantly or by separating donor-derived Fibrinogen chains as disclosed elsewhere in this Application.

200 mg of reduced denatured gamma chain of human fibrinogen dissolved in 8M urea, 40 mM sodium phosphate pH 7.4 was prepared according to the Example 21. The protein was concentrated to ˜50 mg/mL using a 10 kDa molecular weight cut-off Amicon Ultra 15 centrifugation device (Millipore Catalog number (UFC901024) at 4,000 RCF in a refrigerated centrifuge. To ensure complete reduction of cysteine residues in the protein, tris(2-carboxyethyl)phosphine hydrochloride (TCEP) was added to the final concentration of 30 mM and the concentrated protein solution was transferred to a 3 mL Slide-A-Lyzer dialysis cassette (molecular weight cut-off of 20 kDa; Thermo Fisher catalog #66003) and dialyzed against 4 L of 8 M urea, 40 mM sodium phosphate pH 7.4 overnight at +4° C. in a sealed container. The dialyzed protein solution was removed from dialysis cassette, transferred to a 15 mL conical tube and kept on ice during subsequent manipulations. Protein concentration was calculated to be 41.1±0.21 mg/mL at this point by measuring absorbance of the protein solution at 280 nm with a spectrophotometer. Concentration of free thiols was measured using Ellman's assay and was found to be 6.0±0.3 mM, or about 72% of theoretical maximum (8.41 mM) calculated on the bases of measured protein concentration and molecular weight and cysteine content of the gamma subunit of human fibrinogen (data from UNIPROT database, www.uniprot.org).

Simplified scaffolding formulations were prepared from reduced, denatured gamma subunit of fibrinogen by crosslinking it with synthetic PEG-norbornenes in the course of photoinitiated thiol-ene reaction. Because the reduced denatured fibrinogen provides sufficient source of free thiols for the reaction to occur, the dithiol peptides utilized in other Examples were omitted here for simplicity. Four similar pre-polymer formulations were prepared, each containing 29 mg/mL reduced, denatured gamma chain of fibrinogen, 0.01% photoinitiator NAP, and the following PEG-norbornenes at the concentrations sufficient to achieve 1:1 stoichiometric ratio between the norbornenes and free thiols: 4-arm PEG 20 kDa tetranorbornene (formulation Gamma+20k-4), linear PEG 20 kDa dinorbornene (formulation Gamma+20KL), linear PEG 10 kDa dinorbornene (formulation Gamma+10KL) and linear PEG 6 kDa dinorbornene (formulation Gamma+6KL). The concentration of denaturant (urea) was between 6.5 and 7.8 M, sufficient to completely denature fibrinogen chains. 50 uL hydrogels were photopolymerized from these pre-polymer formulations in triplicate, by exposing the formulations to 20 mW/cm² 385 nm UV light for 90 seconds. All formulations formed robust hydrogels. Upon completion of polymerization, denaturant was removed from the hydrogels via passive diffusion, by incubating each hydrogel with 1 mL phosphate-buffered saline at ambient temperature for a few hours, followed by replacement of the buffer with fresh 1 mL and additional incubation to reach swelling equilibrium.

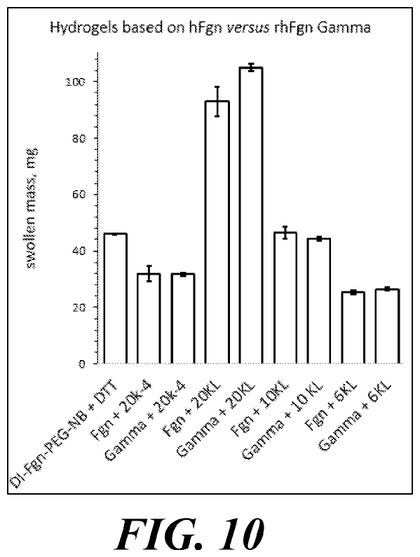

All swollen hydrogels were weighed, replicate observations averaged, and the resulting data are shown in FIG. 10 in comparison with the results obtained with reduced denatured donor-derived fibrinogen obtained in the Example 21 above. It is obvious that physical properties of hydrogels obtained by crosslinking reduced denatured gamma chain of human fibrinogen are essentially indistinguishable from the properties of hydrogels obtained by crosslinking full reduced, denatured human fibrinogen at the same total protein concentration. Therefore, all observed trends and conclusions about network structure of these hydrogels are identical to the conclusions derived in the example 21 and are listed below:

The following trends were observed and found to be in full agreement with Flory-Rehner theory of swelling of covalently crosslinked polymeric networks (see Metters, A. and Hubbell, J. 2005. Biomacromolecules 6, 1, pp 290-301 for a recent reference):

1. The degree of swelling, as measured by the equilibrium mass of the hydrogels, increased with increase in molecular weight of the linear chains between crosslinks. This was evident from a comparison between series Gamma+6KL, Gamma+10KL and Gamma+20KL, where increase in molecular weight of the linear PEG components from 6 to 10 to 20 kDa correlated with increase in the equilibrium swollen mass.

2. The degree of swelling decreased with increase in the branch order of the network. This was evident from comparing series Gamma+10KL and Gamma+20k-4, which had identical weight concentrations of the protein and polyethylene glycol, but different branch order. 20 kDa 4-arm PEG tetranorbornene contained the same molecular weight per a norbornene residue as the 10 kDa PEG dinorbornene (5 kDa), but had a higher branch order, resulting in smaller degree of hydrogel swelling at equilibrium.

Additional conclusions regarding the protein component of the network:

3. The protein chains inside the hydrogels did bind each other, even if the hydrogel was crosslinked in the presence of denaturant that is subsequently removed. This was evidenced by the fact that Gamma+6KL and Gamma+20k-4 formulations actually shrunk upon reaching equilibrium (equilibrium swollen mass of 30 mg and below, compared to ˜50 mg mass expected if the volume of the hydrogel remained the same as at the time of photopolymerization). This shrinking was most likely due to protein chains binding each other as the denaturant is removed; similar protein-protein interactions were also expected in the series Gamma+10KL and Gamma+20KL, but the swelling there was offset by much higher content of the hydrophilic polymer (PEG) comprising network.

4. The network structure of the hydrogels prepared under non-denaturing conditions was very similar to the network structure of the hydrogels prepared under denaturing conditions once the denaturant was removed and equilibrium swelling was achieved. One can imagine a situation where a PEGylated protein self-assembles into soluble high-order structures under non-denaturing conditions prior to photopolymerization, and photo-crosslinking generates PEG-based mesh around such structures (series DI-Fgn-PEG-NB+DTT). If one crosslinks the same components under denaturing conditions, where protein-protein interactions are disrupted due to the presence of the denaturant, one may expect a different, more homogeneous network structure, where individual protein chains are trapped in the PEG-based mesh away from each other, and perhaps are restricted from re-assembling into the higher-order structures when the denaturant is removed, thus resulting in lesser degree of swelling. The observation #3 presented above argues that in this case the protein chains are not restricted from re-assembling after the crosslinking is completed. Comparison of the equilibrium swelling of the series DI-Fgn-PEG-NB+DTT and Gamma+10KL and Gamma+6KL provided further insight. The network DI-Fgn-PEG-NB+DTT was prepared under non-denaturing conditions, by crosslinking ˜3.7 kDa PEG-norbornene extensions with dithiothreitol, resulting essentially in linear PEG-based chains of ˜7.5 kDa between protein chains. The networks Gamma+10KL and Gamma+6KL were prepared under denaturing conditions, with 10 kDa and 6 kDa PEG chains bridging the same protein chains as in DI-Fgn-PEG-NB+DTT. According to observations #1 and #3 above we expected the equilibrium swollen mass of the DI-Fgn-PEG-NB+DTT network to fall between the masses of Gamma+10KL and Gamma+6KL—if the resulting network structures were the same, and it did. The fact that it was closer to Gamma+10KL than Gamma+6KL was likely due to differences in solvent interaction parameter between linear PEGs (Gamma+10KL and Gamma+6KL) and linear PEG interrupted with the dinorbornene-thioether linkage (DI-Fgn-PEG-NB+DTT).

### Example 23—Lyophilized Kits

It is anticipated that manufacturing and storage of a complex, protein-based pre-polymer solution for bed-side application in clinic may require development of a lyophilized version of the pre-polymer. Lyophilized formulations of drugs and biologics typically have longer shelf-life and simplified storage conditions, compared to ready-to-use solutions that often need to be stored at cryogenic temperatures in order to retain potency and avoid protein degradation and aggregation that commonly occurs in concentrated solutions upon storage. Lyophilized formulations can often be stored at ambient temperature or at +4° C. (e.g. in an inexpensive refrigerator commonly found around kitchens and medical offices) and ready-to-use solutions can be generated from the lyophilized formulations in minutes, upon addition of sterile saline or water immediately prior to medical use.

Successful development of a lyophilized formulation depends on identifying a lyoprotectant compatible with the formulation that is an excipient or a combination of excipients that protects the active ingredient during freezing, water removal (lyophilization itself) and long-term storage without loss of the potency or biological or chemical activity of the cargo. Ideally, a good lyoprotectant also enables fast rehydration of the formulation prior to use.

Some of the most common lyoprotectants used to develop lyophilized formulations of drugs and biologics include mixtures of non-reducing sugars such as sucrose or trehalose and non-ionic detergents such as Tween 20 or Tween 80 and others. Theoretical frameworks explaining physical, chemical cryo- and lyoprotective functions of these excipients have been developed in recent years (M. A. Mensink et al. European Journal of Pharmaceutics and Biopharmaceutics 114 (2017) 288-295; W. Wang. International Journal of Pharmaceutics 203 (2000) 1-60). However, in practical terms, the search for an ideal lyoprotectant for a specific cargo is still routinely performed by trial-and-error. In this Example we disclose development of a lyoprotectant for the scaffolding pre-polymer formulations containing DI-Fgn-PEG-NB. It is anticipated that lyoprotectants for the scaffolding pre-polymer formulations containing individual chains of DI-Fgn-PEG-NB can be identified using similar fast screening procedure.

In a first round, feasibility of lyophilizing concentrated solution of DI-Fgn-PEG-NB was evaluated. Four 200 uL samples in 2 mL cryogenic vials were prepared, each containing 35.4 mg/mL DI-Fgn-PEG-NB, 25 mM sodium acetate buffer pH 5.0 and the following excipients: none (sample Ac-0), 0.2 M sucrose (sample Ac-S) and 0.2 M sucrose plus 0.1% wt/vol Tween 80 (samples Ac-ST and CTL). Samples Ac-0, Ac-S and Ac-ST were flash-frozen by immersing them into an isopropanol-dry ice bath (˜−70° C.), and then lyophilized at 70 mTorr pressure in a bench-top laboratory freeze-dryer (Labconco FreeZone 2.5 L −50 C Freeze-Dryer). Sample CTL was not frozen and stored at +4° C. as a positive control.

Upon completion of lyophilization, freeze-dried samples were transferred to ambient temperature and pressure and visually inspected. Samples Ac-S and AC-ST appeared as white porous “cakes” each occupying about 200 uL volume, while sample Ac-0 has collapsed into a yellowish clump. These samples were then re-hydrated by addition of 180 uL ultrapure water, followed by gentle rocking on a nutator for 15 minutes. Visual observation revealed that sample Ac-ST formed a clear transparent solution identical in appearance to sample CTL, sample Ac-S contained a small amount of yellowish insolubles and sample Ac-0 remained a yellowish clump, with a halo of partially rehydrated gel-like material surrounding an apparently dry core. These observations suggest that lyophilization of DI-Fgn-PEG-NB solution in the presence of 0.2 M sucrose and 0.1% wt/vol Tween 80 results in a sample that can be readily re-hydrated into its original, fully dissolved state, while omission of one or both of these components significantly impedes rehydration of the lyophilized sample.

To test whether lyophilization and rehydration has impacted chemical reactivity of the DI-Fgn-PEG-NB, samples Ac-ST and CTL were each supplemented with 22.2 uL of 110 mg/mL 4-arm PEG-10 kDa-tetrathiol (NOF, Sunbright PTE-100 SH) solution in water and 1 uL of 1% wt/vol photoinitiator NAP. This provided a 4-arm polymeric crosslinker at a 1:1 stoichiometric ratio of free thiol to the input norbornene concentration. Samples were irradiated at 20 mW/cm² with 385 nm UV light for 30 s. Both samples readily formed clear, transparent hydrogels of identical stiffness, as evaluated by careful compression with a plastic spatula. This observation confirms that lyophilization and rehydration of DI-Fgn-PEG-NB in the presence of 0.2 M sucrose and 0.1% wt/vol Tween 80 does not interfere with its chemical reactivity.

To evaluate influence of pH and buffering salts on lyophilization and rehydration of DI-Fgn-PEG-NB, the abovementioned experiments were performed with 25 mM sodium acetate buffer replaced with 25 mM sodium citrate buffer, pH 6.0 or with phosphate-buffered saline (PBS), pH 7.4. Replacing the buffer and pH did not result in any notable change in outcome, including rehydration and photopolymerization trends described above, suggesting that the key ingredients necessary for lyophilization and rehydration of DI-Fgn-PEG-NB are indeed 0.2 M sucrose and 0.10% wt/vol Tween 80.

In the next round, feasibility of lyophilization and rehydration of a more complex, full scaffolding pre-polymer formulation containing DI-Fgn-PEG-NB, PEG-norbornenes, dithiol peptides and photoinitiator NAP was evaluated. Two 250 uL samples each containing DIFgn-PEG-NB at 15 mg/mL, 20K4A PEG-NB at 1% wt/vol, 6KL PEG-NB at 1% wt/vol, polypeptide hCysMMPA (SEQ ID NO: 19) at 3.75 mM (7.5 mM free thiol concentration), NAP at 0.05% wt/vol and phosphate-buffered saline pH 7.4 at 0.5× concentration, sucrose at 0.2 M and Tween 80 at 0.1% wt/vol were prepared and freeze-dried as described above. The resulting samples appeared as white porous “cakes” each occupying about 250 uL volume. The samples were re-hydrated by addition of 235 uL of ultrapure water to each, followed by gentle rocking on a nutator for 15 minutes. Both samples formed clear transparent solutions at this point, suggesting that lyoprotectant identified above for the lyophilization of DI-Fgn-PEG-NB is suitable for lyophilization of a more complex scaffolding formulation as well. 200 uL of each sample was photopolymerized by exposure to 20 mW/cm² 385 nm UV light for 30 s, at which point each hydrogel was placed into 1 mL of ultrapure water and incubated overnight at ambient temperature to reach equilibrium swelling (samples “lyophilized”).

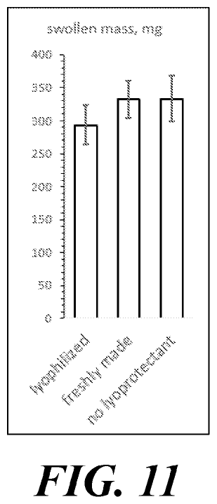

For comparison, two pre-polymer formulations, one identical to the lyoprotectant-containing full scaffolding formulation described above (“freshly made”) and another one omitting sucrose and Tween 80 (“no lyoprotectant”) were prepared fresh from the concentrated stocks of the reagents and 200 uL hydrogels photopolymerized in duplicate and swollen as described above. All swollen hydrogels were weighed, replicate observations averaged and resulting measurements are shown in FIG. 11. It is apparent, given the error in the measurement, that lyophilized and rehydrated formulation polymerizes to yield hydrogels with the same swelling behavior as identical formulation that was not lyophilized, as well as a similar formulation that does not contain lyoprotectant. These observations indicate that neither lyoprotectant itself, nor the process of freeze-drying followed by rehydration interferes with reactivity of the pre-polymer components and does not affect the structure of the resulting biopolymer network.

### Example 24—Synthesis of Fibrinogen-Based Direct-to-Polymer Microparticles

The synthesis of Fibrinogen-based scaffolding microparticles directly from reduced and denatured Fgn was performed following the method disclosed in Example 9, using a modified pre-polymer formulation Fgn+10KL disclosed in the Example 21.

A microfluidic droplet generation chip was used to create water-in-oil emulsion droplets that were then cured with 385 nm light. Briefly, 10 mL of the pre-polymer solution Fgn+10KL (described in Example 21, above) and 50 mL of hexanes containing Span 80 (1% wt./vol. final) were prepared as the water (pre-polymer solution) and oil (hexanes+Span 80) phases. An Elvesys Flow Control System was then used to feed the water and oil phases into a Dolomite Droplet T-Junction Chip (260 μm etch depth) flow ratios of approximately 1:3 (water:oil). This creates individual water-in-oil droplets that remain separated as they flow through an outflow tubing (0.5 mm internal diameter), as observed with a high-speed microscope camera (Darwin). It must be noted that flow rates must be continually monitored and adjusted to maintain stable droplet formation and prevent droplet aggregation while traveling through the outflow tubing. A 385 nm light (about 20 mW/cm²) was placed over the outflow tubing to polymerize the droplets before collection. Flow rates were chosen to result in a travel time for the droplets of 30-45 seconds under the polymerizing light before collection of the outflow into 50 mL Falcon tubes containing 5 mL of distilled water. Polymerized beads were then cleaned by repeated centrifugation (5 minutes at 1500×g) and washing steps with distilled water until the theoretical concentration of urea in the beads was less than 0.1 mM based on bead:wash volumes. The beads were then stored in 0.5×PBS at 4 C.

The quality and size distribution of the prepared microparticles and dependence of their size on the adjustable process parameters, such as internal diameter of tubing used in the preparation, was investigated by light microscopy as described in Example 9 and was found to be indistinguishable from data obtained for microparticles containing full DI-Fgn-PEG-NB.

### Example 25—Synthesis of Fibrinogen Gamma-Based Direct-to-Polymer Microparticles

The synthesis of Fibrinogen-based scaffolding microparticles directly from reduced and denatured Fgn was performed following the method disclosed in Example 9, using a modified pre-polymer formulation Gamma+10KL disclosed in the Example 22.

A microfluidic droplet generation chip was used to create water-in-oil emulsion droplets that were then cured with 385 nm light. Briefly, 10 mL of the pre-polymer solution Gamma+10KL (described in Example 22, above) and 50 mL of hexanes containing Span 80 (1% wt./vol. final) were prepared as the water (pre-polymer solution) and oil (hexanes+Span 80) phases. An Elvesys Flow Control System was then used to feed the water and oil phases into a Dolomite Droplet T-Junction Chip (260 μm etch depth) flow ratios of approximately 1:3 (water:oil). This created individual water-in-oil droplets that remained separated as they flowed through an outflow tubing (0.5 mm internal diameter), as observed with a high-speed microscope camera (Darwin). It must be noted that flow rates must be continually monitored and adjusted to maintain stable droplet formation and prevent droplet aggregation while traveling through the outflow tubing. A 385 nm light (about 20 mW/cm²) was placed over the outflow tubing to polymerize the droplets before collection. Flow rates were chosen to result in a travel time for the droplets of 30-45 seconds under the polymerizing light before collection of the outflow into 50 mL Falcon tubes containing 5 mL of distilled water. Polymerized beads were then cleaned by repeated centrifugation (5 minutes at 1500×g) and washing steps with distilled water until the theoretical concentration of urea in the beads was less than 0.1 mM based on bead:wash volumes. The beads were then stored in 0.5×PBS at 4 C.

The quality and size distribution of the prepared microparticles and dependence of their size on the adjustable process parameters, such as internal diameter of tubing used in the preparation, was investigated by light microscopy as described in Example 9 and was found to be indistinguishable from data obtained for microparticles containing full DI-Fgn-PEG-NB.

### BIOLOGICAL EXAMPLES

### Example B1—In Vitro Cellular Invasion Assay

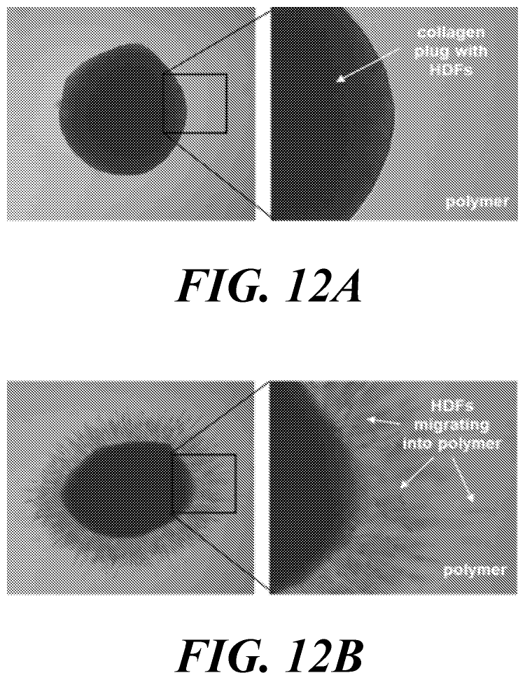

An in vitro cellular invasion assay was used study how changes in scaffolding composition affected the scaffolding properties (i.e. cellular migration) of the biomaterial. Briefly, cells were suspended in Type I Collagen and allowed to contract for 24-48 hours under standard cell culture conditions. The collagen-cell plugs were placed into 48-well plates and then suspended in a precursor solution, which was polymerized around the plug by exposure to light. Media supplemented with platelet releasates was placed into each well, and the invasion of out of the collagen plug and into the hydrogel matrix was monitored by light microscopy for 24-72 hours. Examples of data obtained from this assay are presented in FIGS. 12A and 12B. The cell-seeded plug (dark object) was then encapsulated within an optically clear photopolymer. Under suitable conditions, cells will migrate out of the collagen plug and into the surrounding material, which can be observed by standard light microscopy.

### Preparation of Collagen Solution:

Immediately before use, Rat tail Type I Collagen (Fisher: CB-40236) was diluted with serum-free DMEM (with L-glutamine and 4.5 g/L glucose) containing sodium bicarbonate to make a working collagen solution with 2.5 mg/ml collagen and 1% wt./vol. sodium bicarbonate. The collagen remains in a liquid state when kept under acidic conditions at refrigerated temperatures. Therefore, it is important to keep these reagents on ice until use and mix only before needed to resuspend cell pellets.

### Preparation of Collagen Plugs:

Human Dermal Fibroblasts (HDF) were expanded under standard culture conditions before being trypsinized, collected into tubes, and counted using a hemocytometer. The cells were then pelleted by centrifugation at 800×g and resuspended in a collagen solution (described above) at a concentration of 2.5-3×10\hat{ }6 cells/ml. The collagen-cell suspension was then aliquoted (25 μL/well) into v-bottom polypropylene 96-well plates. The plates were incubated at 37 C for 30 minutes to allow the collagen solution to solidify before adding 200 μL of growth media (DMEM with L-glutamine and 4.5 g/L glucose+10% v/v FBS) to each well. The addition of media usually causes the collagen plug to detach from the bottom of the well, allowing plug contraction. If plugs were not observed to detach from the bottom of the well at the time of adding growth media, a pipet tip was used to gently free the plug from the bottom of the plate. The cell-seeded collagen plugs were incubated 37 C for 1-3 days before use in this assay.

### Collagen Plug Encapsulation:

After incubating the cell-seeded collagen plugs for 1-3 days, each plug was transferred by pipet into a 48-well suspension culture plates (wide-mouthed or cut-off pipet tips were used to accommodate plug diameters). All media was then aspirated from the well, taking care to avoid disrupting the plug. 250 μL of the precursor solution was added to each well, and a pipet tip was used to gently move each plug to the center of its well. The mixture was then polymerized to form a hydrogel matrix surrounding the collagen plug in the well by exposure to light (385 nm, 19 mW/cm2, 30 seconds). Following polymerization, 200 d of growth media (DMEM with L-glutamine and 4.5 g/L glucose+10% v/v FBS) was added to each well, and plates were incubated at 37 C.

### Cellular Invasion:

Cellular invasion into the hydrogel matrix was observed by standard light microscopy. Images were taken with a Leica EC3 camera attached to a Motic AE31 microscope. Cell-seeded collagen plugs were not translucent and were observed as dark, roundish shapes in the center of each image as can be seen in FIG. 12A. The hydrogel matrix was translucent and was clearly distinguishable from the collagen plug as can be seen in FIG. 12A. Cells migrating out from the collagen plugs appear as dark, thin projections from the plug as can be seen in FIG. 12B.

The PEG-NB components in the precursor solution as used and described in this Example contained both a linear 6KL PEG-NB and a multiple arm PEG-NB. In some cases, a four-arm 20K4A PEG-NB was used as the multiple arm PEG-NB; in some cases, a six-arm 30K6A PEG-NA was used as the multiple arm PEG-NB.

FIG. 12C presents a picture of cellular infiltration assay experiment for materials with 20K4A PEG-NB, and FIG. 12D presents a picture of cellular infiltration assay experiment for materials with 30K6A PEG-NB. No difference in terms of cellular invasion was observed between these two cases.

This demonstrated that the variation in the number of arms of multiple arm PEG-NB did not change cellular invasion of the photocured scaffolds. The 20K4A PEG-NB and 30K6A PEG-NB were just two examples of multiple armed PEG-NB. In some embodiments, the 20K4A PEG-NB can be replaced by other PEG-NBs with different number of arms, wherein the number is an integer selected from the group consisting of 3, 4, 5, 6, 7, 8, 9, and 10. In some embodiments, n is 3. In some embodiments, n is 4. In some embodiments, n is 6. In some embodiments, n is 8.

### Example B2—Acute, Full-Thickness Wound Healing Model

A porcine, full-thickness wound healing model was used to evaluate the ability of hydrogel formulations to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

Hydrogel scaffolds were either applied as a liquid to the wound site and photopolymerized in situ or applied in a pre-polymerized microsphere format. For in situ polymerizations, a 385 nm light at 20 mW/cm2 was directed over the wound for 30 seconds, a time determined to be sufficient for complete polymerization of the matrix. Tegaderm dressings were applied to protect each wound and reduce the chance of infection. Because differences in healing rates vary over the surface area tested, treatments locations were randomized between animals. Untreated, control wounds received no scaffolding material and were covered with Tegaderm. When all wounds had been treated, protective dressings and jackets were used to prevent animals from disturbing the sites.

### Histological Evaluation:

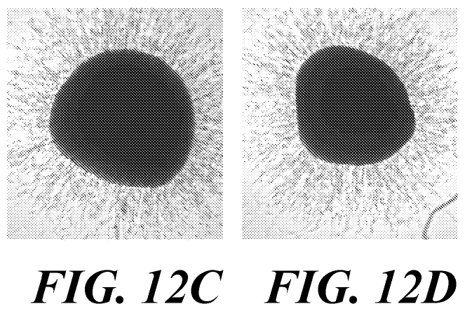

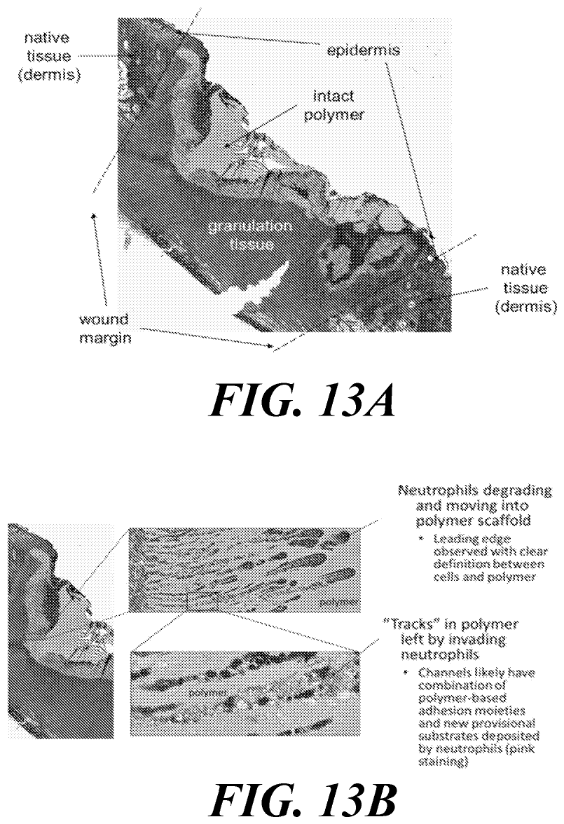

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 13A and FIG. 13B. FIG. 13A identifies key features of a partially healed wound. FIG. 13B illustrates cells migrating into the hydrogel matrix in vivo. Harvesting tissue at different days post-wounding makes it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

### Example B3—Influence of Fibrinogen Component on In Vitro Scaffolding Properties

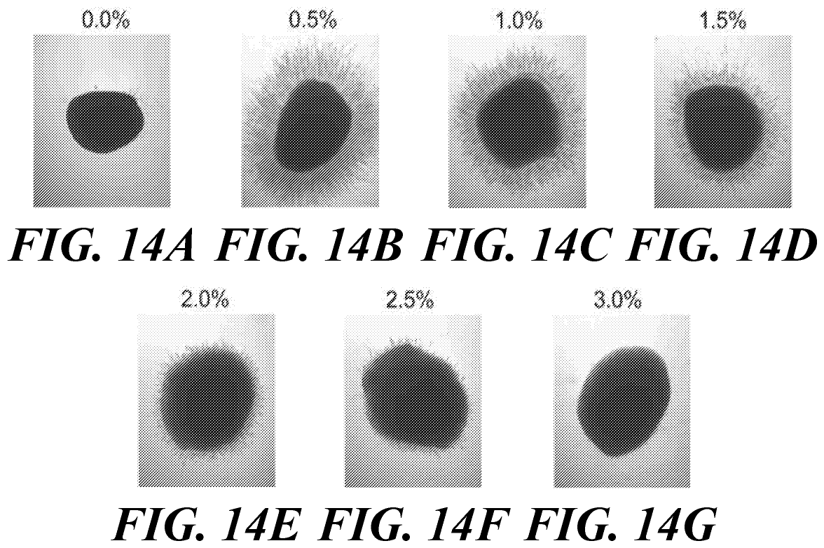

To characterize the scaffolding properties of the hydrogel materials forming the hydrogel microparticles described herein, a cellular invasion assay was performed to test a range of DIFgn-PEG-NB concentrations for the fibrinogen component of the scaffolding formulation. As shown in FIGS. 14A-14G, changing the final concentration of DIFgn-PEG-NB in the scaffolding formulation significantly impacted the level of HDF invasion into the scaffold. Polymers that did not include DIFgn-PEG-NB (0% wt./vol.) showed no cellular infiltration (FIG. 14A). With the addition of 0.5% wt./vol. DIFgn-PEG-NB, robust cellular invasion was observed (FIG. 14B). Interestingly, increasing DIFgn-PEG-NB concentration had the effect of reducing both the number of cells within the material and distance of cellular migration from the collagen plug (FIGS. 14C-14G). These data indicate that, while DIFgn-PEG-NB is necessary for the cellular scaffolding property of the matrix, the increased network density that accompanies increased DIFgn-PEG-NB concentrations acts to limit cellular movement in the material. In other words, at higher concentrations, DIFgn-PEG-NB creates a tighter matrix that reduces the effective network pore size, thereby limiting, or preventing, cellular movement. Because the scaffold is degradable, cells were ultimately able to move into the material, but movement is slowed as cells must first spend additional time degrading the hydrogel to create space for movement.

### Example B4—Influence of Fibrinogen Component Ratio on Degradability and Scaffolding Properties

Example B3 was used to demonstrate the scaffolding function of the hydrogel formulation, it also highlights that higher concentrations of DIFgn-PEG-NB slow cellular infiltration, likely due to the increased network density.

The influence of concentrations of DIFgn-PEG-NB on the tissue scaffold's degradability and scaffolding properties were evaluated in vivo.

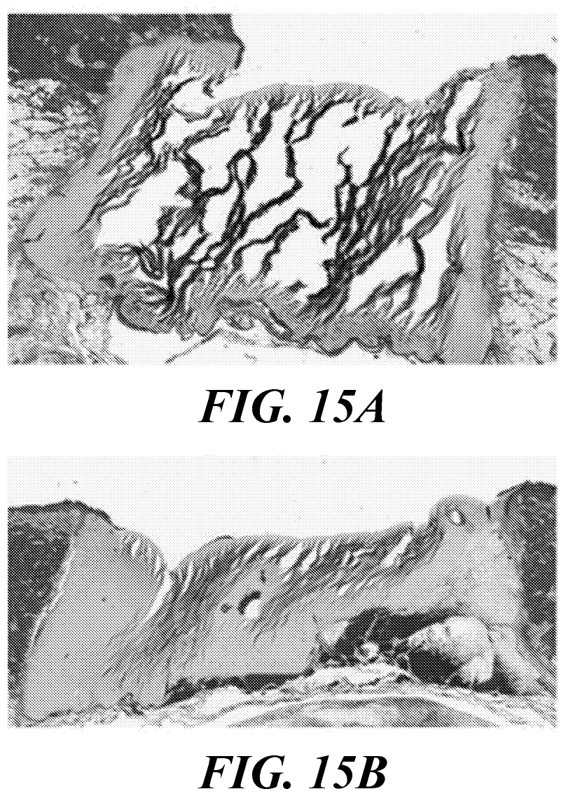

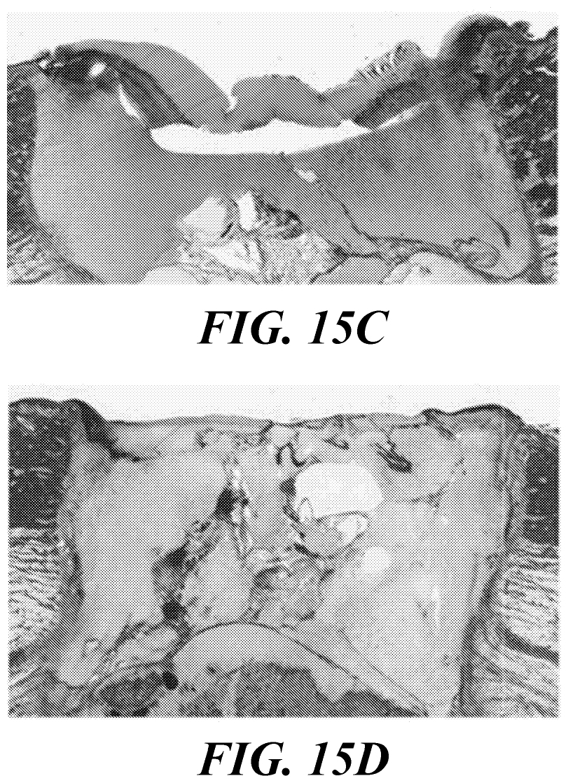

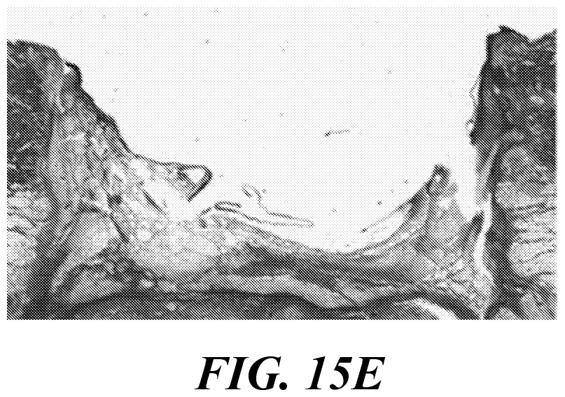

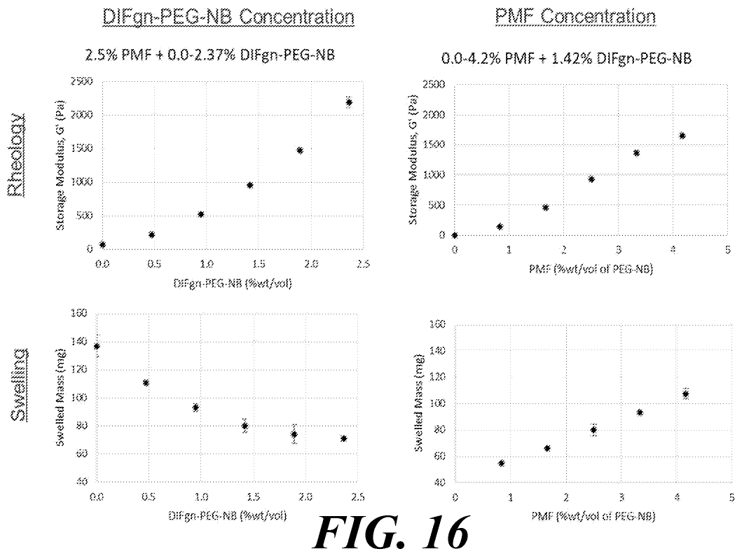

At concentrations of DIFgn-PEG-NB of 3.0% wt./vol. (FIG. 15A), the hydrogel remained intact after 7 days and showed limited evidence of degradation. Cellular infiltration was limited to wound edges. This observation confirms that the higher concentration results in increased network density which limits cellular movement in the material.

At concentrations of DIFgn-PEG-NB of 2.25% wt./vol. (FIG. 15B), the hydrogel remained intact after 7 days. Clear evidence of cellular migration within the hydrogel matrix was observed. Cellular infiltration remained slightly limited.

At concentrations of DIFgn-PEG-NB of 1.5% wt./vol. (FIG. 15C), the hydrogel remained intact after 7 days. Clear evidence of increased cellular infiltration and scaffolding was observed.

In this study, increasing levels of cellular invasion into the hydrogel were observed as the concentration of DIFgn-PEG-NB was lowered from 3% wt./vol. (FIG. 15A) to 1.5% wt./vol. (FIG. 15C). Neutrophil migration into the 1.5% DIFgn-PEG-NB matrix (FIG. 15C) was clearly observed as a wave of red-stained cells reaching near the top of the polymer.

At concentrations of DIFgn-PEG-NB of 0.75% wt./vol. (FIG. 15D), cells had moved into the wound volume, but the hydrogel appeared to have been mostly degraded, leaving no sustained tissue scaffolding.

At concentrations of DIFgn-PEG-NB of 0% wt./vol. (FIG. 15E), the hydrogel material liquefied within a few days after application without significant new tissue deposition.

These data indicate that a balance must be struck with the concentration of DIFgn-PEG-NB in the matrix formulation. High concentrations of DIFgn-PEG-NB increased the structural stability of the hydrogel but slowed cellular infiltration. Low concentrations of DIFgn-PEG-NB permitted rapid cellular infiltration but lost their structural integrity if degradation of the material occurred too quickly and failed to support cellular migration until new tissue was deposited to create an extracellular matrix.

### Example B5—Influence of Component Ratios on Physical Properties

To examine the effects of the fibrinogen component (DIFgn-PEG-NB) and of the polymer component and protease-cleavable component on the physical properties of the resultant matrix, a range of concentrations of each of these formulation components were tested in rheology and swelling assays. The polymer component consisted a one-to-one mass ratio mixture of a 4-arm 20 kDa PEG-tetranorbornene (20K4A PEG-NB) and a 6 kDa PEG-dinorbornene (6KL PEG-NB). The protease-cleavable component consisted of a one-to-one molar ratio of peptides of SEQ ID NO. 7 and SEQ ID NO. 19. The polymer component and protease-cleavable component were introduced in a 1:1 molar ratio of thiol:ene. Taken together in this ratio, the polymer component and protease-cleavable component were referred to as PMF. It is understood that when PMF concentration was varied, the ratio of polymer component to protease-cleavable component was maintained constant. For rheological experiments, the storage moduli of distinct formulations were measured using a Discovery HR3 rheometer (TA Instruments) equipped with a quartz plate through which light could be directed to initiate polymerization while simultaneously measuring the physical properties of the hydrogel sample. Each formulation was irradiated with the 385 nm light at 20 mM/cm², and the elastic properties of the resulting hydrogel was measured in real time by the Discovery HR3 rheometer (TA Instruments) equipped with a UV-transparent quartz plate and a 8-mm flat plate tool (parallel geometry) positioned at 0.25 mm from the quartz plate. 20 μL of the pre-polymer solution was applied between the tool and quartz plate, extra solution removed by careful wicking and UV was turned on within 5 s of the beginning of data collection. Shear rheometry was performed in the oscillating fast sampling mode, with 1% strain applied at 10 rad/s frequency. Hydrogel formation was evidenced by increase in the elastic shear modulus G′ which then reached plateau values within 20-60 s. In the top two plots shown in FIG. 16, storage modulus is plotted against the concentration of either DIFgn-PEG-NB or PMF while maintaining the concentration of the other component constant (PMF and DIFgn-PEG-NB, respectively). In both cases, increasing the concentration increased the storage modulus, or physical stiffness, of the resultant hydrogel in a concentration-dependent manner.

Several important observations can be made when looking more closely at these data. The first is that DIFgn-PEG-NB-only and PMF-only polymers were relatively soft, reducing their suitability for clinical applications. The DIFgn-PEG-NB-only formulations do create hydrogel networks, but the storage modulus is often at or below the limit of detection for the rheometer that was used in these experiments. The PMF-only formulations had measurable storage moduli in the range of 50-90 Pa but were still considered too soft for clinical applications. The second observation that can be made with these data is that increasing the concentration of DIFgn-PEG-NB has an accelerating effect on the storage modulus of the polymer. Unlike increasing PMF concentration, which results in a roughly linear increase in the storage modulus, increasing the concentration of DIFgn-PEG-NB results in a nonlinear increase in the storage modulus. Though is not clear what is causing this effect, and without wishing to be bound by theory, it is possible that increase concentrations of DIFgn-PEG-NB increase the prevalence of noncovalent interactions between the DIFgn-PEG-NB monomers.

To evaluate the effects of DIFgn-PEG-NB and PMF concentrations on swelling, a simple swelled-mass assay was used. Briefly, formulations were mixed and aliquoted into the tips of cut-off 1 mL syringes. Light was used to polymerize these formulations within the syringe, and the plugs were then pushed out and into tubes containing 0.5×PBS, pH 7.4, where they were incubated at room temperature overnight. Each plug was then removed from the tube, and excess liquid was removed before weighing. Because each plug was created with 50 μL aliquots of formulation, an initial mass for each plug was assumed to be 50 mg.

The results of this experiment indicate the two competing effects were present in this hydrogel system. Increasing the concentration of DIFgn-PEG-NB resulted in a nonlinear reduction in hydrogel swelling, suggesting that DIFgn-PEG-NB has a positive effect on swelling but that this effect reaches a plateau at higher concentrations. In contrast, increasing the concentration of the PMF components resulted in a linear increase in swelled mass of the polymer, a result that is somewhat counterintuitive in that increasing network density should result in a stiffer network that is more resistant to swelling. However, PMF is a PEG-based material, and PEG's hygroscopic nature would have the effect of drawing in water, with more PEG increasing the osmotic pressure of the material and resultant swelling.

The results of the above experiments are shown in FIG. 16.

### Example B6—Importance of Covalent Integration of the Fibrinogen Component

The importance of the DIFgn-PEG-NB acting as a structural backbone to the polymer network was observed in in a study in which the scaffolding function of formulations containing DIFgn-PEG-NB or a fibrinogen derivate incapable of covalent incorporation within the hydrogel, namely DIFgn-PEG-MME.

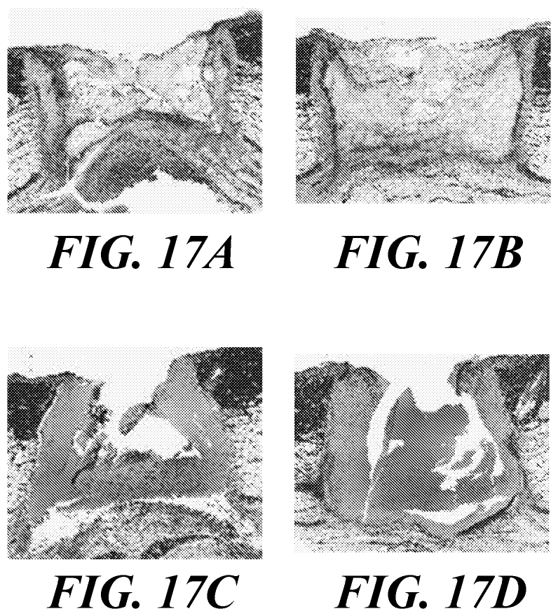

In vitro, both forms of PEGylated fibrinogen supported cellular migration in the cell invasion assay. That is, PEGylated fibrinogen that does not covalently incorporate into the thiol-ene network but is only encapsulated within the network upon polymerization (DIFgn-PEG-MME) performs well as a substrate for cellular migration in vitro. In vivo, however, DIFgn-PEG-MME-based formulations degraded too quickly, causing the structural aspect of the polymer to be lost, thereby preventing scaffolding function. In the representative images in FIGS. 17A-17D, formulations with equal concentrations of either DIFgn-PEG-MME or DIFgn-PEG-NB show distinctly different abilities to act as scaffolds in full-thickness wounds. Whereas the polymers with DIFgn-PEG-MME were no longer intact after seven days as shown in FIGS. 17A and 17B, the polymers with DIFgn-PEG-NB were still present in the wounds and had significant neutrophil invasion as shown in 8C and 8D. These data indicate that the DIFgn-PEG-NB serves an important structural role in maintaining the integrity of the network long enough for cellular invasion and new tissue deposition.

One aspect that may be contributing to the effect observed in this study is that DIFgn-PEG-NB also serves an important role in preventing polymer swelling. In addition to adding additional covalent and noncovalent cross-links that stabilize the scaffold within wounds, incorporating DIFgn-PEG-NB into the backbone of the network reduces the tendency of the polymer to take on water and swell. This property of DIFgn-PEG-NB is demonstrated in the following section.

### Example B7—Microspheres for Cellular Migration—Degradation Independent Mechanism

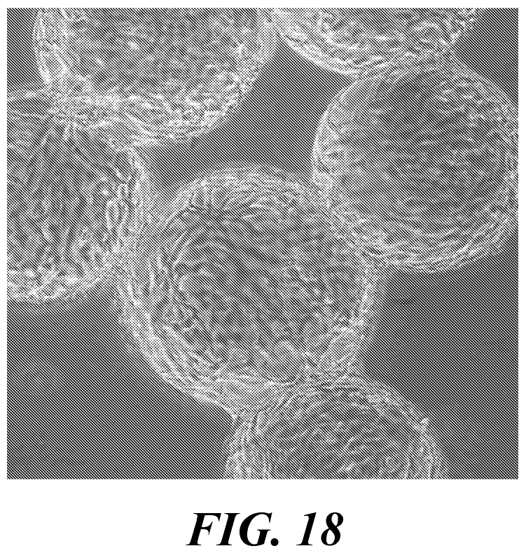

Cultured Human Dermal Fibroblasts (HDFs) were mixed in media with hydrogel microspheres in a 1.5 mL Eppendorf tube for 30 minutes, and then the contents of the tube were transferred into a 24-well suspension culture plate and incubated overnight at 37° C. in a CO₂ incubator. The microspheres were then transferred to a new 24-well plate, leaving behind as many non-adherent cells as possible. HDF's that had adhered to the microspheres had begun to spread out on the surfaces and were transferred with microspheres into the new plates. After 72 hours, the cells had proliferated on the microspheres and spread out to cover the entire surface of each microsphere. An image taken from a standard light microscope of a cell-covered hydrogel microsphere is shown in FIG. 18 and clearly demonstrates that the pre-polymerized, microsphere format supports surface attachment of cells.

### Example B8—Comparison of Microspheres Vs. In Situ Polymerization

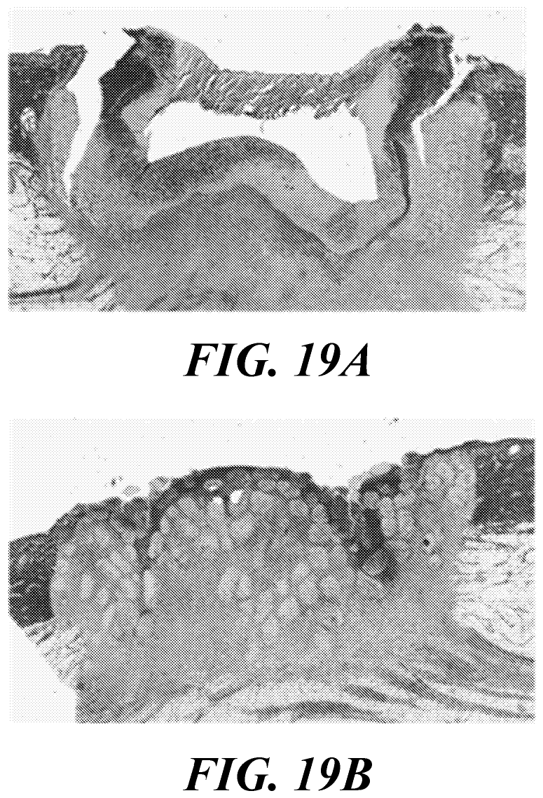

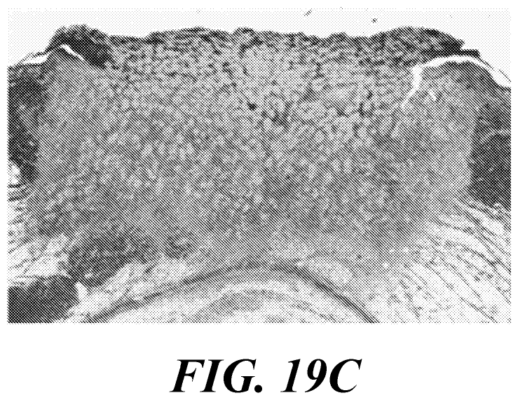

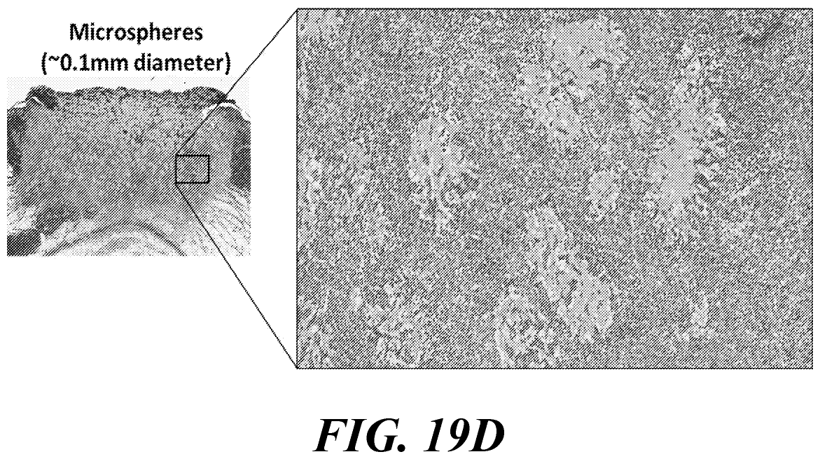

To evaluate the scaffolding properties of hydrogel microspheres in wound healing, two distinct sizes of microspheres (FIG. 19B corresponds to microspheres of about 0.5 mm diameter;

FIG. 19C corresponds to microspheres of about 0.1 mm diameter) were compared with identical hydrogel formulations that had been polymerized in situ in bulk (FIG. 19A). All hydrogel materials shown in FIGS. 19A-19D were prepared with a fibrinogen component content of 1.5% wt./vol. Significant cellularity was observed between the hydrogel microspheres, indicating that cells were able to migrate around the hydrogel scaffold in a degradation-independent manner (FIG. 19B and FIG. 19C). Of note, the progression of neutrophils to the top of the wound (deep red layer of cells) was nearly complete in microsphere-treated wounds, whereas the neutrophil layer in the in situ conditions appeared delayed (FIG. 19A), likely because it must degrade the hydrogel to create a path for migration.

In general, the progression of wound healing in the microsphere-treated wounds appeared significantly advanced. In addition to the successful migration of the neutrophil layer to the top of the wound, substantial levels of granulation tissue had accumulated in the wound bed when compared to the in situ polimerized hydrogel treatment. Complete replacement of the hydrogel had not occurred, as the outlines of microparticles can be easily visualized within the granulation bed in FIG. 19D. However, these remaining microparticles showed signs of degradation and cellular infiltration as would be expected given the degradation-dependent scaffolding properties of the formulation.

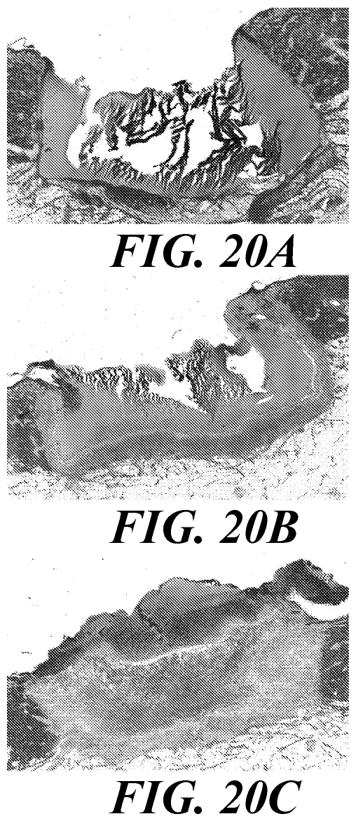

FIG. 20A presents a histological study in an acute, full-thickness wound healing model for materials comprising 1.5% wt./vol. of fibrinogen component, for bulk in situ polymerized formulations 3 days after application. FIG. 20B presents a histological study in an acute, full-thickness wound healing model for materials comprising 1.5% wt./vol. of fibrinogen component, for bulk in situ polymerized formulations 8 days after application. FIG. 20C presents a histological study in an acute, full-thickness wound healing model for materials comprising 1.5% wt./vol. of fibrinogen component, for bulk in situ polymerized formulations 14 days after application.

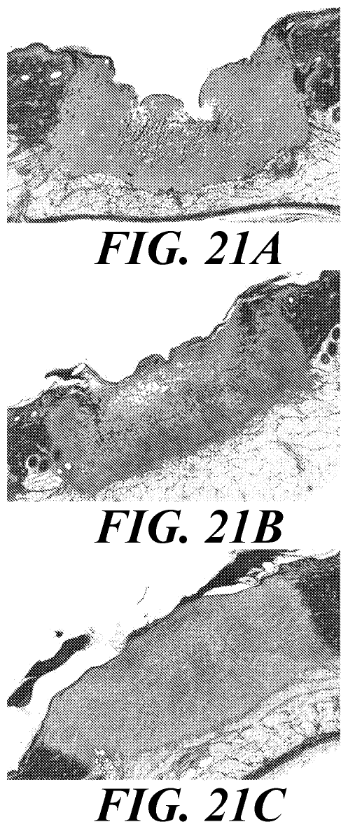

FIG. 21A presents a histological study in an acute, full-thickness wound healing model for hydrogel microspheres comprising 1.5% wt./vol. of fibrinogen component, having a diameter of about 0.1 mm to about 0.2 mm, 3 days after application. FIG. 21B presents a histological study in an acute, full-thickness wound healing model for hydrogel microspheres having a diameter of about 0.1 mm to about 0.2 mm, 8 days after application. FIG. 21C presents a histological study in an acute, full-thickness wound healing model for hydrogel microspheres having a diameter of about 0.1 mm to about 0.2 mm, 14 days after application.

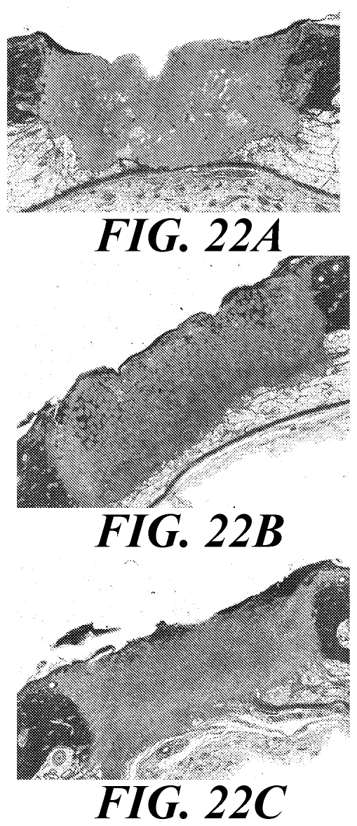

FIG. 22A presents a histological study in an acute, full-thickness wound healing model for hydrogel microspheres having a diameter of about 0.2 mm to about 0.3 mm, 3 days after application. FIG. 22B presents a histological study in an acute, full-thickness wound healing model for hydrogel microspheres having a diameter of about 0.2 mm to about 0.3 mm, 8 days after application. FIG. 22C presents a histological study in an acute, full-thickness wound healing model for hydrogel microspheres having a diameter of about 0.2 mm to about 0.3 mm, 14 days after application.

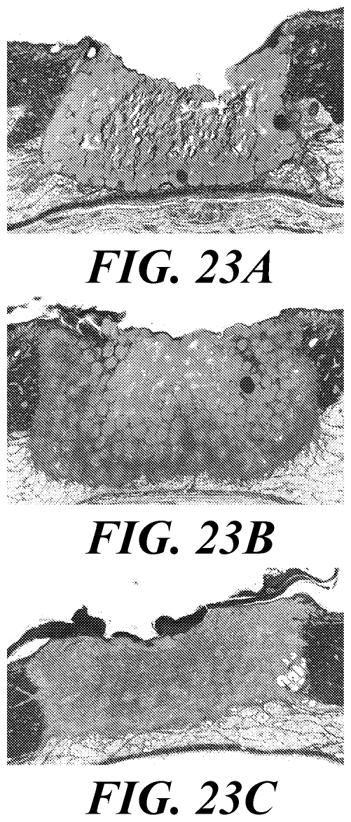

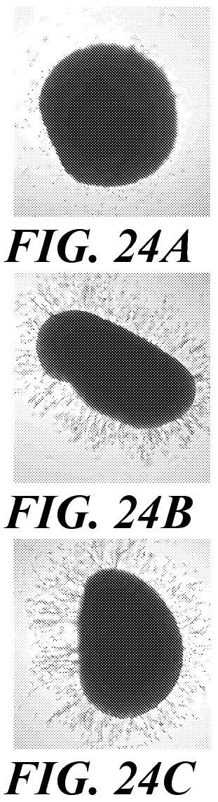

FIG. 23A presents a histological study in an acute, full-thickness wound healing model for hydrogel microspheres having a diameter of about 0.5 mm to about 0.7 mm, 3 days after application. FIG. 23B presents a histological study in an acute, full-thickness wound healing model for hydrogel microspheres having a diameter of about 0.5 mm to about 0.7 mm, 8 days after application. FIG. 23C presents a histological study in an acute, full-thickness wound healing model for hydrogel microspheres having a diameter of about 0.5 mm to about 0.7 mm, 14 days after application.

All references throughout, such as publications, patents, patent applications and published patent applications, are incorporated herein by reference in their entireties.

In the descriptions herein, it is understood that every description, variation, embodiment or aspect of a facet may be combined with every description, variation, embodiment or aspect of other facets the same as if each and every combination of descriptions is specifically and individually listed.

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as defined by the claims.

### Example B9—Application of Microspheres to a Chronic Wound

A patient with a chronic wound condition such as diabetic foot ulcer receives the treatment in the course of the visit to a licensed health care provider. The wound is inspected and if necessary surgically debrided according to the current standard of care. The packed microsphere slurry in sterile saline is applied from a pre-packed syringe via a stainless-steel needle of appropriate size to fill the tissue void in the debrided wound. Small amount of excess liquid (saline from the slurry and/or exudate from the wound) is carefully removed from the filled surface with an appropriate device (e.g. by carefully dabbing sterile gauze around the wound edges). The filled wound is sealed with adhesive dressing of appropriate size, such as Tegaderm. Additional dressings such as gauze are applied at the discretion of healthcare provider.

This example illustrates application of microspheres in real-life medical setting for the treatment of a wound.

### Example B10—Application of Microspheres to a Tunneling or Buried Defect

In this example the scaffolding microparticle slurry is used to treat an internal tissue injury or defect, such as a cartilage or bone injury, a tunneling wound or a soft tissue defect requiring a cosmetic enhancement. A defect or an injury is diagnosed by the current state-of-the-art techniques, such as visual and biomechanical observations by a qualified health care provider, tomography, x-ray and is potentially supplemented with laboratory work to diagnose a specific condition. If necessary, the defect is surgically debrided according to the current standard of care with the aid of endoscopic device inserted through a small incision in the skin and intermediate tissues. The packed microsphere slurry in sterile saline is applied from a pre-packed syringe via a stainless-steel needle of appropriate size or an appropriate channel in the endoscopic device to fill the tissue void in the debrided defect, or as necessary to achieve desired cosmetic correction. Small amount of excess liquid (saline from the slurry and/or exudate from the debrided defect) is carefully removed with appropriate endoscopic suction device. At the discretion of the health care provider, the microparticle-filled defect is covered by an adhesion barrier to avoid formation of post-surgical adhesions around the area of surgical intervention. The skin incision used to introduce the endoscopic device and the needle delivering microparticle slurry is sealed with adhesive dressing, such as Tegaderm. Additional dressings such as gauze are applied at the discretion of healthcare provider.

### Example B11—In Vitro Cellular Invasion Assay Using Fgn-Gamma-PEG-NB

An in vitro cellular invasion assay was used study how changes in scaffolding composition affected the scaffolding properties (i.e. cellular migration) of the biomaterial. Briefly, cells were suspended in Type I Collagen and allowed to contract for 24-48 hours under standard cell culture conditions. The contracted collagen-cell plugs were then transferred individually into the wells of a 48-well tissue culture plate and covered with polymer precursor solutions having define formulations. The cell-seeded collagen plugs were move to the center of the well with a sterile pipet tip, ensuring that the plug was fully immersed in the pre-polymer solution and that it would be visible by light microscopy. Polymerization of the biomaterial scaffolds around the plugs was achieved by exposing the well to a photo-initiating light, effectively encapsulating the collagen plug within the polymer. Media supplemented with growth factor-rich platelet releasates was placed into each well, and the invasion of cells out of the collagen plug and into the hydrogel matrix was monitored by light microscopy for 24-72 hours. Examples of data obtained from this assay are presented in FIGS. 24 A-C where the cell-seeded plug (dark object) call be observed within an optically clear photopolymer. Under suitable scaffolding conditions, cells will migrate out of the collagen plug and into the surrounding material, which can be observed by standard light microscopy.

### Preparation of Collagen Solution:

Immediately before use, Rat tail Type I Collagen (Fisher: CB-40236) was diluted with serum-free DMEM (with L-glutamine and 4.5 g/L glucose) containing sodium bicarbonate to make a working collagen solution with 2.5 mg/ml collagen and 1% wt./vol. sodium bicarbonate. The collagen remains in a liquid state when kept under acidic conditions at refrigerated temperatures. Therefore, it is important to keep these reagents on ice until use and mix only before needed to resuspend cell pellets.

### Preparation of Collagen Plugs:

Human Dermal Fibroblasts (HDF) were expanded under standard culture conditions before being trypsinized, collected into tubes, and counted using a hemocytometer. The cells were then pelleted by centrifugation at 800×g and resuspended in the collagen solution (described above) at a concentration of 2-3×10\hat{ }6 cells/ml. The collagen-cell suspension was then aliquoted (25 μL/well) into v-bottom polypropylene 96-well plates. The plates were incubated at 37 C for 30 minutes to allow the collagen solution to solidify before adding 200 μL of growth media (DMEM with L-glutamine and 4.5 g/L glucose+10% v/v FBS) to each well. The addition of media usually causes the collagen plug to detach from the bottom of the well, allowing plug contraction. If plugs were not observed to detach from the bottom of the well at the time of adding growth media, a pipet tip was used to gently free the plug from the bottom of the plate. The cell-seeded collagen plugs were incubated 37 C for 1-3 days before use in this assay.

### Collagen Plug Encapsulation:

After incubating the cell-seeded collagen plugs for 1-3 days, each plug was transferred by pipet into 48-well suspension culture plates (wide-mouthed or cut-off pipet tips were used to accommodate plug diameters). All media was then aspirated from the well, taking care to avoid disrupting the plug. 250 μL of the precursor solution was added to each well, and a pipet tip was used to gently move each plug to the center of its well. The mixture was then polymerized to form a hydrogel matrix surrounding the collagen plug in the well by exposure to light (385 nm, 19 mW/cm2, 30 seconds). In this example, the degradable polymer incorporated Fgn-gamma-PEG-NB as the only component with cell adhesion sites. Following polymerization, 200 d of growth media (DMEM with L-glutamine and 4.5 g/L glucose+10% v/v FBS) was added to each well, and plates were incubated at 37 C.

### Cellular Invasion:

Cellular invasion into the hydrogel matrix was observed by standard light microscopy. Images were taken with a Leica EC3 camera attached to a Motic AE31 microscope. Cell-seeded collagen plugs were not translucent and were observed as dark, roundish shapes in the center of each image as can be seen in FIG. 24A. The Fgn-gamma-PEG-NB containing hydrogel matrix was translucent and was clearly distinguishable from the collagen plug as can be seen in FIG. 24A. Cells migrating out from the collagen plugs appear as dark, thin projections from the plug as can be seen in FIG. 24B-C. Control formulations only lacking Fgn-gamma-PEG-NB showed no signs of cellular migration into the polymer as seen in FIG. 24A.

### Example B12—Wound Healing Assay with Slab Scaffold Using Fgn-Gamma-PEG-NB

A porcine, full-thickness wound healing model was used to evaluate the ability of Fgn-gamma-PEG-NB containing hydrogel formulations to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

Fgn-gamma-PEG-NB containing hydrogel scaffolds were applied as a liquid to the wound site and photopolymerized in situ. A 385 nm light at 20 mW/cm2 was directed over the wound for 30 seconds, a time determined to be sufficient for complete polymerization of the matrix. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

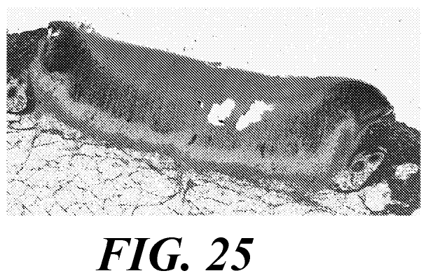

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 25. FIG. 25 illustrates cells migrating into the hydrogel matrix in vivo. Harvesting tissue at different days post-wounding makes it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

### Example B13—Wound Healing Assay with Microparticle Scaffold Using Fgn-Gamma-PEG-NB

A porcine, full-thickness wound healing model was used to evaluate the ability of microparticles containing Fgn-gamma-PEG-NB to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

Pre-polymerized microspheres containing Fgn-gamma-PEG-NB were applied to the wound. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

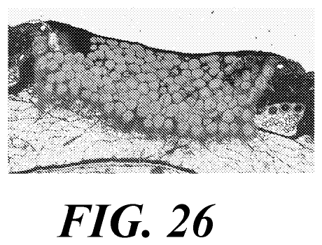

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 26. FIG. 26 illustrates cells migrating into the hydrogel matrix in vivo. Harvesting tissue at different days post-wounding makes it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

### Example B14—Cellular Invasion Assay with Slab Scaffold Using-Beta-PEG-NB

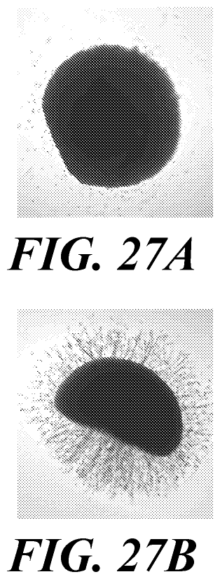

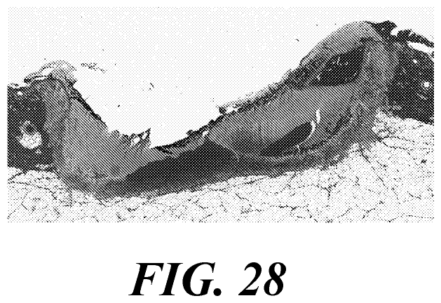

An in vitro cellular invasion assay was used study how changes in scaffolding composition affected the scaffolding properties (i.e. cellular migration) of the biomaterial. Briefly, cells were suspended in Type I Collagen and allowed to contract for 24-48 hours under standard cell culture conditions. The contracted collagen-cell plugs were then transferred individually into the wells of a 48-well tissue culture plate and covered with polymer precursor solutions having define formulations. The cell-seeded collagen plugs were move to the center of the well with a sterile pipet tip, ensuring that the plug was fully immersed in the pre-polymer solution and that it would be visible by light microscopy. Polymerization of the biomaterial scaffolds around the plugs was achieved by exposing the well to a photo-initiating light, effectively encapsulating the collagen plug within the polymer. Media supplemented with growth factor-rich platelet releasates was placed into each well, and the invasion of cells out of the collagen plug and into the hydrogel matrix was monitored by light microscopy for 24-72 hours. Examples of data obtained from this assay are presented in FIGS. 27 A-B where the cell-seeded plug (dark object) call be observed within an optically clear photopolymer. Under suitable scaffolding conditions, cells will migrate out of the collagen plug and into the surrounding material, which can be observed by standard light microscopy, shown in FIG. 27B.

### Preparation of Collagen Solution:

Immediately before use, Rat tail Type I Collagen (Fisher: CB-40236) was diluted with serum-free DMEM (with L-glutamine and 4.5 g/L glucose) containing sodium bicarbonate to make a working collagen solution with 2.5 mg/ml collagen and 1% wt./vol. sodium bicarbonate. The collagen remains in a liquid state when kept under acidic conditions at refrigerated temperatures. Therefore, it is important to keep these reagents on ice until use and mix only before needed to resuspend cell pellets.

### Preparation of Collagen Plugs:

Human Dermal Fibroblasts (HDF) were expanded under standard culture conditions before being trypsinized, collected into tubes, and counted using a hemocytometer. The cells were then pelleted by centrifugation at 800×g and resuspended in the collagen solution (described above) at a concentration of 2-3×10\hat{ }6 cells/ml. The collagen-cell suspension was then aliquoted (25 μL/well) into v-bottom polypropylene 96-well plates. The plates were incubated at 37 C for 30 minutes to allow the collagen solution to solidify before adding 200 μL of growth media (DMEM with L-glutamine and 4.5 g/L glucose+10% v/v FBS) to each well. The addition of media usually causes the collagen plug to detach from the bottom of the well, allowing plug contraction. If plugs were not observed to detach from the bottom of the well at the time of adding growth media, a pipet tip was used to gently free the plug from the bottom of the plate. The cell-seeded collagen plugs were incubated 37 C for 1-3 days before use in this assay.

### Collagen Plug Encapsulation:

After incubating the cell-seeded collagen plugs for 1-3 days, each plug was transferred by pipet into 48-well suspension culture plates (wide-mouthed or cut-off pipet tips were used to accommodate plug diameters). All media was then aspirated from the well, taking care to avoid disrupting the plug. 250 μL of the precursor solution was added to each well, and a pipet tip was used to gently move each plug to the center of its well. The mixture was then polymerized to form a hydrogel matrix surrounding the collagen plug in the well by exposure to light (385 nm, 19 mW/cm2, 30 seconds). In this example, the degradable polymer incorporated Fgn-beta-PEG-NB as the only component with cell adhesion sites. Following polymerization, 200 d of growth media (DMEM with L-glutamine and 4.5 g/L glucose+10% v/v FBS) was added to each well, and plates were incubated at 37 C.

### Cellular Invasion:

Cellular invasion into the Fgn-beta-PEG-NB containing hydrogel matrix was observed by standard light microscopy. Images were taken with a Leica EC3 camera attached to a Motic AE31 microscope. Cell-seeded collagen plugs were not translucent and were observed as dark, roundish shapes in the center of each image as can be seen in FIG. 27A. The Fgn-beta-PEG-NB containing hydrogel matrix was translucent and was clearly distinguishable from the collagen plug as can be seen in FIG. 27A. Cells migrating out from the collagen plugs appear as dark, thin projections from the plug as can be seen in FIG. 27B.

### Example B15—Wound Healing Assay Using Slab Scaffold Using Fgn-Beta-PEG-NB

A porcine, full-thickness wound healing model was used to evaluate the ability of Fgn-beta-PEG-NB containing hydrogel formulations to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

Fgn-beta-PEG-NB containing hydrogel scaffolds were applied as a liquid to the wound site and photopolymerized in situ. A 385 nm light at 20 mW/cm2 was directed over the wound for 30 seconds, a time determined to be sufficient for complete polymerization of the matrix. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 28. FIG. 28 illustrates cells migrating into the hydrogel matrix in vivo. Harvesting tissue at different days post-wounding made it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding

### Example B16—Wound Healing Assay Using Microparticle Scaffold Using Fgn-Beta-PEG-NB

A porcine, full-thickness wound healing model was used to evaluate the ability of microparticles containing Fgn-beta-PEG-NB to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

Pre-polymerized microspheres containing Fgn-beta-PEG-NB were applied to the wound. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. Harvesting tissue at different days post-wounding made it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

While the gamma and alpha subunits of fibrinogen contain an RGD motif, the beta subunit does not. The field has identified the RGD sequence as being necessary for cells to invade into a polymer, however, this is clearly not the only sequence that is able to support cellular invasion, as the beta subunit works in a cellular invasion assay in fact better than the gamma subunit.

### Example B17—Wound Healing Assay Using DTP Microparticle Scaffold Using Di-Fgn-PEG-NB

A porcine, full-thickness wound healing model was used to evaluate the ability of microparticles containing DTP Fgn-PEG-NB to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

Pre-polymerized DTP microparticles containing Fgn-PEG-NB were applied to the wound. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

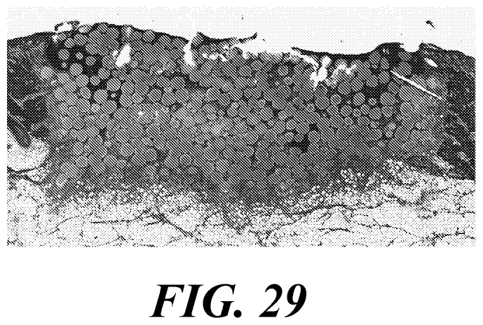

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 29. FIG. 29 illustrates cells migrating into the wound in vivo. Harvesting tissue at different days post-wounding makes it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

### Example B18—Wound Healing Assay Using DTP Microparticle Scaffold Using Fgn-Gamma-PEG-NB

A porcine, full-thickness wound healing model was used to evaluate the ability of microparticles containing DTP Fgn-gamma-PEG-NB to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

Pre-polymerized DTP microparticles containing Fgn-gamma-PEG-NB were applied to the wound. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

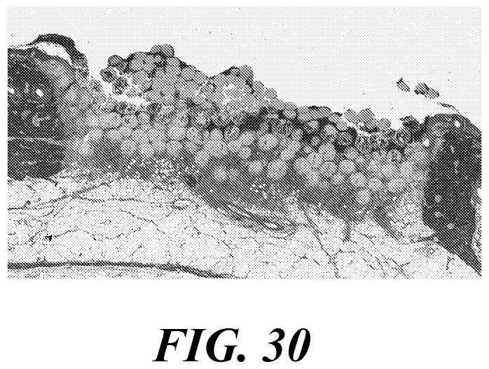

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 30. FIG. 30 illustrates cells migrating into the hydrogel matrix in vivo. Harvesting tissue at different days post-wounding makes it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

### Example B19—Acute, Full-Thickness Wound Healing Model Using DI-Fgn-PEG-NB and Only MMP Degradable Crosslinker

A porcine, full-thickness wound healing model was used to evaluate the ability of DI-Fgn-PEG-NB containing hydrogel formulations with only an MMP degradable linker to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

DI-Fgn-PEG-NB and MMP only containing hydrogel scaffolds were applied as a liquid to the wound site and photopolymerized in situ. A 385 nm light at 20 mW/cm² was directed over the wound for 30 seconds, a time determined to be sufficient for complete polymerization of the matrix. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

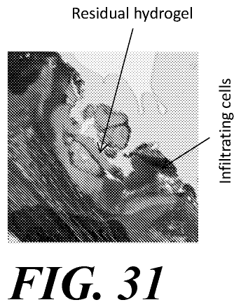

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 31. This Figure illustrates cells migrating into the hydrogel matrix in vivo. Harvesting tissue at different days post-wounding makes it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

### Example B20—Acute, Full-Thickness Wound Healing Model Using DI-Fgn-PEG-NB and Plasmin Degradable Linker Only

A porcine, full-thickness wound healing model was used to evaluate the ability of DI-Fgn-PEG-NB and plasmin degradable linker only hydrogel formulations to scaffold cellular migration into the wound volume from the surrounding tissues. Briefly, 10 mm circular biopsy punches were used to create full-thickness wounds between the shoulders and hind legs of Yorkshire pigs, with a minimum distance of 15 mm of unwounded skin separating each wound. Wounds were not made over bony protuberances, and sterile gauze, applied with pressure, was used achieve hemostasis in each wound before applying treatment materials. All animal procedures were performed with an approved IACUC protocol, with appropriate measures taken to prevent or alleviate pain in the animals.

### Scaffold Application:

DI-Fgn-PEG-NB and plasmin only degradable linker containing hydrogel scaffolds were applied as a liquid to the wound site and photopolymerized in situ. A 385 nm light at 20 mW/cm² was directed over the wound for 30 seconds, a time determined to be sufficient for complete polymerization of the matrix. Sterile dressings were applied to protect each wound and reduce the chance of infection. Because differences in healing rates vary over the surface area tested, treatments locations were randomized between animals. Untreated, control wounds received no scaffolding material and were covered with sterile dressings. When all wounds had been treated, protective dressings and jackets were used to prevent animals from disturbing the sites.

### Histological Evaluation:

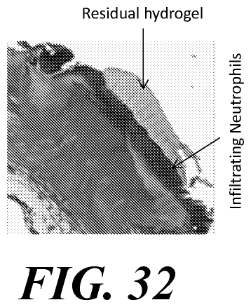

Tissue collection was performed at study endpoints. Depending on the nature of the study, animals were either euthanized or placed under anesthesia before excising each wound with surrounding tissue. Excised tissue was placed into 10% buffered formalin and fixed before processing. Before sectioning tissue blocks were trimmed and bisected such that the slides used for pathology would be cut from the center of each wound. Tissue sections were stained using common staining protocols. Namely, H&E and Trichrome stains were used to observe distinct cell types within the wound volume and hydrogel materials. An example of a Trichrome-stained tissue section is provided in FIG. 32. This Figure illustrates cells migrating into the hydrogel matrix in vivo. Harvesting tissue at different days post-wounding makes it possible to observe unique stages of the wound healing process. When evaluating the ability of hydrogel formulations to scaffold cellular migration and tissue regeneration, early time points were chosen because they provided snapshots of the polymerized formulation while sufficient material remained in the wound to see evidence of scaffolding.

### Example B21—In Vitro Cellular Invasion Assay Using Two Fgn Subunits

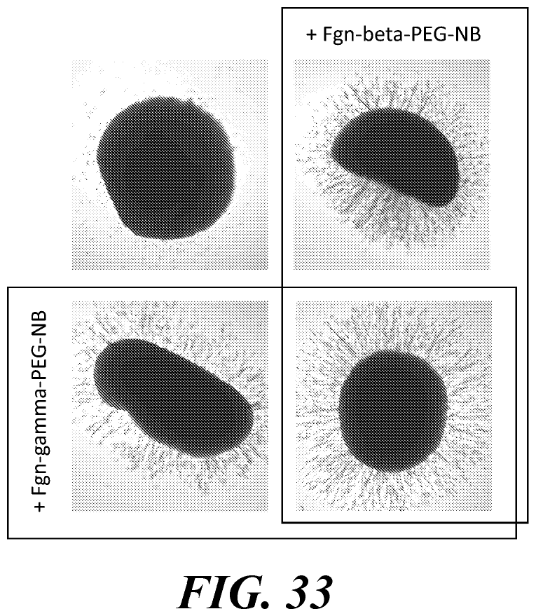

An in vitro cellular invasion assay was used study how mixing two subunits of Fgn together affected the scaffolding properties (i.e. cellular migration) of the biomaterial. Briefly, cells were suspended in Type I Collagen and allowed to contract for 24-48 hours under standard cell culture conditions. The contracted collagen-cell plugs were then transferred individually into the wells of a 48-well tissue culture plate and covered with polymer precursor solutions having define formulations. The cell-seeded collagen plugs were move to the center of the well with a sterile pipet tip, ensuring that the plug was fully immersed in the pre-polymer solution and that it would be visible by light microscopy. Polymerization of the biomaterial scaffolds around the plugs was achieved by exposing the well to a photo-initiating light, effectively encapsulating the collagen plug within the polymer. Media supplemented with growth factor-rich platelet releasates was placed into each well, and the invasion of cells out of the collagen plug and into the hydrogel matrix was monitored by light microscopy for 24-72 hours. Examples of data obtained from this assay are presented in FIG. 33 where the cell-seeded plug (dark object) call be observed within an optically clear photopolymer. Under suitable scaffolding conditions, cells will migrate out of the collagen plug and into the surrounding material, which can be observed by standard light microscopy, shown in FIG. 33.

### Preparation of Collagen Solution:

Immediately before use, Rat tail Type I Collagen (Fisher: CB-40236) was diluted with serum-free DMEM (with L-glutamine and 4.5 g/L glucose) containing sodium bicarbonate to make a working collagen solution with 2.5 mg/ml collagen and 1% wt./vol. sodium bicarbonate. The collagen remains in a liquid state when kept under acidic conditions at refrigerated temperatures. Therefore, it is important to keep these reagents on ice until use and mix only before needed to resuspend cell pellets.

### Preparation of Collagen Plugs:

Human Dermal Fibroblasts (HDF) were expanded under standard culture conditions before being trypsinized, collected into tubes, and counted using a hemocytometer. The cells were then pelleted by centrifugation at 800×g and resuspended in the collagen solution (described above) at a concentration of 2-3×10\hat{ }6 cells/ml. The collagen-cell suspension was then aliquoted (25 μL/well) into v-bottom polypropylene 96-well plates. The plates were incubated at 37 C for 30 minutes to allow the collagen solution to solidify before adding 200 μL of growth media (DMEM with L-glutamine and 4.5 g/L glucose+10% v/v FBS) to each well. The addition of media usually causes the collagen plug to detach from the bottom of the well, allowing plug contraction. If plugs were not observed to detach from the bottom of the well at the time of adding growth media, a pipet tip was used to gently free the plug from the bottom of the plate. The cell-seeded collagen plugs were incubated 37 C for 1-3 days before use in this assay.

### Collagen Plug Encapsulation:

After incubating the cell-seeded collagen plugs for 1-3 days, each plug was transferred by pipet into 48-well suspension culture plates (wide-mouthed or cut-off pipet tips were used to accommodate plug diameters). All media was then aspirated from the well, taking care to avoid disrupting the plug. 250 μL of the precursor solution was added to each well, and a pipet tip was used to gently move each plug to the center of its well. The mixture was then polymerized to form a hydrogel matrix surrounding the collagen plug in the well by exposure to light (385 nm, 19 mW/cm2, 30 seconds). In this example, the degradable polymer incorporated Fgn-beta-PEG-NB and Fgn-gamma-PEG-NB, although any combination of two subunits could be used. Following polymerization, 200 d of growth media (DMEM with L-glutamine and 4.5 g/L glucose+10% v/v FBS) was added to each well, and plates were incubated at 37 C.

### Cellular Invasion:

Cellular invasion into the Fgn-beta-PEG-NB and Fgn-gamma-PEG-NB containing hydrogel matrix was observed by standard light microscopy. Images were taken with a Leica EC3 camera attached to a Motic AE31 microscope. Cell-seeded collagen plugs were not translucent and were observed as dark, roundish shapes in the center of each image as can be seen in FIG. 33. The Fgn-beta-PEG-NB and Fgn-gamma-PEG-NB containing hydrogel matrix was translucent and was clearly distinguishable from the collagen plug as can be seen in FIG. 33. Cells migrating out from the collagen plugs appear as dark, thin projections from the plug as can be seen in FIG. 33.

### Example B22—In Vitro Cellular Invasion Assay Using Three Fgn Subunits

An in vitro cellular invasion assay is used to study how mixing three subunits of Fgn, Fgn-alpha-PEG-NB, Fgn-beta-PEG-NB and Fgn-gamma-PEG-NB, which are recombinantly expressed individually and purified, then PEGylated either separately or together affected the scaffolding properties (i.e. cellular migration) of the biomaterial. Briefly, suspend cells in Type I Collagen and allow to contract for 24-48 hours under standard cell culture conditions. Transfer the contracted collagen-cell plugs individually into the wells of a 48-well tissue culture plate and cover with polymer precursor solutions having define formulations, in this example, containing Fgn-alpha-PEG-NB, Fgn-beta-PEG-NB and Fgn-gamma-PEG-NB. Move the cell-seeded collagen plugs to the center of the well with a sterile pipet tip, ensuring that the plug is fully immersed in the pre-polymer solution and that it is visible by light microscopy. Polymerize the biomaterial scaffolds around the plugs by exposing the well to a photo-initiating light, effectively encapsulating the collagen plug within the polymer. Add media supplemented with growth factor-rich platelet releasates into each well, and the invasion of cells out of the collagen plug and into the hydrogel matrix is monitored by light microscopy for 24-72 hours. Under suitable scaffolding conditions, cells will migrate out of the collagen plug and into the surrounding material, which can be observed by standard light microscopy.

### Preparation of Collagen Solution:

Immediately before use, Rat tail Type I Collagen (Fisher: CB-40236) is diluted with serum-free DMEM (with L-glutamine and 4.5 g/L glucose) containing sodium bicarbonate to make a working collagen solution with 2.5 mg/ml collagen and 1% wt./vol. sodium bicarbonate. The collagen remains in a liquid state when kept under acidic conditions at refrigerated temperatures. Therefore, it is important to keep these reagents on ice until use and mix only before needed to resuspend cell pellets.

### Preparation of Collagen Plugs:

Expand Human Dermal Fibroblasts (HDF) under standard culture conditions before trypsinizing, collecting into tubes, and counting using a hemocytometer. Pellet the cells by centrifugation at 800×g and resuspend in the collagen solution (described above) at a concentration of 2-3×10\hat{ }6 cells/ml. The collagen-cell suspension is then aliquoted (25 μL/well) into v-bottom polypropylene 96-well plates. Incubate the plates at 37 C for 30 minutes to allow the collagen solution to solidify before adding 200 μL of growth media (DMEM with L-glutamine and 4.5 g/L glucose+10% v/v FBS) to each well. The addition of media usually causes the collagen plug to detach from the bottom of the well, allowing plug contraction. If plugs are not observed to detach from the bottom of the well at the time of adding growth media, use a pipet tip to gently free the plug from the bottom of the plate. Incubate the cell-seeded collagen plugs 37 C for 1-3 days before use in this assay.

### Collagen Plug Encapsulation:

After incubating the cell-seeded collagen plugs for 1-3 days, transfer each plug by pipet into 48-well suspension culture plates (use wide-mouthed or cut-off pipet tips to accommodate plug diameters). Aspirate all media from the well, taking care to avoid disrupting the plug. Add 250 μL of the precursor solution to each well, and use a pipet tip to gently move each plug to the center of its well. Polymerize the mixture to form a hydrogel matrix surrounding the collagen plug in the well by exposure to light (385 nm, 19 mW/cm², 30 seconds). In this example, the degradable polymer incorporates Fgn-alpha-PEG-NB, Fgn-beta-PEG-NB and Fgn-gamma-PEG-NB. Following polymerization, add 200 μl of growth media (DMEM with L-glutamine and 4.5 g/L glucose+10% v/v FBS) to each well, and incubate plates at 37 C.

### Cellular Invasion:

Observe cellular invasion into the Fgn-alpha-PEG-NB, Fgn-beta-PEG-NB and Fgn-gamma-PEG-NB containing hydrogel matrix by standard light microscopy. Take images with an appropriate microscope. The Fgn-beta-PEG-NB and Fgn-gamma-PEG-NB containing hydrogel matrix is translucent and is clearly distinguishable from the collagen plug. Cells migrating out from the collagen plugs appear as dark, thin projections from the plug.

### Example B23—Acute, Full-Thickness Wound Healing Model Using Two Fgn Subunits

A porcine, full-thickness wound healing model is used to evaluate the ability of any two Fgn subunit containing hydrogel formulations to scaffold cellular migration into the wound volume from the surrounding tissues. Briefly, 10 mm circular biopsy punches are used to create full-thickness wounds between the shoulders and hind legs of Yorkshire pigs, with a minimum distance of 15 mm of unwounded skin separating each wound. Wounds are not made over bony protuberances. Use sterile gauze, applied with pressure, to achieve hemostasis in each wound before applying treatment materials. All animal procedures are performed with an approved IACUC protocol, with appropriate measures taken to prevent or alleviate pain in the animals.

### Scaffold Application:

Apply two Fgn subunit containing hydrogel scaffolds as a liquid to the wound site and photopolymerize in situ. Direct a 385 nm light at 20 mW/cm² over the wound for 30 seconds, a time which is sufficient for complete polymerization of the matrix. Apply sterile dressings to protect each wound and reduce the chance of infection. Because differences in healing rates vary over the surface area, randomize treatments locations between animals. Untreated, control wounds receive no scaffolding material and cover with sterile dressings. Treat all wounds and apply protective dressings and jackets to prevent animals from disturbing the sites.

### Histological Evaluation:

Collect tissue at study endpoints. Depending on the nature of the study, either euthanize animals or place under anesthesia before excising each wound with surrounding tissue. Place excised tissue into 10% buffered formalin and fix before processing. Trim tissue blocks before sectioning such that the slides used for pathology would be cut from the center of each wound. Stain tissue sections using common staining protocols. Namely, use H&E and Trichrome stains to observe distinct cell types within the wound volume and hydrogel materials. Harvesting tissue at different days post-wounding makes it possible to observe unique stages of the wound healing process. When evaluating the ability of hydrogel formulations to scaffold cellular migration and tissue regeneration, use early time points because they provided snapshots of the polymerized formulation while sufficient material remains in the wound to see evidence of scaffolding.

### Example B24—Acute, Full-Thickness Wound Healing Model Using Three Fgn Subunits

A porcine, full-thickness wound healing model is used to evaluate the ability of all three Fgn subunits, recombinantly expressed separately, purified and PEGylated either separately or together in hydrogel formulations to scaffold cellular migration into the wound volume from the surrounding tissues. Briefly, 10 mm circular biopsy punches are used to create full-thickness wounds between the shoulders and hind legs of Yorkshire pigs, with a minimum distance of 15 mm of unwounded skin separating each wound. Wounds are not made over bony protuberances. Use sterile gauze, applied with pressure, to achieve hemostasis in each wound before applying treatment materials. All animal procedures are performed with an approved IACUC protocol, with appropriate measures taken to prevent or alleviate pain in the animals.

### Scaffold Application:

Apply Fgn-alpha-PEG-NB, Fgn-beta-PEG-NB and Fgn-gamma-PEG-NB containing hydrogel scaffolds as a liquid to the wound site and photopolymerize in situ. Direct a 385 nm light at 20 mW/cm2 over the wound for 30 seconds, a time which is sufficient for complete polymerization of the matrix. Apply sterile dressings to protect each wound and reduce the chance of infection. Because differences in healing rates vary over the surface area, randomize treatments locations between animals. Untreated, control wounds receive no scaffolding material and cover with sterile dressings. Treat all wounds and apply protective dressings and jackets to prevent animals from disturbing the sites.

### Histological Evaluation:

Collect tissue at study endpoints. Depending on the nature of the study, either euthanize animals or place under anesthesia before excising each wound with surrounding tissue. Place excised tissue into 10% buffered formalin and fix before processing. Trim tissue blocks before sectioning such that the slides used for pathology would be cut from the center of each wound. Stain tissue sections using common staining protocols. Namely, use H&E and Trichrome stains to observe distinct cell types within the wound volume and hydrogel materials. Harvesting tissue at different days post-wounding makes it possible to observe unique stages of the wound healing process. When evaluating the ability of hydrogel formulations to scaffold cellular migration and tissue regeneration, use early time points because they provided snapshots of the polymerized formulation while sufficient material remains in the wound to see evidence of scaffolding.

All references throughout, such as publications, patents, patent applications and published patent applications, are incorporated herein by reference in their entireties.

In the descriptions herein, it is understood that every description, variation, embodiment or aspect of a facet may be combined with every description, variation, embodiment or aspect of other facets the same as if each and every combination of descriptions is specifically and individually listed.

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as defined by the claims.

1. A hydrogel biomaterial comprising
at least one linker component;
at least one protease-cleavable component; and
a fibrinogen component derivatized with a plurality of polymeric linkers;
wherein at least a portion of the at least one linker component, at least a portion of the at least one protease-cleavable component, and at least a portion of the fibrinogen component are crosslinked via a plurality of crosslink units each independently comprising a carbon-sulfur covalent bond.

2. The hydrogel biomaterial of claim 1, the at least one linker component comprises at least one polymer component.

3. The hydrogel biomaterial of claim 2, wherein the at least one polymer component is connected to the at least one protease-cleavable component via a first crosslink unit and the fibrinogen component is connected to the at least one protease-cleavable component via a second crosslink unit.

4. The hydrogel biomaterial of claim 3, wherein the first crosslink unit is selected from the group consisting of 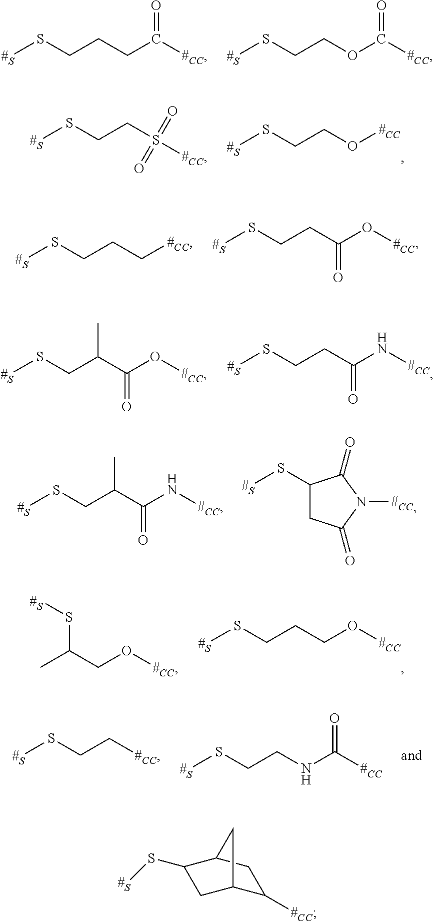
wherein #s represents the attachment point to the at least one protease-cleavable component and #cc represents the attachment point to the at least one polymer component.

5. The hydrogel biomaterial of claim 3 or 4, wherein the first crosslink unit is 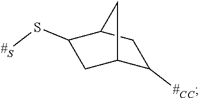
wherein #s represents the attachment point to the at least one protease-cleavable component and #cc represents the attachment point to the at least one polymer component.

6. The hydrogel biomaterial of any one of claims 3 to 5, wherein the second crosslink unit is selected from the group consisting of 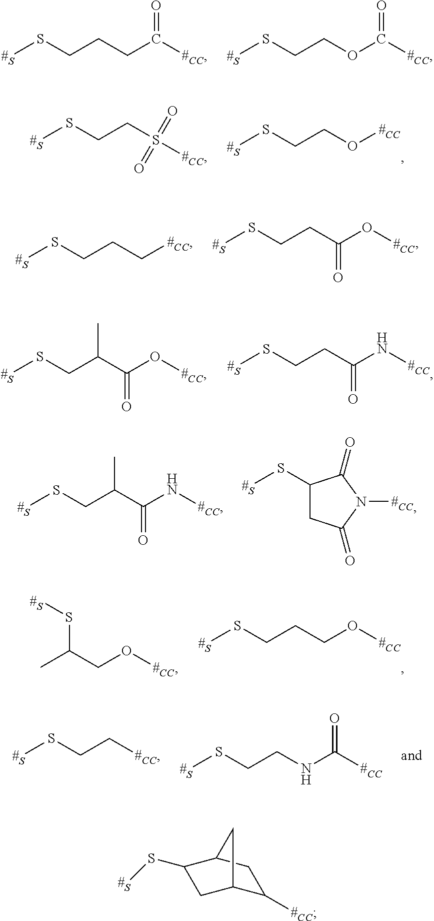
wherein #s represents the attachment point to the at least one protease-cleavable component and #cc represents the attachment point to a polymeric linker of the fibrinogen component.

7. The hydrogel biomaterial of any one of claims 3 to 6, wherein the second crosslink unit is 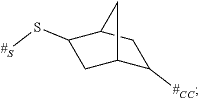
wherein #s represents the attachment point to the at least one protease-cleavable component and #cc represents the attachment point to a polymeric linker of the fibrinogen component.

8. The hydrogel biomaterial of any one of claims 2 to 7, wherein the at least one polymer component comprises a linear polymer component.

9. The hydrogel biomaterial of claim 8, wherein the linear polymer component comprises a linear polymeric moiety, wherein the linear polymeric moiety is selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

10. The hydrogel biomaterial of claim 8 or 9, wherein the linear polymer component comprises a linear polymeric moiety, wherein the linear polymeric moiety is a poly(ethylene glycol) moiety having a molecular weight of between about 1 kDa and about 40 kDa.

11. The hydrogel biomaterial of any one of claims 2 to 7, wherein the at least one polymer component comprises a branched polymer component.

12. The hydrogel biomaterial of claim 11, wherein the branched polymer component comprises a branched polymeric moiety with n polymeric arms, wherein n is an integer selected from the group consisting of 3, 4, 5, 6, 7, 8, 9, and 10.

13. The hydrogel biomaterial of claim 12, wherein the branched polymer component comprises a branched polymeric moiety with n polymeric arms, wherein n is 4.

14. The hydrogel biomaterial of claim 12 or 13, wherein each of the n polymeric arms is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

15. The hydrogel biomaterial of any one of claims 12 to 14, wherein the branched polymeric moiety has a molecular weight of between about 1 kDa and about 40 kDa.

16. The hydrogel biomaterial of any one of claims 12 to 15, wherein the branched polymer component comprises a 4-arm poly(ethylene glycol) moiety.

17. The hydrogel biomaterial of claim 16, wherein the 4-arm poly(ethylene glycol) moiety has a molecular weight of about 20 kDa.

18. The hydrogel biomaterial of any one of claims 2 to 17, wherein the at least one protease-cleavable component is a synthetic peptide.

19. The hydrogel biomaterial of any one of claims 2 to 18, wherein each of the at least one protease-cleavable component comprises a peptide chain comprising a sequence selected from the group consisting of SEQ ID NOs: 1-19.

20. The hydrogel biomaterial of any one of claims 2 to 19, wherein the hydrogel biomaterial comprises two protease-cleavable components, wherein one protease-cleavable component is a matrix metalloproteinase (MMP) cleavable component, and the other is a plasmin cleavable component.

21. The hydrogel biomaterial of claim 20, wherein the hydrogel biomaterial comprises two protease-cleavable components, wherein the matrix metalloproteinase (MMP) cleavable peptide is a peptide of SEQ ID NO: 19, and the plasmin cleavable peptide is a peptide of SEQ ID NO: 7.

22. The hydrogel biomaterial of any one of claims 2 to 21, wherein the fibrinogen component derivatized with a plurality of polymeric linkers has been derivatized at cysteine residues.

23. The hydrogel biomaterial of any one of claims 2 to 22, wherein the fibrinogen component derivatized with a plurality of polymeric linkers has been derivatized at cysteine residues and comprises between 30 moles and 58 moles of polymeric linkers per mole of derivatized fibrinogen.

24. The hydrogel biomaterial of any one of claims 2 to 23, wherein each individual polymeric linker of the plurality of polymeric linkers is independently selected from the group consisting of poly(lactic acid), poly(glycolic acid), polyacrylamide, poly(N-alkylacrylamide), poly(2-oxazoline), polyethylenimine, poly(acrylic acid), polymethacrylate, poly(alkyl acrylate), poly(ethylene glycol), poly(propylene glycol), poly(vinyl alcohol), poly(vinylpyrrolidone), poloxamine, polyanhydride, polyorthoester, poly(hydroxy acid), polydioxanone, polycarbonate, polyaminocarbonate, poly(ethyl oxazoline), carboxymethyl cellulose, hydroxyalkylated cellulose, polynucleotide, polyamino acid, polypeptide, polysaccharide, polysucrose, carbohydrate, hyaluronic acid, dextran, heparan sulfate, chondroitin sulfate, heparin, alginate, and copolymers thereof, and blends of the foregoing.

25. The hydrogel biomaterial of any one of claims 2 to 24, wherein each individual polymeric linker of the plurality of polymeric linkers comprises a poly(ethylene glycol) moiety, wherein the poly(ethylene glycol) moiety has a molecular weight of about 3.5 kDa.

26. The hydrogel biomaterial of any one of claims 2 to 25, wherein the hydrogel biomaterial comprises comprising:
0-50% wt. of a linear polymer component comprising a linear PEG moiety, wherein the linear PEG moiety has an average molecular weight of 6 kDa;
0.1-50% wt. of a branched polymer component comprising a 4-armed PEG moiety, wherein the 4-armed PEG moiety has an average molecular weight of 20 kDa;
0-6% wt. of a first protease-cleavable component comprising a IMP-cleavable peptide K(hC)GPQGIAGQ(hC)K (SEQ ID NO: 19);
0-6% wt. of a second protease-cleavable component comprising a plasmin-cleavable peptide (hC)ALKVLKG(hC)G-amide (SEQ ID NO: 7);
0-10% wt. of a fibrinogen component consisting of a fibrinogen molecule derivatized with a plurality of polymeric linkers each comprising a 3.5 kDa linear PEG; and
water.

27. The hydrogel biomaterial of claim 26, the hydrogel biomaterial comprises about 0.75% wt. of the fibrinogen component.

28. The hydrogel biomaterial of claim 26, the hydrogel biomaterial comprises about 1.50% wt. of the fibrinogen component.

29. The hydrogel biomaterial of claim 26, the hydrogel biomaterial comprises about 3.00% wt. of the fibrinogen component.

30. The hydrogel biomaterial of claim 1, the at least one linker component comprises at least one multivalent linker component.

31. The hydrogel biomaterial of claim 30, wherein the at least one multivalent linker component comprises a polythiol component.

32. The hydrogel biomaterial of claim 30, wherein the at least one multivalent linker component is selected from the group consisting of DL-dithiothreitol, L-dithiothreitol, D-dithiothreitol, dithioerythritol, pentaerythritol tetrakis(3-mercaptopropionate) (PETMP), pentaerythritol tetrakis(2-mercaptoacetate), trimethylolpropane tris(3-mercaptopropionate), trimethylolpropane tris(2-mercaptoacetate), tris[2-(3-mercaptopropionyloxy)ethyl] isocyanurate, trithiocyanuric acid, 1,3,4-thiadiazole-2,5-dithiol, 1,2-ethaneditiol, 1,3-propanedithiol, 2,3-dimercapto-1-propanol, 1,4-butanedithiol, 1,5-pentanedithiol, 1,6-hexanedithiol, 1,8-octanedithiol, 1,9-nonanedithiol, 1,11-undecanedithiol, 1,16-hexadecanedithiol, 2,2′-(ethylenedioxy)diethanethiol, tetra(ethylene glycol) dithiol, hexa(ethylene glycol) dithiol, poly(ethylene glycol) dithiol, ethylene glycol dimercaptopropionate, 2,5-dimercaptomethyl-1,4-dithiane, 2-mercaptoethylsulfide (also known as 2,2′-thiobis(ethane-1-thiol) and 2,2′-thiodiethanethiol), ethylene glycol bis-mercaptoacetate, 2,3-dimercaptopropanesulfonic acid or a salt thereof (such as sodium 2,3-dimercaptopropanesulfonate), meso-2,3-dimercaptosuccinic acid or a salt thereof, 2,2′-oxybis(ethane-1-thiol) (also known as 2-mercaptoethyl), 2,3-(dimercaptoethylthio)-1-mercaptopropane, 1,2,3-trimercaptopropane, toluene-3,4-dithiol or an dithiol isomer thereof, xylylenedithiol or a dithiol isomer thereof (such as 1,2-benzenedimethanethiol, 1,3-benzenedimethanethiol, or 1,4-benzenedimethanethiol), 4,4′-bis(mercaptomethyl)biphenyl, a synthetic or natural oligopeptide containing 2, 3, 4 or more free thiols in the form of cysteine, homocysteine residue or thiol-bearing chemical moieties introduced via chemical synthesis as side chain or terminal modifications, and a mixture of any of the foregoing.

33. A hydrogel biomaterial, wherein the hydrogel biomaterial is prepared by a polymerization reaction of a mixture comprising:
at least one polymer component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers, wherein each polymeric linker comprises a reactive ene group, and provided that at least one of the first reactive group and the second reactive group is a reactive thiol group.

34. A hydrogel biomaterial wherein the hydrogel biomaterial is prepared by a polymerization reaction of an aqueous mixture comprising:
at least one polymer component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial has a storage modulus of between about 100 Pa and about 10,000 Pa; and
provided that at least one of the first reactive group and the second reactive group is a reactive thiol group.

35. A hydrogel biomaterial wherein the hydrogel biomaterial is prepared by a polymerization reaction of an aqueous mixture comprising:
at least one polymer component comprising at least two of a first reactive group;
wherein the first reactive group is selected from the group consisting of a reactive thiol group and a reactive ene group;
at least one protease-cleavable component comprising at least two of a second reactive group;
wherein the second reactive group is selected from the group consisting of a reactive thiol group and a reactive ene group;
a fibrinogen component derivatized with a plurality of polymeric linkers with reactive ene groups;
wherein the hydrogel biomaterial does not substantially swell when exposed to a protease capable of cleaving the protease-cleavable component;
and
provided that at least one of the first reactive group and the second reactive group is a reactive thiol group.

36. A hydrogel biomaterial comprising
at least one protease-cleavable component; and
a fibrinogen component derivatized with a plurality of polymeric linkers;
wherein at least a portion of the at least one protease-cleavable component and at least a portion of the fibrinogen component are crosslinked via a plurality of crosslink units each independently comprising a carbon-sulfur covalent bond.

37. A hydrogel biomaterial wherein the hydrogel biomaterial is prepared by a polymerization reaction of a mixture comprising:
at least one protease-cleavable component comprising reactive thiol groups;
a fibrinogen component derivatized with a plurality of polymeric linkers with reactive ene groups, wherein the fibrinogen component does not possess the clotting activity of a native fibrinogen molecule.

38. A hydrogel biomaterial wherein the hydrogel biomaterial is prepared by a polymerization reaction of a mixture comprising:
at least one protease-cleavable component comprising reactive thiol groups; and
a fibrinogen component derivatized with a plurality of polymeric linkers with reactive ene groups;
wherein the hydrogel biomaterial has a storage modulus of between about 100 Pa and about 10,000 Pa.

39. A hydrogel biomaterial wherein the hydrogel biomaterial is prepared by a polymerization reaction of an aqueous mixture comprising:
at least one protease-cleavable component comprising reactive thiol groups;
a fibrinogen component derivatized with a plurality of polymeric linkers with reactive ene groups;
wherein the hydrogel biomaterial does not substantially swell when exposed to a protease capable of cleaving the protease-cleavable component.

40. A hydrogel biomaterial comprising
at least one linker component; and
a fibrinogen component derivatized with a plurality of polymeric linkers;
wherein at least a portion of the at least one polymer component and at least a portion of the fibrinogen component are crosslinked via a plurality of crosslink units each independently comprising a carbon-sulfur covalent bond.

41. The hydrogel biomaterial of claim 40, wherein the at least one linker component is at least one polymer component.

42. The hydrogel biomaterial of claim 40, wherein the at least one linker component is at least one multivalent linker component.

43. A hydrogel biomaterial wherein the hydrogel biomaterial is prepared by a polymerization reaction of a mixture comprising:
at least one polymer component comprising reactive thiol groups;
a fibrinogen component derivatized with a plurality of polymeric linkers with reactive ene groups, wherein the fibrinogen component does not possess the clotting activity of a native fibrinogen molecule.

44. A hydrogel biomaterial wherein the hydrogel biomaterial is prepared by a polymerization reaction of a mixture comprising:
at least one polymer component comprising reactive thiol groups; and
a fibrinogen component derivatized with a plurality of polymeric linkers with reactive ene groups;
wherein the hydrogel biomaterial has a storage modulus of between about 100 Pa and about 10,000 Pa.

45. A hydrogel biomaterial wherein the hydrogel biomaterial is prepared by a polymerization reaction of an aqueous mixture comprising:
at least one polymer component comprising reactive thiol groups; and
a fibrinogen component derivatized with a plurality of polymeric linkers with reactive ene groups;
wherein the hydrogel biomaterial does not substantially swell when exposed to a protease capable of cleaving the fibrinogen component.

46. The hydrogel biomaterial of any one of claims 1 to 45, wherein the hydrogel biomaterial is a hydrogel microparticle, wherein the hydrogel microparticle has a longest dimension of between about 0.05 mm and about 1.0 mm.

47. The hydrogel biomaterial of claim 46, wherein the hydrogel biomaterial is a hydrogel microsphere.

48. The hydrogel biomaterial of claim 47, wherein the hydrogel microsphere has a diameter of between about 0.05 mm and about 1.0 mm.

49. The hydrogel biomaterial of any one of claims 1 to 48, wherein the hydrogel biomaterial has a storage modulus of between about 100 Pa and about 10,000 Pa.

50. The hydrogel biomaterial of any one of claims 1 to 49, wherein the hydrogel biomaterial does not substantially swell when exposed to a protease capable of cleaving the protease-cleavable component.

51. The hydrogel biomaterial of any one of claims 1 to 49, wherein the fibrinogen component does not possess the clotting activity of a native fibrinogen molecule.

52. The hydrogel biomaterial of any one of claims 1 to 49, wherein the fibrinogen component is a native fibrinogen molecule.

53. The hydrogel biomaterial of any one of claims 1 to 49, wherein the fibrinogen component is the product or products of denaturing a native fibrinogen molecule.

54. The hydrogel biomaterial of any one of claims 1 to 49, wherein the fibrinogen component is an alpha chain of fibrinogen.

55. The hydrogel biomaterial of any one of claims 1 to 49, wherein the fibrinogen component is a beta chain of fibrinogen.

56. The hydrogel biomaterial of any one of claims 1 to 49, wherein the fibrinogen component is a gamma chain of fibrinogen.

57. The hydrogel biomaterial of any one of claims 1 to 49, wherein the fibrinogen component is a mixture of an alpha chain of fibrinogen and a beta chain of fibrinogen.

58. The hydrogel biomaterial of claim 57, wherein the mixture is an approximately equimolar mixture of an alpha chain of fibrinogen and a beta chain of fibrinogen.

59. The hydrogel biomaterial of any one of claims 1 to 49, wherein the fibrinogen component is a mixture of an alpha chain of fibrinogen and a gamma chain of fibrinogen.

60. The hydrogel biomaterial of claim 59, wherein the mixture is an approximately equimolar mixture of an alpha chain of fibrinogen and a gamma chain of fibrinogen.

61. The hydrogel biomaterial of any one of claims 1 to 49, wherein the fibrinogen component is a mixture of a beta chain of fibrinogen and a gamma chain of fibrinogen.

62. The hydrogel biomaterial of claim 61, wherein the mixture is an approximately equimolar mixture of a beta chain of fibrinogen and a gamma chain of fibrinogen.

63. The hydrogel biomaterial of any one of claims 1 to 49, wherein the fibrinogen component is a mixture of an alpha chain of fibrinogen, a beta chain of fibrinogen, and a gamma chain of fibrinogen.

64. The hydrogel biomaterial of claim 63, wherein the mixture is an equimolar mixture of an alpha chain of fibrinogen, a beta chain of fibrinogen, and a gamma chain of fibrinogen.

65. The hydrogel biomaterial of any one of claims 1 to 64, wherein the fibrinogen component is a primate fibrinogen component, a human fibrinogen component, a bovine fibrinogen component, a horse fibrinogen component, a suid fibrinogen component, a feline fibrinogen component, a canine fibrinogen component, a rodent fibrinogen component, a sheep fibrinogen component, or a chicken fibrinogen component.

66. A composition comprising the hydrogel biomaterial of any of claims 1 to 65 for treating an injury.

67. A composition comprising the hydrogel biomaterial of any of claims 1 to 65 for treating a tissue defect or improving a cosmetic outcome.

68. A composition comprising the hydrogel biomaterial of any of claims 1 to 65 for creating a filler to fill a tissue void.

69. A composition comprising the hydrogel biomaterial of any of claims 1 to 65 for treating pain.

70. A composition comprising the hydrogel biomaterial of any of claims 1 to 65 for treating an orthopedic condition.

71. A composition comprising the hydrogel biomaterial of any of claims 1 to 65 for use in a method for treating an injury.

72. A composition comprising the hydrogel biomaterial of any of claims 1 to 65 for use in a method for treating a tissue defect or improving a cosmetic outcome.

73. A composition comprising the hydrogel biomaterial of any of claims 1 to 65 for use in a method for creating a filler to fill a tissue void.

74. A composition comprising the hydrogel biomaterial of any of claims 1 to 65 for use in a method for treating pain.

75. A composition comprising the hydrogel biomaterial of any of claims 1 to 65 for use in a method for treating an orthopedic condition.

76. A method for treating an injury, comprising administering to a subject in need thereof the hydrogel biomaterial of any of claims 1 to 65.

77. A method for treating a tissue defect or improving a cosmetic outcome, comprising administering to a subject in need thereof the hydrogel biomaterial of any of claims 1 to 65.

78. A method for creating a filler to fill a tissue void, comprising administering to a subject in need thereof the hydrogel biomaterial of any of claims 1 to 65.

79. A method for treating pain, comprising administering to a subject in need thereof the hydrogel biomaterial of any of claims 1 to 65.

80. A method for treating an orthopedic condition, comprising administering to a subject in need thereof the hydrogel biomaterial of any of claims 1 to 65.

81. Use of the hydrogel biomaterial of any of claims 1 to 65 for treating an injury.

82. Use of the hydrogel biomaterial of any of claims 1 to 65 for treating a tissue defect or improving a cosmetic outcome.

83. Use of the hydrogel biomaterial of any of claims 1 to 65 for creating a filler to fill a tissue void.

84. Use of the hydrogel biomaterial of any of claims 1 to 65 for treating pain.

85. Use of the hydrogel biomaterial of any of claims 1 to 65 for treating an orthopedic condition.

86. Use of the hydrogel biomaterial of any of claims 1 to 65 in the manufacture of a medicament for treating an injury.

87. Use of the hydrogel biomaterial of any of claims 1 to 65 in the manufacture of a medicament for treating a tissue defect or improving a cosmetic outcome.

88. Use of the hydrogel biomaterial of any of claims 1 to 65 in the manufacture of a medicament for creating a filler to fill a tissue void.

89. Use of the hydrogel biomaterial of any of claims 1 to 65 in the manufacture of a medicament for treating pain.

90. Use of the hydrogel biomaterial of any of claims 1 to 65 in the manufacture of a medicament for treating an orthopedic condition.

91. A method for making a hydrogel biomaterial comprising
at least one linker component; and
a fibrinogen component;
wherein at least a portion of the at least one linker component, and at least a portion of the fibrinogen component are crosslinked via a plurality of crosslink units each independently comprising a carbon-sulfur covalent bond;
the method comprising:
subjecting a mixture comprising the at least one linker component and the fibrinogen component to a chemical reaction to create carbon-sulfur covalent bonds of the plurality of crosslink units.

92. A method for making a hydrogel biomaterial comprising
at least one polymer component;
at least one protease-cleavable component; and
a fibrinogen component;
wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component, the at least one protease-cleavable component, and the fibrinogen component to a chemical reaction to create the carbon-sulfur covalent bonds of the plurality of crosslink units.

93. A method for making a hydrogel biomaterial comprising
at least one polymer component;
at least one protease-cleavable component; and
a fibrinogen component;
wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive thiol groups, wherein the at least one protease-cleavable component comprises reactive ene groups, and at least a portion of the fibrinogen component wherein the fibrinogen component comprises reactive thiol groups, to a chemical reaction to create the carbon-sulfur covalent bonds of the plurality of crosslink units.

94. A method for making a hydrogel biomaterial comprising
at least one polymer component;
at least one protease-cleavable component; and
a fibrinogen component;
wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive ene groups, wherein the at least one protease-cleavable component comprises reactive ene groups, and at least a portion of the fibrinogen component wherein the fibrinogen component comprises reactive thiol groups, to a chemical reaction to create the carbon-sulfur covalent bonds of the plurality of crosslink units.

95. A method for making a hydrogel biomaterial comprising
at least one polymer component;
at least one protease-cleavable component; and
a fibrinogen component;
wherein at least a portion of the at least one polymer component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive ene groups, wherein the at least one protease-cleavable component comprises reactive thiol groups, and at least a portion of the fibrinogen component wherein the fibrinogen component comprises reactive thiol groups, to a chemical reaction to create the carbon-sulfur covalent bonds of the plurality of crosslink units.

96. A method for making a hydrogel biomaterial comprising
at least one polymer component;
at least one multivalent linker component; and
a fibrinogen component;
wherein at least a portion of the at least one polymer component, at least a portion of the at least one multivalent linker component, and at least a portion of the fibrinogen component are crosslinked via a plurality of crosslink units each independently comprising a carbon-sulfur covalent bond;
the method comprising:
subjecting a mixture comprising the at least one polymer component wherein the at least one polymer component comprises reactive ene groups, wherein the at least one multivalent linker component comprises reactive thiol groups, and at least a portion of the fibrinogen component wherein the fibrinogen component comprises reactive thiol groups, to a chemical reaction to create the carbon-sulfur covalent bonds of the plurality of crosslink units.

97. A method for making a hydrogel biomaterial comprising
at least one multivalent linker component;
at least one protease-cleavable component; and
a fibrinogen component;
wherein at least a portion of the at least one multivalent linker component, at least a portion of the at least one protease-cleavable component, and at least a portion of the fibrinogen component are crosslinked via a plurality of crosslink units each independently comprising a carbon-sulfur covalent bond;
the method comprising:
subjecting a mixture comprising the at least one multivalent linker component wherein the at least one multivalent linker component comprises reactive thiol groups, wherein the at least one protease-cleavable component comprises reactive ene groups, and at least a portion of the fibrinogen component wherein the fibrinogen component comprises reactive thiol groups, to a chemical reaction to create the carbon-sulfur covalent bonds of the plurality of crosslink units.

98. The method of any one of claims 91 to 97, wherein the chemical reaction is a free radical mediated thiol-ene reaction.


SEQUENCE LISTING (22 sequences)

SEQ ID NO: 1 - protein, 4 aa, Artificial Sequence
  Synthetic Construct
CWKC

SEQ ID NO: 2 - protein, 5 aa, Artificial Sequence
  Synthetic Construct
CALKC

SEQ ID NO: 3 - protein, 9 aa, Artificial Sequence
  Synthetic Construct
CALKVLKGC

SEQ ID NO: 4 - protein, 10 aa, Artificial Sequence
  Synthetic Construct
  VARIANT 10: COOH terminus is modified to be a CONH2 group
CALKVLKGCG

SEQ ID NO: 5 - protein, 9 aa, Artificial Sequence
  Synthetic Construct
  VARIANT 1, 9: Xaa = Homocysteine
XALKVLKGX

SEQ ID NO: 6 - protein, 10 aa, Artificial Sequence
  Synthetic Construct
  VARIANT 1, 9: Xaa = Homocysteine
XALKVLKGXG

SEQ ID NO: 7 - protein, 10 aa, Artificial Sequence
  Synthetic Construct
  VARIANT 10: COOH terminus is modified to be a CONH2 group
  VARIANT 1, 9: Xaa = Homocysteine
XALKVLKGXG

SEQ ID NO: 8 - protein, 11 aa, Artificial Sequence
  Synthetic Construct
  VARIANT 2, 10: Xaa = Homocysteine
  VARIANT 11: COOH terminus is modified to be a CONH2 group
KXALKVLKGXG

SEQ ID NO: 9 - protein, 12 aa, Artificial Sequence
  Synthetic Construct
  VARIANT 2, 11: Xaa = Homocysteine
  VARIANT 12: COOH terminus is modified to be a CONH2 group
KXGALKVLKGXG

SEQ ID NO: 10 - protein, 12 aa, Artificial Sequence
  Synthetic Construct
  VARIANT 2, 11: Xaa = Homocysteine
  VARIANT 12: COOH terminus is modified to be a CONH2 group
GXGALKVLKGXG

SEQ ID NO: 11 - protein, 11 aa, Artificial Sequence
  Synthetic Construct
  VARIANT 2, 10: Xaa = Homocysteine
  VARIANT 11: COOH terminus is modified to be a CONH2 group
KXALKVLKGXG

SEQ ID NO: 12 - protein, 12 aa, Artificial Sequence
  Synthetic Construct
  VARIANT 2, 11: Xaa = Homocysteine
  VARIANT 12: COOH terminus is modified to be a CONH2 group
KXPALKVLKGXG

SEQ ID NO: 13 - protein, 11 aa, Artificial Sequence
  Synthetic Construct
  VARIANT 1, 10: Xaa = Homocysteine
  VARIANT 11: COOH terminus is modified to be a CONH2 group
XPALKVLKGXG

SEQ ID NO: 14 - protein, 13 aa, Artificial Sequence
  Synthetic Construct
  VARIANT 2, 12: Xaa = Homocysteine
  VARIANT 13: COOH terminus is modified to be a CONH2 group
KXPGALKVLKGXG

SEQ ID NO: 15 - protein, 16 aa, Artificial Sequence
  Synthetic Construct
KKCGGPQGIAGQGCKK

SEQ ID NO: 16 - protein, 16 aa, Artificial Sequence
  Synthetic Construct
KKCGGPQGIWGQGCKK

SEQ ID NO: 17 - protein, 13 aa, Artificial Sequence
  Synthetic Construct
KCGPLGLWARGCK

SEQ ID NO: 18 - protein, 12 aa, Artificial Sequence
  Synthetic Construct
KCGPQGIAGQCK

SEQ ID NO: 19 - protein, 12 aa, Artificial Sequence
  Synthetic Construct
  VARIANT 2, 11: Xaa = Homocysteine
KXGPQGIAGQXK

SEQ ID NO: 20 - protein, 445 aa, Homo sapiens
MAKIKHHHHHHENLYFQGYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 21 - protein, 479 aa, Homo sapiens
MAKIKHHHHHHENLYFQGQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 22 - protein, 153 aa, Homo sapiens
MAHHHHHHENLYFQGGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKR